(19) United States
(12) Patent Application Publication
(10) Pub. No.: US 2025/0049904 A1
(43) Pub. Date: Feb. 13, 2025

(54) IMMUNOSTIMULATORY COMPOSITIONS

(71) Applicants: VICTORIA LINK LIMITED, Kelburn Wellington (NZ); UNIVERSITY OF MELBOURNE, Melbourne (AU); MALCORP BIODISCOVERIES LIMITED, Wellington (NZ)

(72) Inventors: Gavin Frank Painter, Wellington (NZ); Ian Francis Hermans, Wellington (NZ); Mitch Ganley, Wellington (NZ); William Heath, Parkville, Victoria (AU); Lauren Holz, Parkville (AU); Regan James Anderson, Wellington (NZ); Benjamin Jason Compton, Wellington (NZ); Andrew Marshall, Wellington (NZ)

(21) Appl. No.: 18/723,035

(22) PCT Filed: Dec. 22, 2022
(86) PCT No.: PCT/NZ2022/050178

§ 371 Date: Jun. 21, 2024

(30) Foreign Application Priority Data
Dec. 24, 2021 (AU) 2021904282

(51) Int. Cl. A61K 39/00; A61K 9/127; A61K 9/51; A61K 31/7032; A61K 31/7052; A61K 31/7105; A61K 39/015; A61K 39/39; A61P 35/00; B82Y 5/00

(52) U.S. Cl. CPC A61K 39/0011; A61K 9/1272; A61K 9/5123; A61K 31/7032; A61K 31/7052; A61K 31/7105; A61K 39/015; A61K 39/39; A61P 35/00; A61K 2039/53; A61K 2039/55555; B82Y 5/00

(57) ABSTRACT

The present application is directed to immunomodulatory compositions comprising invariant NKT cell (iNKT cell) agonist compounds that induce expansion of tissue resident memory T-cells (Trm Cells) in combination with an immune stimulator agent that enhances an immune response to a target antigen or a polynucleotide encoding the immune stimulator. The iNKT agonists are α-GalCer analogue compounds modified at the 6 position of the galactose ring. The immune stimulator is an antigen or an mRNA encoding an antigen to create a vaccine formulation. These combinations are used in the treatment of infection or cancer in a subject, or to enrich the number of Trm cells in the liver of a subject.

### RELATED APPLICATIONS

This application claims priority to Australian Provisional Application No. 2021904282 entitled “Immunostimulatory Compositions” filed 24 Dec. 2021, the contents of which are incorporated by reference in their entirety.

### FIELD OF THE INVENTION

This invention relates generally to compositions and methods for treating infections and/or cancer. More specifically, the invention relates to the use of compositions comprising iNKT cell agonists to treat or prevent infections and/or cancer.

### BACKGROUND OF THE INVENTION

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavour to which this specification relates.

The immune system is the defence system of an organism against disease, and has traditionally been divided into two arms called innate and adaptive immunity. Typically, innate immunity refers to rapid defence mechanisms that is set in motion within minutes to hours following an insult. Conversely, the adaptive immune response emerges after several days and relies on the innate immune response for its initiation and subsequent outcome. However, the recent discovery of immune cells displaying merged properties indicates that this distinction is not mutually exclusive. These populations that span the innate-adaptive border of immunity comprise, among others, CD1d-restricted natural killer T cells and MR1-restricted mucosal-associated invariant T cells. At its most basic, however, the immune system distinguishes between the organism's own healthy tissue and foreign agents through its ability to detect a wide variety of infectious agents, such as viruses and bacteria, and other parasitic agents, such as unicellular eukaryotic parasites. Another important role of the immune system is to identify and eliminate tumours. Tumour growth and survival in an organism can be in a large part attributed to a number of mechanisms acquired by tumour cells to evade the immune system.

In the treatment or prevention of certain disease states, it may be beneficial to modulate the activity of the immune system, (i.e., to induce, enhance, or suppress the immune response). For example, in the treatment of certain cancers, it may be desirable to provide treatments that activate and/or allow immune cells to recognize, attack, and destroy tumour cells that have developed immune evasion mechanisms. Activation of the immune system may also be part of a vaccination strategy against an infectious agent or a tumour. In other instances, it may be beneficial to downregulate or suppress the immune response to allow for greater immune tolerance. This is the case in the prevention and treatment of autoimmune diseases, which result from a hyperactive immune system that attacks normal tissues as if they were foreign organisms. Immune suppression is also a desirable treatment method in the prevention of organ transplant rejection.

Natural killer T cells (NKT) are a unique lymphocyte population that expresses a T cell receptor (TCR) as well as NK lineage markers and possesses functional properties of both T and NK cells. Type I NKT cells, often called invariant NKT (iNKT) cells, express an invariant TCRα chain composed of a Vα14-Jα18 chain rearrangement in mice (Vα24-Jα18 in humans), that pairs preferentially with Vβ8.2, 7, and 2 (Vβ11 in humans). NKT cells are defined functionally by their ability to recognize glycolipid antigens presented in the context of the MHC class Ib molecule CD1d. NKT cells bridge the gap between the innate and adaptive immune systems and are equipped to rapidly respond to stimuli to elicit an effective immune response.

### SUMMARY OF THE INVENTION

The present invention is predicated, at least in part, on the discovery that delivery of an iNKT cell agonist together with an antigen resulted in a bias towards tissue-resident memory T cells (T_{RM} cells) in the liver.

In one aspect, the present invention provides an immunomodulatory composition comprising a first agent that comprises an invariant NKT cell (iNKT cell) agonist that activates or induces the expansion of tissue-resident memory T cells (T_{RM} cells) together with a second agent comprising an immune stimulator that stimulates or otherwise enhances an immune response to a target antigen in a subject or a polynucleotide sequence encoding an immune stimulator that stimulates or otherwise enhances an immune response to the target antigen in a subject.

In some embodiments, the first agent modifies the phenotype of an iNKT cell in the subject. By way of an illustrative example, the modified phenotype may comprise a downregulation of T cell receptor expression and or increased expression of PD-1 on the surface of the iNKT cell.

In some embodiments, the iNKT cell is in the liver of the subject.

In some of the same embodiments and some other embodiments, the iNKT cell agonist induces TRM cell enrichment or accumulation in the liver of the subject. Upon administration of the iNKT cell agonist to a subject, the ratio of TRM cells to central memory T (T_{CM}) cells and/or effector memory (T_{EM}) cells present in the liver is increased. Suitably, at least 25%, 30%, 35%, 40%, 45%, 50%, 55%, 60%, 65%, or 70%, 75%, or greater than 75% of the total liver T cell population are TRM cells.

In some embodiments, the iNKT cell agonist is a derivative of α-GalCer, wherein the derivation is at the 6 position of the galactose ring.

More particularly, in the iNKT cell agonist comprises a Compound of Formula (I):

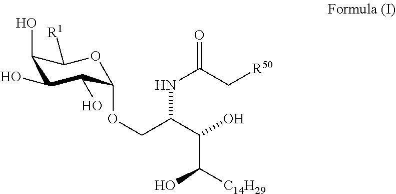

or a pharmaceutically acceptable salt thereof, wherein:
R¹ is selected from hydrogen, halo, alkyl, alkenyl, alkynyl, aryl, acyl, carbocyclyl, heterocyclyl, heteroaryl, alkyloxy, alkenyloxy, alkynyloxy, aryloxy, acyloxy, carbocyclyloxy, heterocyclyloxy, heteroaryloxy, alkylthio, alkenylthio, alkynylthio, arylthio, acylthio, carbocyclylthio, heterocyclylthio, heteroarylthio, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkyloxyacylalkyl, alkylcarbocyclyloxy, alkylheterocyclyloxy, alkylheteroaryloxy, alkylthioalkyl, alkenylthioalkyl, alkynylthioalkyl, alkenylthioalkenyl, alkenylthioalkynyl, alkynylthioalkynyl, arylthioalkyl, alkylacylthio, alkylcarbocyclylthio, alkylheterocyclylalkyl, alkenylheterocyclylalkyl, alkynylheterocyclylalkyl, alkenylheterocyclylalkenyl, alkenylheterocyclylalkynyl, alkynylheterocyclylalkynyl, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, arylacyloxy, arylcarbocyclyloxy, arylheterocyclyloxy, arylheteroaryloxy, alkylthioaryl, alkenylthioaryl, alkynylthioaryl, arylthioaryl, arylacylthio, arylcarbocyclylthio, arylheterocyclylthio, arylheteroarylthio, alkylamino, alkenylamino, alkynylamino, arylamino, acylamino, carbocyclylamino, heterocyclylamino, heteroarylamino, alkylaminoalkyl, alkenylaminoalkyl, alkynylaminoalkyl, alkenylaminoalkenyl, alkenylaminoalkynyl, alkynylaminoalkynyl, arylaminoalkyl, alkylacylamino, alkylcarbocyclylamino, alkylheterocyclylamino, alkylheteroarylamino, alkylaminoaryl, alkenylaminoaryl, alkynylaminoaryl, arylaminoaryl, arylacylamino, arylcarbocyclylamino, arylheterocyclylamino, arylheteroarylamino, alkylamido, alkenylamido, alkynylamido, arylamido, acylamido, carbocyclylamido, heterocyclylamido, heteroarylamido, alkylamidoalkyl, alkenylamidoalkyl, alkynylamidoalkyl, alkenylamidoalkenyl, alkenylamidoalkynyl, alkynylamidoalkynyl, arylamidoalkyl, alkylacylamido, alkylcarbocyclylamido, alkylheterocyclylamido, alkylheteroarylamido, alkylamidoaryl, alkenylamidoaryl, alkynylamidoaryl, arylamidoaryl, arylacylamido, arylcarbocyclylamido, arylheterocyclylamido, arylheteroarylamido, alkylcarbamyl, alkenylcarbamyl, alkynylcarbamyl, arylcarbamyl, acylcarbamyl, carbocyclylcarbamyl, heterocyclylcarbamyl, heteroarylcarbamyl, alkylcarbamylalkyl, alkenylcarbamylalkyl, alkynylcarbamylalkyl, alkenylcarbamylalkenyl, alkenylcarbamylalkynyl, alkynylcarbamylalkynyl, arylcarbamylalkyl, alkylacylcarbamyl, alkylcarbocyclylcarbamyl, alkylheterocyclylcarbamyl, alkylheteroarylcarbamyl, alkylcarbamylaryl, alkenylcarbamylaryl, alkynylcarbamylaryl, arylcarbamylaryl, arylacylcarbamyl, arylcarbocyclylcarbamyl, arylheterocyclylcarbamyl, arylheteroarylcarbamyl, alkyldisulfidyl, alkenyldisulfidyl, alkynyldisulfidyl, aryldisulfidyl, acyldisulfidyl, carbocyclyldisulfidyl, heterocyclyldisulfidyl, heteroaryldisulfidyl, alkyldisulfidylalkyl, alkenyldisulfidylalkyl, alkynyldisulfidylalkyl, alkenyldisulfidylalkenyl, alkenyldisulfidylalkynyl, alkynyldisulfidylalkynyl, aryldisulfidylalkyl, alkylacyldisulfidyl, alkylcarbocyclyldisulfidyl, alkylheterocyclyldisulfidyl, alkylheteroaryldisulfidyl, alkyldisulfidylaryl, alkenyldisulfidylaryl, alkynyldisulfidylaryl, aryldisulfidylaryl, arylacyldisulfidyl, arylcarbocyclyldisulfidyl, arylheterocyclyldisulfidyl, and arylheteroaryldisulfidyl, and wherein R1 is optionally substituted;
and wherein R¹ is not —CH₂OH;
R⁵⁰ is selected from optionally substituted alkyl, alkenyl, alkynyl.

In some more specific embodiments, R¹ is selected from C₁-C₁₈ alkyl, C₂-C₁₈ alkenyl, C₂-C₁₈ alkynyl, C₆-C₁₈ aryl, C₁-C₁₈ acyl, C₃-C₁₈ carbocyclyl, C₂-C₁₈ heterocyclyl, C₃-C₁₈ heteroaryl, C₁-C₁₈ alkyloxy, C₂-C₁₈ alkenyloxy, C₂-C₁₈ alkynyloxy, C₆-C₁₈ aryloxy, C₁-C₁₈ acyloxy, C₃-C₁₈ carbocyclyloxy, C₂-C₁₈ heterocyclyloxy, C₃-C₁₈ heteroaryloxy, C₁-C₁₈ alkylthio, C₂-C₁₈ alkenylthio, C₂-C₁₈ alkynylthio, C₆-C₁₈ arylthio, C₁-C₁₈ acylthio, C₃-C₁₈ carbocyclylthio, C₂-C₁₈ heterocyclylthio, C₃-C₁₈ heteroarylthio, C₃-C₁₈ alkylalkenyl, C₃-C₁₈ alkylalkynyl C₇-C₂₄ alkylaryl, C₂-C₁₈ alkylacyl, C₄-C₁₈ alkylcarbocyclyl, C₃-C₁₈ alkylheterocyclyl, C₄-C₁₈ alkylheteroaryl, C₂-C₁₈ alkyloxyalkyl, C₃-C₁₈ alkenyloxyalkyl, C₃-C₁₈ alkynyloxyalkyl, C₇-C₂₄ aryloxyalkyl, C₂-C₁₈ alkylacyloxy, C₂₋₁₈ alkyloxyacyl C₂₋₁₈ alkyl, C₄-C₁₈ alkylcarbocyclyloxy, C₃-C₁₈ alkylheterocyclyloxy, C₄-C₁₈ alkylheteroaryloxy, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₄-C₁₈ alkylheterocyclylalkyl, C₄-C₁₈ alkenylheterocyclylalkyl, C₄-C₁₈ alkynylheterocyclylalkyl, C₅-C₁₈ alkenylheterocyclylalkenyl, C₅-C₁₈ alkenylheterocyclylalkynyl, C₅-C₁₈ alkynylheterocyclylalkynyl, C₃-C₁₈ alkylheterocyclylthio, C₄-C₁₈ alkylheteroarylthio, C₄-C₁₈ alkylalkenylalkyl, C₄-C₁₈ alkylalkynylalkyl, C₈-C₂₄ alkylarylalkyl, C₃-C₁₈ alkylacylalkyl, C₁₃-C₂₄ arylalkylaryl, C₁₄-C₂₄ arylalkenylaryl, C₁₄-C₂₄ arylalkynylaryl, C₁₃-C₂₄ arylacylaryl, C₇-C₁₈ arylacyl, C₉-C₁₈ arylcarbocyclyl, C₈-C₁₈ arylheterocyclyl, C₉-C₁₈ arylheteroaryl, C₈-C₁₈ alkenyloxyaryl, C₈-C₁₈ alkynyloxyaryl, C₁₂-C₂₄ aryloxyaryl, C₇-C₁₈ arylacyloxy, C₉-C₁₈ arylcarbocyclyloxy, C₈-C₁₈ arylheterocyclyloxy, C₉-C₁₈ arylheteroaryloxy, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylthio, C₂-C₁₈ alkenylthio, C₂-C₁₈ alkynylthio, C₈-C₁₈ arylthio, C₁-C₁₈ acylthio, C₃-C₁₈ carbocyclylthio, C₂-C₁₈ heterocyclylthio, C₃-C₁₈ heteroarylthio, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₉ alkylacylthio, C₄-C₁₈ alkylcarbocyclylthio, C₃-C₁₈ alkylheterocyclylthio, C₄-C₁₈ alkylheteroarylthio, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylamino, C₂-C₁₈ alkenylamino, C₂-C₁₈ alkynylamino, C₆-C₁₈ arylamino, C₁-C₁₈ acylamino, C₃-C₁₈ carbocyclylamino, C₂-C₁₈ heterocyclylamino, C₃-C₁₈ heteroarylamino, C₂-C₁₈ alkylaminoalkyl, C₃-C₁₈ alkenylaminoalkyl, C₃-C₁₈ alkynylaminoalkyl, C₄-C₁₈ alkenylaminoalkenyl, C₄-C₁₈ alkenylaminoalkynyl, C₄-C₁₈ alkynylaminoalkynyl, C₇-C₂₄ arylaminoalkyl, C₂-C₁₈ alkylacylamino, C₄-C₁₈ alkylcarbocyclylamino, C₃-C₁₈ alkylheterocyclylamino, C₄-C₁₈ alkylheteroarylamino, C₈-C₁₈ alkylaminoaryl, C₈-C₁₈ alkenylaminoaryl, C₈-C₁₉ alkynylaminoaryl, C₁₂-C₂₄ arylaminoaryl, C₇-C₁₈ arylacylamino, C₉-C₁₈ arylcarbocyclylamino, C₈-C₁₈ arylheterocyclylamino, C₉-C₁₈ arylheteroarylamino, C₁-C₁₈ alkylamido, C₂-C₁₈ alkenylamido, C₂-C₁₈ alkynylamido, C₆-C₁₈ arylamido, C₁-C₁₈ acylamido, C₃-C₁₈ carbocyclylamido, C₂-C₁₈ heterocyclylamido, C₃-C₁₈ heteroarylamido, C₂-C₁₈ alkylamidoalkyl, C₃-C₁₈ alkenylamidoalkyl, C₃-C₁₃ alkynylamidoalkyl, C₄-C₁₈ alkenylamidoalkenyl, C₄-C₁₈ alkenylamidoalkynyl, C₄-C₁₈ alkynylamidoalkynyl, C₇-C₂₄ arylamidoalkyl, C₂-C₁₈ alkylacylamido, C₄-C₁₈ alkylcarbocyclylamido, C₃-C₁₈ alkylheterocyclylamido, C₄-C₁₈ alkylheteroarylamido, C₈-C₁₈ alkylamidoaryl, C₈-C₁₈ alkenylamidoaryl, C₈-C₁₈ alkynylamidoaryl, C₁₂-C₂₄ arylamidoaryl, C₇-C₁₈ arylacylamido, C₈-C₁₈ arylcarbocyclylamido, C₈-C₁₈ arylheterocyclylamido, C₉-C₁₈ arylheteroarylamido, C₁-C₁₈ alkylcarbamyl, C₂-C₁₈ alkenylcarbamyl, C₂-C₁₈ alkynylcarbamyl, C₆-C₁₈ arylcarbamyl, C₁-C₁₈ acylcarbamyl, C₃-C₁₈ carbocyclylcarbamyl, C₂-C₁₈ heterocyclylcarbamyl, C₃-C₁₈ heteroarylcarbamyl, C₂-C₁₈ alkylcarbamylalkyl, C₃-C₁₈ alkenylcarbamylalkyl, C₃-C₁₈ alkynylcarbamylalkyl, C₄-C₁₈ alkenylcarbamylalkenyl, C₄-C₁₈ alkenylcarbamylalkynyl, C₄-C₁₈ alkynylcarbamylalkynyl, C₇-C₂₄ arylcarbamylalkyl, C₂-C₁₈ alkylacylcarbamyl, C₄-C₁₈ alkylcarbocyclylcarbamyl, C₃-C₁₈ alkylheterocyclylcarbamyl, C₄-C₁₈ alkylheteroarylcarbamyl, C₈-C₁₈ alkylcarbamylaryl, C₈-C₁₈ alkenylcarbamylaryl, C₈-C₁₈ alkynylcarbamylaryl, C₁₂-C₂₄ arylcarbamylaryl, C₇-C₁₈ arylacylcarbamyl, C₉-C₁₈ arylcarbocyclylcarbamyl, C₈-C₁₈ arylheterocyclylcarbamyl, C₉-C₁₈ arylheteroarylcarbamyl, C₁-C₁₈ alkyldisulfidyl, C₂-C₁₈ alkenyldisulfidyl, C₂-C₁₈ alkynyldisulfidyl, C₆-C₁₈ aryldisulfidyl, C₁-C₁₈ acyldisulfidyl, C₃-C₁₈ carbocyclyldisulfidyl, C₂-C₁₈ heterocyclyldisulfidyl, C₃-C₁₈ heteroaryldisulfidyl, C₂-C₁₈ alkyldisulfidylalkyl, C₃-C₁₈ alkenyldisulfidylalkyl, C₃-C₁₈ alkynyldisulfidylalkyl, C₄-C₁₈ alkenyldisulfidylalkenyl, C₄-C₁₈ alkenyldisulfidylalkynyl, C₄-C₁₈ alkynyldisulfidylalkynyl, C₇-C₂₄ aryldisulfidylalkyl, C₂-C₁₈ alkylacyldisulfidyl, C₄-C₁₈ alkylcarbocyclyldisulfidyl, C₃-C₁₈ alkylheterocyclyldisulfidyl, C₄-C₁₈ alkylheteroaryldisulfidyl, C₈-C₁₈ alkyldisulfidylaryl, C₈-C₁₈ alkenyldisulfidylaryl, C₈-C₁₈ alkynyldisulfidylaryl, C₁₂-C₂₄ aryldisulfidylaryl, C₇-C₁₈ arylacyldisulfidyl, C₉-C₁₈ arylcarbocyclyldisulfidyl, C₈-C₁₈ arylheterocyclyldisulfidyl, and C₉-C₁₈ arylheteroaryldisulfidyl, and wherein R¹ is optionally substituted;
and wherein R¹ is not —CH₂OH.

In some even more specific embodiments, R¹ is selected from alkyl (e.g., C₁-C₁₈, C₁-C₆, C₁-C₅, C₈-C₁₈, or C₉-C₁₈), aryl (e.g., C₆-C₁₈), heteroaryl (e.g., C₃-C₁₈), carbocyclyl (e.g., C₃-C₁₈), heterocyclyl (e.g., C₂-C₁₈), alkylaryl (e.g., C₇-C₂₄), alkylheteroaryl (e.g., C₄-C₁₈), alkylcarbocyclyl (e.g., C₄-C₁₈), alkylthioalkyl (e.g., wherein each alkyl is C₁-C₁₈), arylthioalkyl (e.g., wherein each alkyl is C₇-C₁₈), alkylaminoalkyl (e.g., wherein each alkyl is C₁-C₁₈), alkylamidoalkyl (e.g., wherein each alkyl is C₁-C₁₈), alkylheterocyclylalkyl (e.g., C₃-C₁₈), alkylthio (e.g., C₁-C₁₈; such as thiomethyl) and alkylheterocyclyl (e.g., C₃-C₁₈);
and wherein R¹ is not —CH₂OH.

In some specific embodiments, R⁵⁰ is selected from optionally substituted C₁₈₋₂₆ alkyl, C₁₈₋₂₆ alkenyl, C₁₃₋₂₆ alkynyl. In some even more specific embodiments, R⁵⁰ is —R⁵¹-amido-R⁵² (such as —R⁵¹—NHC(O)—R⁵², or —R⁵¹—C(O)NH—R⁵²) wherein R⁵¹ and R⁵² are each independently selected from alkyl, alkenyl, alkynyl. For example, R⁵¹ and R⁵² may be each independently selected from C₆-C₁₆ alkyl, C₅-C₁₆ alkenyl, C₆-C₁₆ alkynyl.

In some other embodiments, the iNKT agonist comprises a Compound of Formula (II):

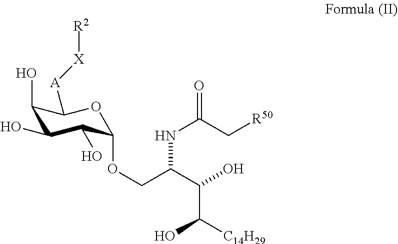

or a pharmaceutically acceptable salt thereof, wherein:
A is selected from alkyl, alkenyl, and alkynyl;
X selected from —CR¹⁰R¹¹—, —O—, —S—, —NR^{a}—, —NR^{a}C(O)—, —NR^{a}C(O)O—, —S—S—, heterocyclyl, heteroaryl, wherein R¹⁰ and R¹¹ where present are independently selected from H, halo, C₁₋₂alkyl, and wherein R^{a} may be selected from hydrogen, alkyl, alkenyl, alkynyl, aryl, carbocyclyl, heteroaryl, heterocyclyl, arylalkyl, and acyl;
R² is selected from hydrogen, halo, alkyl, alkenyl, alkynyl, aryl, acyl, carbocyclyl, heterocyclyl, heteroaryl, alkyloxy, alkenyloxy, alkynyloxy, aryloxy, acyloxy, carbocyclyloxy, heterocyclyloxy, heteroaryloxy, alkylthio, alkenylthio, alkynylthio, arylthio, acylthio, carbocyclylthio, heterocyclylthio, heteroarylthio, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkyloxyacylalkyl, alkylcarbocyclyloxy, alkylheterocyclyloxy, alkylheteroaryloxy, alkylthioalkyl, alkenylthioalkyl, alkynylthioalkyl, alkenylthioalkenyl, alkenylthioalkynyl, alkynylthioalkynyl, arylthioalkyl, alkylacylthio, alkylcarbocyclylthio, alkylheterocyclylalkyl, alkenylheterocyclylalkyl, alkynylheterocyclylalkyl, alkenylheterocyclylalkenyl, alkenylheterocyclylalkynyl, alkynylheterocyclylalkynyl, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, arylacyloxy, arylcarbocyclyloxy, arylheterocyclyloxy, arylheteroaryloxy, alkylthioaryl, alkenylthioaryl, alkynylthioaryl, arylthioaryl, arylacylthio, arylcarbocyclylthio, arylheterocyclylthio, arylheteroarylthio, alkylamino, alkenylamino, alkynylamino, arylamino, acylamino, carbocyclylamino, heterocyclylamino, heteroarylamino, alkylaminoalkyl, alkenylaminoalkyl, alkynylaminoalkyl, alkenylaminoalkenyl, alkenylaminoalkynyl, alkynylaminoalkynyl, arylaminoalkyl, alkylacylamino, alkylcarbocyclylamino, alkylheterocyclylamino, alkylheteroarylamino, alkylaminoaryl, alkenylaminoaryl, alkynylaminoaryl, arylaminoaryl, arylacylamino, arylcarbocyclylamino, arylheterocyclylamino, arylheteroarylamino, alkylamido, alkenylamido, alkynylamido, arylamido, acylamido, carbocyclylamido, heterocyclylamido, heteroarylamido, alkylamidoalkyl, alkenylamidoalkyl, alkynylamidoalkyl, alkenylamidoalkenyl, alkenylamidoalkynyl, alkynylamidoalkynyl, arylamidoalkyl, alkylacylamido, alkylcarbocyclylamido, alkylheterocyclylamido, alkylheteroarylamido, alkylamidoaryl, alkenylamidoaryl, alkynylamidoaryl, arylamidoaryl, arylacylamido, arylcarbocyclylamido, arylheterocyclylamido, arylheteroarylamido, alkylcarbamyl, alkenylcarbamyl, alkynylcarbamyl, arylcarbamyl, acylcarbamyl, carbocyclylcarbamyl, heterocyclylcarbamyl, heteroarylcarbamyl, alkylcarbamylalkyl, alkenylcarbamylalkyl, alkynylcarbamylalkyl, alkenylcarbamylalkenyl, alkenylcarbamylalkynyl, alkynylcarbamylalkynyl, arylcarbamylalkyl, alkylacylcarbamyl, alkylcarbocyclylcarbamyl, alkylheterocyclylcarbamyl, alkylheteroarylcarbamyl, alkylcarbamylaryl, alkenylcarbamylaryl, alkynylcarbamylaryl, arylcarbamylaryl, arylacylcarbamyl, arylcarbocyclylcarbamyl, arylheterocyclylcarbamyl, arylheteroarylcarbamyl, alkyldisulfidyl, alkenyldisulfidyl, alkynyldisulfidyl, aryldisulfidyl, acyldisulfidyl, carbocyclyldisulfidyl, heterocyclyldisulfidyl, heteroaryldisulfidyl, alkyldisulfidylalkyl, alkenyldisulfidylalkyl, alkynyldisulfidylalkyl, alkenyldisulfidylalkenyl, alkenyldisulfidylalkynyl, alkynyldisulfidylalkynyl, aryldisulfidylalkyl, alkylacyldisulfidyl, alkylcarbocyclyldisulfidyl, alkylheterocyclyldisulfidyl, alkylheteroaryldisulfidyl, alkyldisulfidylaryl, alkenyldisulfidylaryl, alkynyldisulfidylaryl, aryldisulfidylaryl, arylacyldisulfidyl, arylcarbocyclyldisulfidyl, arylheterocyclyldisulfidyl, and arylheteroaryldisulfidyl, and wherein R² is optionally substituted;
and wherein -A-X—R² is not —CH₂OH;
R⁵⁰ is selected from optionally substituted alkyl, alkenyl, alkynyl.

In some embodiments of this type, R^{a} may be independently selected from hydrogen, C₁-C₈ alkyl, C₂-C₈ alkenyl, C₂-C₈ alkynyl, C₆-C₁₈ aryl, C₃-C₁₈ carbocyclyl, C₃-C₁₈ heteroaryl, C₂-C₁₈ heterocyclyl, C₇-C₂₄arylalkyl, and C₁-C₈ acyl.

In some embodiments of this type, X is S or —NR^{a}—, wherein R^{a} may be selected from hydrogen, alkyl, alkenyl, alkynyl, aryl, carbocyclyl, heteroaryl, heterocyclyl, arylalkyl, and acyl. More preferably R^{a} may be independently selected from hydrogen, C₁-C₈ alkyl, C₂-C₈ alkenyl, C₂-C₈ alkynyl, C₆-C₁₈ aryl, C₃-C₁₈ carbocyclyl, C₃-C₁₈ heteroaryl, C₂-C₁₈ heterocyclyl, C₇-C₂₄ arylalkyl, and C₁-C₈ acyl.

In some embodiments of this type A is —CR¹²R¹³—, wherein R¹² and R¹³ where present are independently selected from H, halo, C₁₋₂alkyl. More preferably A is —CH₂—.

Preferably, A is —CH₂—.

In some more specific embodiments, R² is selected from C₁-C₁₈ alkyl, C₂-C₁₈ alkenyl, C₂-C₁₈ alkynyl, C₆-C₁₈ aryl, C₁-C₁₈ acyl, C₃-C₁₈ carbocyclyl, C₂-C₁₈ heterocyclyl, C₃-C₁₈ heteroaryl, C₁-C₁₈ alkyloxy, C₂-C₁₈ alkenyloxy, C₂-C₁₈ alkynyloxy, C₆-C₁₈ aryloxy, C₁-C₁₈ acyloxy, C₃-C₁₈ carbocyclyloxy, C₂-C₁₈ heterocyclyloxy, C₃-C₁₈ heteroaryloxy, C₁-C₁₈ alkylthio, C₂-C₁₈ alkenylthio, C₂-C₁₈ alkynylthio, C₆-C₁₈ arylthio, C₁-C₁₈ acylthio, C₃-C₁₈ carbocyclylthio, C₂-C₁₈ heterocyclylthio, C₃-C₁₈ heteroarylthio, C₃-C₁₈ alkylalkenyl, C₃-C₁₈ alkylalkynyl C₇-C₂₄ alkylaryl, C₂-C₁₈ alkylacyl, C₄-C₁₈ alkylcarbocyclyl, C₃-C₁₈ alkylheterocyclyl, C₄-C₁₈ alkylheteroaryl, C₂-C₁₈ alkyloxyalkyl, C₃-C₁₈ alkenyloxyalkyl, C₃-C₁₈ alkynyloxyalkyl, C₇-C₂₄ aryloxyalkyl, C₂-C₁₈ alkylacyloxy, C₂-C₁₈ alkyloxyacyl C₂-C₁₈ alkyl, C₄-C₁₈ alkylcarbocyclyloxy, C₃-C₁₈ alkylheterocyclyloxy, C₄-C₁₈ alkylheteroaryloxy, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₄-C₁₈ alkylheterocyclylalkyl, C₄-C₁₈ alkenylheterocyclylalkyl, C₄-C₁₈ alkynylheterocyclylalkyl, C₅-C₁₈ alkenylheterocyclylalkenyl, C₅-C₁₈ alkenylheterocyclylalkynyl, C₅-C₁₈ alkynylheterocyclylalkynyl, C₃-C₁₈ alkylheterocyclylthio, C₄-C₁₈ alkylheteroarylthio, C₄-C₁₈ alkylalkenylalkyl, C₄-C₁₈ alkylalkynylalkyl, C₈-C₂₄ alkylarylalkyl, C₃-C₁₈ alkylacylalkyl, C₁₃-C₂₄ arylalkylaryl, C₁₄-C₂₄ arylalkenylaryl, C₁₄-C₂₄ arylalkynylaryl, C₁₃-C₂₄ arylacylaryl, C₇-C₁₈ arylacyl, C₉-C₁₈ arylcarbocyclyl, C₈-C₁₈ arylheterocyclyl, C₉-C₁₈ arylheteroaryl, C₈-C₁₈ alkenyloxyaryl, C₈-C₁₈ alkynyloxyaryl, C₁₂-C₂₄ aryloxyaryl, C₇-C₁₈ arylacyloxy, C₈—C₁₈ arylcarbocyclyloxy, C₈-C₁₈ arylheterocyclyloxy, C₉-C₁₈ arylheteroaryloxy, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylthio, C₂-C₁₈ alkenylthio, C₂-C₁₈ alkynylthio, C₆-C₁₈ arylthio, C₁-C₁₈ acylthio, C₃-C₁₈ carbocyclylthio, C₂-C₁₈ heterocyclylthio, C₃-C₁₈ heteroarylthio, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₃-C₁₈ alkylheterocyclylthio, C₄-C₁₈ alkylheteroarylthio, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylamino, C₂-C₁₈ alkenylamino, C₂-C₁₈ alkynylamino, C₆-C₁₈ arylamino, C₁-C₁₈ acylamino, C₃-C₁₈ carbocyclylamino, C₂-C₁₈ heterocyclylamino, C₃-C₁₈ heteroarylamino, C₂-C₁₈ alkylaminoalkyl, C₃-C₁₈ alkenylaminoalkyl, C₃-C₁₈ alkynylaminoalkyl, C₄-C₁₈ alkenylaminoalkenyl, C₄-C₁₈ alkenylaminoalkynyl, C₄-C₁₈ alkynylaminoalkynyl, C₇-C₂₄ arylaminoalkyl, C₂-C₁₈ alkylacylamino, C₄-C₁₈ alkylcarbocyclylamino, C₃-C₁₈ alkylheterocyclylamino, C₄-C₁₈ alkylheteroarylamino, C₈-C₁₈ alkylaminoaryl, C₈-C₁₈ alkenylaminoaryl, C₈-C₁₈ alkynylaminoaryl, C₁₂-C₂₄ arylaminoaryl, C₇-C₁₈ arylacylamino, C₉-C₁₈ arylcarbocyclylamino, C₈-C₁₈ arylheterocyclylamino, C₉-C₁₈ arylheteroarylamino, C₁-C₁₈ alkylamido, C₂-C₁₈ alkenylamido, C₂-C₁₈ alkynylamido, C₆-C₁₈ arylamido, C₁-C₁₈ acylamido, C₃-C₁₈ carbocyclylamido, C₂-C₁₈ heterocyclylamido, C₃-C₁₈ heteroarylamido, C₂-C₁₈ alkylamidoalkyl, C₃-C₁₈ alkenylamidoalkyl, C₃-C₁₈ alkynylamidoalkyl, C₄-C₁₈ alkenylamidoalkenyl, C₄-C₁₈ alkenylamidoalkynyl, C₄-C₁₈ alkynylamidoalkynyl, C₇-C₂₄ arylamidoalkyl, C₂-C₁₈ alkylacylamido, C₄-C₁₈ alkylcarbocyclylamido, C₃-C₁₈ alkylheterocyclylamido, C₄-C₁₈ alkylheteroarylamido, C₈-C₁₈ alkylamidoaryl, C₈-C₁₈ alkenylamidoaryl, C₈-C₁₈ alkynylamidoaryl, C₁₂-C₂₄ arylamidoaryl, C₇-C₁₈ arylacylamido, C₉-C₁₈ arylcarbocyclylamido, C₈-C₁₈ arylheterocyclylamido, C₉-C₁₈ arylheteroarylamido, C₁-C₁₈ alkylcarbamyl, C₂-C₁₈ alkenylcarbamyl, C₂-C₁₈ alkynylcarbamyl, C₆-C₁₈ arylcarbamyl, C₁-C₁₈ is acylcarbamyl, C₃-C₁₈ carbocyclylcarbamyl, C₂-C₁₈ heterocyclylcarbamyl, C₃-C₁₈ heteroarylcarbamyl, C₂-C₁₈ alkylcarbamylalkyl, C₃-C₁₈ alkenylcarbamylalkyl, C₃-C₁₈ alkynylcarbamylalkyl, C₄-C₁₈ alkenylcarbamylalkenyl, C₄-C₁₈ alkenylcarbamylalkynyl, C₄-C₁₈ alkynylcarbamylalkynyl, C₇-C₂₄ arylcarbamylalkyl, C₂-C₁₈ alkylacylcarbamyl, C₄-C₁₈ alkylcarbocyclylcarbamyl, C₃-C₁₈ alkylheterocyclylcarbamyl, C₄-C₁₈ alkylheteroarylcarbamyl, C₈-C₁₈ alkylcarbamylaryl, C₈-C₁₈ alkenylcarbamylaryl, C₈-C₁₈ alkynylcarbamylaryl, C₁₂-C₂₄ arylcarbamylaryl, C₇-C₁₈ arylacylcarbamyl, C₉-C₁₈ arylcarbocyclylcarbamyl, C₈-C₁₈ arylheterocyclylcarbamyl, C₉-C₁₈ arylheteroarylcarbamyl, C₁-C₁₈ alkyldisulfidyl, C₂-C₁₈ alkenyldisulfidyl, C₂-C₁₈ alkynyldisulfidyl, C₆-C₁₈ aryldisulfidyl, C₁-C₁₈ acyldisulfidyl, C₃-C₁₈ carbocyclyldisulfidyl, C₂-C₁₈ heterocyclyldisulfidyl, C₃-C₁₈ heteroaryldisulfidyl, C₂-C₁₈ alkyldisulfidylalkyl, C₃-C₁₈ alkenyldisulfidylalkyl, C₃-C₁₈ alkynyldisulfidylalkyl, C₄-C₁₈ alkenyldisulfidylalkenyl, C₄-C₁₈ alkenyldisulfidylalkynyl, C₄-C₁₈ alkynyldisulfidylalkynyl, C₇-C₂₄ aryldisulfidylalkyl, C₂-C₁₈ alkylacyldisulfidyl, C₄-C₁₈ alkylcarbocyclyldisulfidyl, C₃-C₁₈ alkylheterocyclyldisulfidyl, C₄-C₁₈ alkylheteroaryldisulfidyl, C₈-C₁₈ alkyldisulfidylaryl, C₈-C₁₈ alkenyldisulfidylaryl, C₈-C₁₈ alkynyldisulfidylaryl, C₁₂-C₂₄ aryldisulfidylaryl, C₇-C₁₈ arylacyldisulfidyl, C₉-C₁₈ arylcarbocyclyldisulfidyl, C₈-C₁₈ arylheterocyclyldisulfidyl, and C₉-C₁₈ arylheteroaryldisulfidyl, and wherein R² is optionally substituted;
and wherein -A-X—R² is not —CH₂OH.

In some preferred embodiments, R² is selected from alkyl (e.g., C₁-C₁₈, C₁-C₆, C₁-C₅, C₈-C₁₈, or C₉-C₁₈), aryl (e.g., C₆-C₁₈), heteroaryl (e.g., C₃-C₁₈), carbocyclyl (e.g., C₃-C₁₈), heterocyclyl (e.g., C₂-C₁₈), alkylaryl (e.g., C₇-C₂₄), alkylheteroaryl (e.g., C₄-C₁₈), alkylcarbocyclyl (e.g., C₄-C₁₈), alkylthioalkyl (e.g., wherein each alkyl is C₁-C₁₈), arylthioalkyl (e.g., wherein each alkyl is C₇-C₁₈), alkylaminoalkyl (e.g., wherein each alkyl is C₁-C₁₈), alkylamidoalkyl (e.g., wherein each alkyl is C₁-C₁₈), alkylheterocyclylalkyl (e.g., C₃-C₁₈), alkylthio (e.g., C₁-C₁₈; such as thiomethyl) and alkylheterocyclyl (e.g., C₃-C₁₈).

In some specific embodiments, R⁵⁰ is selected from optionally substituted C₁₈₋₂₆ alkyl, C₁₈₋₂₆ alkenyl, C₁₈₋₂₆ alkynyl. In some even more specific embodiments, R⁵⁰ is —R⁵¹-amido-R⁵² (such as —R⁵¹—NHC(O)—R⁵², or —R⁵¹—C(O)NH—R⁵²) wherein R⁵¹ and R⁵² are each independently selected from alkyl, alkenyl, alkynyl. For example, R⁵¹ and R⁵² may be each independently selected from C₆-C₁₆ alkyl, C₅-C₁₆ alkenyl, C₆-C₁₆ alkynyl.

In some other embodiments, the iNKT cell agonist is a Compound of Formula (III):

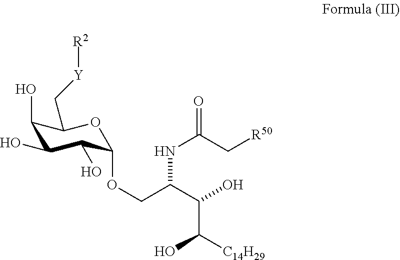

or a pharmaceutically acceptable salt thereof, wherein:
Y is selected from —S—, —NR^{a}—, —NR^{a}C(O)—, —NR^{a}C(O)O—, —S—S—, heterocyclyl, heteroaryl, wherein R^{a} may be selected from hydrogen, alkyl, alkenyl, alkynyl, aryl, carbocyclyl, heteroaryl, heterocyclyl, arylalkyl, and acyl;
R² is selected from hydrogen, halo, alkyl, alkenyl, alkynyl, aryl, acyl, carbocyclyl, heterocyclyl, heteroaryl, alkyloxy, alkenyloxy, alkynyloxy, aryloxy, acyloxy, carbocyclyloxy, heterocyclyloxy, heteroaryloxy, alkylthio, alkenylthio, alkynylthio, arylthio, acylthio, carbocyclylthio, heterocyclylthio, heteroarylthio, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkyloxyacylalkyl, alkylcarbocyclyloxy, alkylheterocyclyloxy, alkylheteroaryloxy, alkylthioalkyl, alkenylthioalkyl, alkynylthioalkyl, alkenylthioalkenyl, alkenylthioalkynyl, alkynylthioalkynyl, arylthioalkyl, alkylacylthio, alkylcarbocyclylthio, alkylheterocyclylalkyl, alkenylheterocyclylalkyl, alkynylheterocyclylalkyl, alkenylheterocyclylalkenyl, alkenylheterocyclylalkynyl, alkynylheterocyclylalkynyl, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, arylacyloxy, arylcarbocyclyloxy, arylheterocyclyloxy, arylheteroaryloxy, alkylthioaryl, alkenylthioaryl, alkynylthioaryl, arylthioaryl, arylacylthio, arylcarbocyclylthio, arylheterocyclylthio, arylheteroarylthio, alkylamino, alkenylamino, alkynylamino, arylamino, acylamino, carbocyclylamino, heterocyclylamino, heteroarylamino, alkylaminoalkyl, alkenylaminoalkyl, alkynylaminoalkyl, alkenylaminoalkenyl, alkenylaminoalkynyl, alkynylaminoalkynyl, arylaminoalkyl, alkylacylamino, alkylcarbocyclylamino, alkylheterocyclylamino, alkylheteroarylamino, alkylaminoaryl, alkenylaminoaryl, alkynylaminoaryl, arylaminoaryl, arylacylamino, arylcarbocyclylamino, arylheterocyclylamino, arylheteroarylamino, alkylamido, alkenylamido, alkynylamido, arylamido, acylamido, carbocyclylamido, heterocyclylamido, heteroarylamido, alkylamidoalkyl, alkenylamidoalkyl, alkynylamidoalkyl, alkenylamidoalkenyl, alkenylamidoalkynyl, alkynylamidoalkynyl, arylamidoalkyl, alkylacylamido, alkylcarbocyclylamido, alkylheterocyclylamido, alkylheteroarylamido, alkylamidoaryl, alkenylamidoaryl, alkynylamidoaryl, arylamidoaryl, arylacylamido, arylcarbocyclylamido, arylheterocyclylamido, arylheteroarylamido, alkylcarbamyl, alkenylcarbamyl, alkynylcarbamyl, arylcarbamyl, acylcarbamyl, carbocyclylcarbamyl, heterocyclylcarbamyl, heteroarylcarbamyl, alkylcarbamylalkyl, alkenylcarbamylalkyl, alkynylcarbamylalkyl, alkenylcarbamylalkenyl, alkenylcarbamylalkynyl, alkynylcarbamylalkynyl, arylcarbamylalkyl, alkylacylcarbamyl, alkylcarbocyclylcarbamyl, alkylheterocyclylcarbamyl, alkylheteroarylcarbamyl, alkylcarbamylaryl, alkenylcarbamylaryl, alkynylcarbamylaryl, arylcarbamylaryl, arylacylcarbamyl, arylcarbocyclylcarbamyl, arylheterocyclylcarbamyl, arylheteroarylcarbamyl, alkyldisulfidyl, alkenyldisulfidyl, alkynyldisulfidyl, aryldisulfidyl, acyldisulfidyl, carbocyclyldisulfidyl, heterocyclyldisulfidyl, heteroaryldisulfidyl, alkyldisulfidylalkyl, alkenyldisulfidylalkyl, alkynyldisulfidylalkyl, alkenyldisulfidylalkenyl, alkenyldisulfidylalkynyl, alkynyldisulfidylalkynyl, aryldisulfidylalkyl, alkylacyldisulfidyl, alkylcarbocyclyldisulfidyl, alkylheterocyclyldisulfidyl, alkylheteroaryldisulfidyl, alkyldisulfidylaryl, alkenyldisulfidylaryl, alkynyldisulfidylaryl, aryldisulfidylaryl, arylacyldisulfidyl, arylcarbocyclyldisulfidyl, arylheterocyclyldisulfidyl, and arylheteroaryldisulfidyl, and wherein R² is optionally substituted;
R⁵⁰ is selected from optionally substituted alkyl, alkenyl, alkynyl.

In some embodiments of this type, R^{a} is hydrogen.

In some embodiments, Y may be selected from —S—, —NH—, —NHC(O)—, —NHC(O)O— and —S—S—, triazolyl.

In some more specific embodiments, R² in Formula (III) is selected from C₁-C₁₈ alkyl, C₂-C₁₈ alkenyl, C₂-C₁₈ alkynyl, C₆-C₁₈ aryl, C₁-C₁₈ acyl, C₃-C₁₈ carbocyclyl, C₂-C₁₈ heterocyclyl, C₃-C₁₈ heteroaryl, C₁-C₁₈ alkyloxy, C₂-C₁₈ alkenyloxy, C₂-C₁₈ alkynyloxy, C₆-C₁₈ aryloxy, C₁-C₁₈ acyloxy, C₃-C₁₈ carbocyclyloxy, C₂-C₁₈ heterocyclyloxy, C₃-C₁₈ heteroaryloxy, C₁-C₁₈ alkylthio, C₂-C₁₈ alkenylthio, C₂-C₁₈ alkynylthio, C₆-C₁₈ arylthio, C₁-C₁₈ acylthio, C₃-C₁₈ carbocyclylthio, C₂-C₁₈ heterocyclylthio, C₃-C₁₈ heteroarylthio, C₃-C₁₈ alkylalkenyl, C₃-C₁₈ alkylalkynyl C₇-C₂₄ alkylaryl, C₂-C₁₈ alkylacyl, C₄-C₁₈ alkylcarbocyclyl, C₃-C₁₈ alkylheterocyclyl, C₄-C₁₈ alkylheteroaryl, C₂-C₁₈ alkyloxyalkyl, C₃-C₁₈ alkenyloxyalkyl, C₃-C₁₈ alkynyloxyalkyl, C₇-C₂₄ aryloxyalkyl, C₂-C₁₈ alkylacyloxy, C₂-C₁₈ alkyloxyacyl C₂₋₁₈ alkyl, C₄-C₁₈ alkylcarbocyclyloxy, C₃-C₁₈ alkylheterocyclyloxy, C₄-C₁₈ alkylheteroaryloxy, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈alkylcarbocyclylthio, C₄—C₁₈ alkylheterocyclylalkyl, C₄-C₁₈ alkenylheterocyclylalkyl, C₄-C₁₈ alkynylheterocyclylalkyl, C₅-C₁₈ alkenylheterocyclylalkenyl, C₅-C₁₈ alkenylheterocyclylalkynyl, C₅-C₁₈ alkynylheterocyclylalkynyl, C₃-C₁₈ alkylheterocyclylthio, C₄-C₁₈ alkylheteroarylthio, C₄-C₁₈ alkylalkenylalkyl, C₄-C₁₈ alkylalkynylalkyl, C₈-C₂₄ alkylarylalkyl, C₃-C₁₉ alkylacylalkyl, C₁₃-C₂₄ arylalkylaryl, C₁₄-C₂₄ arylalkenylaryl, C₁₄-C₂₄ arylalkynylaryl, C₁₃-C₂₄ arylacylaryl, C₇-C₁₈ arylacyl, C₉-C₁₈ arylcarbocyclyl, C₈-C₁₈ arylheterocyclyl, C₉-C₁₈ arylheteroaryl, C₈-C₁₈ alkenyloxyaryl, C₈-C₁₈ alkynyloxyaryl, C₁₂-C₂₄ aryloxyaryl, C₇-C₁₈ arylacyloxy, C₉-C₁₈ arylcarbocyclyloxy, C₈-C₁₈ arylheterocyclyloxy, C₉-C₁₈ arylheteroaryloxy, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylthio, C₂-C₁₈ alkenylthio, C₂-C₁₈ alkynylthio, C₈-C₁₈ arylthio, C₁-C₁₈ acylthio, C₃-C₁₈ carbocyclylthio, C₂-C₁₈ heterocyclylthio, C₃-C₁₈ heteroarylthio, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₃-C₁₈ alkylheterocyclylthio, C₄-C₁₈ alkylheteroarylthio, C₈-C₁₈alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylamino, C₂-C₁₈ alkenylamino, C₂-C₁₈ alkynylamino, C₆-C₁₈ arylamino, C₁-C₁₉ acylamino, C₃-C₁₉ carbocyclylamino, C₂-C₁₈ heterocyclylamino, C₃-C₁₈ heteroarylamino, C₂-C₁₈ alkylaminoalkyl, C₃-C₁₈ alkenylaminoalkyl, C₃-C₁₈ alkynylaminoalkyl, C₄-C₁₉ alkenylaminoalkenyl, C₄-C₁₈ alkenylaminoalkynyl, C₄-C₁₈ alkynylaminoalkynyl, C₇-C₂₄ arylaminoalkyl, C₂-C₁₈ alkylacylamino, C₄-C₁₈ alkylcarbocyclylamino, C₃-C₁₈ alkylheterocyclylamino, C₄-C₁₈ alkylheteroarylamino, C₈-C₁₈ alkylaminoaryl, C₈-C₁₈ alkenylaminoaryl, C₈-C₁₈ alkynylaminoaryl, C₁₂-C₂₄ arylaminoaryl, C₇-C₁₈ arylacylamino, C₉-C₁₈ arylcarbocyclylamino, C₈-C₁₈ arylheterocyclylamino, C₉-C₁₈ arylheteroarylamino, C₁-C₁₉ alkylamido, C₂-C₁₉ alkenylamido, C₂-C₁₈alkynylamido, C₆-C₁₈ arylamido, C₁-C₁₈ acylamido, C₃-C₁₈ carbocyclylamido, C₂-C₁₈ heterocyclylamido, C₃-C₁₈ heteroarylamido, C₂-C₁₈ alkylamidoalkyl, C₃-C₁₈ alkenylamidoalkyl, C₃-C₁₈ alkynylamidoalkyl, C₄-C₁₈ alkenylamidoalkenyl, C₄-C₁₈ alkenylamidoalkynyl, C₄-C₁₈ alkynylamidoalkynyl, C₇-C₂₄ arylamidoalkyl, C₂-C₁₈ alkylacylamido, C₄-C₁₈ alkylcarbocyclylamido, C₃-C₁₈ alkylheterocyclylamido, C₄-C₁₈ alkylheteroarylamido, C₈-C₁₈ alkylamidoaryl, C₈-C₁₈ alkenylamidoaryl, C₈-C₁₈ alkynylamidoaryl, C₁₂-C₂₄ arylamidoaryl, C₇-C₁₈ arylacylamido, C₉-C₁₈ arylcarbocyclylamido, C₈-C₁₈ arylheterocyclylamido, C₉-C₁₈ arylheteroarylamido, C₁-C₁₈ alkylcarbamyl, C₂-C₁₈ alkenylcarbamyl, C₂-C₁₈ alkynylcarbamyl, C₅-C₁₈ arylcarbamyl, C₁-C₁₈ acylcarbamyl, C₃-C₁₈ carbocyclylcarbamyl, C₂-C₁₈ heterocyclylcarbamyl, C₃-C₁₈ heteroarylcarbamyl, C₂-C₁₈ alkylcarbamylalkyl, C₃-C₁₈ alkenylcarbamylalkyl, C₃-C₁₈ alkynylcarbamylalkyl, C₄-C₁₈ alkenylcarbamylalkenyl, C₄-C₁₈ alkenylcarbamylalkynyl, C₄-C₁₈ alkynylcarbamylalkynyl, C₇-C₂₄ arylcarbamylalkyl, C₂-C₁₈ alkylacylcarbamyl, C₄-C₁₈ alkylcarbocyclylcarbamyl, C₃-C₁₈ alkylheterocyclylcarbamyl, C₄-C₁₈ alkylheteroarylcarbamyl, C₈-C₁₈ alkylcarbamylaryl, C₈-C₁₈ alkenylcarbamylaryl, C₈-C₁₈ alkynylcarbamylaryl, C₁₂-C₂₄ arylcarbamylaryl, C₇-C₁₈ arylacylcarbamyl, C₉-C₁₈ arylcarbocyclylcarbamyl, C₈-C₁₈ arylheterocyclylcarbamyl, C₉-C₁₈ arylheteroarylcarbamyl, C₁-C₁₈ alkyldisulfidyl, C₂-C₁₈ alkenyldisulfidyl, C₂-C₁₈ alkynyldisulfidyl, C₆-C₁₈ aryldisulfidyl, C₁-C₁₈ acyldisulfidyl, C₃-C₁₈ carbocyclyldisulfidyl, C₂-C₁₈ heterocyclyldisulfidyl, C₃-C₁₈ heteroaryldisulfidyl, C₂-C₁₈ alkyldisulfidylalkyl, C₃-C₁₈ alkenyldisulfidylalkyl, C₃-C₁₈ alkynyldisulfidylalkyl, C₄—C₁₈ alkenyldisulfidylalkenyl, C₄-C₁₈ alkenyldisulfidylalkynyl, C₄-C₁₈ alkynyldisulfidylalkynyl, C₇-C₂₄ aryldisulfidylalkyl, C₂-C₁₈ alkylacyldisulfidyl, C₄-C₁₈ alkylcarbocyclyldisulfidyl, C₃-C₁₈ alkylheterocyclyldisulfidyl, C₄-C₁₈ alkylheteroaryldisulfidyl, C₈-C₁₈ alkyldisulfidylaryl, C₈-C₁₈ alkenyldisulfidylaryl, C₈-C₁₈ alkynyldisulfidylaryl, C₁₂-C₂₄ aryldisulfidylaryl, C₇-C₁₈ arylacyldisulfidyl, C₉-C₁₈ arylcarbocyclyldisulfidyl, C₈-C₁₈ arylheterocyclyldisulfidyl, and C₉-C₁₈ arylheteroaryldisulfidyl, and wherein R² is optionally substituted.

In some more preferred embodiments, R² in Formula (III) is selected from alkyl (e.g., C₁-C₁₈, C₁-C₆, C₁-C₁₈, C₈-C₁₈, or C₉-C₁₈), aryl (e.g., C₆-C₁₈), heteroaryl (e.g., C₃-C₁₈), carbocyclyl (e.g., C₃-C₁₈), heterocyclyl (e.g., C₂-C₁₈), alkylaryl (e.g., C₇-C₂₄), alkylheteroaryl (e.g., C₄-C₁₈), alkylcarbocyclyl (e.g., C₄-C₁₈), alkylthioalkyl (e.g., wherein each alkyl is C₁-C₁₈), arylthioalkyl (e.g., wherein each alkyl is C₇-C₁₈), alkylaminoalkyl (e.g., wherein each alkyl is C₁-C₁₈), alkylamidoalkyl (e.g., wherein each alkyl is C₁-C₁₈), alkylheterocyclylalkyl (e.g., C₃-C₁₈), alkylthio (e.g., C₁-C₁₈; such as thiomethyl) and alkylheterocyclyl (e.g., C₃-C₁₈).

In some specific embodiments, R⁵⁰ is selected from optionally substituted C₁₈₋₂₆ alkyl, C₁₈₋₂₆ alkenyl, C₁₈₋₂₆ alkynyl. In some even more specific embodiments, R⁵⁰ is —R⁵¹-amido-R⁵² (such as —R⁵¹—NHC(O)—R⁵², or —R⁵¹—C(O)NH—R⁵²) wherein R⁵¹ and R⁵² are each independently selected from alkyl, alkenyl, alkynyl. For example, R⁵¹ and R⁵² may be each independently selected from C₆-C₁₆ alkyl, C₅-C₁₈ alkenyl, C₆-C₁₆ alkynyl.

Even more preferably, the iNKT cell agonist is a compound selected from the following GROUP I compounds:

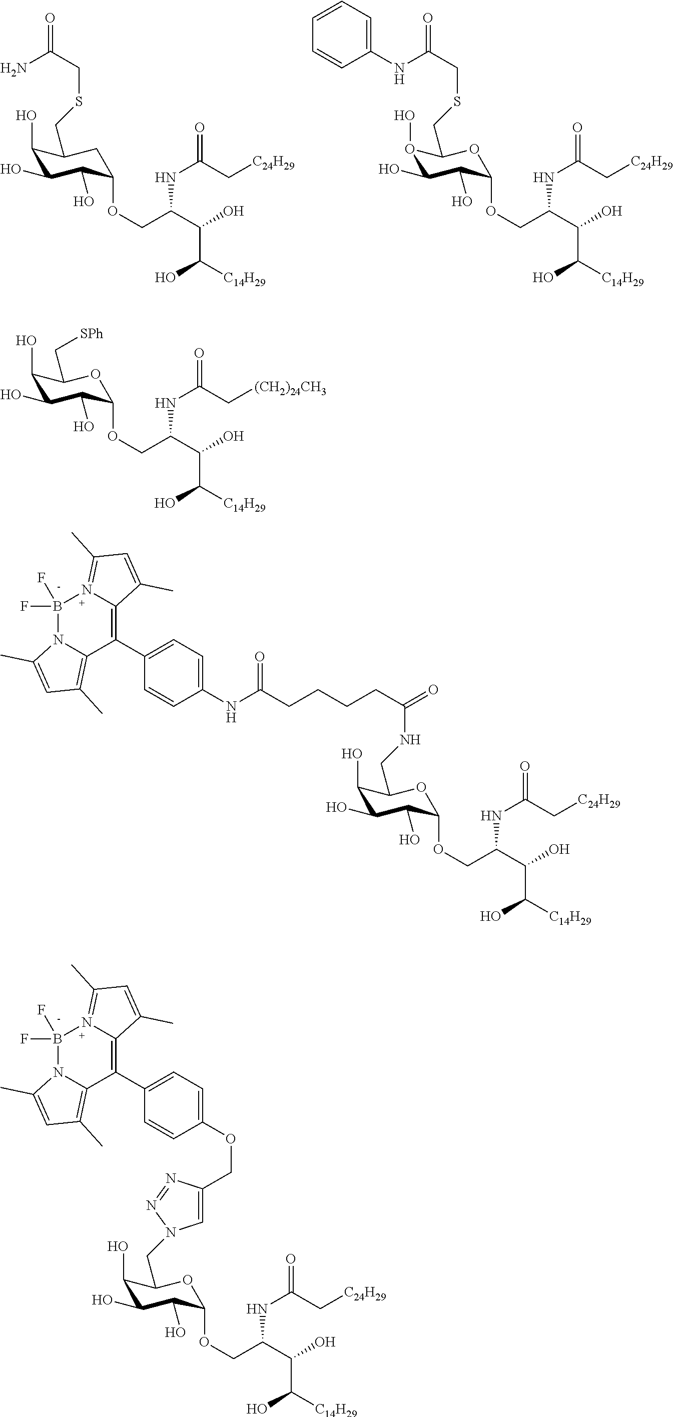

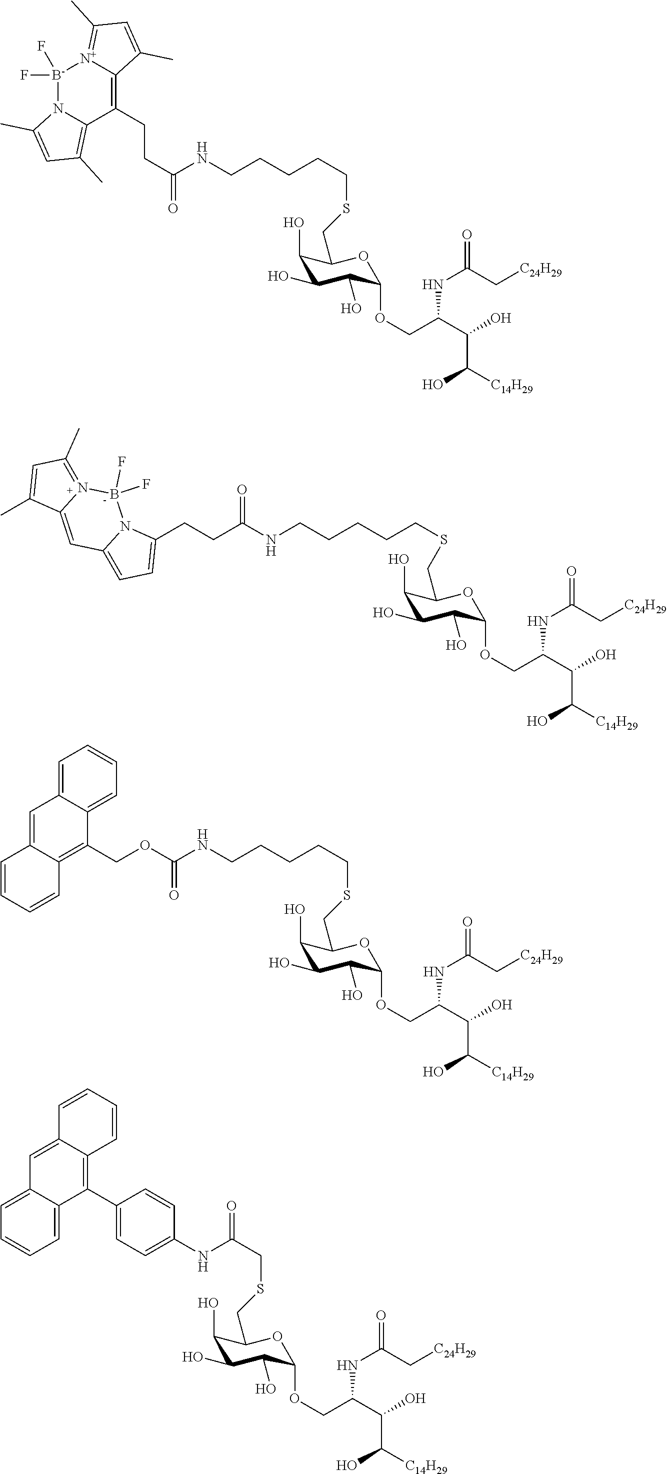

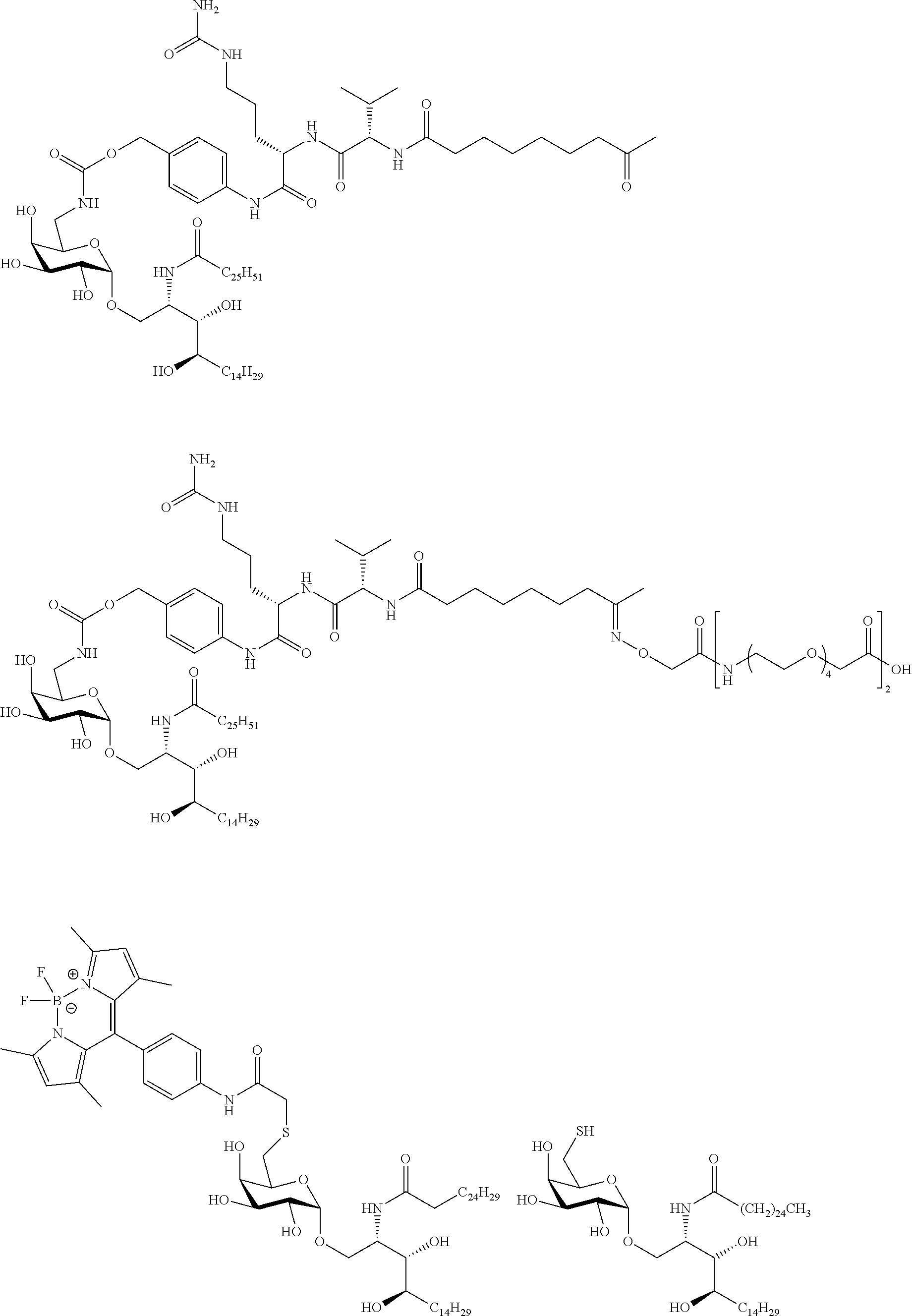

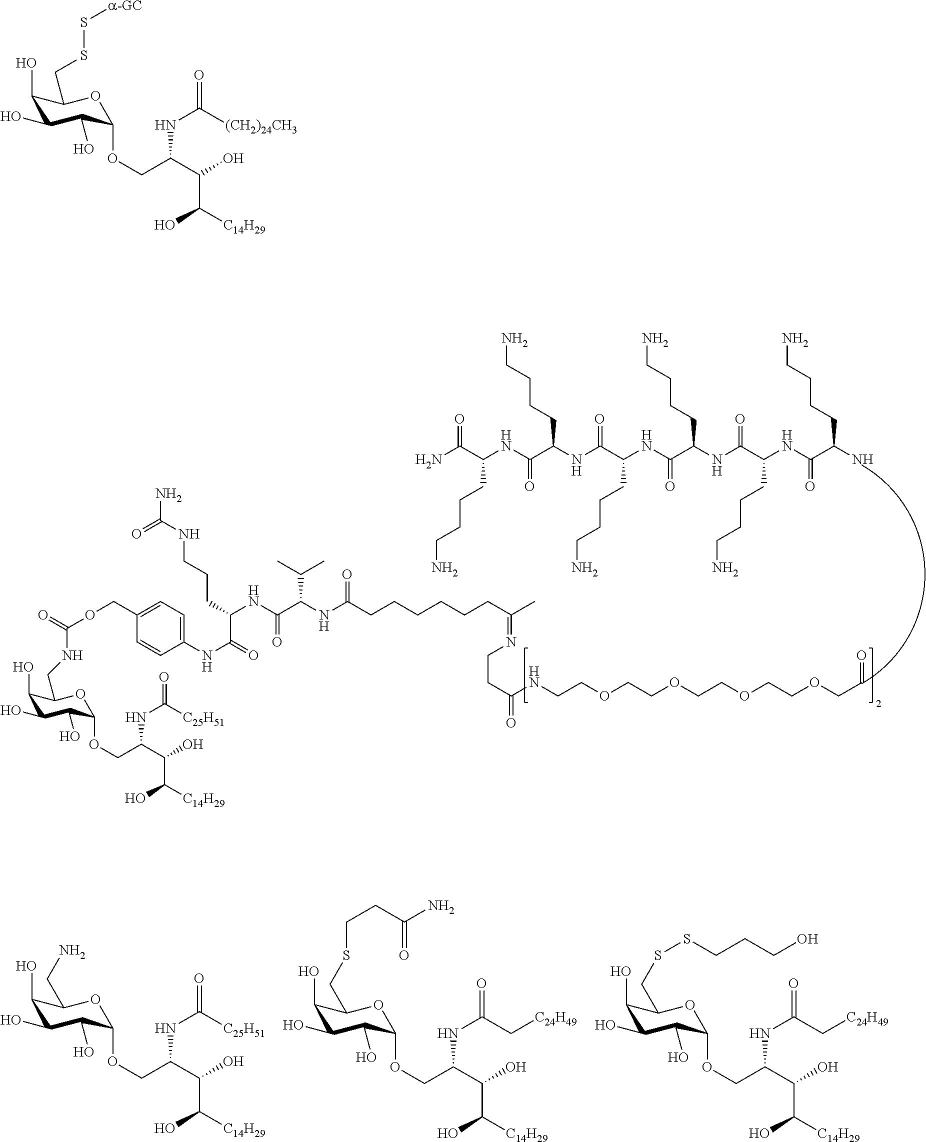

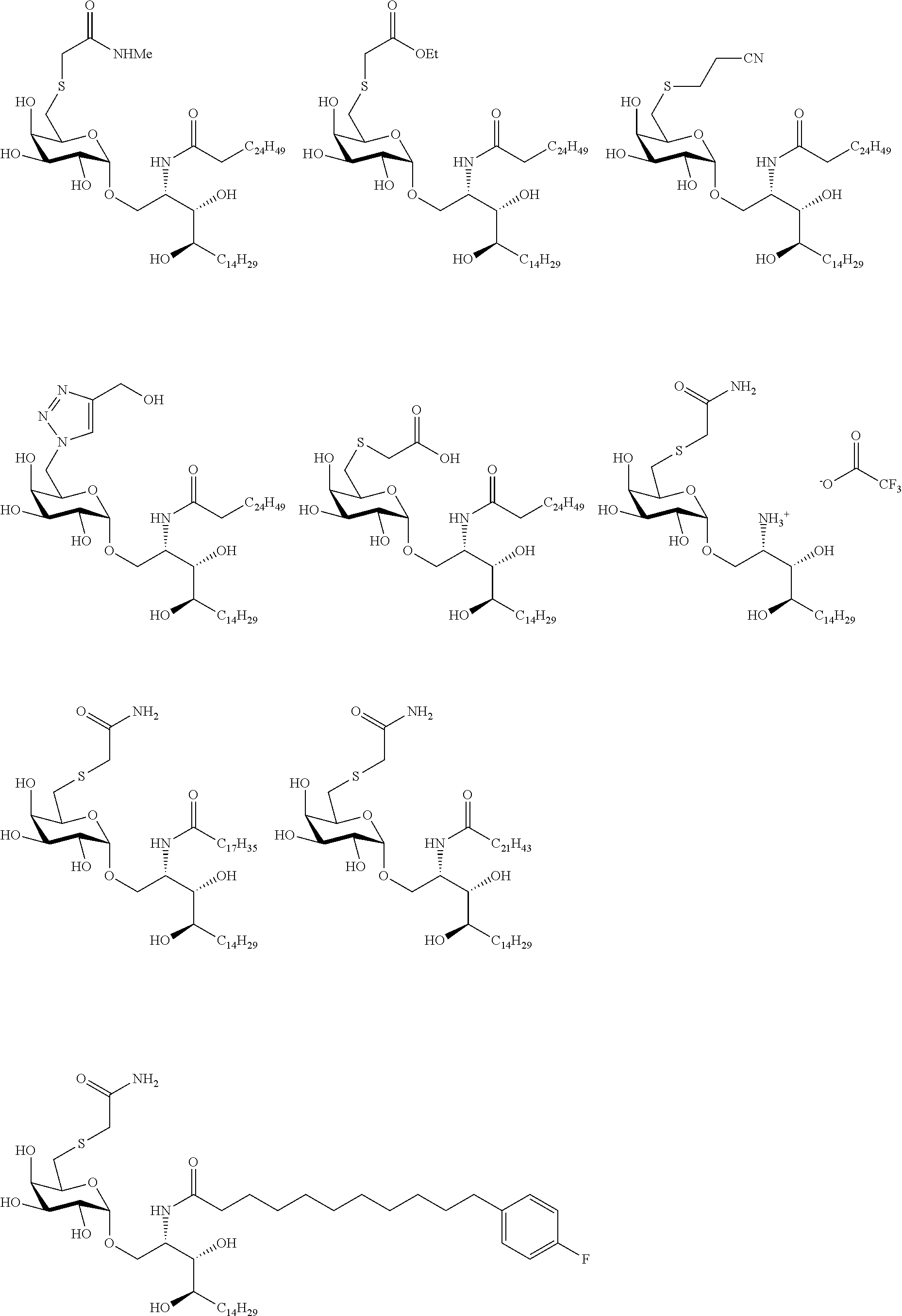

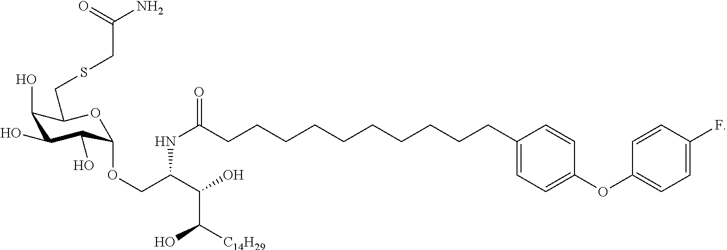

In some embodiments, the immune stimulator is a CD8⁺ T cell epitope.

In some embodiments, the target antigen is selected from a parasite antigen, a virus antigen, a bacterial antigen, and a cancer antigen. By way of an illustrative example, the parasite antigen may be derived from a parasite selected from Plasmodium falciparum, P. vivax, P. malariae, P. ovale, and P. knowlesi. Alternatively, the virus antigen may be a hepatitis antigen (e.g., hepatitis antigen, hepatitis B antigen, and hepatitis C antigen). By way of a further illustrative example, the cancer antigen is a liver cancer antigen.

In some embodiments, the second agent is a polynucleotide sequence, and wherein the polynucleotide is a ribonucleic acid (RNA) molecule. Typically, the polynucleotide is codon-optimised. Several types of codon optimisation are known in the art, including codon optimisation is selected to enhance translation of the encoded polypeptide in the subject, and/or to reduce innate immunity against the polynucleotide molecule.

In some embodiments, the polynucleotide comprises at least one chemical modification. The at least one chemical modification may be selected from the group consisting of pseudouridine, N1-methylpseudouridine, N1-ethylpseudouridine, 2thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2thio-dihydrouridine, 2-thio-pseudouridine, 4-methoxy-2-thio-pseudouridine, 4methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In some alternative embodiments, the polynucleotide is fully modified.

In some embodiments, the composition further comprises a nanoparticle. Typically, the nanoparticle is a lipid nanoparticle (e.g., a liposome, or a lipoplex). In some embodiments of this type, the first agent is contained at least partially within the surface of the lipid nanoparticle and the second agent is encapsulated within the nanoparticle or complexed to the surface of the nanoparticle.

Suitably, the lipid nanoparticle has a diameter of between about 50 nm and 500 nm.

Typically, the nanoparticle comprises a cationic lipid, a PEG-modified lipid, a sterol, and/or a non-cationic lipid. In some embodiments of this type, the cationic lipid is an ionizable cationic lipid.

In some embodiments, the ionizable cationic lipid is selected from the group comprising or consisting of 1,2-di-O-octadecenyl-3-trimethylammonium propane (DOTMA), N-[1-(2,3-dioleyloxy)propyl]-N,N,N-trimethylammonium chloride (DOTMA), 1,2-Dioleoyl-3-Trimethylammonium-Propane (DOTAP), 5-carboxyspermylglycinedioctadecylamide (DOGS), 2,3-dioleyloxy-N-[2(spermine-carboxamido)ethyl]-N,N-dimethyl-1-propanaminium (DOSPA), 1,2-Dioleoyl-3-Dimethylammonium-Propane (DODAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), heptatriaconta-6,9,28,31-tetraen19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dilinolenyloxy-N,N-dimethyl-3-aminopropane (DLenDMA), N-dioleyl-N,N-dimethyl ammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis, cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminoethyl-[1,3]-dioxolane (DLin-K-XTC2-DMA), and C12-200.

In some embodiments, the nanoparticle comprises a dioleoylphosphatidylethanolamine (DOPE) lipid.

In another aspect, the present invention provides a pharmaceutical composition comprising the composition as described above and elsewhere herein, and a pharmaceutically acceptable carrier, diluent, or excipient.

Exemplary compositions of the present invention include vaccines. Suitably, the vaccine is formulated for intramuscular, subcutaneous, or intravenous administration.

In yet another aspect, the present invention provides a composition for inducing an immune response to a target antigen in a subject, the composition comprising at least one isolated RNA molecule encoding an immune stimulator that stimulates or otherwise enhances an immune response to the target antigen in a subject, and a lipid nanoparticle, wherein the lipid nanoparticle comprises an iNKT agonist, wherein the iNKT agonist induces proliferation or accumulation of T_{RM} cells in the subject.

In yet another aspect, the invention provides compound selected from the following GROUP II compounds:

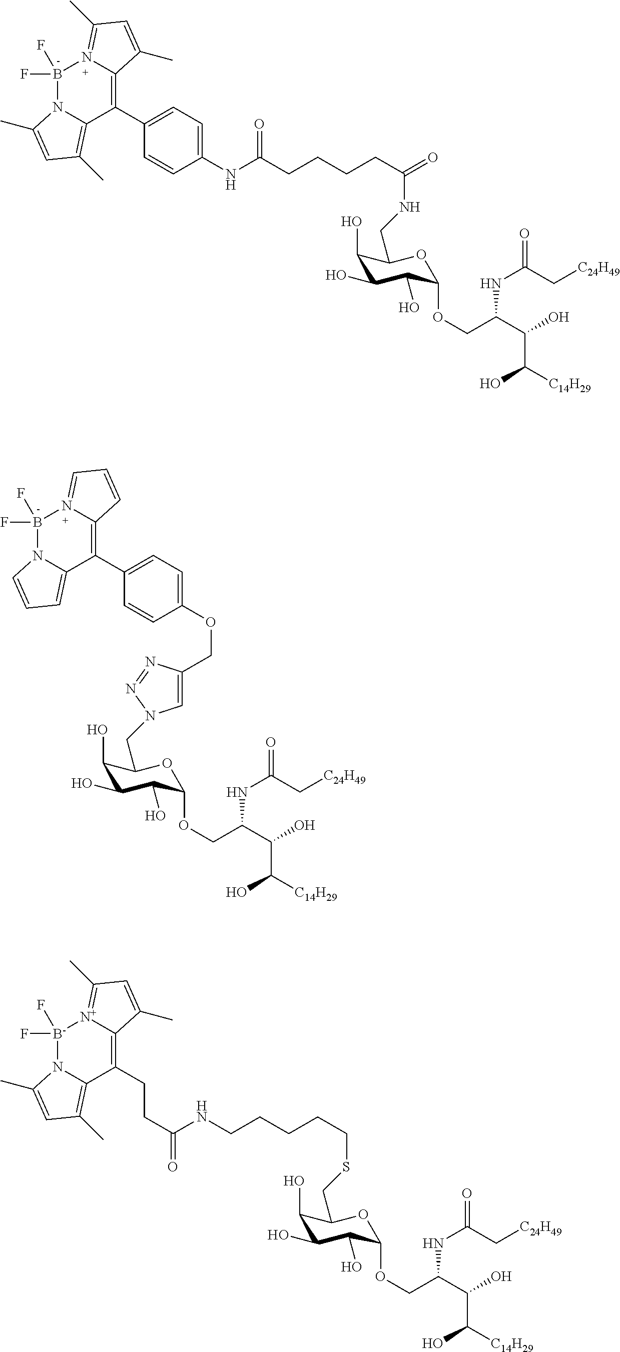

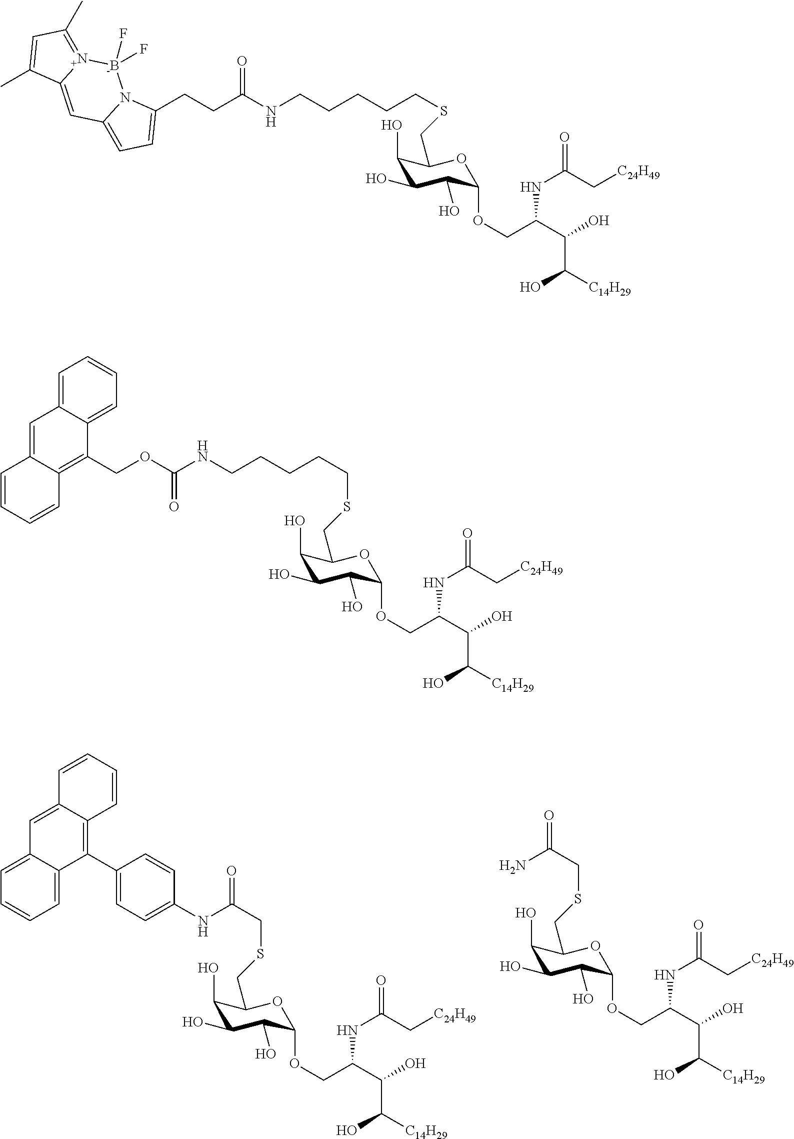

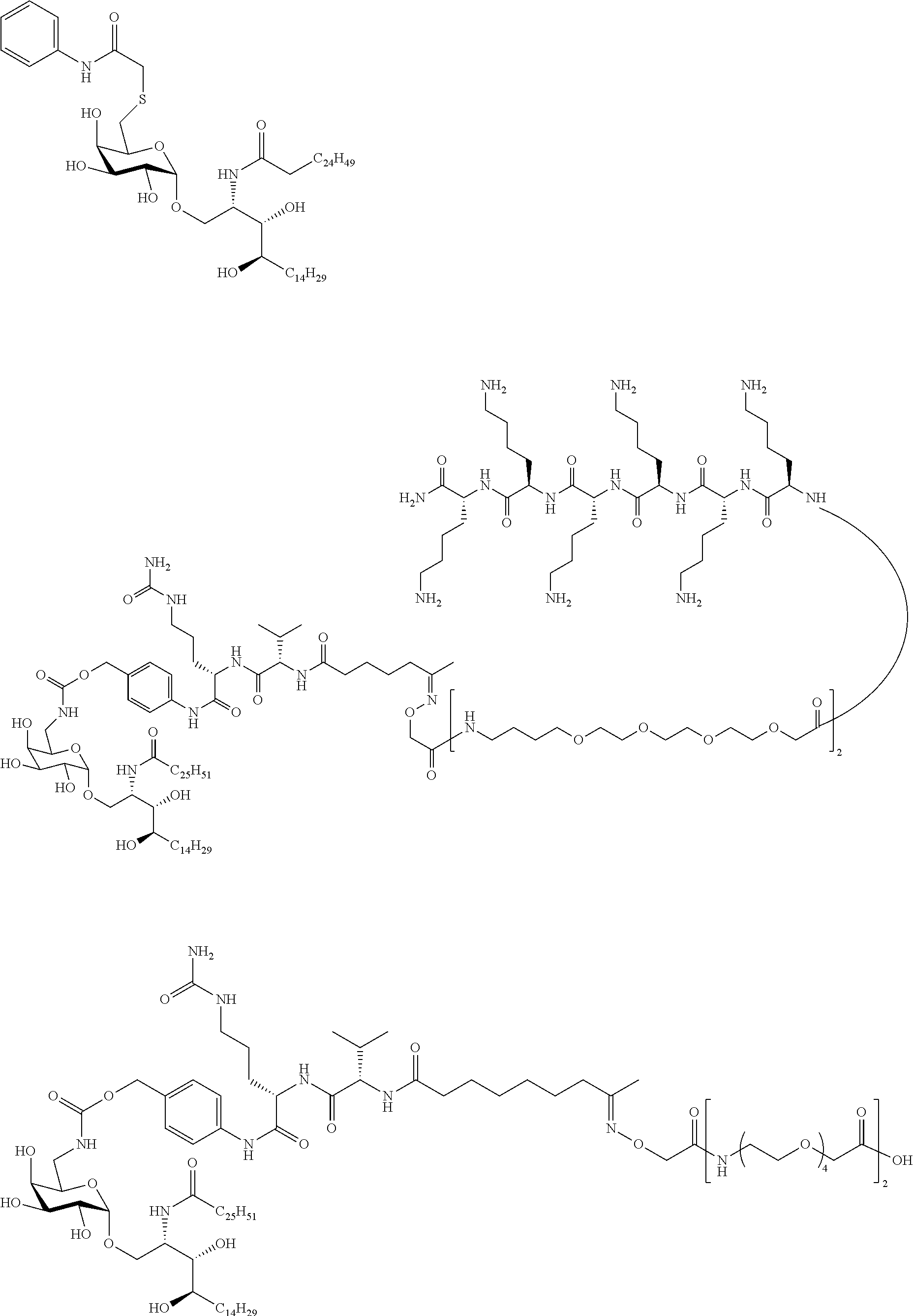

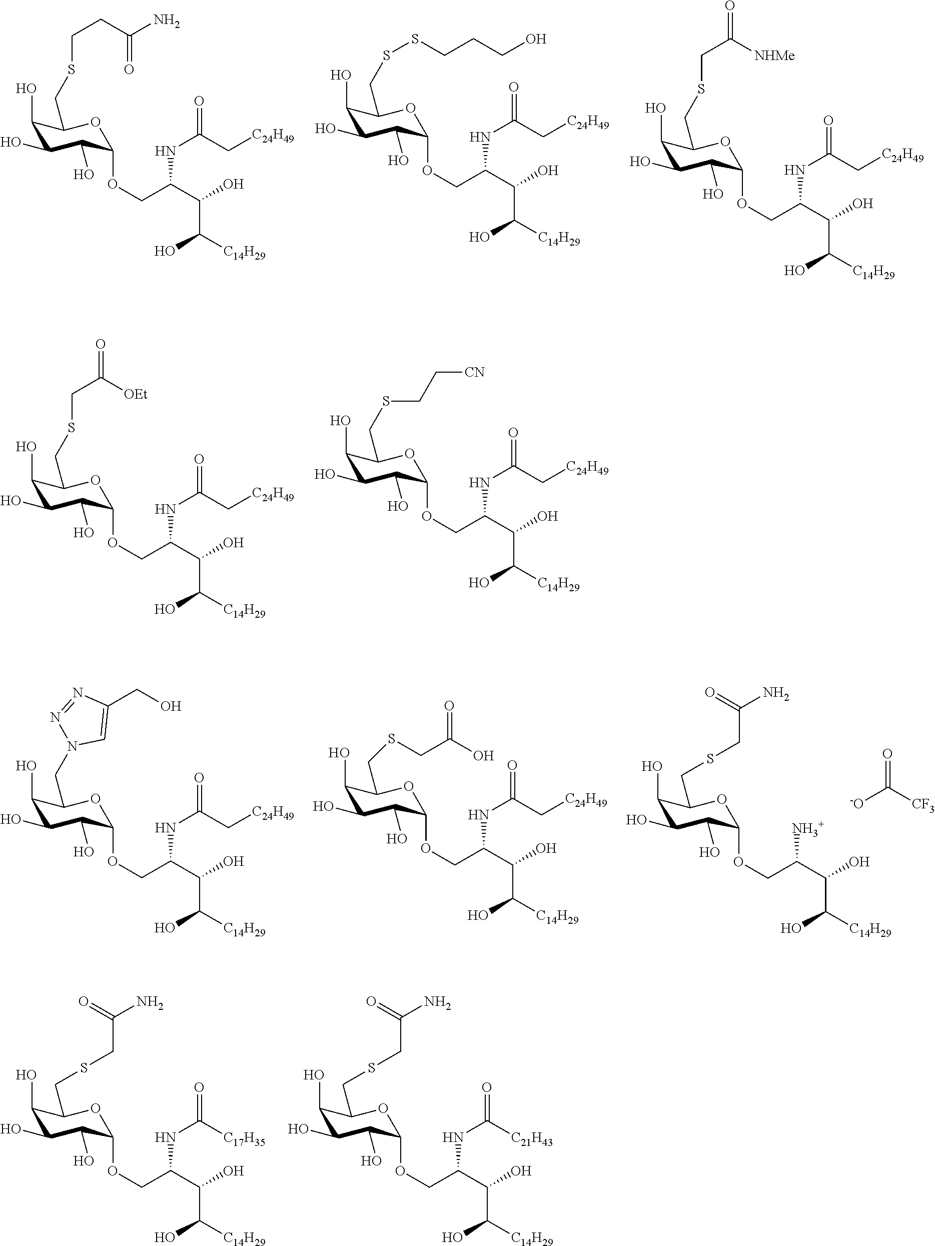

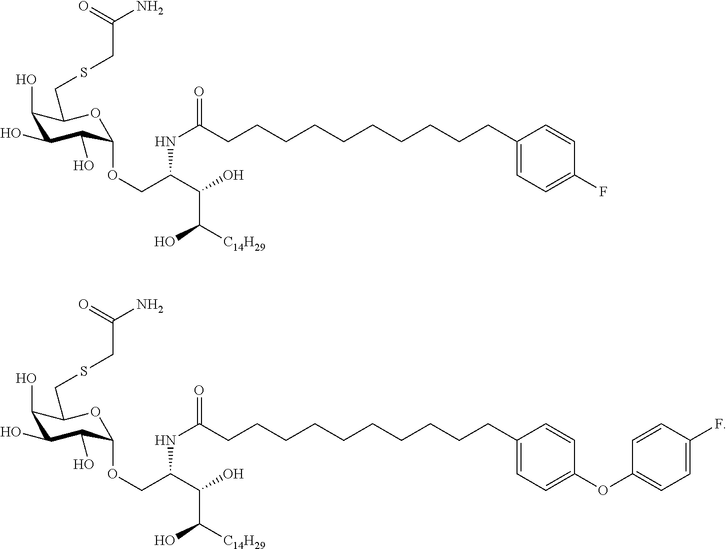

In certain embodiments, the immune response stimulated by the immune stimulator is a T-cell-mediated response. A further aspect of the present invention provides methods for treating or preventing a disease or condition associated with the presence or aberrant expression of a target antigen in a subject.

In still yet another aspect, the present invention provides a method of treating an infection in a subject, the method comprising administering to the subject a composition that comprises a first agent that comprises an iNKT cell agonist that activates or induces the production of tissue-resident memory T cells (T_{RM}) together with a second agent comprising an immune stimulator that stimulates or otherwise enhances an immune response to a target antigen in a subject or a polynucleotide sequence encoding an immune stimulator that stimulates or otherwise enhances an immune response to the target antigen in a subject, to thereby treat the infection in the subject.

In yet another aspect, the invention provides a method of treating a cancer or tumour in a subject, the method comprising administering to the subject a composition that comprises a first agent that comprises an iNKT cell agonist that activates or induces the production of tissue-resident memory T cells (T_{RM}), together with a second agent comprising an immune stimulator that stimulates or otherwise enhances an immune response to a target antigen in a subject or a polynucleotide sequence encoding an immune stimulator that stimulates or otherwise enhances an immune response to the target antigen in a subject, to thereby treat the cancer or tumour in the subject.

In another aspect, the invention provides a method of enriching the number or proportion of T_{RM} cells in the liver of a subject, the method comprising administering to the subject a composition that comprises a first agent that comprises an iNKT cell agonist that activates or induces the production of tissue-resident memory T cells (T_{RM}), together with a second agent comprising an immune stimulator that stimulates or otherwise enhances an immune response to a target antigen in a subject or a polynucleotide sequence encoding an immune stimulator that stimulates or otherwise enhances an immune response to the target antigen in a subject, to thereby enrich the number or proportion of T_{RM} cells in the liver of the subject.

In some embodiments of this type, the iNKT cell agonist does not significantly activate or induce the production of T_{RM} cells in the spleen

### BRIEF DESCRIPTION OF THE FIGURES

FIG. 1 provides a graphical representation showing that LPX-mRNA vaccines containing CI058 induce high levels of liver T_{RM} cells. Liposomes were prepared with DOTMA and DOPE lipids and complexed with mRNA encoding chicken ovalbumin (OVA) to give vaccine LPX-mOVA. Additional vaccines were prepared by including either the prototypical NKT cell agonist α-GalCer (LPX-mOVA-α-GalCer) or the derivative CI058, which has BODIPY attached via an —S in the 6 position of the galactose (LPX-mOVA-CI058). Recipient C57BL/6 mice were transferred 50,000 naïve OVA-specific TCR-transgenic CD8+ T cells (OT-I cells) to enhance the precursor frequency of antigen-specific T cells to aid detection of vaccine-induced responses. A day later, groups of mice (n=5-6) were vaccinated intravenously (0.08 nmol) with the different vaccines; a naive mouse was left untreated. After 28 days, mice were killed and liver and spleen collected for analysis of induced T cell response by flow cytometry. Antigen-specific cells were identified using fluorescent MHC/peptide tetramers containing the targeted CD8⁺ T cell epitope recognised by OT-I cells, and expression of Ly5.1, a congenic marker expressed by the OT-I mouse strain and not recipient C57BL/6 mice. The antigen-specific cells were further characterised as resident-memory T cells (T_{RM} cells; CD8⁺ Ly5.1⁺ CD44⁻ CD69⁻ CD62L^{low} cells), effector memory T cells (T_{EM} cells; CD8⁺ Ly5.1⁺ CD44⁺ CD69⁻ CD62L^{low} cells) or central memory cells (T_{CM} cells; CD8⁺ Ly5.1⁺ CD44⁺ CD69⁻ CD62Lhigh cells). (A) Percentages of T_{RM}, T_{EM} and T_{CM} OT-I cells in each treatment group in liver. Mean±SEM for each T cell subset are shown; statistical analysis relates only to pairwise comparisons of the percentage of T_{RM} cells between the treatment groups. Black bars: T_{RM} cells; white bars: T_{EM} cells; grey bars: T_{CM} cells. *P<0.05, **P<0.01, ***P<0.001, ****P<0.0001. One-way ANOVA. (B) Percentages of R_{M}, T_{EM} and T_{CM} OT-I cells in each treatment group in spleen. (C) Mean number of T_{RM} cells in liver for each treatment group (±SEM); dots represent individual mice. Black bars: T_{RM} cells; white bars: T_{EM} cells; grey bars: T_{CM} cells. *P<0.05, **P<0.01, ***P<0.001, ****P<0.0001. One-way ANOVA.

FIG. 2 provides a graphical representation showing that LPX-mRNA vaccines containing CI058 induce superior liver T_{RM} cell responses compared to vaccines containing α-GalCer. Dose response of LPX-mOVA-α-GalCer versus LPX-mOVA-CI058 vaccines. Analysis was in C57BL/6 mice, without transfer of OT-I cells, so endogenous T cell responses were monitored on a normal T cell repertoire. Groups of 5 mice were used, and vaccines were administered intravenously. After 28 days, mice were killed and liver tissue collected for analysis of induced T cell response by flow cytometry. Antigen-specific cells were identified using fluorescent MHC/peptide tetramers, and further characterised as resident-memory T cells (T_{RM} CD8⁺ CD44⁺ CD69⁺ CXCR6⁺ CD49a⁺ CD62L^{low} cells), effector memory T cells (T_{EM} CD8⁺ CD44⁺ CD69⁻ KLRG1⁺ CXCR3⁺ CD62L^{low} cells) or central memory cells (T_{CM} CD8⁺ CD44⁺ CD69⁻ CD62L^{high} cells). At the same timer, NKT cells in the liver were also evaluated using fluorescent CD1d/α-GalCer tetramers. These were characterised for expression of different markers of activation, including PD-1 (only the latter is shown here). White circles: LPX-mOVA-α-GalCer; black circles: LPX-mOVA-CI058. (A) Graph showing mean number of T_{RM} cells in liver for each treatment group (±SEM) overdose range. ****P<0.0001. (B) Graph showing mean level of expression of the “invariant” NKT cell-associated TCR for each treatment group (±SEM), defined as level of binding of fluorescent CD1d/α-GalCer tetramers and expressed as mean fluorescence index. ****P<0.0001 (C) Graph showing mean level of expression of PD-1 on NKT cells for each treatment group (±SEM). **P<0.01

FIG. 3 provides a graphical representation showing that LPX-mRNA vaccines containing CI058 induce functional liver TRM responses that protect from Plasmodium berghei infection. The function of T_{RM} induced with LPX vaccines containing CI058 was tested in a mouse model of malaria infection, which is known to require liver T_{RM} cells to provide protection against infection. Analysis was in C57BL/6 mice with LPX-mOVA versus LPX-mOVA-CI058 vaccines, with prior transfer of OT-I cells to enhance T cell precursor frequency, and challenge was with P. berghei sporozoites from a parasite strain modified to express OVA. (A) Groups of mice (n=12-15) were subject to vaccination only. Percentages of T_{RM}, T_{EM} and T_{CM} OT-I cells in each treatment group determined in liver 30 days later, assessed as in FIG. 1. Black bars: T_{RM} cells; white bars: T_{EM} cells; grey bars: T_{CM} cells. Mean±SEM for each T cell subset are shown; statistical analysis relates only to pairwise comparison of the percentage of T_{RM} cells between the treatment groups. *P<0.05, **P<0.01, ***P<0.001, ****P<0.0001. (B) Percentages of T_{RM}, T_{EM} and T_{CM} OT-I cells in each treatment group in spleen. Black bars: T_{RM} cells; white bars: T_{EM} cells; grey bars: T_{CM} cells. (C) Mean number of TRM in liver for each treatment group (±SEM); dots represent individual mice. (D) Groups of mice were subject to vaccination followed by challenge with 200 OVA-expressing P. berghei sporozoites 30 days later, and parasitemia was measured 6, 7,8, 10 and 12 days after infection. Mice with two consecutive days of visible parasites in the blood were culled. The mean percentage of red blood cells containing parasites at day 7 post primary, malaria challenge for each treatment group is shown (±SEM); each dot represents an individual mouse. ****P<0.0001. (E) The percentage of mice that succumbed (black boxes) or were protected (white boxes) after sporozoite challenge is shown, with mouse numbers shown in brackets. (F) Analysis of responses to vaccines encoding a defined P. berghei antigen, RPL6, with or without inclusion of CI058. The experiment was conducted in C57BL/6 mice without any transfer of TCR transgenic T cells, so the results therefore reflect endogenous T cell responses on a normal T cell repertoire. Groups of mice (n=12-15) were subject to vaccination, and then percentages of T_{RM} cells, T_{EM} cells and T_{CM} cells in each treatment group were evaluated in liver 30 days later by flow cytometry using MHC/peptide tetramers containing a defined CD8 T cell epitope from RLP6, using gating strategy in FIG. 2. Black bars: T_{RM} cells; white bars: T_{EM} cells; grey bars: T_{CM} cells. Mean±SEM for each T cell subset are shown; statistical analysis relates only to pairwise comparison of the percentage of T_{RM} cells between the treatment groups. (G) Percentages of T_{RM}, T_{EM} and T_{CM} OT-I cells in each treatment group in spleen. Black bars: T_{RM} cells; white bars: T_{EM} cells; grey bars: T_{CM} cells. (H) Mean number of T_{RM} cells in liver for each treatment group (±SEM); dots represent individual mice. (I) Groups of mice (n=5) were subject to vaccination with RPL6-containing vaccines as indicated (versus naïve controls) followed by challenge with 200 unmodified P. berghei sporozoites 30 days later. The mean percentage of red blood cells containing parasites at day 7 post primary malaria challenge for each treatment group is shown (±SEM) (J) The percentage of mice that succumbed (black boxes) or were protected (white boxes) after sporozoite challenge is shown, with mouse numbers shown in brackets.

FIG. 4 provides graphical evidence that LPX-mRNA vaccines containing CI058 do not induce liver T_{RM} cell responses in mice that are deficient in NKT cells. Groups (n=3) of C57BL/6 mice or TRAJ18-deficient mice were vaccinated with LPX-mOVA-CI058, or injected with PBS vehicle. The TRAJ18-deficient strain cannot complete the TCR rearrangement required to form the TCR-alpha chain used by type I NKT cells, and therefore are deficient in these cells, while the remaining T cell repertoire is largely intact. No prior transfer of OT-I cells was conducted, so results reflect endogenous T cell responses to vaccination. Mean number of T_{RM} cells in liver 28 days after vaccination for each treatment group (±SEM) are shown; dots represent individual mice. **P<0.01,

FIG. 5 provides graphical representations that LPX-mRNA vaccines containing CI058 are superior liver T_{RM} cell-inducers to vaccines containing defined NKT agonist prodrugs. To examine whether a prodrug mechanism is involved in enhanced T_{RM} cell induction, LPX-mOVA-CI058 vaccines were compared to vaccines containing different NKT cell agonist prodrugs. These prodrugs are based on an α-GalCer structure where the acyl chain has been mis-orientated to form an inactive ligand until in vivo enzymatic activity releases the active agonist. Compounds with a range acyl chain lengths were used (C26, C20, C8, C0), which have different avidity for CD1d. (A) Groups of mice (n=6) were transferred OT-I cells, and then a day later were vaccinated with indicated vaccines. The percentages of T_{RM}, T_{EM} and T_{CM} OT-I cells in each treatment group were evaluated in liver 30 days later. Black bars: T_{RM} cells; white bars: T_{EM} cells; grey bars: T_{CM} cells. Mean±SEM for each T cell subset are shown. Statistical analysis relates only to pairwise comparison of the percentage of TRM between the treatment groups. All comparisons to LPX-mOVA-CI058 were significant. ****P<0.0001. (B) Mean number of T_{RM} cells in liver for each treatment group (±SEM); dots represent individual mice. ****P<0.0001. (C) The number of T_{R}M cells in the livers of each mouse in the experiment were correlated with NKT cell phenotype defined by level of expression of the TCR, as defined by level of binding of fluorescent CD1d/α-GalCer tetramers. Correlation analysis p<0.0001; R2=0.4062.

FIG. 6 provides a graphical representation showing that LPX-mRNA vaccines containing α-GalCer-based structures with modifications via 6'S-substitution enhance proportion of liver T_{RM} cells induced. Various 6'S-substituted compounds were incorporated into LPX-mOVA vaccines and tested for capacity induce liver T_{RM} cells. (A, B) Groups of mice (n=5-6) were transferred OT-I cells, and then a day later were vaccinated with indicated vaccines. The percentages of T_{RM}, T_{E}M and T_{CM} OT-I cells in each treatment group were evaluated in liver 30 days later. Mean±SEM for each T cell subset are shown. Statistical analysis relates only to pairwise comparison of the percentage of T_{RM} cells between the treatment groups. Black bars: T_{R}M cells; white bars: T_{EM} cells; grey bars: T_{CM} cells.*P<0.05, **P<0.01, ***P<0.001, ****P<0.0001. (C) Mean number of T_{RM} cells in liver for each treatment group (±SEM); dots represent individual mice (n=5-6: Note that non-responders defined as having count below highest level in PBS controls, were assumed to be intravenous vaccination failures, and excluded from statistical analysis; no more than one animal was excluded on this basis per group). *P<0.05, **P<0.01, ***P<0.001, ****P<0.0001. (D) As in C, (n=5). **P<0.01.

FIG. 7 provides a graphical representation showing that LPX-mRNA vaccines containing α-GalCer-based structures with modifications via 6′ N-substitution enhance proportion of liver T_{RM} cells induced. Various 6′ N-substituted compounds were incorporated into LPX-mOVA vaccines and tested for capacity induce liver T_{RM} cells. (A, B) Groups of mice (n=5-6) were transferred OT-I cells, and then a day later were vaccinated with indicated vaccines. The percentages of T_{RM}, T_{EM} and T_{CM} OT-I cells in each treatment group were evaluated in liver 30 days later. Black bars: T_{R}M cells; white bars: T_{E}M cells; grey bars: T_{CM} cells. Mean±SEM for each T cell subset are shown. Statistical analysis relates only to pairwise comparison of the percentage of TRM between the treatment groups. ****P<0.0001. (C) Mean number of TRM in liver for each treatment group (±SEM); dots represent individual mice (n=5). ****P<0.0001 (D) As in C, (n=6). **P<0.01.

FIG. 8 provides a graphical representation demonstrating that mRNA vaccines prepared as lipid nanoparticles (LNP) that contain CI058 induce liver T_{RM} cells. Vaccines were prepared with mRNA encoding OVA encapsulated in LNPs (LNP-mOVA-CI058). Groups of mice (n=5) were vaccinated twice, with an interval of either two weeks, or eight weeks, and then liver and spleen were collected for analysis of T cell responses 28 days after the last dose. (A) Percentages of antigen-specific T_{RM} cells, T_{E}M cells and T_{CM} cells are shown for liver. Black bars: T_{RM} cells; white bars: T_{E}M cells; grey bars: T_{CM} cells. (B) Percentages of antigen-specific T_{R}M cells, T_{E}M cells and T_{CM} cells are shown for spleen. Black bars: T_{R}M cells; white bars: T_{E}M cells; grey bars: T_{CM} cells. (C) Mean number of T_{R}M cells in liver for each treatment group (±SEM); dots represent individual mice. *P<0.05.

FIG. 9 provides a graphical representation demonstrating that mRNA vaccines prepared as lipid nanoparticles (LNP) that contain CI536 induce liver TRM cells. Vaccines were prepared with mRNA encoding OVA encapsulated in LNPs (LNP-mOVA-CI536). Groups (n=9-10) of naïve C57BL/6 mice (i.e., no OT-I transfer) were vaccinated once, or twice with an interval of two weeks, with the liver and spleen collected for analysis of T cell responses 28 days after the last dose. (A) Numbers of antigen-specific TRM cells for liver. Black bars: TRM cells; white bars: TEM cells; grey bars: TCM cells. (B) Numbers of antigen-specific TRM cells for spleen. Black bars: TRM cells; white bars: TEM cells; grey bars: TCM cells. (C) Mean number of TRM cells in liver for each treatment group (±SEM); dots represent individual mice. Data were log transformed and analysed by One-way ANOVA with Tukey's post test **P<0.01, ***P<0.001, ****P<0.0001.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “a cell” means one cell or more than one cell.

The term “about” as used herein refers to the usual error range for the respective value readily known to the skilled person in this technical field. Reference to “about” a value or parameter herein includes (and describes) embodiments that are directed to that value or parameter per se.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and desirably together in the same formulation. By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 cm, preferably from within about 0.5 to about 5 cm. The term “separately” as used herein means that the agents are administered at an interval, for example at an interval of about a day to several weeks or months. The active agents may be administered in either order. The term “sequentially” as used herein means that the agents are administered in sequence, for example at an interval or intervals of minutes, hours, days or weeks. If appropriate the active agents may be administered in a regular repeating cycle.

The term “agent” includes a compound that induces a desired pharmacological and/or physiological effect. The term also encompasses pharmaceutically acceptable and pharmacologically active ingredients of those compounds specifically mentioned herein including but not limited to salts, esters, amides, prodrugs, active metabolites, analogues and the like. When the above term is used, then it is to be understood that this includes the active agent per se as well as pharmaceutically acceptable, pharmacologically active salts, esters, amides, prodrugs, metabolites, analogues, etc. The term “agent” is not to be construed narrowly but extends to small molecules, proteinaceous molecules such as peptides, polypeptides and proteins as well as compositions comprising them and genetic molecules such as RNA, DNA and mimetics and chemical analogues thereof as well as cellular agents. The term “agent” includes a cell that is capable of producing and secreting a polypeptide referred to herein as well as a polynucleotide comprising a nucleotide sequence that encodes that polypeptide. Thus, the term “agent” extends to nucleic acid constructs including vectors such as viral or non-viral vectors, expression vectors and plasmids for expression in and secretion in a range of cells.

The “amount” or “level” of a biomarker is a detectable level in a sample. These can be measured by methods known to one skilled in the art and also disclosed herein. The expression level or amount of biomarker assessed can be used to determine the response to treatment.

As used herein, “and/or” refers to and encompasses any and all possible combinations of one or more of the associated listed items, as well as the lack of combinations when interpreted in the alternative (or).

By “antigen” is meant all, or part of, a protein, peptide, or other molecule or macromolecule capable of eliciting an immune response in a vertebrate animal, especially a mammal. Such antigens are also reactive with antibodies from animals immunised with that protein, peptide, or other molecule or macromolecule.

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

By “corresponds to” or “corresponding to” is meant an amino acid sequence that displays substantial sequence similarity or identity to a reference amino acid sequence. In general, the amino acid sequence will display at least about 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to at least a portion of the reference amino acid sequence.

An “effective amount” is at least the minimum amount required to effect a measurable improvement or prevention of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioural symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of an infection, an effective amount of the drug may have the effect in reducing pathogen (bacterium, virus, etc.) titres in the circulation or tissue; reducing the number of pathogen infected cells; inhibiting (i.e., slow to some extent or desirably stop) pathogen infection of organs; inhibit (i.e., slow to some extent and desirably stop) pathogen growth; and/or relieving to some extent one or more of the symptoms associated with the infection. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

The term “expression” with respect to a gene sequence refers to transcription of the gene to produce a RNA transcript (e.g., mRNA, antisense RNA, siRNA, shRNA, miRNA, etc.) and, as appropriate, translation of a resulting mRNA transcript to a protein. Thus, as will be clear from the context, expression of a coding sequence results from transcription and translation of the coding sequence. Conversely, expression of a non-coding sequence results from the transcription of the non-coding sequence.

The terms “level of expression” or “expression level” in general are used interchangeably and generally refer to the amount of a biomarker in a sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., post-translational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (e.g., transfer and ribosomal RNAs).

“Elevated expression”, “elevated expression levels”, or “elevated levels” refers to an increased expression or increased levels of a biomarker in an individual or part of an individual (e.g., a cell, tissue or organ) relative to a control, such as an individual or individuals who are not suffering from the disease or disorder (e.g., T cell dysfunctional disorder) or parts thereof (e.g., a cell, tissue or organ) or an internal control (e.g., housekeeping biomarker).

“Reduced expression”, “reduced expression levels”, or “reduced levels” refers to a decreased expression or decreased levels of a biomarker in an individual or part of an individual (e.g., a cell, tissue or organ) relative to a control, such as an individual or individuals who are not suffering from the disease or disorder (e.g., T-cell dysfunctional disorder) or parts thereof (e.g., a cell, tissue or organ) or an internal control (e.g., housekeeping biomarker). In some embodiments, reduced expression is little or no expression.

The term “immune effector cells” in the context of the present invention relates to cells which exert effector functions during an immune reaction. For example, such cells secrete cytokines and/or chemokines, kill microbes, secrete antibodies, recognize infected or cancerous cells, and optionally eliminate such cells. For example, immune effector cells comprise T-cells (cytotoxic T-cells, helper T-cells), B-cells, natural killer (NK) cells, lymphokine-activated killer (LAK) cells, neutrophils, macrophages, and dendritic cells.

The term “immune effector functions” in the context of the present invention includes any functions mediated by components of the immune system that result, for example, in the killing of virally infected cells or tumour cells, or in the inhibition of tumour growth and/or inhibition of tumour development, including: inhibition of tumour dissemination and metastasis. Preferably, the immune effector functions in the context of the present invention are T cell mediated effector functions. Such functions comprise in the case of a helper T-cell (CD4⁺ T cell) the recognition of an antigen or an antigen peptide derived from an antigen in the context of MHC class II molecules by T cell receptors, the release of cytokines and/or the activation of CD8⁺ lymphocytes (CTLs) and/or B-cells, and in the case of CTL the recognition of an antigen or an antigen peptide derived from an antigen in the context of MHC class I molecules by T cell receptors, the elimination of cells presented in the context of MHC class I molecules, i.e., cells characterized by presentation of an antigen with class I MHC, for example, via apoptosis or perforin-mediated cell lysis, production of cytokines such as IFN-γ and TNF-α, and specific cytolytic killing of antigen expressing target cells.

The term “immune response” refers to any detectable response to a particular substance (such as an antigen or immunogen) by the immune system of a host mammal, such as innate immune responses (e.g., activation of Toll receptor signalling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids).

The term “infection” refers to invasion of body tissues by disease-causing microorganisms, their multiplication and the reaction of body tissues to these microorganisms and the toxins they produce. “Infection” includes but are not limited to infections by viruses, prions, bacteria, viroids, parasites, protozoans and fungi.

As used herein, “instructional material” includes a publication, a recording, a diagram, or any other medium of expression which can be used to communicate the usefulness of the compositions and methods of the invention. The instructional material of the kit of the invention may, for example, be affixed to a container which contains the therapeutic or diagnostic agents of the invention or be shipped together with a container which contains the therapeutic or diagnostic agents of the invention.

The term “lipid” refers to a group of organic compounds that are derivatives of fatty acids (e.g., esters) and are generally characterized by being insoluble in water but soluble in many organic solvents. Lipids are usually divided into at least three classes: (1) “simple lipids” which include fats and oils, as well as waxes; (2) “compound lipids” which include glycolipids and phospholipids; and (3) “derived lipids” such as steroids.

As used herein, the term “nanoparticle” refers to any particle having a diameter making the particle suitable for systemic (in particular, intramuscular or intravenous) administration, typically having a diameter of less than about 500 nanometres (nm). The term “lipid nanoparticle”, also referred to as “LNP” is not restricted to a particular morphology, and includes any morphology generated when a cationic lipid and optionally one or more further lipids are combined, e.g., in an aqueous environment and/or in the presence of a nucleic acid (e.g., an RNA). For example, a liposome, a lipid complex, a lipoplex and the like are within the scope of a lipid nanoparticle (LNP).

The terms “patient”, “subject”, “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. A preferred subject is a human in need of eliciting an immune response, including an immune response with enhanced T cell activation. However, it will be understood that the aforementioned terms do not imply that symptoms are present.

The term “pharmaceutical composition” or “pharmaceutical formulation” refers to a preparation which is in such form as to permit the biological activity of the active ingredient(s) to be effective, and which contains no additional components which are unacceptably toxic to a subject to which the composition or formulation would be administered. Such formulations are sterile. “Pharmaceutically acceptable” excipients (vehicles, additives) are those which can reasonably be administered to a subject mammal to provide an effective dose of the active ingredient employed.

As used herein, the terms “prevent”, “prevented”, or “preventing”, refer to a prophylactic treatment which increases the resistance of a subject to developing the disease or condition or, in other words, decreases the likelihood that the subject will develop the disease or condition as well as a treatment after the disease or condition has begun in order to reduce or eliminate it altogether or prevent it from becoming worse. These terms also include within their scope preventing the disease or condition from occurring in a subject which may be predisposed to the disease or condition but has not yet been diagnosed as having it.

The term “sample” as used herein includes any biological specimen that may be extracted, untreated, treated, diluted or concentrated from a subject. Samples may include, without limitation, biological fluids such as whole blood, serum, red blood cells, white blood cells, plasma, saliva, urine, stool (i.e., faeces), tears, sweat, sebum, nipple aspirate, ductal lavage, tumour exudates, synovial fluid, ascitic fluid, peritoneal fluid, amniotic fluid, cerebrospinal fluid, lymph, fine needle aspirate, amniotic fluid, any other bodily fluid, cell lysates, cellular secretion products, inflammation fluid, semen and vaginal secretions. Samples may include tissue samples and biopsies, tissue homogenates and the like. Advantageous samples may include ones comprising any one or more biomarkers as taught herein in detectable quantities. Suitably, the sample is readily obtainable by minimally invasive methods, allowing the removal or isolation of the sample from the subject. In certain embodiments, the sample contains blood, especially peripheral blood, or a fraction or extract thereof. Typically, the sample comprises blood cells such as mature, immature or developing leukocytes, including lymphocytes, polymorphonuclear leukocytes, neutrophils, monocytes, reticulocytes, basophils, coelomocytes, hemocytes, eosinophils, megakaryocytes, macrophages, dendritic cells natural killer cells, or fraction of such cells (e.g., a nucleic acid or protein fraction). In specific embodiments, the sample comprises leukocytes including peripheral blood mononuclear cells (PBMC).

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys, and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by an appropriate method. For example, sequence identity analysis may be carried out using the DNASIS computer program (Version 2.5 for windows; available from Hitachi Software engineering Co., Ltd., South San Francisco, California, USA) using standard defaults as used in the reference manual accompanying the software.

As used herein a “small molecule” refers to a compound that has a molecular weight of less than 3 kilodalton (kDa), and typically less than 1.5 kDa, and more preferably less than about 1 kDa. Small molecules may be nucleic acids, peptides, polypeptides, peptidomimetics, carbohydrates, lipids or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the invention to identify compounds that modulate a bioactivity. A “small organic molecule” is an organic compound (or organic compound complexed with an inorganic compound (e.g., metal)) that has a molecular weight of less than 3 kDa, less than 1.5 kDa, or even less than about 1 kDa.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridisable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 15 mM sodium chloride/1.5 mM sodium citrate/0.1% sodium dodecyl sulphate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 75 mM sodium citrate), 50 mM sodium phosphate (pH 6.8), 0.1% sodium pyrophosphate, 5×Denhardt's solution, sonicated salmon sperm DNA (50 pg/mL), 0.1% SDS, and 10% dextran sulphate at 42° C., with a 10 minute wash at 42° C. in 0.2×SSC (sodium chloride/sodium citrate) followed by a 10 minute high-stringency wash consisting of 0.1×SSC containing EDTA at 55° C.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with a T cell dysfunctional disorder are mitigated or eliminated, including, but are not limited to, reducing the proliferation of (or destroying) cancerous cells, reducing pathogen infection, decreasing symptoms resulting from the disease, increasing the quality of life of those suffering from the disease, decreasing the dose of other medications required to treat the disease, and/or prolonging survival of individuals.

As used herein, the term “alkyl”, used either alone or in compound words denotes straight chain, branched or cyclic alkyl, preferably C₁₋₂₀ alkyl, e.g., C₁₋₁₀ or C₁₋₆. Examples of straight chain and branched alkyl include methyl, ethyl, n-propyl, isopropyl, n-butyl, sec-butyl, tert-butyl, n-pentyl, 1,2-dimethylpropyl, 1,1-dimethyl-propyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethyl-pentyl, 1,2,3-trimethylbutyl, 1,1,2-trimethylbutyl, 1,1,3-trimethylbutyl, octyl, 6-methylheptyl, 1-methylheptyl, 1,1,3,3-tetramethylbutyl, nonyl, 1-, 2-, 3-, 4-, 5-, 6- or 7-methyloctyl, 1-, 2-, 3-, 4- or 5-ethylheptyl, 1-, 2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1,2-pentylheptyl and the like. Examples of cyclic alkyl include mono- or polycyclic alkyl groups such as cyclopropyl, cyclobutyl, cyclopentyl, cyclohexyl, cycloheptyl, cyclooctyl, cyclononyl, cyclodecyl and the like. Where an alkyl group is referred to generally as “propyl”, “butyl” etc, it will be understood that this can refer to any of straight, branched and cyclic isomers where appropriate. An alkyl group may be optionally substituted by one or more optional substituents as herein defined.

The term “alkenyl” as used herein denotes groups formed from straight chain, branched or cyclic hydrocarbon residues containing at least one carbon to carbon double bond including ethylenically mono-, di- or polyunsaturated alkyl or cycloalkyl groups as previously defined, preferably C₂₋₂₀ alkenyl (e.g., C₂₋₁₀ or C₂₋₆). Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl. An alkenyl group may be optionally substituted by one or more optional substituents as herein defined.

As used herein the term “alkynyl” denotes groups formed from straight chain, branched or cyclic hydrocarbon residues containing at least one carbon-carbon triple bond including ethylenically mono-, di- or polyunsaturated alkyl or cycloalkyl groups as previously defined. Unless the number of carbon atoms is specified the term preferably refers to C₂₋₂₀ alkynyl (e.g., C₂₋₁₀ or C₂-6). Examples include ethynyl, 1-propynyl, 2-propynyl, and butynyl isomers, and pentynyl isomers. An alkynyl group may be optionally substituted by one or more optional substituents as herein defined.

As the context may require, reference to a group such as alkyl, alkenyl, alkynyl may mean that such nominally monovalent species must be divalent species. For the avoidance of doubt, in such contexts, the monovalent groups should be understood to indicate the divalent equivalent. For example, in some contexts “alkyl” may be used to denote an alkylene group. Likewise in some contexts “alkenyl” may be used to denote an alkenylene group. Likewise in some contexts “alkynyl” may be used to denote an alkynylene group.

In some instances, a group may be referred to as “alk” such as in “alkaryl” in which case the “alk” denotes any of alkyl, alkenyl, alkynyl.

The term “halogen” (“halo”) denotes fluorine, chlorine, bromine or iodine (fluoro, chloro, bromo or iodo).

The term “aryl” (or “carboaryl”) denotes any of single, polynuclear, conjugated and fused residues of aromatic hydrocarbon ring systems. Examples of aryl include phenyl, biphenyl, terphenyl, quaterphenyl, naphthyl, tetrahydronaphthyl, anthracenyl, dihydroanthracenyl, benzanthracenyl, dibenzanthracenyl, phenanthrenyl, fluorenyl, pyrenyl, idenyl, azulenyl, chrysenyl. Preferred aryl include phenyl, anthracenyl and naphthyl. An aryl group may or may not be optionally substituted by one or more optional substituents as herein defined.

The term “carbocyclyl” includes any of non-aromatic monocyclic, polycyclic, fused or conjugated hydrocarbon residues, preferably C₃₋₂₀ (e.g., C₃₋₁₀ or C₃₋₈). The rings may be saturated, e.g., cycloalkyl, or may possess one or more double bonds (cycloalkenyl) and/or one or more triple bonds (cycloalkynyl). Particularly preferred carbocyclyl moieties are 5-6-membered or 9-10 membered ring systems. Suitable examples include cyclopropyl, cyclobutyl, cyclopentyl, cyclohexyl, cycloheptyl, cyclooctyl, cyclononyl, cyclodecyl, cyclopentenyl, cyclohexenyl, cyclooctenyl, cyclopentadienyl, cyclohexadienyl, cyclooctatetraenyl, indanyl, decalinyl and indenyl. A carbocyclyl group may be optionally substituted by one or more optional substituents as herein defined.

The term “heteroatom” or “hetero” as used herein in its broadest sense refers to any atom other than a carbon atom which may, for example, be a member of a cyclic organic group. Particular examples of heteroatoms include nitrogen, oxygen, sulfur, phosphorous, boron, silicon, selenium and tellurium, more particularly nitrogen, oxygen and sulfur.

The term “heterocyclyl” when used alone or in compound words includes any of monocyclic, polycyclic, fused or conjugated hydrocarbon residues, preferably C₃₋₂₀ (e.g., C₃₋₁₀ or C₃₋₈) wherein one or more carbon atoms are replaced by a heteroatom so as to provide a non-aromatic residue. Suitable heteroatoms include O, N, S, P and Se, particularly O, N and S. Where two or more carbon atoms are replaced, this may be by two or more of the same heteroatom or by different heteroatoms. The heterocyclyl group may be saturated or partially unsaturated, i.e., possess one or more double bonds.

Particularly preferred heterocyclyl are 5-6 and 9-10 membered heterocyclyl. Suitable examples of heterocyclyl groups may include azridinyl, oxiranyl, thiiranyl, azetidinyl, oxetanyl, thietanyl, 2H-pyrrolyl, pyrrolidinyl, pyrrolinyl, piperidyl, piperazinyl, morpholinyl, indolinyl, imidazolidinyl, imidazolinyl, pyrazolidinyl, thiomorpholinyl, dioxanyl, tetrahydrofuranyl, tetrahydropyranyl, tetraliydropyrrolyl, tetrahydrothiophenyl, pyrazolinyl, dioxalanyl, thiazolidinyl, isoxazolidinyl, dihydropyranyl, oxazinyl, thiazinyl, thiomorpholinyl, oxathianyl, dithianyl, trioxanyl, thiadiazinyl, dithiazinyl, trithianyl, azepinyl, oxepinyl, thiepinyl, indenyl, indanyl, 3H-indolyl, isoindolinyl, 4H-quinolazinyl, chromenyl, chromanyl, isochromanyl, pyranyl and dihydropyranyl. A heterocyclyl group may be optionally substituted by one or more optional substituents as herein defined.

The term “heteroaryl” includes any of monocyclic, polycyclic, fused or conjugated hydrocarbon residues, wherein one or more carbon atoms are replaced by a heteroatom so as to provide an aromatic residue. Preferred heteroaryl have 3-20 ring atoms, e.g., 3-10. Particularly preferred heteroaryl are 5-6 and 9-10 membered. Suitable heteroatoms include, O, N, B, S, P and Se, particularly O, N, B and S, particularly O, N, and S. Where two or more carbon atoms are replaced, this may be by two or more of the same heteroatom or by different heteroatoms. Suitable examples of heteroaryl groups may include pyridyl, pyrrolyl, thienyl, imidazolyl, furanyl, benzothienyl, isobenzothienyl, benzofuranyl, isobenzofuranyl, indolyl, isoindolyl, pyrazolyl, pyrazinyl, pyrimidinyl, pyridazinyl, indolizinyl, quinolyl, isoquinolyl, phthalazinyl, 1,5-naphthyridinyl, quinozalinyl, quinazolinyl, quinolinyl, oxazolyl, thiazolyl, isothiazolyl, isoxazolyl, triazolyl, oxadialzolyl, oxatriazolyl, triazinyl, and furazanyl. A heteroaryl group may be optionally substituted by one or more optional substituents as herein defined.

The term “acyl” either alone or in compound words denotes a group containing the moiety C═O. Preferred acyl includes C(O)—R^{e}, wherein R^{e} is hydrogen or an alkyl, alkenyl, alkynyl, aryl, heteroaryl, carbocyclyl, or heterocyclyl residue. Examples of acyl include formyl, straight chain or branched alkanoyl (e.g., C₁₋₂₀) such as acetyl, propanoyl, butanoyl, 2-methylpropanoyl, pentanoyl, 2,2-dimethylpropanoyl, hexanoyl, heptanoyl, octanoyl, nonaiioyl, decanoyl, undecanoyl, dodecanoyl, tridecanoyl, tetradecanoyl, pentadecanoyl, hexadecanoyl, heptadecanoyl, octadecanoyl, nonadecanoyl and icosanoyl; cycloalkylcarbonyl such as cyclopropylcarbonyl cyclobutylcarbonyl, cyclopentylcarbonyl and cyclohexylcarbonyl; aroyl such as benzoyl, toluoyl and naphthoyl; aralkanoyl such as phenylalkanoyl (e.g., phenylacetyl, phenylpropanoyl, phenylbutanoyl, phenylisobutylyl, phenylpentanoyl and phenylhexanoyl) and naphthylalkanoyl (e.g., naphthylacetyl, naphthylpropanoyl and naphthylbutanoyl); aralkenoyl such as phenylalkenoyl (e.g., phenylpropenoyl, phenylbutenoyl, phenylmethacryloyl, phenylpentenoyl and phenylhexenoyl and naphthylalkenoyl (e.g., naphthylpropenoyl, naphthylbutenoyl and naphthylpentenoyl); aryloxyalkanoyl such as phenoxyacetyl and phenoxypropionyl; arylthiocarbamoyl such as phenylthiocarbamoyl; arylglyoxyloyl such as phenylglyoxyloyl and naphthylglyoxyloyl; arylsulfonyl such as phenylsulfonyl and napthylsulfonyl; heterocycliccarbonyl; heterocyclicalkanoyl such as thienylacetyl, thienylpropanoyl, thienylbutanoyl, thienylpentanoyl, thienylhexanoyl, thiazolylacetyl, thiadiazolylacetyl and tetrazolylacetyl; heterocyclicalkenoyl such as heterocyclicpropenoyl, heterocyclicbutenoyl, heterocyclicpentenoyl and heterocyclichexenoyl; and heterocyclicglyoxyloyl such as thiazolyglyoxyloyl and thienylglyoxyloyl. Where present, the R^{e} residue may be optionally substituted as described herein.

The term “sulfoxide”, either alone or in a compound word, refers to a group —S(O)Rⁱ wherein Rⁱ is selected from hydrogen, alkyl, alkenyl, alkynyl, aryl, heteroaryl, heterocyclyl, carbocyclyl, and aralkyl. Examples of preferred Rⁱ include C₁₋₂₀ alkyl, phenyl and benzyl.

The term “sulfonyl”, either alone or in a compound word, refers to a group S(O)₂—R^{j}, wherein Rj is selected from hydrogen, alkyl, alkenyl, alkynyl, aryl, heteroaryl, heterocyclyl, carbocyclyl and aralkyl. Examples of preferred R^{j} include C₁₋₂₀ alkyl, phenyl and benzyl.

The term “sulfonamide”, either alone or in a compound word, refers to a group S(O)NR^{k}R^{k} wherein each R^{k} is independently selected from hydrogen, alkyl, alkenyl, alkynyl, aryl, heteroaryl, heterocyclyl, carbocyclyl, and aralkyl. Examples of preferred R^{k} include C₁₋₂₀ alkyl, phenyl and benzyl. In a preferred embodiment at least one R^{k} is hydrogen. In another form, both R^{k} are hydrogen.

The term, “amino” is used here in its broadest sense as understood in the art and includes groups of the formula NR^{C}R^{d} wherein R^{C} and R^{d} may be any independently selected from hydrogen, alkyl, alkenyl, alkynyl, aryl, carbocyclyl, heteroaryl, heterocyclyl, arylalkyl, and acyl. R^{c} and R^{d}, together with the nitrogen to which they are attached, may also form a monocyclic, or polycyclic ring system e.g., a 3-10 membered ring, particularly, 5-6 and 9-10 membered systems. Examples of “amino” include NH₂, NHalkyl (e.g., C₁₋₂₀ alkyl), NHaryl (e.g., NHphenyl), NHaralkyl (e.g., NHbenzyl), NHacyl (e.g., NHC(O)C₁₋₂₀ alkyl, NHC(O)phenyl), Nalkylalkyl (wherein each alkyl, for example C₁₋₂₀, may be the same or different) and 5 or 6 membered rings, optionally containing one or more same or different heteroatoms (e.g., O, N and S).

The term “amido” is used here in its broadest sense as understood in the art and includes groups having the formula C(O)NR^{f}R^{f}, wherein each R^{f} is independently selected from hydrogen, alkyl, alkenyl, alkynyl, aryl, carbocyclyl, heteroaryl, heterocyclyl, arylalkyl, and acyl. Examples of amido include C(O)NH₂, C(O)NHalkyl (e.g., C₁₋₂₀ alkyl), C(O)NHaryl (e.g., C(O)NHphenyl), C(O)NHaralkyl (e.g., C(O)NHbenzyl), C(O)NHacyl (e.g., C(O)NHC(O)C₁₋₂₀ alkyl, C(O)NHC(O)phenyl), C(O)Nalkylalkyl (wherein each alkyl, for example C₁₋₂₀, may be the same or different) and 5 or 6 membered rings, optionally containing one or more same or different heteroatoms (e.g., O, N and S).

The term “carboxy ester” is used here in its broadest sense as understood in the art and includes groups having the formula CO₂R^{g}, wherein R^{g} may be selected from groups including alkyl, alkenyl, alkynyl, aryl, carbocyclyl, heteroaryl, heterocyclyl, aralkyl, and acyl. Examples of carboxy ester include CO₂C₁₋₂₀alkyl, CO₂aryl (e.g., CO₂phenyl), CO₂aralkyl (e.g., CO₂ benzyl).

As used herein, the term “aryloxy” refers to an “aryl” group attached through an oxygen bridge. Examples of aryloxy substituents include phenoxy, biphenyloxy, naphthyloxy and the like.

As used herein, the term “acyloxy” refers to an “acyl” group wherein the “acyl” group is in turn attached through an oxygen atom. Examples of “acyloxy” include hexylcarbonyloxy (heptanoyloxy), cyclopentylcarbonyloxy, benzoyloxy, 4-chlorobenzoyloxy, decylcarbonyloxy (undecanoyloxy), propylcarbonyloxy (butanoyloxy), octylcarbonyloxy (nonanoyloxy), biphenylcarbonyloxy (e.g., 4-phenylbenzoyloxy), naphthylcarbonyloxy (e.g., 1-naphthoyloxy) and the like.

As used herein, the term “alkyloxycarbonyl” refers to an “alkyloxy” group attached through a carbonyl group. Examples of “alkyloxycarbonyl” groups include butylformate, sec-butylformate, hexylformate, octylformate, decylformate, cyclopentylformate and the like.

In this specification “optionally substituted” is taken to mean that a group may or may not be substituted or fused (so as to form a condensed polycyclic group) with one, two, three or more of organic and inorganic groups, independently selected from substituent(s) including: alkyl, alkenyl, alkynyl, carbocyclyl, aryl, heterocyclyl, heteroaryl, acyl, aralkyl, alkaryl, alkheterocyclyl, alkheteroaryl, alkcarbocyclyl, halo, haloalkyl, haloalkenyl, haloalkynyl, haloaryl, halocarbocyclyl, haloheterocyclyl, haloheteroaryl, haloacyl, haloaryalkyl, hydroxy, hydroxyalkyl, hydroxyalkenyl, hydroxyalkynyl, hydroxycarbocyclyl, hydroxyaryl, hydroxyheterocyclyl, hydroxyheteroaryl, hydroxyacyl, hydroxyaralkyl, alkoxyalkyl, alkoxyalkenyl, alkoxyalkynyl, alkoxycarbocyclyl, alkoxyaryl, alkoxyheterocyclyl, alkoxyheteroaryl, alkoxyacyl, alkoxyaralkyl, alkoxy, alkenyloxy, alkynyloxy, aryloxy, carbocyclyloxy, aralkyloxy, heteroaryloxy, heterocyclyloxy, acyloxy, haloalkoxy, haloalkenyloxy, haloalkynyloxy, haloaryloxy, halocarbocyclyloxy, haloaralkyloxy, haloheteroaryloxy, haloheterocyclyloxy, haloacyloxy, nitro, nitroalkyl, nitroalkenyl, nitroalkynyl, nitroaryl, nitroheterocyclyl, nitroheteroayl, nitrocarbocyclyl, nitroacyl, nitroaralkyl, amino (e.g., NH₂), alkylamino, dialkylamino, alkenylamino, alkynylamino, arylamino, diarylamino, aralkylamino, diaralkylamkio, acylamino, diacylamino, heterocyclamino, heteroarylamino, carboxy, carboxyester, amido, alkylsulphonyloxy, arylsulphenyloxy, alkylsulphenyl, arylsulphenyl, thio, alkylthio, alkenylthio, alkynylthio, arylthio, aralkylthio, carbocyclylthio, heterocyclylthio, heteroarylthio, acylthio, sulfoxide, sulfonyl, sulfonamide, aminoalkyl, aminoalkenyl, aminoalkynyl, aminocarbocyclyl, aminoaryl, aminoheterocyclyl, aminoheteroaryl, aminoacyl, aminoaralkyl, thioalkyl, thioalkenyl, thioalkynyl, thiocarbocyclyl, thioaryl, thioheterocyclyl, thioheteroaryl, thioacyl, thioaralkyl, carboxyalkyl, carboxyalkenyl, carboxyalkynyl, carboxycarbocyclyl, carboxyaryl, carboxyheterocyciyl, carboxyheteroaryl, carboxyacyl, carboxyaralkyl, carboxyesteralkyl, carboxyesteralkenyl, carboxyesteralkynyl, carboxyestercarbocyclyl, carboxyesteraryl, carboxyesterheterocyclyl, carboxyesterheteroaryl, carboxyesteracyl, carboxyesteraralkyl, amidoalkyl, amidoalkenyl, amidoalkynyl, amidocarbocyclyl, amidoaryl, amidoheterocyclyl, amidoheteroaryl, amidoacyl, amidoaralkyl, formylalkyl, formylalkenyl, formylalkynyl, formylcarbocyclyl, formylaiyl, formylheterocyclyl, formylheteroaryl, formylacyl, formylaralkyl, acylalkyl, acylalkenyl, acylalkynyl, acylcarbocyclyl, acylaryl, acylheterocyclyl, acylheteroaryl, acylacyl, acylaralkyl, sulfoxidealkyl, sulfoxidealkenyl, sulfoxidealkynyl, sulfoxidecarbocyclyl, sulfoxidearyl, sulfoxideheterocyclyl, sulfoxideheteroaryl, sulfoxideacyl, sulfoxidearalkyl, sulfonylalkyl, sulfonylalkenyl, sulfonylalkynyl, sulfonylcarbocyclyl, sulfonylaryl, sulfonylheterocyclyl, sulfonylheteroaryl, sulfonylacyl, sulfonylaralkyl, sulfonamidoalkyl, sulfonamidoalkenyl, sulfonamidoalkynyl, sulfonamidocarbocyclyl, sulfonamidoaryl, sulfonamidoheterbcyclyl, sulfonamidoheteroaryl, sulfonamidoacyl, sulfonamidoaralkyl, nitroalkyl, nitroalkenyl, nitroalkynyl, nitrocarbocyclyl, nitroaryl, nitroheterocyclyl, nitroheteroaryl, nitroacyl, nitroaralkyl, cyano, sulfate, phosphate, triarylmethyl, triarylamino, oxadiazole, and carbazole groups, including/as well as a group represented by any one of the following formulae:

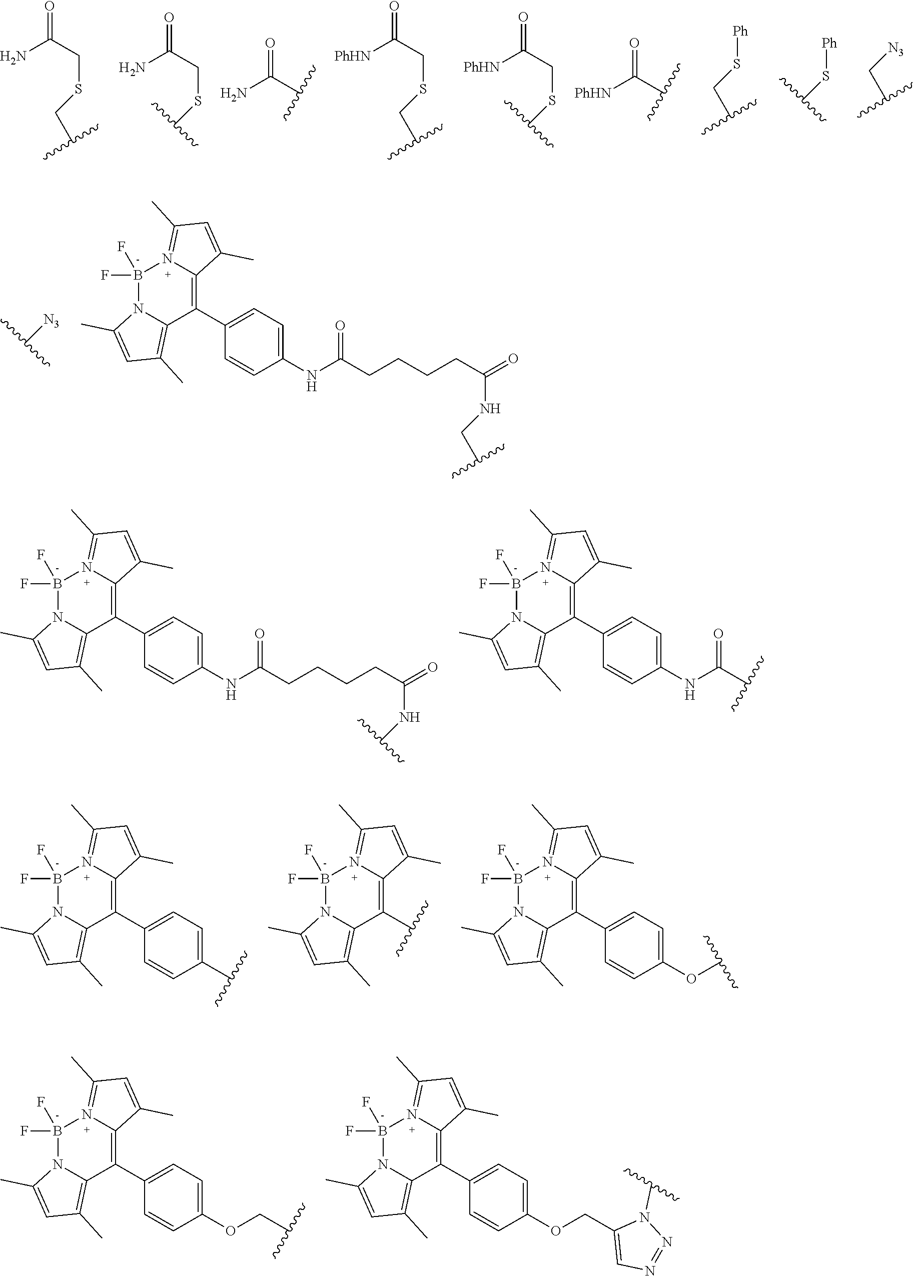

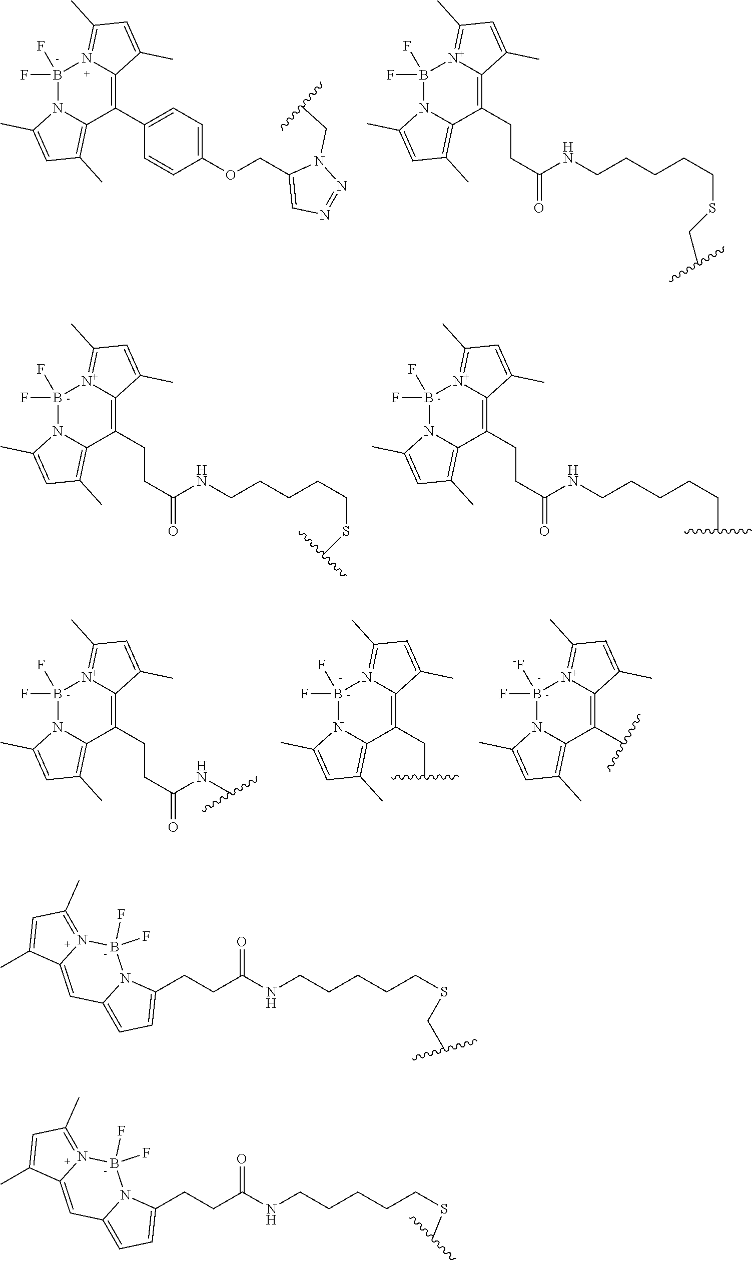

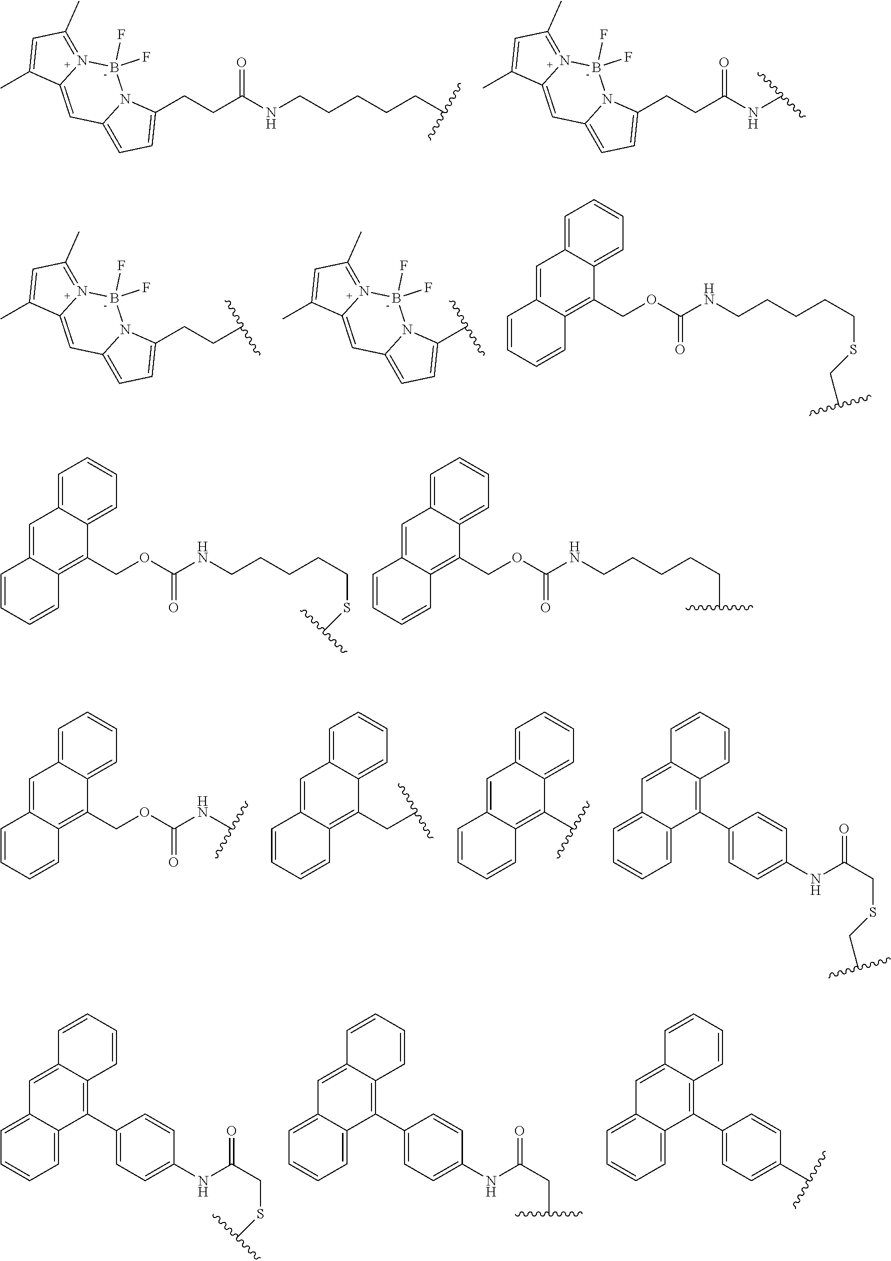

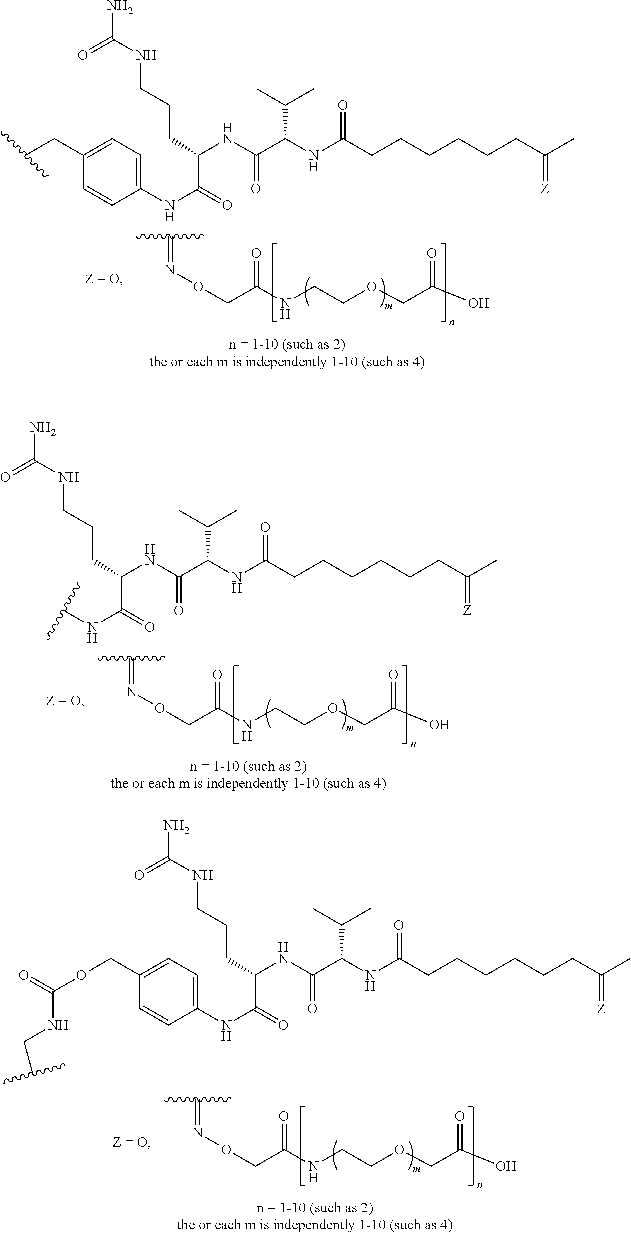

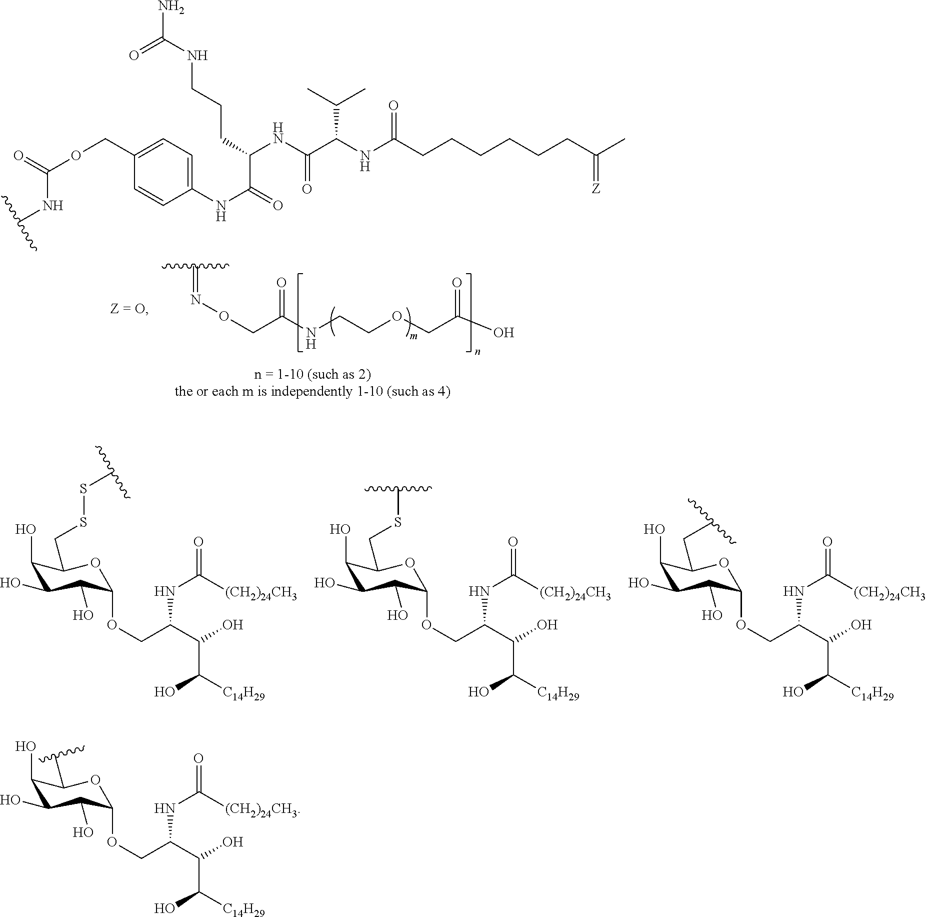

Optional substitution may also be taken to refer to where a —CH₂-group in a chain or ring is replaced by a group selected from —O—, —S—, —NR^{h}—, —C(O)— (i.e., carbonyl), —C(O)O— (i.e., ester), and —C(O)NR^{h}— (i.e., amide), O—C(O)—NR^{h}— (i.e., carbamyl), where R^{h} is as defined herein for, and independently selected from, R^{a}. For example, R^{h} may be selected from hydrogen, alkyl, alkenyl, alkynyl, aryl, heteroaryl, heterocyclyl, carbocyclyl, and aralkyl. Examples of preferred R^{h} include hydrogen, C₁₋₂₀ alkyl, phenyl and benzyl.

Preferred optional substituents include alkyl, (e.g., C₁₋₆ alkyl such as methyl, ethyl, propyl, butyl, cyclopropyl, cyclobutyl, cyclopentyl or cyclohexyl), hydroxyalkyl (e.g., hydroxymethyl, hydroxyethyl, hydroxypropyl), alkoxyalkyl (e.g., methoxymethyl, methoxyethyl, methoxypropyl, ethoxymethyl, ethoxy ethyl, ethoxypropyl etc) alkoxy (e.g., C₁₋₆ alkoxy such as methoxy, ethoxy, propoxy, butoxy, cyclopropoxy, cyclobutoxy), halo, trifluoromethyl, trichloromethyl, tribromomethyl, hydroxy, phenyl (which itself may be further substituted e.g., by C₁₋₆ alkyl, halo, hydroxy, hydroxyC₁₋₆ alkyl, C₁₋₆ alkoxy, haloC₁₋₆alkyl, cyano, nitro OC(O)C₁₋₆alkyl, and amino), benzyl (wherein benzyl itself may be further substituted e.g., by C₁₋₆ alkyl, halo, hydroxy, hydroxyC₁₋₆alkyl, C₁₋₆ alkoxy, haloC₁₋₆ alkyl, cyano, nitro OC(O)C₁₋₆ alkyl, and amino), phenoxy (wherein phenyl itself may be further substituted e.g., by C₁₋₆ alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), benzyloxy (wherein benzyl itself may be further substituted e.g., by C₁₋₆ alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), amino, alkylamino (e.g., C₁₋₆ alkyl, such as methylamino, ethylamino, propylamino etc), dialkylamino (e.g., C₁₋₆ alkyl, such as dimethylamino, diethylamino, dipropylamino), acylamino (e.g., NHC(O)CH₃), phenylamino (wherein phenyl itself may be further substituted e.g., by C₁₋₆ alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), nitro, formyl, —C(O)— alkyl (e.g., C₁₋₆ alkyl, such as acetyl), O—C(O)-alkyl (e.g., C₁₋₆alkyl, such as acetyloxy), benzoyl (wherein the phenyl group itself may be further substituted e.g., by C₁₋₅ alkyl, halo, hydroxy hydroxyC₁₋₆ alkyl, C₁₋₆ alkoxy, haloC₁₋₆ alkyl, cyano, nitro OC(O)C₁₋₆alkyl, and amino), replacement of CH₂ with C═O, CO₂H, CO₂alkyl (e.g., C₁₋₆ alkyl such as methyl ester, ethyl ester, propyl ester, butyl ester), CO₂phenyl (wherein phenyl itself may be further substituted e.g., by C₁₋₆ alkyl, halo, hydroxy, hydroxyl C₁₋₆ alkyl, C₁₋₆ alkoxy, halo C₁₋₆ alkyl, cyano, nitro OC(O)C₁₋₆ alkyl, and amino), CONH₂, CONHphenyl (wherein phenyl itself may be further substituted e.g., by C₁₋₆ alkyl, halo, hydroxy, hydroxyl C₁₋₆ alkyl, C₁₋₆ alkoxy, halo C₁₋₆ alkyl, cyano, nitro OC(O)C₁₋₆ alkyl, and amino), CONHbenzyl (wherein benzyl itself may be further substituted e.g., by C₁₋₆ alkyl, halo, hydroxy hydroxyl C₁₋₆ alkyl, C₁₋₆ alkoxy, halo C₁₋₆ alkyl, cyano, nitro OC(O)C₁₋₆ alkyl, and amino), CONHalkyl (e.g., C₁₋₆ alkyl such as methyl ester, ethyl ester, propyl ester, butyl amide) CONHdialkyl (e.g., C₁₋₆ alkyl) aminoalkyl (e.g., HN C₁₋₆ alkyl-, C₁₋₆alkylHN—C₁₋₆ alkyl- and (C₁₋₆ alkyl)₂N—C₁₋₆ alkyl-), thioalkyl (e.g., HS C₁₋₆ alkyl-), carboxyalkyl (e.g., HO₂CC₁₋₆ alkyl-), carboxyesteralkyl (e.g., C₁₋₆ alkylO₂CC₁₋₆alkyl-), amido (e.g., H₂N(O)C—, PhNH(O)C—), amidoalkyl (e.g., H₂N(O)CC₁₋₆ alkyl-, H(C₁₋₆ alkyl)N(O)CC₁₋₆ alkyl-), formylalkyl (e.g., OHCC₁₋₆alkyl-), acylalkyl (e.g., C₁₋₆ alkyl(O)CC₁₋₆ alkyl-), nitroalkyl (e.g., O₂NC₁₋₆ alkyl-), sulfoxidealkyl (e.g., R(O)SC₁₋₆ alkyl, such as C₁₋₆ alkyl(O)SC₁₋₆ alkyl-), sulfonylalkyl (e.g., R(O)₂SC₁₋₆ alkyl- such as C₁₋₆ alkyl(O)₂SC₁₋₆ alkyl-), sulfonamidoalkyl (e.g., H₂N(O)SC₁₋₆ alkyl, H(C₁₋₆ alkyl)N(O)SC₁₋₆ alkyl-), triarylmethyl, triarylamino, oxadiazole, and carbazole, including/as well as a group represented by any one of the following formulae:

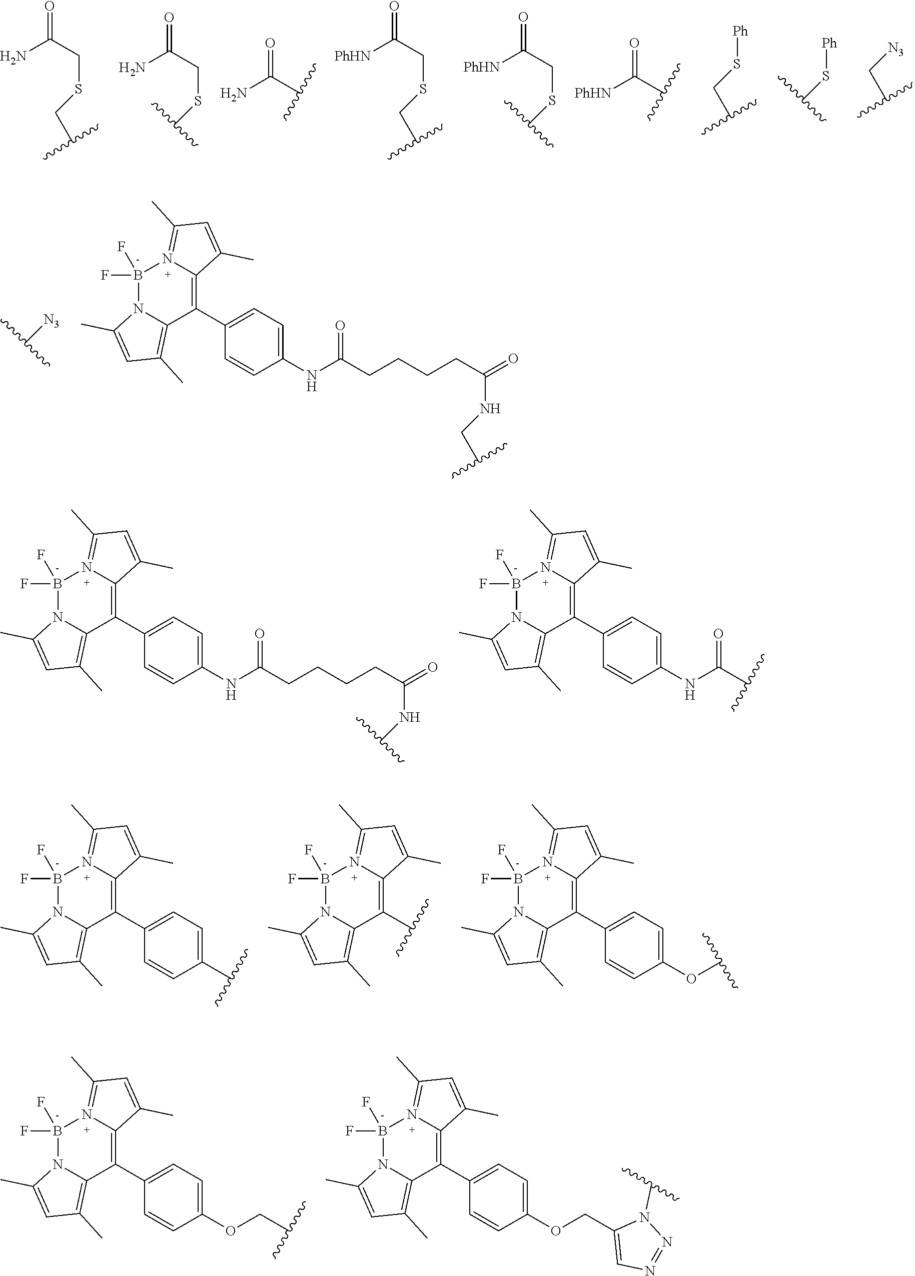

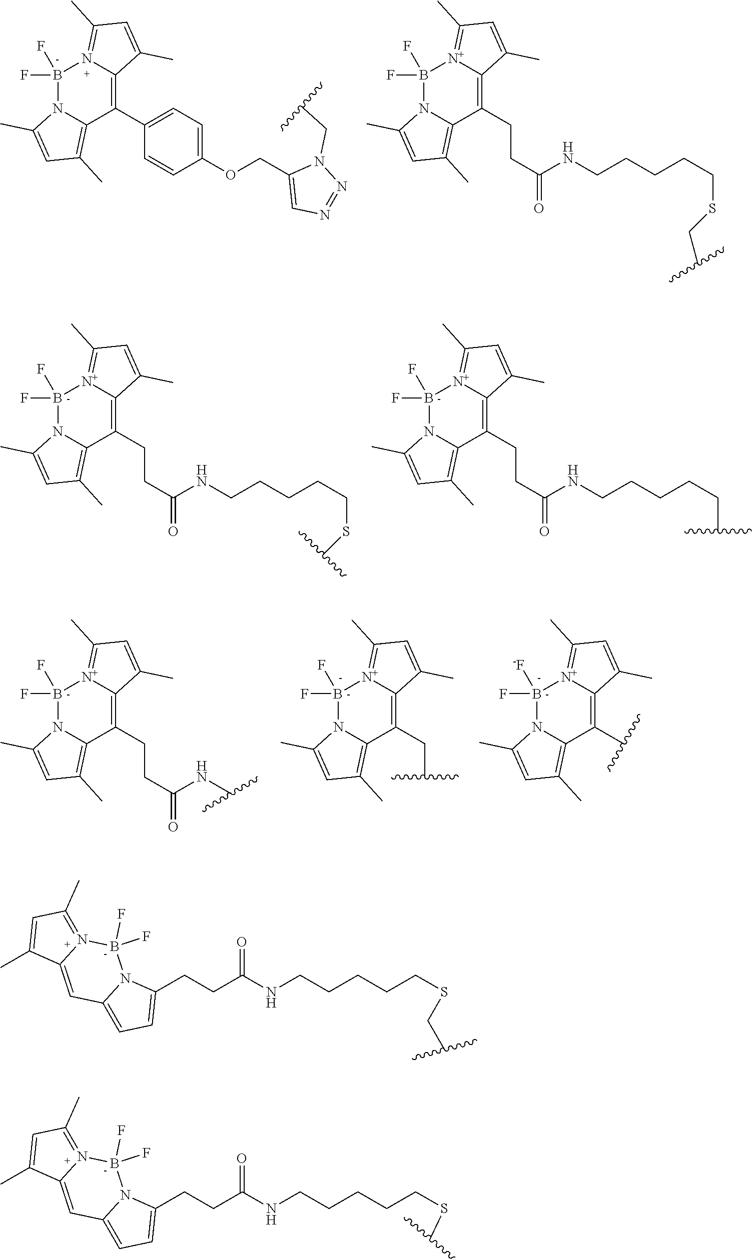

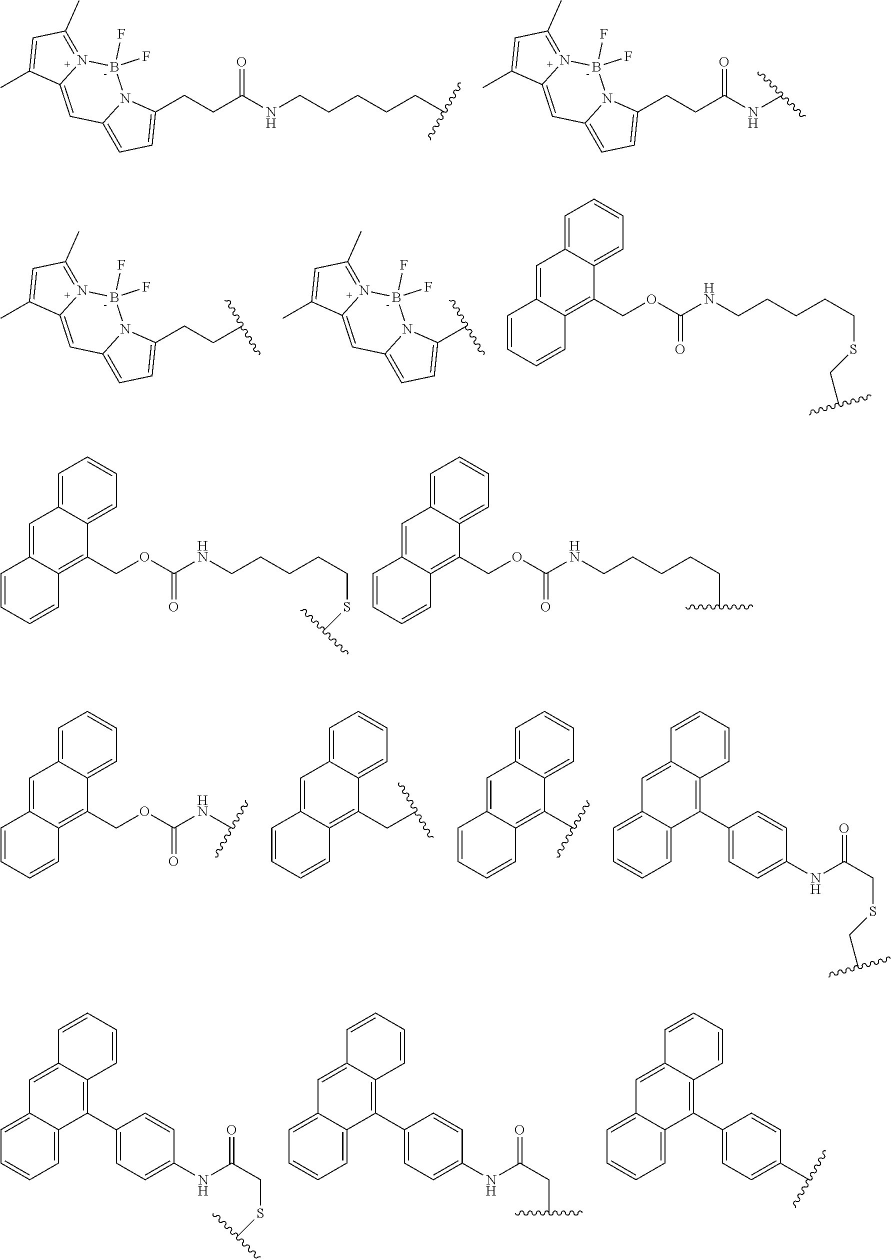

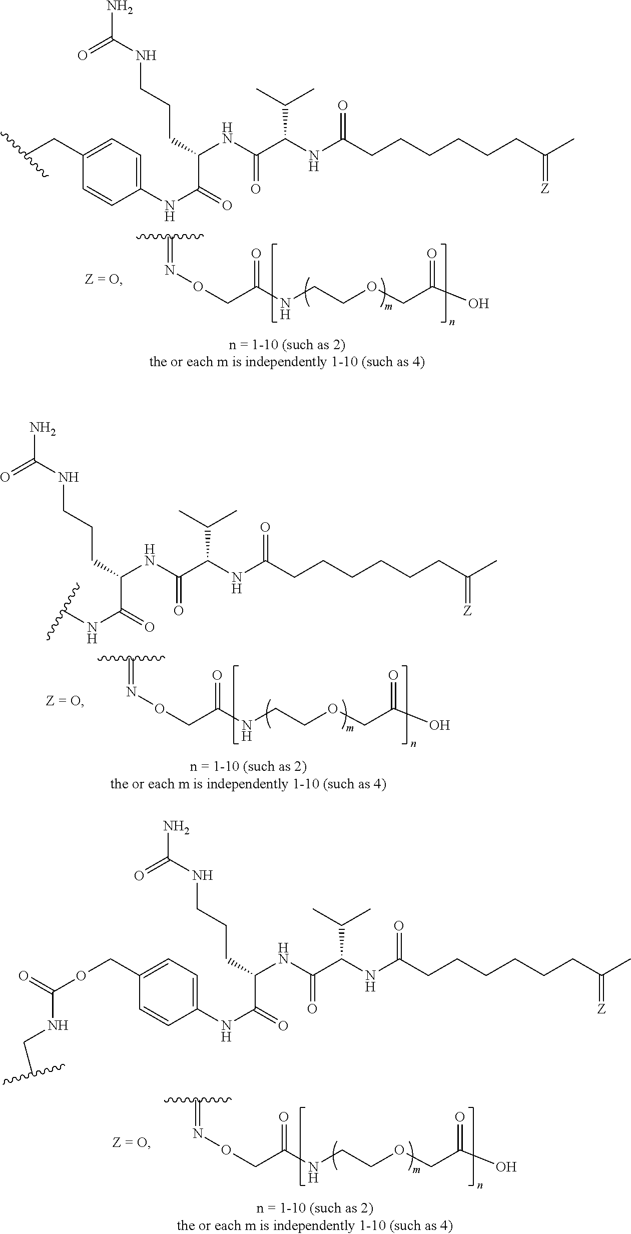

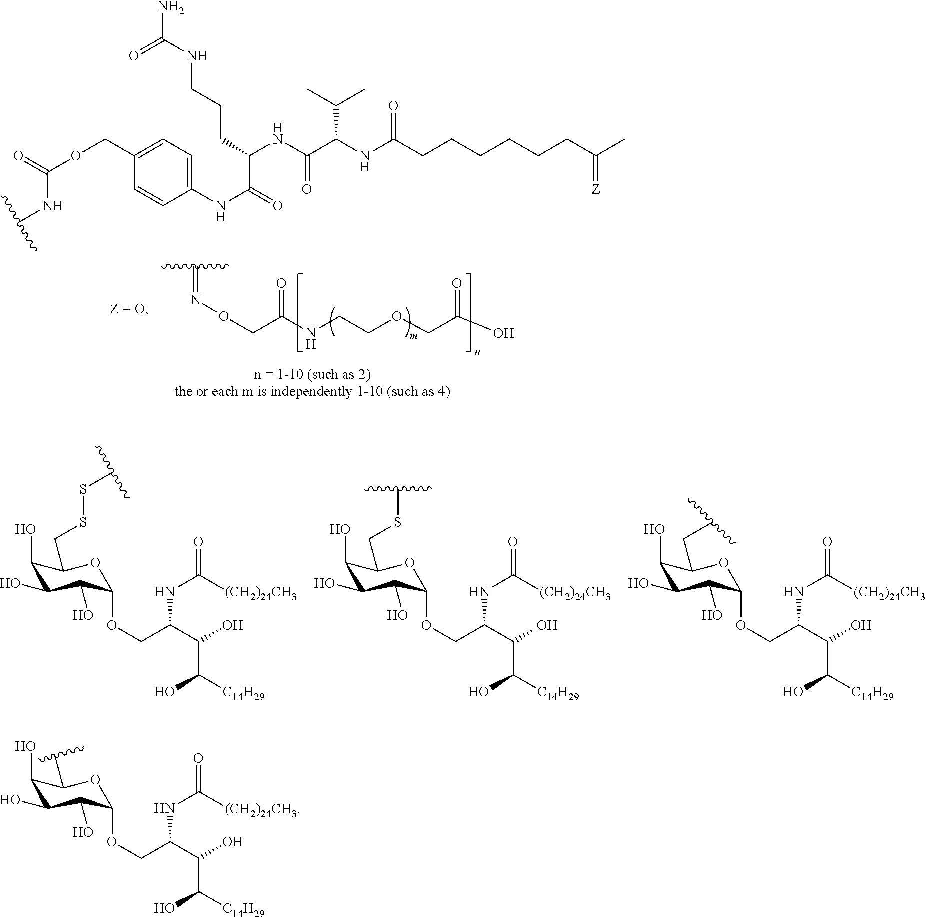

Pharmaceutical acceptable salts of the compounds disclosed herein are also included in the invention. In cases where a compound provided herein is sufficiently basic or acidic to form stable nontoxic acid or base salts, use (including preparation and administration) of the compounds as pharmaceutically acceptable salts may be appropriate.

Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate or α-glycerophosphate. Inorganic salts may also be formed, including hydrochloride, sulfate, nitrate, bicarbonate and carbonate salts.

Pharmaceutically-acceptable base addition salts can be prepared from inorganic and organic bases. Salts from inorganic bases can include, but are not limited to, sodium, potassium, lithium, ammonium, calcium and magnesium salts. Salts derived from organic bases can include, but are not limited to, salts of primary, secondary or tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocycloalkyl amines, diheterocycloalkyl amines, triheterocycloalkyl amines or mixed di- and tri-amines where at least two of the substituents on the amine can be different and can be alkyl, substituted alkyl, alkenyl, substituted alkenyl, cycloalkyl, substituted cycloalkyl, cycloalkenyl, substituted cycloalkenyl, aryl, heteroaryl, heterocycloalkyl and the like. Also included are amines where the two or three substituents, together with the amino nitrogen, form a heterocycloalkyl and heteroaryl group. Non-limiting examples of amines can include, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine and the like. Other carboxylic acid derivatives can be useful, for example, carboxylic acid amides, including carboxamides, lower alkyl carboxamides or dialkyl carboxamides and the like.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Each embodiment described herein is to be applied mutatis mutandis to each and every embodiment unless specifically stated otherwise.

iNKT Cell Agonists

The immunostimulatory compositions of the invention suitably comprise an first agent that comprises an iNKT cell agonist that activates or induces the enrichment of tissue-resident memory T cells (T_{RM} cells).

In some preferred embodiments, the iNKT cell agonist promotes a bias towards the production T_{RM} cells. Such bias may be measured as a percentage of T_{RM} cells in the total memory T cell population present in a tissue of the subject. By way of an example, the iNKT cell agonist may result in greater than about 30%, 35%, 40%, 45%, 50%, 55%, 60%, 61%, 62%, 63%, 64% 65%, 66%, 67%, 68%, 69% or 70% of the total memory T cell population in the being T_{RM} cells. In some particularly preferred embodiments, the iNKT agonist results in greater than 70% of the total memory T cell population in the liver being T_{RM} cells.

Alternatively, the enrichment of T_{RM} cells may be measured by determining the ratio of T_{RM} cells to T_{CM} and T_{EM} cells (i.e., T_{RM} cells:T_{CM}±T_{EM} cells) in a tissue of a subject. For example, the ratio of T_{RM} cells to T_{CM} and T_{EM} cells (i.e., T_{RM} cells:T_{CM}±T_{EM} cells) in the liver of the subject may be greater than about 1:1. Alternatively, the ratio of T_{RM} cells to T_{CM} and T_{EM} cells (i.e., T_{RM} cells:T_{CM}±T_{EM} cells) in the liver of a subject may be greater than about 3:2. By way of a further example, the ratio of T_{RM} cells to T_{CM} and T_{EM} cells (i.e., T_{RM} cells:T_{CM}+T_{EM} cells) in the liver of a subject may be greater than about 2.3:1

Alternatively, the T cell bias may be measured by determining the ratio in a T cell population between the number of T_{RM} cells and the number of at least one other T cell type. For example. In some embodiments, T cell bias may be determined by measuring in a T cell population the ratio between the number of T_{RM} cells and the number of T_{EM} cells. Alternatively, T cell bias may be determined by measuring in a T cell population the ratio between the number of T_{RM} cells and the number of T_{CM} cells. In some particular embodiments, the T cell bias may be determined by measuring in a T cell population the ratio between the number of T_{RM} cells, the number of T_{EM} cells, and the number of T_{CM} cells. Alternatively, the T cell bias may be determined by measuring in a T cell population the ratio between the number of T_{RM} cells and the combined number of T_{EM} cells and T_{CM} cells.

In some embodiments, the tissue in which the T cell bias occurs is selected from one or both of the spleen and liver. In some particularly preferred embodiments, the tissue in which the T cell bias occurs is the liver.

Various methods of measuring the identity or number of T cells of a particular type are well known in the art and equally as applicable for this purpose. By way of an example, the T cell bias may be measures using flow cytometry-based MHC multimer staining methods, flow cytometry-based intracellular cytokine staining (ICS), and Enzyme-Linked ImmunoSpot (ELISPOT) methods.

In one aspect the invention provides a Compound of Formula (I):

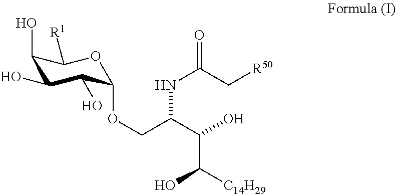

or a pharmaceutically acceptable salt thereof, wherein:
R¹ is selected from hydrogen, halo, alkyl, alkenyl, alkynyl, aryl, acyl, carbocyclyl, heterocyclyl, heteroaryl, alkyloxy, alkenyloxy, alkynyloxy, aryloxy, acyloxy, carbocyclyloxy, heterocyclyloxy, heteroaryloxy, alkylthio, alkenylthio, alkynylthio, arylthio, acylthio, carbocyclylthio, heterocyclylthio, heteroarylthio, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkyloxyacylalkyl, alkylcarbocyclyloxy, alkylheterocyclyloxy, alkylheteroaryloxy, alkylthioalkyl, alkenylthioalkyl, alkynylthioalkyl, alkenylthioalkenyl, alkenylthioalkynyl, alkynylthioalkynyl, arylthioalkyl, alkylacylthio, alkylcarbocyclylthio, alkylheterocyclylalkyl, alkenylheterocyclylalkyl, alkynylheterocyclylalkyl, alkenylheterocyclylalkenyl, alkenylheterocyclylalkynyl, alkynylheterocyclylalkynyl, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, arylacyloxy, arylcarbocyclyloxy, arylheterocyclyloxy, arylheteroaryloxy, alkylthioaryl, alkenylthioaryl, alkynylthioaryl, arylthioaryl, arylacylthio, arylcarbocyclylthio, arylheterocyclylthio, arylheteroarylthio, alkylamino, alkenylamino, alkynylamino, arylamino, acylamino, carbocyclylamino, heterocyclylamino, heteroarylamino, alkylaminoalkyl, alkenylaminoalkyl, alkynylaminoalkyl, alkenylaminoalkenyl, alkenylaminoalkynyl, alkynylaminoalkynyl, arylaminoalkyl, alkylacylamino, alkylcarbocyclylamino, alkylheterocyclylamino, alkylheteroarylamino, alkylaminoaryl, alkenylaminoaryl, alkynylaminoaryl, arylaminoaryl, arylacylamino, arylcarbocyclylamino, arylheterocyclylamino, arylheteroarylamino, alkylamido, alkenylamido, alkynylamido, arylamido, acylamido, carbocyclylamido, heterocyclylamido, heteroarylamido, alkylamidoalkyl, alkenylamidoalkyl, alkynylamidoalkyl, alkenylamidoalkenyl, alkenylamidoalkynyl, alkynylamidoalkynyl, arylamidoalkyl, alkylacylamido, alkylcarbocyclylamido, alkylheterocyclylamido, alkylheteroarylamido, alkylamidoaryl, alkenylamidoaryl, alkynylamidoaryl, arylamidoaryl, arylacylamido, arylcarbocyclylamido, arylheterocyclylamido, arylheteroarylamido, alkylcarbamyl, alkenylcarbamyl, alkynylcarbamyl, arylcarbamyl, acylcarbamyl, carbocyclylcarbamyl, heterocyclylcarbamyl, heteroarylcarbamyl, alkylcarbamylalkyl, alkenylcarbamylalkyl, alkynylcarbamylalkyl, alkenylcarbamylalkenyl, alkenylcarbamylalkynyl, alkynylcarbamylalkynyl, arylcarbamylalkyl, alkylacylcarbamyl, alkylcarbocyclylcarbamyl, alkylheterocyclylcarbamyl, alkylheteroarylcarbamyl, alkylcarbamylaryl, alkenylcarbamylaryl, alkynylcarbamylaryl, arylcarbamylaryl, arylacylcarbamyl, arylcarbocyclylcarbamyl, arylheterocyclylcarbamyl, arylheteroarylcarbamyl, alkyldisulfidyl, alkenyldisulfidyl, alkynyldisulfidyl, aryldisulfidyl, acyldisulfidyl, carbocyclyldisulfidyl, heterocyclyldisulfidyl, heteroaryldisulfidyl, alkyldisulfidylalkyl, alkenyldisulfidylalkyl, alkynyldisulfidylalkyl, alkenyldisulfidylalkenyl, alkenyldisulfidylalkynyl, alkynyldisulfidylalkynyl, aryldisulfidylalkyl, alkylacyldisulfidyl, alkylcarbocyclyldisulfidyl, alkylheterocyclyldisulfidyl, alkylheteroaryldisulfidyl, alkyldisulfidylaryl, alkenyldisulfidylaryl, alkynyldisulfidylaryl, aryldisulfidylaryl, arylacyldisulfidyl, arylcarbocyclyldisulfidyl, arylheterocyclyldisulfidyl, and arylheteroaryldisulfidyl, and wherein R¹ is optionally substituted;
and wherein R¹ is not —CH₂OH;
R⁵⁰ is selected from optionally substituted alkyl, alkenyl, alkynyl.

Preferably R¹ is selected from C₁-C₁₈ alkyl, C₂-C₁₈ alkenyl, C₂-C₁₈ alkynyl, C₆-C₁₈ aryl, C₁-C₁₈ acyl, C₃-C₁₈ carbocyclyl, C₂-C₁₈ heterocyclyl, C₃-C₁₈ heteroaryl, C₁-C₁₈ alkyloxy, C₂-C₁₈ alkenyloxy, C₂-C₁₈ alkynyloxy, C₆-C₁₈ aryloxy, C₁-C₁₈ acyloxy, C₃-C₁₈ carbocyclyloxy, C₂-C₁₈ heterocyclyloxy, C₃-C₁₆ heteroaryloxy, C₁-C₁₈ alkylthio, C₂-C₁₈ alkenylthio, C₂-C₁₈ alkynylthio, C₆-C₁₈ arylthio, C₁-C₁₈ acylthio, C₃-C₁₈ carbocyclylthio, C₂-C₁₈ heterocyclylthio, C₃-C₁₈ heteroarylthio, C₃-C₁₈ alkylalkenyl, C₃-C₁₈ alkylalkynyl C₇-C₂₄ alkylaryl, C₂-C₁₈ alkylacyl, C₄-C₁₈ alkylcarbocyclyl, C₃-C₁₈ alkylheterocyclyl, C₄-C₁₈ alkylheteroaryl, C₂-C₁₈ alkyloxyalkyl, C₃-C₁₈ alkenyloxyalkyl, C₃-C₁₈ alkynyloxyalkyl, C₇-C₂₄ aryloxyalkyl, C₂-C₁₈ alkylacyloxy, C₂₋₁₈ alkyloxyacyl C₂₋₁₈ alkyl, C₄-C₁₈ alkylcarbocyclyloxy, C₃-C₁₈ alkylheterocyclyloxy, C₄-C₁₈ alkylheteroaryloxy, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₄-C₁₉ alkylheterocyclylalkyl, C₄-C₁₈ alkenylheterocyclylalkyl, C₄-C₁₈ alkynylheterocyclylalkyl, C₅-C₁₈ alkenylheterocyclylalkenyl, C₅-C₁₈ alkenylheterocyclylalkynyl, C₅-C₁₈ alkynylheterocyclylalkynyl, C₃-C₁₈ alkylheterocyclylthio, C₄-C₁₈ alkylheteroarylthio, C₄-C₁₈ alkylalkenylalkyl, C₄-C₁₈ alkylalkynylalkyl, C₈-C₂₄ alkylarylalkyl, C₃-C₁₈ alkylacylalkyl, C₁₃-C₂₄ arylalkylaryl, C₁₄-C₂₄ arylalkenylaryl, C₁₄-C₂₄ arylalkynylaryl, C₁₃-C₂₄ arylacylaryl, C₇-C₁₈ arylacyl, C₉-C₁₈ arylcarbocyclyl, C₈-C₁₈ arylheterocyclyl, C₈-C₁₈ arylheteroaryl, C₈-C₁₈ alkenyloxyaryl, C₈-C₁₈ alkynyloxyaryl, C₁₂-C₂₄ aryloxyaryl, C₇-C₁₈ arylacyloxy, C₉-C₁₈ arylcarbocyclyloxy, C₈-C₁₈ arylheterocyclyloxy, C₉-C₁₈ arylheteroaryloxy, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylthio, C₂-C₁₈ alkenylthio, C₂-C₁₈ alkynylthio, C₆-C₁₈ arylthio, C₁-C₁₈ acylthio, C₃-C₁₈ carbocyclylthio, C₂-C₁₈ heterocyclylthio, C₃-C₁₈ heteroarylthio, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₃-C₁₈ alkylheterocyclylthio, C₄-C₁₈ alkylheteroarylthio, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylamino, C₂-C₁₈ alkenylamino, C₂-C₁₈ alkynylamino, C₆-C₁₈ arylamino, C₁-C₁₈ acylamino, C₃-C₁₈ carbocyclylamino, C₂-C₁₈ heterocyclylamino, C₃-C₁₈ heteroarylamino, C₂-C₁₈ alkylaminoalkyl, C₃-C₁₈ alkenylaminoalkyl, C₃-C₁₈ alkynylaminoalkyl, C₄-C₁₈ alkenylaminoalkenyl, C₄-C₁₈ alkenylaminoalkynyl, C₄-C₁₈ alkynylaminoalkynyl, C₇-C₂₄ arylaminoalkyl, C₂-C₁₈ alkylacylamino, C₄-C₁₈ alkylcarbocyclylamino, C₃-C₁₈ alkylheterocyclylamino, C₄-C₁₈ alkylheteroarylamino, C₈-C₁₈ alkylaminoaryl, C₈-C₁₈ alkenylaminoaryl, C₈-C₁₈ alkynylaminoaryl, C₁₂-C₂₄ arylaminoaryl, C₇-C₁₈ arylacylamino, C₉-C₁₈ arylcarbocyclylamino, C₈-C₁₈ arylheterocyclylamino, C₉-C₁₈ arylheteroarylamino, C₁-C₁₈ alkylamido, C₂-C₁₈ alkenylamido, C₂-C₁₈ alkynylamido, C₆-C₁₈ arylamido, C₁-C₁₈ acylamido, C₃-C₁₈ carbocyclylamido, C₂-C₁₈ heterocyclylamido, C₃-C₁₈ heteroarylamido, C₂-C₁₈ alkylamidoalkyl, C₃-C₁₈ alkenylamidoalkyl, C₃-C₁₈ alkynylamidoalkyl, C₄-C₁₈ alkenylamidoalkenyl, C₄-C₁₈ alkenylamidoalkynyl, C₄-C₁₈ alkynylamidoalkynyl, C₇-C₂₄ arylamidoalkyl, C₂-C₁₈ alkylacylamido, C₄-C₁₈ alkylcarbocyclylamido, C₃-C₁₈ alkylheterocyclylamido, C₄-C₁₈ alkylheteroarylamido, C₈-C₁₈ alkylamidoaryl, C₈-C₁₈ alkenylamidoaryl, C₈-C₁₉ alkynylamidoaryl, C₁₂-C₂₄ arylamidoaryl, C₇-C₁₈ arylacylamido, C₉-C₁₈ arylcarbocyclylamido, C₈-C₁₈ arylheterocyclylamido, C₉-C₁₈ arylheteroarylamido, C₁-C₁₈ alkylcarbamyl, C₂-C₁₈ alkenylcarbamyl, C₂-C₁₈ alkynylcarbamyl, C₅-C₁₈ arylcarbamyl, C₁-C₁₈ acylcarbamyl, C₃-C₁₈ carbocyclylcarbamyl, C₂-C₁₈ heterocyclylcarbamyl, C₃-C₁₈ heteroarylcarbamyl, C₂-C₁₈ alkylcarbamylalkyl, C₃-C₁₈ alkenylcarbamylalkyl, C₃-C₁₈ alkynylcarbamylalkyl, C₄-C₁₈ alkenylcarbamylalkenyl, C₄-C₁₈ alkenylcarbamylalkynyl, C₄-C₁₈ alkynylcarbamylalkynyl, C₇-C₂₄ arylcarbamylalkyl, C₂-C₁₈ alkylacylcarbamyl, C₄-C₁₈ alkylcarbocyclylcarbamyl, C₃-C₁₈ alkylheterocyclylcarbamyl, C₄-C₁₈ alkylheteroarylcarbamyl, C₈-C₁₈ alkylcarbamylaryl, C₈-C₁₈ alkenylcarbamylaryl, C₈-C₁₈ alkynylcarbamylaryl, C₁₂-C₂₄ arylcarbamylaryl, C₇-C₁₈ arylacylcarbamyl, C₉-C₁₈ arylcarbocyclylcarbamyl, C₈-C₁₈ arylheterocyclylcarbamyl, C₉-C₁₈ arylheteroarylcarbamyl, C₁-C₁₈ alkyldisulfidyl, C₂—C₁₈ alkenyldisulfidyl, C₂-C₁₈ alkynyldisulfidyl, C₆-C₁₈ aryldisulfidyl, C₁-C₁₈ acyldisulfidyl, C₃-C₁₈ carbocyclyldisulfidyl, C₂-C₁₈ heterocyclyldisulfidyl, C₃-C₁₈ heteroaryldisulfidyl, C₂-C₁₈ alkyldisulfidylalkyl, C₃-C₁₈ alkenyldisulfidylalkyl, C₃-C₁₈ alkynyldisulfidylalkyl, C₄-C₁₈ alkenyldisulfidylalkenyl, C₄-C₁₈ alkenyldisulfidylalkynyl, C₄-C₁₈ alkynyldisulfidylalkynyl, C₇-C₂₄ aryldisulfidylalkyl, C₂-C₁₈ alkylacyldisulfidyl, C₄-C₁₈ alkylcarbocyclyldisulfidyl, C₃-C₁₈ alkylheterocyclyldisulfidyl, C₄-C₁₈ alkylheteroaryldisulfidyl, C₈-C₁₈ alkyldisulfidylaryl, C₈-C₁₈ alkenyldisulfidylaryl, C₈-C₁₈ alkynyldisulfidylaryl, C₁₂-C₂₄ aryldisulfidylaryl, C₇-C₁₈ arylacyldisulfidyl, C₉-C₁₈ arylcarbocyclyldisulfidyl, C₈-C₁₈ arylheterocyclyldisulfidyl, and C₉-C₁₈ arylheteroaryldisulfidyl, and wherein R¹ is optionally substituted;
and wherein R¹ is not —CH₂OH.

For avoidance of any doubt, a prefix denoting the number of carbons in a group, such as “C₈-C₂₄” in “C₈-C₂₄ alkylarylalkyl” may refer to: the number of carbon atoms in the group (e.g., alkylarylalkyl group); or may refer to the number of carbon atoms independently selected for any moiety within that group (e.g., the “alkyl” group). Preferably a prefix denoting the number of carbons in a group, such as “C₈-C₂₄” in “C₈-C₂₄ alkylarylalkyl” refers to the number of carbon atoms in the groupe (such as the whole alkylarylalkyl group).

Still more preferably, R¹ is selected from alkyl (e.g., C₁-C₁₈, C₁-C₆, C₁-C₈, C₈-C₁₈, or C₉-C₁₈), aryl (e.g., C₆-C₁₈), heteroaryl (e.g., C₃-C₁₈), carbocyclyl (e.g., C₃-C₁₈), heterocyclyl (e.g., C₂-C₁₈), alkylaryl (e.g., C₇-C₂₄), alkylheteroaryl (e.g., C₄-C₁₈), alkylcarbocyclyl (e.g., C₄-C₁₈), alkylthioalkyl (e.g., wherein each alkyl is C₁-C₁₈), arylthioalkyl (e.g., wherein each alkyl is C₇-C₁₈), alkylaminoalkyl (e.g., wherein each alkyl is C₁-C₁₈), alkylamidoalkyl (e.g., wherein each alkyl is C₁-C₁₈), alkylheterocyclylalkyl (e.g., C₃-C₁₈), alkylthio (e.g., C₁-C₁₈; such as thiomethyl) and alkylheterocyclyl (e.g., C₃-C₁₈);
and wherein R¹ is not —CH₂OH.

For avoidance of any doubt, where a given R¹ contains two or more moieties (e.g., alkylaryl), each of such moieties may be optionally substituted with one, two, three or more optional substituents as herein defined.

For avoidance of any doubt, where a given R¹ contains two or more moieties (e.g., alkylaryl), the order of the moieties is not intended to be limited to the order in which they are presented. Thus, an R¹ group with two moieties defined as [group A] [group B] (e.g., alkylaryl) is intended to also be a reference to an R¹ group with the two moieties defined as [group B] [group A] (e.g., arylalkyl). All orders of the two or more moieties are contemplated by the present invention.

R¹ may be branched and/or optionally substituted. For example, where the or each R¹ group comprises an optionally substituted alkyl moiety, a preferred optional substituent includes where a —CH₂— group in the alkyl chain is replaced by a group selected from —O—, —S—, —NR^{a}—, —C(O)— (i.e., carbonyl), —C(O)O— (i.e., ester), and —C(O)NR^{a}— (i.e., amide), where R^{a} may be selected from hydrogen, alkyl, alkenyl, alkynyl, aryl, carbocyclyl, heteroaryl, heterocyclyl, arylalkyl, and acyl. For example, R^{a} may be selected from hydrogen, C₁-C₈ alkyl, C₂-C₈ alkenyl, C₂-C₈ alkynyl, C₆-C₁₈ aryl, C₃-C₁₈ carbocyclyl, C₃-C₁₈ heteroaryl, C₂-C₁₈ heterocyclyl, C₇-C₂₄ arylalkyl, and C₁-C₈ acyl.

In some specific embodiments, R⁵⁰ is selected from optionally substituted C₁₈₋₂₆ alkyl, C₁₈₋₂₆ alkenyl, C₁₈₋₂₆ alkynyl. In some even more specific embodiments, R⁵⁰ is —R⁵¹-amido-R⁵² (such as —R⁵¹—NHC(O)—R⁵², or —R⁵¹—C(O)NH—R⁵²) wherein R⁵¹ and R⁵² are each independently selected from alkyl, alkenyl, alkynyl. For example, R⁵¹ and R⁵² may be each independently selected from C₆-C₁₆ alkyl, C₆-C₁₆ alkenyl, C₆-C₁₆ alkynyl.

In one aspect the invention provides a Compound of Formula (II):

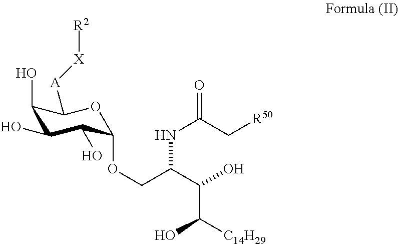

or a pharmaceutically acceptable salt thereof, wherein:
A is alkyl, alkenyl, alkynyl;
X selected from —CR¹⁰R¹¹—, —O—, —S—, —NR^{a}—, —NR^{a}C(O)—, —NR^{a}C(O)O—, —S—S—, heterocyclyl, heteroaryl, wherein R¹⁰ and R¹¹ where present are independently selected from H, halo, C₁₋₂alkyl, and wherein R^{a} may be selected from hydrogen, alkyl, alkenyl, alkynyl, aryl, carbocyclyl, heteroaryl, heterocyclyl, arylalkyl, and acyl;
R² is selected from hydrogen, halo, alkyl, alkenyl, alkynyl, aryl, acyl, carbocyclyl, heterocyclyl, heteroaryl, alkyloxy, alkenyloxy, alkynyloxy, aryloxy, acyloxy, carbocyclyloxy, heterocyclyloxy, heteroaryloxy, alkylthio, alkenylthio, alkynylthio, arylthio, acylthio, carbocyclylthio, heterocyclylthio, heteroarylthio, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkyloxyacylalkyl, alkylcarbocyclyloxy, alkylheterocyclyloxy, alkylheteroaryloxy, alkylthioalkyl, alkenylthioalkyl, alkynylthioalkyl, alkenylthioalkenyl, alkenylthioalkynyl, alkynylthioalkynyl, arylthioalkyl, alkylacylthio, alkylcarbocyclylthio, alkylheterocyclylalkyl, alkenylheterocyclylalkyl, alkynylheterocyclylalkyl, alkenylheterocyclylalkenyl, alkenylheterocyclylalkynyl, alkynylheterocyclylalkynyl, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, arylacyloxy, arylcarbocyclyloxy, arylheterocyclyloxy, arylheteroaryloxy, alkylthioaryl, alkenylthioaryl, alkynylthioaryl, arylthioaryl, arylacylthio, arylcarbocyclylthio, arylheterocyclylthio, arylheteroarylthio, alkylamino, alkenylamino, alkynylamino, arylamino, acylamino, carbocyclylamino, heterocyclylamino, heteroarylamino, alkylaminoalkyl, alkenylaminoalkyl, alkynylaminoalkyl, alkenylaminoalkenyl, alkenylaminoalkynyl, alkynylaminoalkynyl, arylaminoalkyl, alkylacylamino, alkylcarbocyclylamino, alkylheterocyclylamino, alkylheteroarylamino, alkylaminoaryl, alkenylaminoaryl, alkynylaminoaryl, arylaminoaryl, arylacylamino, arylcarbocyclylamino, arylheterocyclylamino, arylheteroarylamino, alkylamido, alkenylamido, alkynylamido, arylamido, acylamido, carbocyclylamido, heterocyclylamido, heteroarylamido, alkylamidoalkyl, alkenylamidoalkyl, alkynylamidoalkyl, alkenylamidoalkenyl, alkenylamidoalkynyl, alkynylamidoalkynyl, arylamidoalkyl, alkylacylamido, alkylcarbocyclylamido, alkylheterocyclylamido, alkylheteroarylamido, alkylamidoaryl, alkenylamidoaryl, alkynylamidoaryl, arylamidoaryl, arylacylamido, arylcarbocyclylamido, arylheterocyclylamido, arylheteroarylamido, alkylcarbamyl, alkenylcarbamyl, alkynylcarbamyl, arylcarbamyl, acylcarbamyl, carbocyclylcarbamyl, heterocyclylcarbamyl, heteroarylcarbamyl, alkylcarbamylalkyl, alkenylcarbamylalkyl, alkynylcarbamylalkyl, alkenylcarbamylalkenyl, alkenylcarbamylalkynyl, alkynylcarbamylalkynyl, arylcarbamylalkyl, alkylacylcarbamyl, alkylcarbocyclylcarbamyl, alkylheterocyclylcarbamyl, alkylheteroarylcarbamyl, alkylcarbamylaryl, alkenylcarbamylaryl, alkynylcarbamylaryl, arylcarbamylaryl, arylacylcarbamyl, arylcarbocyclylcarbamyl, arylheterocyclylcarbamyl, arylheteroarylcarbamyl, alkyldisulfidyl, alkenyldisulfidyl, alkynyldisulfidyl, aryldisulfidyl, acyldisulfidyl, carbocyclyldisulfidyl, heterocyclyldisulfidyl, heteroaryldisulfidyl, alkyldisulfidylalkyl, alkenyldisulfidylalkyl, alkynyldisulfidylalkyl, alkenyldisulfidylalkenyl, alkenyldisulfidylalkynyl, alkynyldisulfidylalkynyl, aryldisulfidylalkyl, alkylacyldisulfidyl, alkylcarbocyclyldisulfidyl, alkylheterocyclyldisulfidyl, alkylheteroaryldisulfidyl, alkyldisulfidylaryl, alkenyldisulfidylaryl, alkynyldisulfidylaryl, aryldisulfidylaryl, arylacyldisulfidyl, arylcarbocyclyldisulfidyl, arylheterocyclyldisulfidyl, and arylheteroaryldisulfidyl, and wherein R² is optionally substituted;
and wherein -A-X—R² is not —CH₂OH;
R⁵⁰ is selected from optionally substituted alkyl, alkenyl, alkynyl.

Preferably, where present, the or each R^{a} may be independently selected from hydrogen, C₁-C₈ alkyl, C₂-C₈ alkenyl, C₂-C₈ alkynyl, C₆-C₁₈ aryl, C₃-C₁₈ carbocyclyl, C₃-C₁₈ heteroaryl, C₂-C₁₈ heterocyclyl, C₇-C₂₄arylalkyl, and C₁-C₈ acyl.

Preferably X is S or —NR^{a}—, wherein R^{a} may be selected from hydrogen, alkyl, alkenyl, alkynyl, aryl, carbocyclyl, heteroaryl, heterocyclyl, arylalkyl, and acyl. More preferably R^{a} may be independently selected from hydrogen, C₁-C₈ alkyl, C₂-C₈ alkenyl, C₂-C₈ alkynyl, C₆-C₁₈ aryl, C₃-C₁₈ carbocyclyl, C₃-C₁₈ heteroaryl, C₂-C₁₈ heterocyclyl, C₇-C₂₄arylalkyl, and C₁-C₈ acyl.

Preferably A is —CR¹²R¹³—, wherein R¹² and R¹³ where present are independently selected from H, halo, C₁₋₂ alkyl. More preferably A is —CH₂—.

Preferably R² is selected from C₁-C₁₈ alkyl, C₂-C₁₈ alkenyl, C₂-C₁₈ alkynyl, C₆-C₁₈ aryl, C₁-C₁₈ acyl, C₃-C₁₈ carbocyclyl, C₂-C₁₈ heterocyclyl, C₃-C₁₈ heteroaryl, C₁-C₁₈ alkyloxy, C₂-C₁₈ alkenyloxy, C₂-C₁₈ alkynyloxy, C₆-C₁₈ aryloxy, C₁-C₁₈ acyloxy, C₃-C₁₈ carbocyclyloxy, C₂-C₁₈ heterocyclyloxy, C₃-C₁₈ heteroaryloxy, C₁-C₁₈ alkylthio, C₂-C₁₈ alkenylthio, C₂-C₁₈ alkynylthio, C₆-C₁₈ arylthio, C₁-C₁₈ acylthio, C₃-C₁₈ carbocyclylthio, C₂-C₁₈ heterocyclylthio, C₃-C₁₈ heteroarylthio, C₃-C₁₈ alkylalkenyl, C₃-C₁₈ alkylalkynyl C₇-C₂₄ alkylaryl, C₂-C₁₈ alkylacyl, C₄-C₁₈ alkylcarbocyclyl, C₃-C₁₈ alkylheterocyclyl, C₄-C₁₈ alkylheteroaryl, C₂-C₁₈ alkyloxyalkyl, C₃-C₁₈ alkenyloxyalkyl, C₃-C₁₈ alkynyloxyalkyl, C₇-C₂₄ aryloxyalkyl, C₂-C₁₈ alkylacyloxy, C₂-18 alkyloxyacyl C₂₋₁₈ alkyl, C₄-C₁₈ alkylcarbocyclyloxy, C₃-C₁₈ alkylheterocyclyloxy, C₄-C₁₈ alkylheteroaryloxy, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₄-C₁₈ alkylheterocyclylalkyl, C₄-C₁₈ alkenylheterocyclylalkyl, C₄-C₁₈ alkynylheterocyclylalkyl, C₅-C₁₈ alkenylheterocyclylalkenyl, C₅-C₁₈ alkenylheterocyclylalkynyl, C₅-C₁₈ alkynylheterocyclylalkynyl, C₃-C₁₈ alkylheterocyclylthio, C₄-C₁₈ alkylheteroarylthio, C₄-C₁₈ alkylalkenylalkyl, C₄-C₁₉ alkylalkynylalkyl, C₈-C₂₄ alkylarylalkyl, C₃-C₁₈ alkylacylalkyl, C₁₃-C₂₄ arylalkylaryl, C₁₄-C₂₄ arylalkenylaryl, C₁₄-C₂₄ arylalkynylaryl, C₁₃-C₂₄ arylacylaryl, C₇-C₁₈ arylacyl, C₉-C₁₈ arylcarbocyclyl, C₈-C₁₈ arylheterocyclyl, C₉-C₁₈ arylheteroaryl, C₈-C₁₈ alkenyloxyaryl, C₈-C₁₈ alkynyloxyaryl, C₁₂-C₂₄ aryloxyaryl, C₇-C₁₈ arylacyloxy, C₉-C₁₈ arylcarbocyclyloxy, C₈-C₁₈ arylheterocyclyloxy, C₉-C₁₈ arylheteroaryloxy, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylthio, C₂-C₁₈ alkenylthio, C₂-C₁₈ alkynylthio, C₆-C₁₈ arylthio, C₁-C₁₈ acylthio, C₃-C₁₈ carbocyclylthio, C₂-C₁₈ heterocyclylthio, C₃-C₁₈ heteroarylthio, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₃-C₁₈ alkylheterocyclylthio, C₄-C₁₈ alkylheteroarylthio, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylamino, C₂-C₁₈ alkenylamino, C₂-C₁₈ alkynylamino, C₆-C₁₈ arylamino, C₁-C₁₉ acylamino, C₃-C₁₉ carbocyclylamino, C₂-C₁₈ heterocyclylamino, C₃-C₁₈ heteroarylamino, C₂-C₁₈ alkylaminoalkyl, C₃-C₁₈ alkenylaminoalkyl, C₃-C₁₈ alkynylaminoalkyl, C₄-C₁₈ alkenylaminoalkenyl, C₄-C₁₈ alkenylaminoalkynyl, C₄-C₁₈ alkynylaminoalkynyl, C₇-C₂₄ arylaminoalkyl, C₂-C₁₈ alkylacylamino, C₄-C₁₈ alkylcarbocyclylamino, C₃-C₁₈ alkylheterocyclylamino, C₄-C₁₈ alkylheteroarylamino, C₈-C₁₈ alkylaminoaryl, C₈-C₁₈ alkenylaminoaryl, C₈-C₁₈ alkynylaminoaryl, C₁₂-C₂₄ arylaminoaryl, C₇-C₁₈ arylacylamino, C₈-C₁₈ arylcarbocyclylamino, C₈-C₁₈ arylheterocyclylamino, C₈-C₁₈ arylheteroarylamino, C₁-C₁₉ alkylamido, C₂-C₁₉ alkenylamido, C₂-C₁₈ alkynylamido, C₆-C₁₈ arylamido, C₁-C₁₈ acylamido, C₃-C₁₈ carbocyclylamido, C₂-C₁₈ heterocyclylamido, C₃-C₁₈ heteroarylamido, C₂-C₁₈ alkylamidoalkyl, C₃-C₁₈ alkenylamidoalkyl, C₃-C₁₈ alkynylamidoalkyl, C₄-C₁₈ alkenylamidoalkenyl, C₄-C₁₈ alkenylamidoalkynyl, C₄-C₁₈ alkynylamidoalkynyl, C₇-C₂₄ arylamidoalkyl, C₂-C₁₈ alkylacylamido, C₄-C₁₈ alkylcarbocyclylamido, C₃-C₁₈ alkylheterocyclylamido, C₄-C₁₈ alkylheteroarylamido, C₈-C₁₈ alkylamidoaryl, C₈-C₁₈ alkenylamidoaryl, C₈-C₁₈ alkynylamidoaryl, C₁₂-C₂₄ arylamidoaryl, C₇-C₁₈ arylacylamido, C₉-C₁₈ arylcarbocyclylamido, C₈-C₁₈ arylheterocyclylamido, C₉-C₁₈ arylheteroarylamido, C₁-C₁₈ alkylcarbamyl, C₂-C₁₈ alkenylcarbamyl, C₂-C₁₈ alkynylcarbamyl, C₅-C₁₈ arylcarbamyl, C₁-C₁₈ acylcarbamyl, C₃-C₁₉ carbocyclylcarbamyl, C₂-C₁₈ heterocyclylcarbamyl, C₃-C₁₈ heteroarylcarbamyl, C₂-C₁₈ alkylcarbamylalkyl, C₃-C₁₈ alkenylcarbamylalkyl, C₃-C₁₈ alkynylcarbamylalkyl, C₄-C₁₈ alkenylcarbamylalkenyl, C₄-C₁₈ alkenylcarbamylalkynyl, C₄-C₁₈ alkynylcarbamylalkynyl, C₇-C₂₄ arylcarbamylalkyl, C₂-C₁₈ alkylacylcarbamyl, C₄-C₁₈ alkylcarbocyclylcarbamyl, C₃-C₁₈ alkylheterocyclylcarbamyl, C₄-C₁₈ alkylheteroarylcarbamyl, C₈-C₁₈ alkylcarbamylaryl, C₈-C₁₈ alkenylcarbamylaryl, C₈-C₁₈ alkynylcarbamylaryl, C₁₂-C₂₄ arylcarbamylaryl, C₇-C₁₈ arylacylcarbamyl, C₉-C₁₈ arylcarbocyclylcarbamyl, C₈-C₁₈ arylheterocyclylcarbamyl, C₉-C₁₈ arylheteroarylcarbamyl, C₁-C₁₈ alkyldisulfidyl, C₂-C₁₈ alkenyldisulfidyl, C₂-C₁₈ alkynyldisulfidyl, C₆-C₁₈ aryldisulfidyl, C₁-C₁₈ acyldisulfidyl, C₃-C₁₈ carbocyclyldisulfidyl, C₂-C₁₈ heterocyclyldisulfidyl, C₃-C₁₈ heteroaryldisulfidyl, C₂-C₁₈ alkyldisulfidylalkyl, C₃-C₁₈ alkenyldisulfidylalkyl, C₃-C₁₈ alkynyldisulfidylalkyl, C₄—C₁₈ alkenyldisulfidylalkenyl, C₄-C₁₈ alkenyldisulfidylalkynyl, C₄-C₁₈ alkynyldisulfidylalkynyl, C₇-C₂₄ aryldisulfidylalkyl, C₂-C₁₈ alkylacyldisulfidyl, C₄-C₁₈ alkylcarbocyclyldisulfidyl, C₃-C₁₈ alkylheterocyclyldisulfidyl, C₄-C₁₈ alkylheteroaryldisulfidyl, C₈-C₁₈ alkyldisulfidylaryl, C₈-C₁₈ alkenyldisulfidylaryl, C₈-C₁₈ alkynyldisulfidylaryl, C₁₂-C₂₄ aryldisulfidylaryl, C₇-C₁₈ arylacyldisulfidyl, C₉-C₁₈ arylcarbocyclyldisulfidyl, C₈-C₁₈ arylheterocyclyldisulfidyl, and C₉-C₁₈ arylheteroaryldisulfidyl, and wherein R² is optionally substituted;
and wherein -A-X—R² is not —CH₂OH.

For avoidance of any doubt, a prefix denoting the number of carbons in a group, such as “C₈-C₂₄” in “C₈-C₂₄ alkylarylalkyl” may refer to: the number of carbon atoms in the group (e.g., alkylarylalkyl group); or may refer to the number of carbon atoms independently selected for any moiety within that group (e.g., the “alkyl” group). Preferably a prefix denoting the number of carbons in a group, such as “C₈-C₂₄” in “C₈-C₂₄ alkylarylalkyl” refers to the number of carbon atoms in the groupe (such as the whole alkylarylalkyl group).

Still more preferably, R² is selected from alkyl (e.g., C₁-C₁₈, C₁-C₆, C₁-C₅, C₈-C₁₈, or C₉-C₁₈), aryl (e.g., C₆-C₁₈), heteroaryl (e.g., C₃-C₁₈), carbocyclyl (e.g., C₃-C₁₈), heterocyclyl (e.g., C₂-C₁₈), alkylaryl (e.g., C₇-C₂₄), alkylheteroaryl (e.g., C₄-C₁₈), alkylcarbocyclyl (e.g., C₄-C₁₈), alkylthioalkyl (e.g., wherein each alkyl is C₁-C₁₈), arylthioalkyl (e.g., wherein each alkyl is C₇-C₁₈), alkylaminoalkyl (e.g., wherein each alkyl is C₁-C₁₈), alkylamidoalkyl (e.g., wherein each alkyl is C₁-C₁₈), alkylheterocyclylalkyl (e.g., C₃-C₁₈), alkylthio (e.g., C₁-C₁₈; such as thiomethyl) and alkylheterocyclyl (e.g., C₃-C₁₈).

For avoidance of any doubt, where a given R² contains two or more moieties (e.g., alkylaryl), each of such moieties may be optionally substituted with one, two, three or more optional substituents as herein defined.

For avoidance of any doubt, where a given R² contains two or more moieties (e.g., alkylaryl), the order of the moieties is not intended to be limited to the order in which they are presented. Thus, an R² group with two moieties defined as [group A] [group B] (e.g., alkylaryl) is intended to also be a reference to an R² group with the two moieties defined as [group B] [group A] (e.g., arylalkyl). All orders of the two or more moieties are contemplated by the present invention.

R² may be branched and/or optionally substituted. For example, where the or each R¹ group comprises an optionally substituted alkyl moiety, a preferred optional substituent includes where a —CH₂— group in the alkyl chain is replaced by a group selected from —O—, —S—, —NR^{a}—, —C(O)— (i.e., carbonyl), —C(O)O— (i.e., ester), and —C(O)NR^{a}— (i.e., amide), where R^{a} may be selected from hydrogen, alkyl, alkenyl, alkynyl, aryl, carbocyclyl, heteroaryl, heterocyclyl, arylalkyl, and acyl. For example, R^{a} may be selected from hydrogen, C₁-C₈ alkyl, C₂-C₈ alkenyl, C₂-C₈ alkynyl, C₆-C₁₈ aryl, C₃-C₁₈ carbocyclyl, C₃-C₁₈ heteroaryl, C₂-C₁₈ heterocyclyl, C₇-C₂₄ arylalkyl, and C₁-C₈ acyl.

In some specific embodiments, R⁵⁰ is selected from optionally substituted C₁₈₋₂₆ alkyl, C₁₈₋₂₆ alkenyl, C₁₈-₂₆ alkynyl. In some even more specific embodiments, R⁵⁰ is —R⁵¹-amido-R⁵² (such as —R⁵¹—NHC(O)—R⁵², or —R⁵¹—C(O)NH—R⁵²) wherein R⁵¹ and R⁵² are each independently selected from alkyl, alkenyl, alkynyl. For example, R⁵¹ and R⁵² may be each independently selected from C₆-C₁₈ alkyl, C₈-C₁₆ alkenyl, C₆-C₁₆ alkynyl.

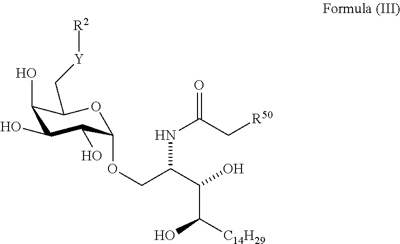

or a pharmaceutically acceptable salt thereof, wherein:
Y is selected from —S—, —NR^{a}—, —NR^{a}C(O)—, —NR^{a}C(O)O—, —S—S—, heterocyclyl, heteroaryl, wherein R^{a} may be selected from hydrogen, alkyl, alkenyl, alkynyl, aryl, carbocyclyl, heteroaryl, heterocyclyl, arylalkyl, and acyl;
R² is selected from hydrogen, halo, alkyl, alkenyl, alkynyl, aryl, acyl, carbocyclyl, heterocyclyl, heteroaryl, alkyloxy, alkenyloxy, alkynyloxy, aryloxy, acyloxy, carbocyclyloxy, heterocyclyloxy, heteroaryloxy, alkylthio, alkenylthio, alkynylthio, arylthio, acylthio, carbocyclylthio, heterocyclylthio, heteroarylthio, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkyloxyacylalkyl, alkylcarbocyclyloxy, alkylheterocyclyloxy, alkylheteroaryloxy, alkylthioalkyl, alkenylthioalkyl, alkynylthioalkyl, alkenylthioalkenyl, alkenylthioalkynyl, alkynylthioalkynyl, arylthioalkyl, alkylacylthio, alkylcarbocyclylthio, alkylheterocyclylalkyl, alkenylheterocyclylalkyl, alkynylheterocyclylalkyl, alkenylheterocyclylalkenyl, alkenylheterocyclylalkynyl, alkynylheterocyclylalkynyl, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, arylacyloxy, arylcarbocyclyloxy, arylheterocyclyloxy, arylheteroaryloxy, alkylthioaryl, alkenylthioaryl, alkynylthioaryl, arylthioaryl, arylacylthio, arylcarbocyclylthio, arylheterocyclylthio, arylheteroarylthio, alkylamino, alkenylamino, alkynylamino, arylamino, acylamino, carbocyclylamino, heterocyclylamino, heteroarylamino, alkylaminoalkyl, alkenylaminoalkyl, alkynylaminoalkyl, alkenylaminoalkenyl, alkenylaminoalkynyl, alkynylaminoalkynyl, arylaminoalkyl, alkylacylamino, alkylcarbocyclylamino, alkylheterocyclylamino, alkylheteroarylamino, alkylaminoaryl, alkenylaminoaryl, alkynylaminoaryl, arylaminoaryl, arylacylamino, arylcarbocyclylamino, arylheterocyclylamino, arylheteroarylamino, alkylamido, alkenylamido, alkynylamido, arylamido, acylamido, carbocyclylamido, heterocyclylamido, heteroarylamido, alkylamidoalkyl, alkenylamidoalkyl, alkynylamidoalkyl, alkenylamidoalkenyl, alkenylamidoalkynyl, alkynylamidoalkynyl, arylamidoalkyl, alkylacylamido, alkylcarbocyclylamido, alkylheterocyclylamido, alkylheteroarylamido, alkylamidoaryl, alkenylamidoaryl, alkynylamidoaryl, arylamidoaryl, arylacylamido, arylcarbocyclylamido, arylheterocyclylamido, arylheteroarylamido, alkylcarbamyl, alkenylcarbamyl, alkynylcarbamyl, arylcarbamyl, acylcarbamyl, carbocyclylcarbamyl, heterocyclylcarbamyl, heteroarylcarbamyl, alkylcarbamylalkyl, alkenylcarbamylalkyl, alkynylcarbamylalkyl, alkenylcarbamylalkenyl, alkenylcarbamylalkynyl, alkynylcarbamylalkynyl, arylcarbamylalkyl, alkylacylcarbamyl, alkylcarbocyclylcarbamyl, alkylheterocyclylcarbamyl, alkylheteroarylcarbamyl, alkylcarbamylaryl, alkenylcarbamylaryl, alkynylcarbamylaryl, arylcarbamylaryl, arylacylcarbamyl, arylcarbocyclylcarbamyl, arylheterocyclylcarbamyl, arylheteroarylcarbamyl, alkyldisulfidyl, alkenyldisulfidyl, alkynyldisulfidyl, aryldisulfidyl, acyldisulfidyl, carbocyclyldisulfidyl, heterocyclyldisulfidyl, heteroaryldisulfidyl, alkyldisulfidylalkyl, alkenyldisulfidylalkyl, alkynyldisulfidylalkyl, alkenyldisulfidylalkenyl, alkenyldisulfidylalkynyl, alkynyldisulfidylalkynyl, aryldisulfidylalkyl, alkylacyldisulfidyl, alkylcarbocyclyldisulfidyl, alkylheterocyclyldisulfidyl, alkylheteroaryldisulfidyl, alkyldisulfidylaryl, alkenyldisulfidylaryl, alkynyldisulfidylaryl, aryldisulfidylaryl, arylacyldisulfidyl, arylcarbocyclyldisulfidyl, arylheterocyclyldisulfidyl, and arylheteroaryldisulfidyl, and wherein R² is optionally substituted;
R⁵⁰ is selected from optionally substituted alkyl, alkenyl, alkynyl.

Preferably R^{a} is hydrogen.

Preferably Y is selected from —S—, —NH—, —NHC(O)—, —NHC(O)O—, —S—S—, triazolyl.

Preferably R² in Formula (III) is selected from C₁-C₁₈ alkyl, C₂-C₁₈ alkenyl, C₂-C₁₈ alkynyl, C₆-C₁₈ aryl, C₁-C₁₈ acyl, C₃-C₁₈ carbocyclyl, C₂-C₁₈ heterocyclyl, C₃-C₁₈ heteroaryl, C₁-C₁₈ alkyloxy, C₂-C₁₈ alkenyloxy, C₂-C₁₈ alkynyloxy, C₆-C₁₈ aryloxy, C₁-C₁₈ acyloxy, C₃-C₁₈ carbocyclyloxy, C₂-C₁₈ heterocyclyloxy, C₃-C₁₈ heteroaryloxy, C₁-C₁₈ alkylthio, C₂-C₁₈ alkenylthio, C₂-C₁₈ alkynylthio, C₆-C₁₈ arylthio, C₁-C₁₈ acylthio, C₃-C₁₈ carbocyclylthio, C₂-C₁₈ heterocyclylthio, C₃-C₁₈ heteroarylthio, C₃-C₁₈ alkylalkenyl, C₃-C₁₈ alkylalkynyl C₇-C₂₄ alkylaryl, C₂-C₁₈ alkylacyl, C₄-C₁₈ alkylcarbocyclyl, C₃-C₁₈ alkylheterocyclyl, C₄-C₁₈ alkylheteroaryl, C₂-C₁₈ alkyloxyalkyl, C₃-C₁₈ alkenyloxyalkyl, C₃-C₁₈ alkynyloxyalkyl, C₇-C₂₄ aryloxyalkyl, C₂-C₁₈ alkylacyloxy, C₂₋₁₈ alkyloxyacyl C₂₋₁₈ alkyl, C₄-C₁₈ alkylcarbocyclyloxy, C₃-C₁₈ alkylheterocyclyloxy, C₄-C₁₈ alkylheteroaryloxy, C₂-C₁₈ alkylthioalkyl, C₃-C₁₈ alkenylthioalkyl, C₃-C₁₈ alkynylthioalkyl, C₄-C₁B alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₄-C₁₈ alkylheterocyclylalkyl, C₄-C₁₈ alkenylheterocyclylalkyl, C₄-C₁₈ alkynylheterocyclylalkyl, C₅-C₁₈ alkenylheterocyclylalkenyl, C₅-C₁₈ alkenylheterocyclylalkynyl, C₅-C₁₈ alkynylheterocyclylalkynyl, C₃-C₁₈ alkylheterocyclylthio, C₄-C₁₈ alkylheteroarylthio, C₄-C₁₈ alkylalkenylalkyl, C₄-C₁₈ alkylalkynylalkyl, C₈-C₂₄ alkylarylalkyl, C₃-C₁₈ alkylacylalkyl, C₁₃-C₂₄ arylalkylaryl, C₁₄-C₂₄ arylalkenylaryl, C₁₄-C₂₄ arylalkynylaryl, C₁₃-C₂₄ arylacylaryl, C₇-C₁₈ arylacyl, C₉-C₁₈ arylcarbocyclyl, C₈-C₁₈ arylheterocyclyl, C₉-C₁₈ arylheteroaryl, C₈-C₁₈ alkenyloxyaryl, C₈-C₁₈ alkynyloxyaryl, C₁₂-C₂₄ aryloxyaryl, C₇-C₁₈ arylacyloxy, C₉-C₁₈ arylcarbocyclyloxy, C₈-C₁₈ arylheterocyclyloxy, C₉-C₁₈ arylheteroaryloxy, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylthio, C₂-C₁₈ alkenylthio, C₂-C₁₈ alkynylthio, C₆-C₁₈ arylthio, C₁-C₁₈ acylthio, C₃-C₁₈ carbocyclylthio, C₂-C₁₈ heterocyclylthio, C₃-C₁₈ heteroarylthio, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₃-C₁₈ alkylheterocyclylthio, C₄-C₁₈ alkylheteroarylthio, C₈-C₁₈ alkylthioaryl, C₈-C₁₈ alkenylthioaryl, C₈—C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylamino, C₂-C₁₈ alkenylamino, C₂-C₁₈ alkynylamino, C₆-C₁₈ arylamino, C₁-C₁₈ acylamino, C₃-C₁₈ carbocyclylamino, C₂-C₁₈ heterocyclylamino, C₃-C₁₈ heteroarylamino, C₂-C₁₈ alkylaminoalkyl, C₃-C₁₈ alkenylaminoalkyl, C₃-C₁₈ alkynylaminoalkyl, C₄-C₁₈ alkenylaminoalkenyl, C₄-C₁₈ alkenylaminoalkynyl, C₄-C₁₈ alkynylaminoalkynyl, C₇-C₂₄ arylaminoalkyl, C₂-C₁₈ alkylacylamino, C₄-C₁₈ alkylcarbocyclylamino, C₃-C₁₈ alkylheterocyclylamino, C₄-C₁₈ alkylheteroarylamino, C₈-C₁₈ alkylaminoaryl, C₈-C₁₈ alkenylaminoaryl, C₈-C₁₈ alkynylaminoaryl, C₁₂-C₂₄ arylaminoaryl, C₇-C₁₈ arylacylamino, C₉-C₁₈ arylcarbocyclylamino, C₈-C₁₈ arylheterocyclylamino, C₉-C₁₈ arylheteroarylamino, C₁-C₁₈ alkylamido, C₂-C₁₈ alkenylamido, C₂-C₁₈ alkynylamido, C₆-C₁₈ arylamido, C₁-C₁₈ acylamido, C₃-C₁₈ carbocyclylamido, C₂-C₁₈ heterocyclylamido, C₃-C₁₈ heteroarylamido, C₂-C₁₈ alkylamidoalkyl, C₃-C₁₈ alkenylamidoalkyl, C₃-C₁₈ alkynylamidoalkyl, C₄-C₁₈ alkenylamidoalkenyl, C₄-C₁₈ alkenylamidoalkynyl, C₄-C₁₈ alkynylamidoalkynyl, C₇-C₂₄ arylamidoalkyl, C₂-C₁₈ alkylacylamido, C₄-C₁₈ alkylcarbocyclylamido, C₃-C₁₈ alkylheterocyclylamido, C₄-C₁₈ alkylheteroarylamido, C₈-C₁₈ alkylamidoaryl, C₈-C₁₈ alkenylamidoaryl, C₈-C₁₈ alkynylamidoaryl, C₁₂-C₂₄ arylamidoaryl, C₇-C₁₈ arylacylamido, C₉-C₁₈ arylcarbocyclylamido, C₈-C₁₈ arylheterocyclylamido, C₉-C₁₈ arylheteroarylamido, C₁-C₁₈ alkylcarbamyl, C₂-C₁₈ alkenylcarbamyl, C₂-C₁₈ alkynylcarbamyl, C₆-C₁₈ arylcarbamyl, C₁-C₁₈ acylcarbamyl, C₃-C₁₈ carbocyclylcarbamyl, C₂-C₁₈ heterocyclylcarbamyl, C₃-C₁₈ heteroarylcarbamyl, C₂-C₁₈ alkylcarbamylalkyl, C₃-C₁₈ alkenylcarbamylalkyl, C₃-C₁₈ alkynylcarbamylalkyl, C₄-C₁₈ alkenylcarbamylalkenyl, C₄-C₁₈ alkenylcarbamylalkynyl, C₄-C₁₈ alkynylcarbamylalkynyl, C₇-C₂₄ arylcarbamylalkyl, C₂-C₁₈ alkylacylcarbamyl, C₄-C₁₈ alkylcarbocyclylcarbamyl, C₃-C₁₈ alkylheterocyclylcarbamyl, C₄-C₁₈ alkylheteroarylcarbamyl, C₈-C₁₈ alkylcarbamylaryl, C₈-C₁₈ alkenylcarbamylaryl, C₈-C₁₈ alkynylcarbamylaryl, C₁₂-C₂₄ arylcarbamylaryl, C₇-C₁₈ arylacylcarbamyl, C₉-C₁₈ arylcarbocyclylcarbamyl, C₈-C₁₈ arylheterocyclylcarbamyl, C₉-C₁₈ arylheteroarylcarbamyl, C₁-C₁₈ alkyldisulfidyl, C₂-C₁₈ alkenyldisulfidyl, C₂-C₁₈ alkynyldisulfidyl, C₆-C₁₈ aryldisulfidyl, C₁-C₁₈ acyldisulfidyl, C₃-C₁₈ carbocyclyldisulfidyl, C₂-C₁₈ heterocyclyldisulfidyl, C₃-C₁₈ heteroaryldisulfidyl, C₂-C₁₈ alkyldisulfidylalkyl, C₃-C₁₈ alkenyldisulfidylalkyl, C₃-C₁₈ alkynyldisulfidylalkyl, C₄-C₁₈ alkenyldisulfidylalkenyl, C₄-C₁₈ alkenyldisulfidylalkynyl, C₄-C₁₈ alkynyldisulfidylalkynyl, C₇-C₂₄ aryldisulfidylalkyl, C₂-C₁₈ alkylacyldisulfidyl, C₄-C₁₈ alkylcarbocyclyldisulfidyl, C₃-C₁₈ alkylheterocyclyldisulfidyl, C₄-C₁₈ alkylheteroaryldisulfidyl, C₈-C₁₈ alkyldisulfidylaryl, C₈-C₁₈ alkenyldisulfidylaryl, C₈-C₁₈ alkynyldisulfidylaryl, C₁₂-C₂₄ aryldisulfidylaryl, C₇-C₁₈ arylacyldisulfidyl, C₉-C₁₈ arylcarbocyclyldisulfidyl, C₈-C₁₈ arylheterocyclyldisulfidyl, and C₉-C₁₈ arylheteroaryldisulfidyl, and wherein R² is optionally substituted.

For avoidance of any doubt, a prefix denoting the number of carbons in a group, such as “C₈-C₂₄” in “C₈-C₂₄ alkylarylalkyl” may refer to: the number of carbon atoms in the group (e.g., alkylarylalkyl group); or may refer to the number of carbon atoms independently selected for any moiety within that group (e.g., the “alkyl” group). Preferably a prefix denoting the number of carbons in a group, such as “C₈-C₂₄” in “C₈-C₂₄ alkylarylalkyl” refers to the number of carbon atoms in the groupe (such as the whole alkylarylalkyl group).

Still more preferably, R² in Formula (III) is selected from alkyl (e.g., C₁-C₁₈, C₁-C₆, C₁-C₅, C₈-C₁₈, or C₉-C₁₃), aryl (e.g., C₆-C₁₈), heteroaryl (e.g., C₃-C₁₈), carbocyclyl (e.g., C₃-C₁₈), heterocyclyl (e.g., C₂-C₁₈), alkylaryl (e.g., C₇-C₂₄), alkylheteroaryl (e.g., C₄-C₁₈), alkylcarbocyclyl (e.g., C₄-C₁₈), alkylthioalkyl (e.g., wherein each alkyl is C₁-C₁₈), arylthioalkyl (e.g., wherein each alkyl is C₇-C₁₈), alkylaminoalkyl (e.g., wherein each alkyl is C₁-C₁₈), alkylamidoalkyl (e.g., wherein each alkyl is C₁-C₁₈), alkylheterocyclylalkyl (e.g., C₃-C₁₈), alkylthio (e.g., C₁-C₁₈; such as thiomethyl) and alkylheterocyclyl (e.g., C₃-C₁₈).

For avoidance of any doubt, where a given R² contains two or more moieties (e.g., alkylaryl), each of such moieties may be optionally substituted with one, two, three or more optional substituents as herein defined.

For avoidance of any doubt, where a given R² contains two or more moieties (e.g., alkylaryl), the order of the moieties is not intended to be limited to the order in which they are presented. Thus, an R² group with two moieties defined as [group A] [group B] (e.g., alkylaryl) is intended to also be a reference to an R² group with the two moieties defined as [group B] [group A] (e.g., arylalkyl). All orders of the two or more moieties are contemplated by the present invention.

R² may be branched and/or optionally substituted. For example, where the or each R¹ group comprises an optionally substituted alkyl moiety, a preferred optional substituent includes where a —CH₂— group in the alkyl chain is replaced by a group selected from —O—, —S—, —NR^{a}—, —C(O)— (i.e., carbonyl), —C(O)O— (i.e., ester), and —C(O)NR^{a}— (i.e., amide), where R^{a} may be selected from hydrogen, alkyl, alkenyl, alkynyl, aryl, carbocyclyl, heteroaryl, heterocyclyl, arylalkyl, and acyl. For example, R^{a} may be selected from hydrogen, C₁-C₈ alkyl, C₂-C₈ alkenyl, C₂-C₈ alkynyl, C₆-C₁₈ aryl, C₃-C₁₈ carbocyclyl, C₃-C₁₈ heteroaryl, C₂-C₁₈ heterocyclyl, C₇-C₂₄ arylalkyl, and C₁-C₈ acyl.

In some specific embodiments, R⁵⁰ is selected from optionally substituted C₁₈₋₂₆ alkyl, C₁₈₋₂₆ alkenyl, C₁₈-₂₆ alkynyl. In some even more specific embodiments, R⁵⁰ is —R⁵¹-amido-R⁵² (such as —R⁵¹—NHC(O)—R⁵², or —R⁵¹—C(O)NH—R⁵²) wherein R⁵¹ and R⁵² are each independently selected from alkyl, alkenyl, alkynyl. For example, R⁵¹ and R⁵² may be each independently selected from C₆-C₁₆ alkyl, C₆-C₁₆ alkenyl, C₆-C₁₆ alkynyl.

In one aspect the invention provides a compound selected from the following GROUP I compounds:

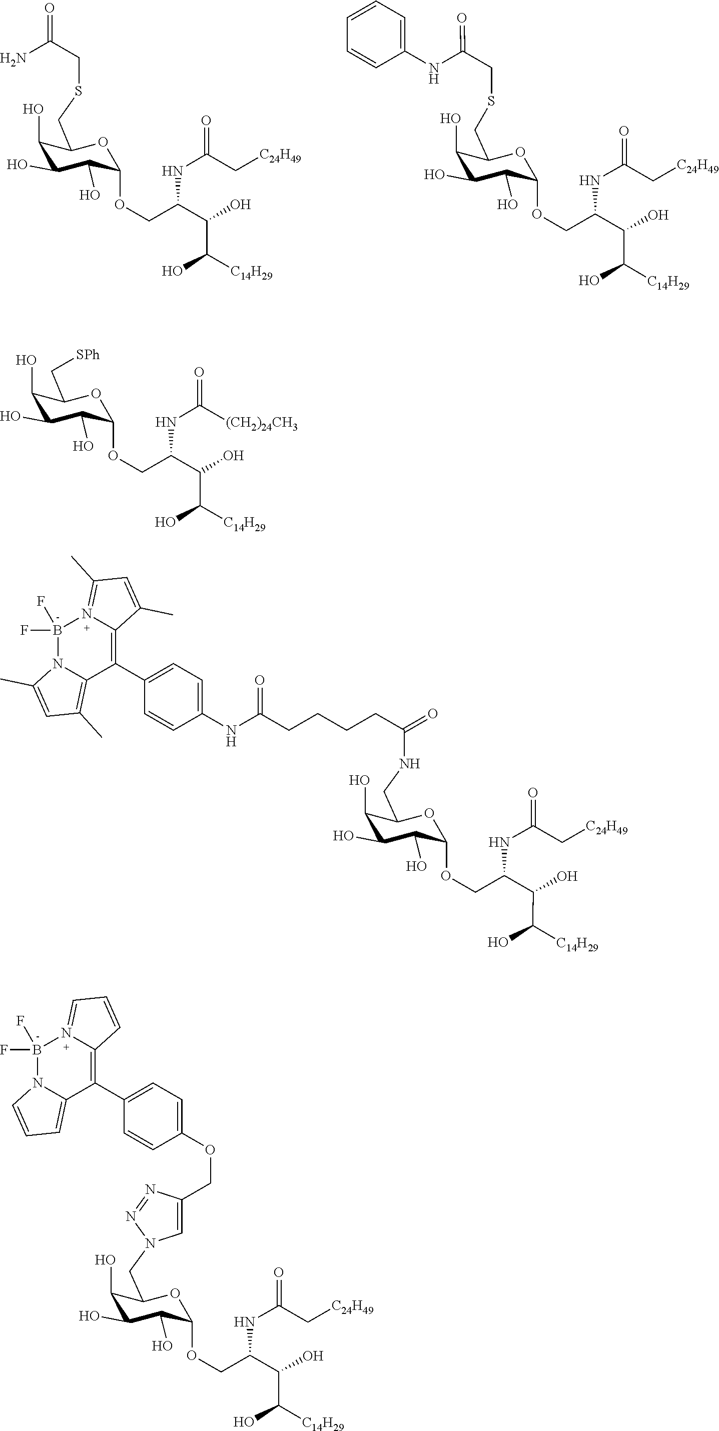

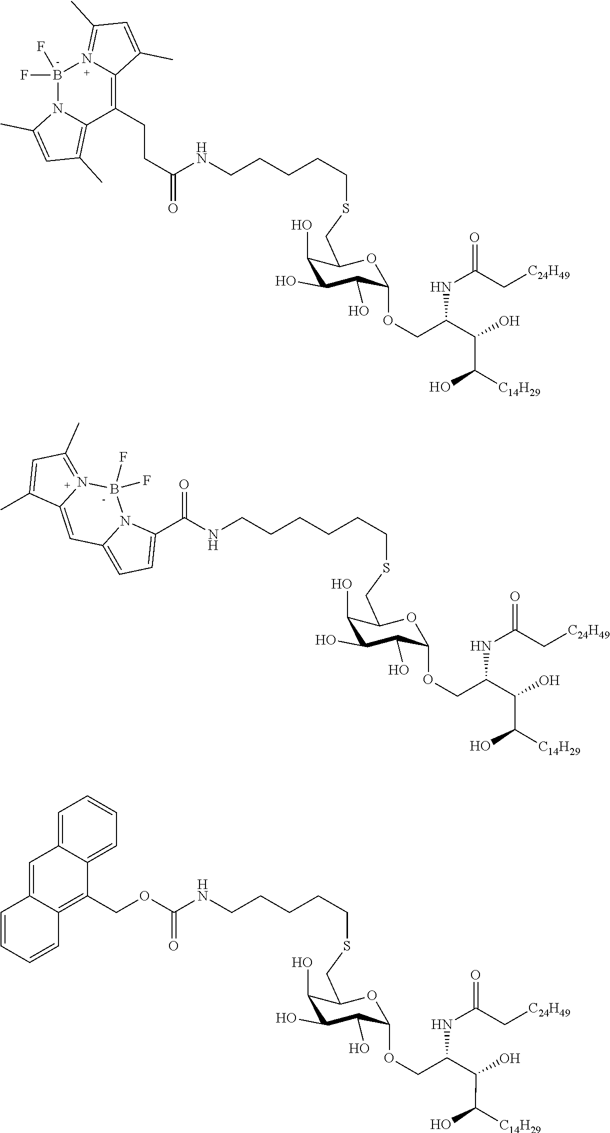

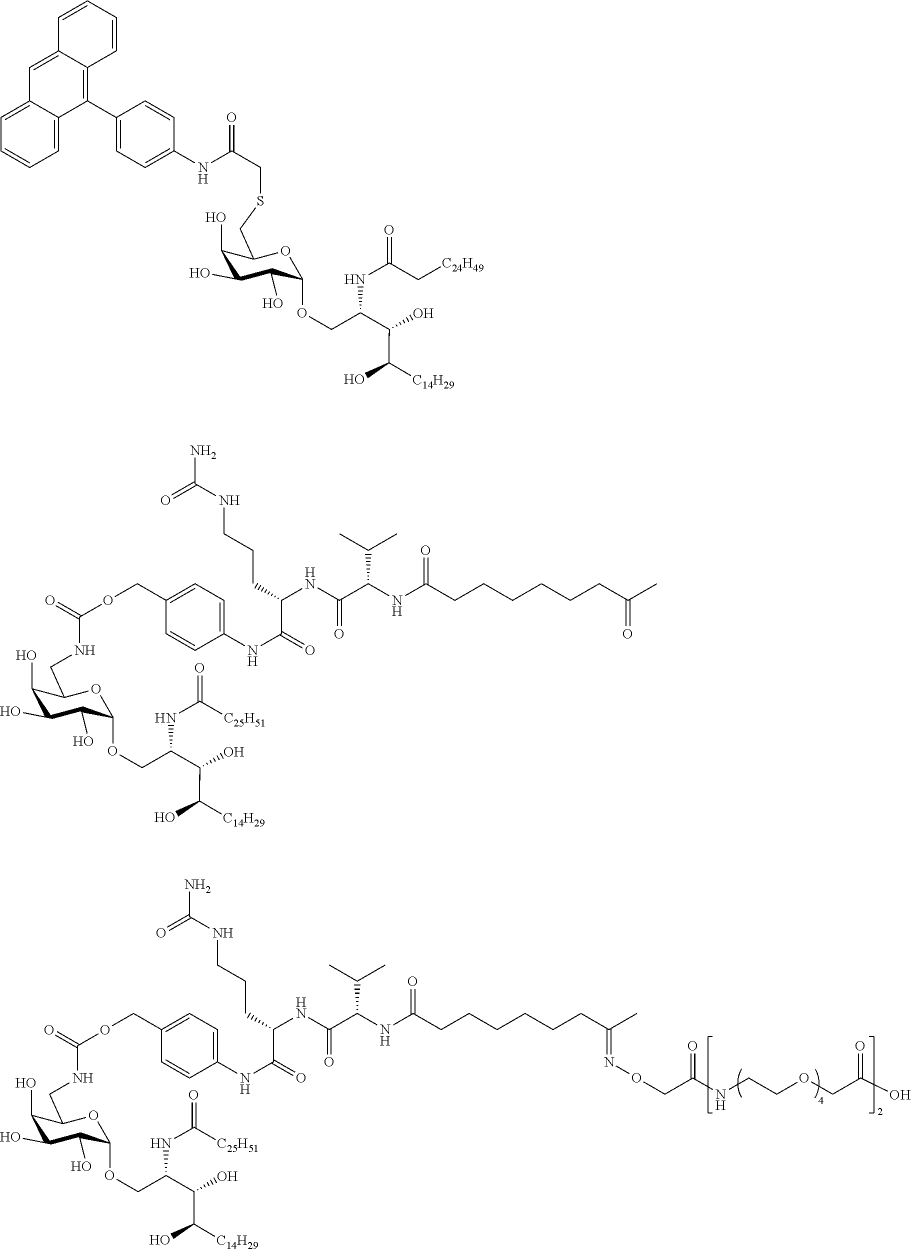

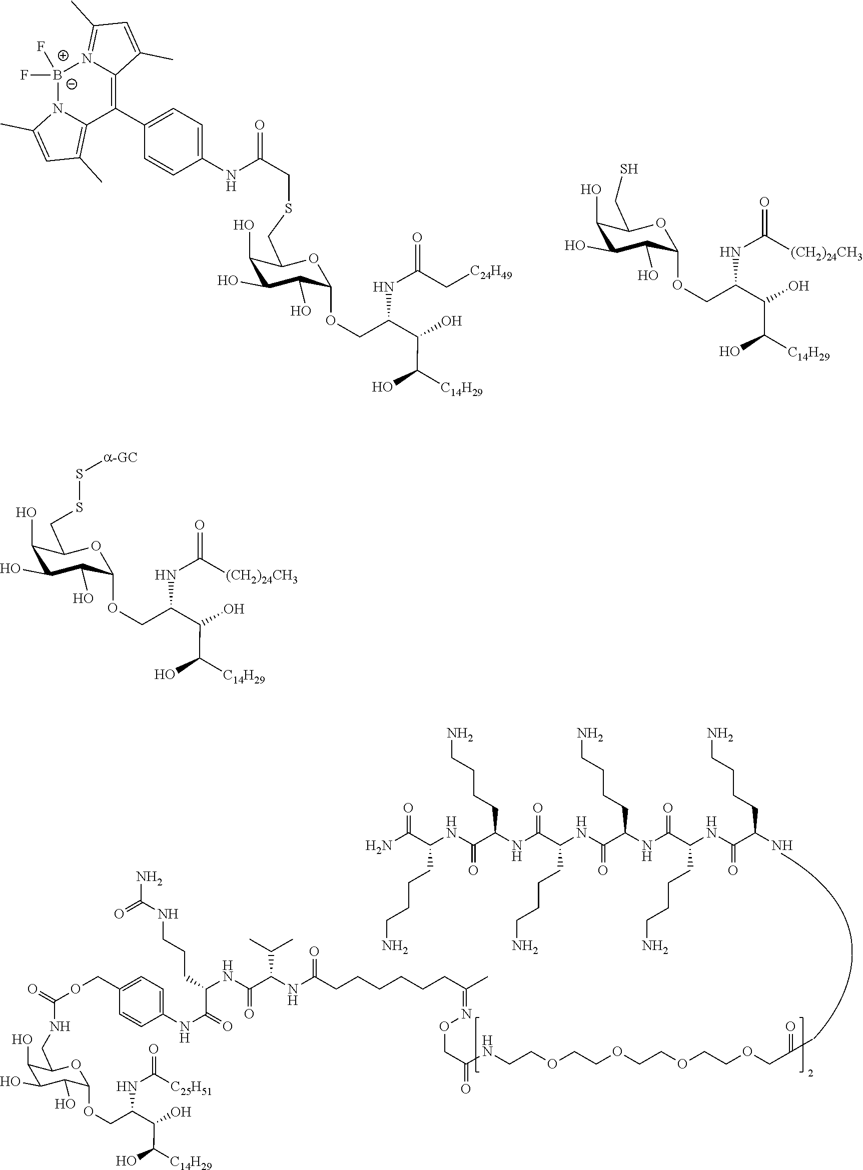

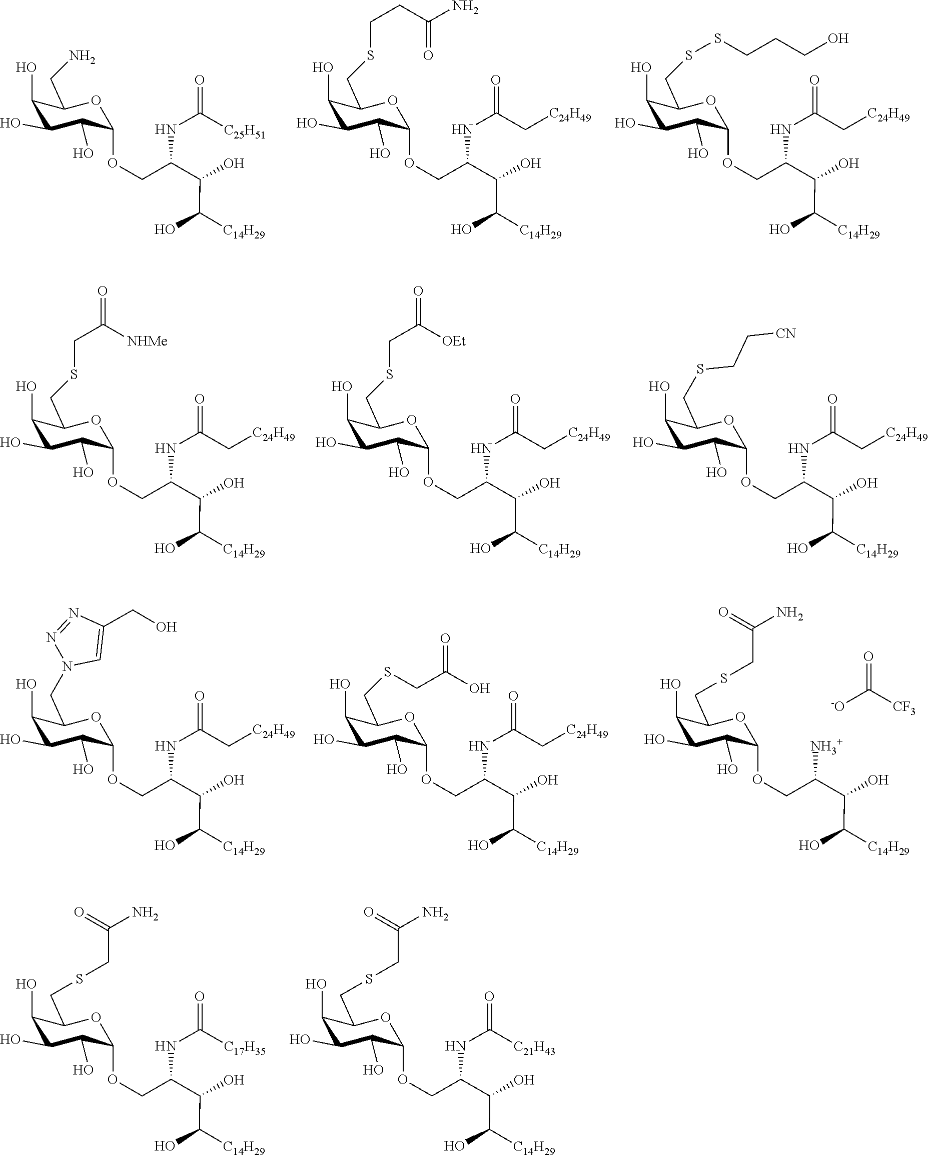

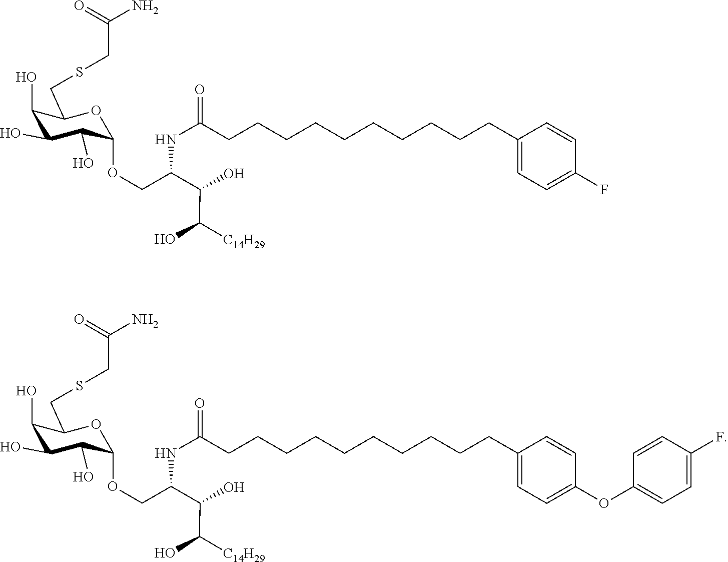

In one aspect the invention provides a compound selected from the following GROUP II compounds:

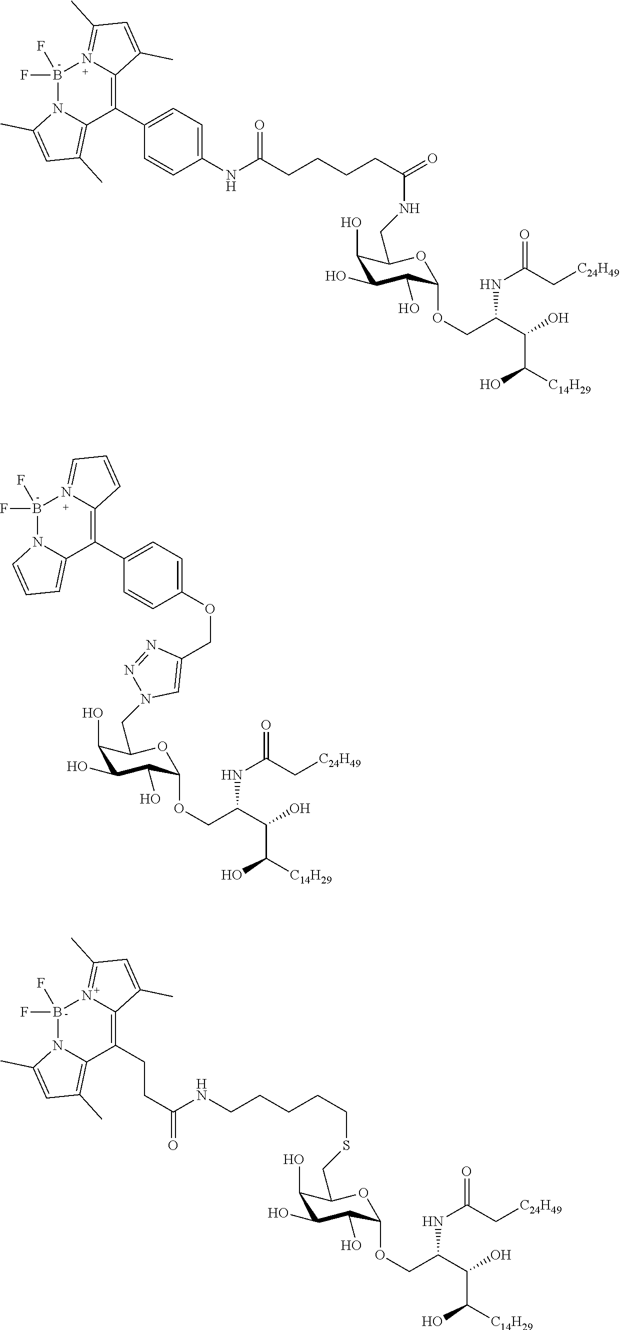

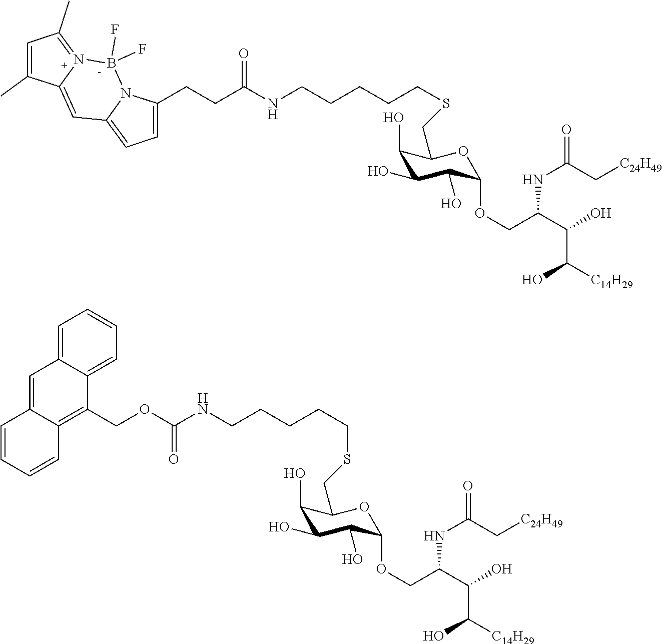

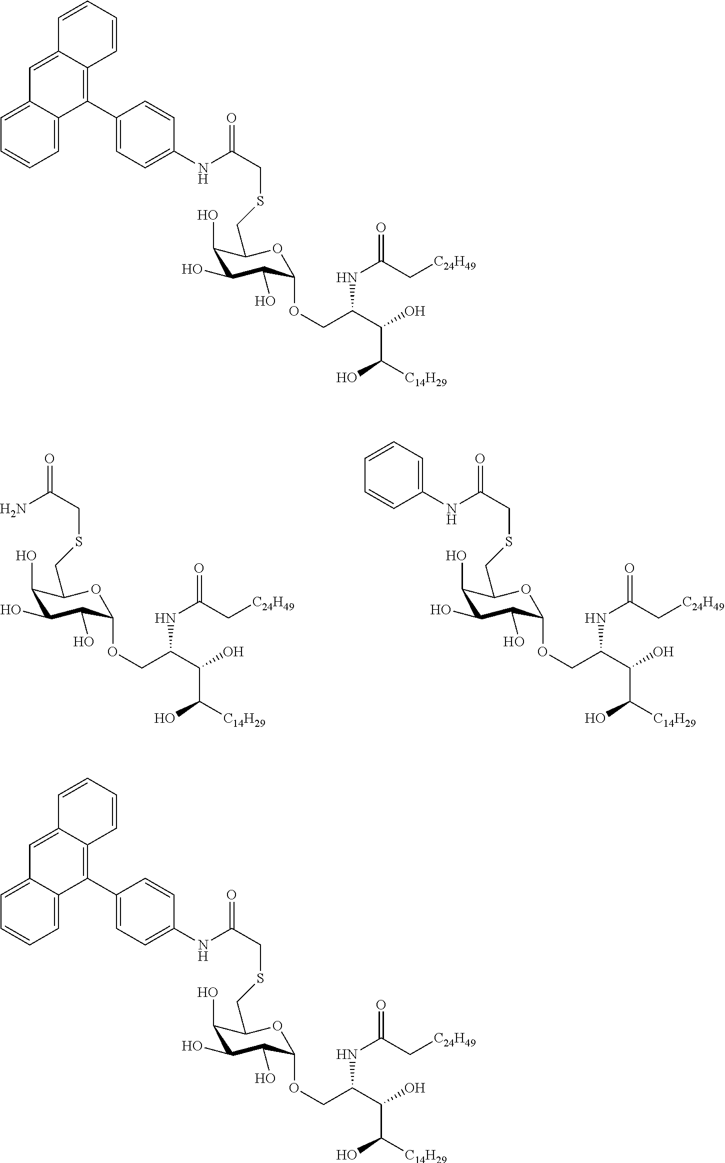

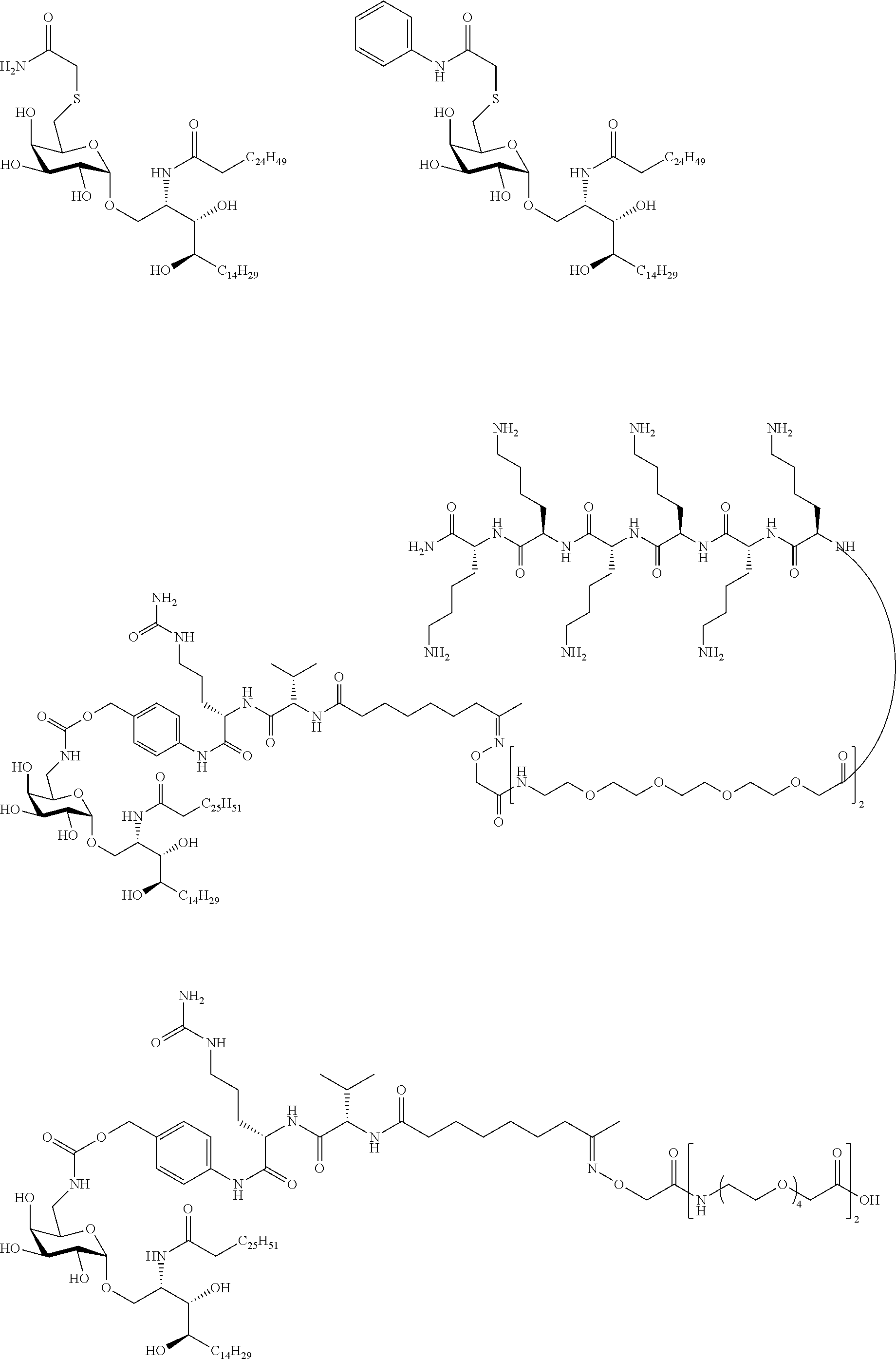

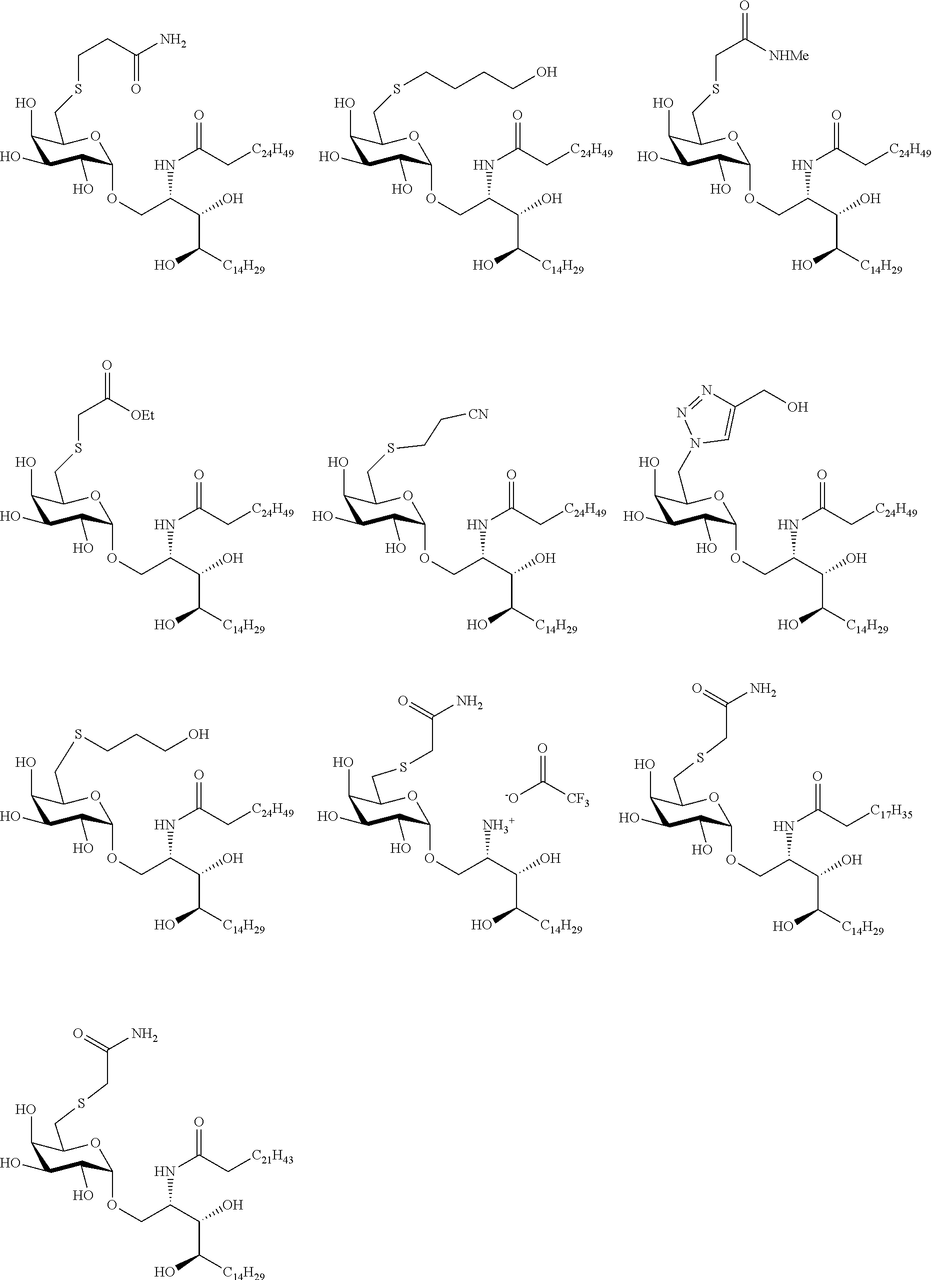

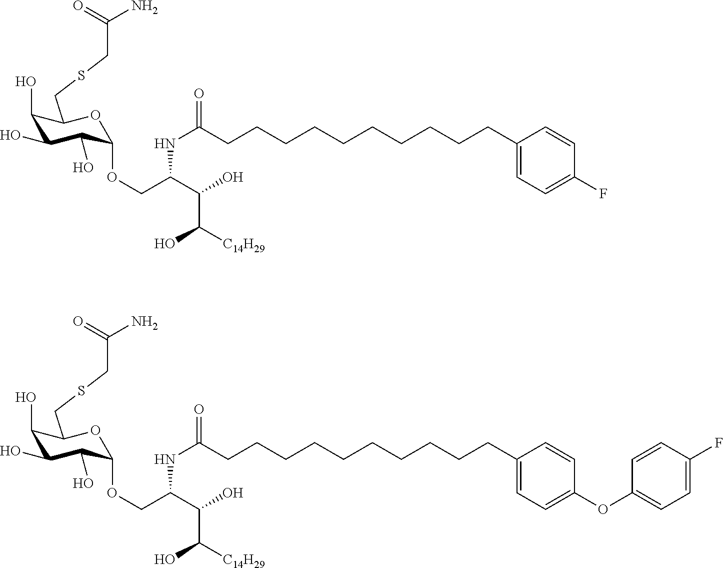

### Immune Stimulating Agents

### 1.1 Peptide Antigens

The present invention contemplates the use in the compositions of the invention of an immune stimulator comprising any antigen that corresponds to at least a portion of a target antigen of interest for stimulating an immune response to the target antigen. The antigen that corresponds to at least a portion of the target antigen may be in soluble form (e.g., a peptide or polypeptide) when expressed.

Target antigens useful in the present invention are typically proteinaceous molecules, representative examples of which include polypeptides and peptides. Target antigens may be selected from endogenous antigens produced by a host or exogenous antigens that are foreign to the host. Suitable endogenous antigens include, but are not restricted to, cancer or tumour antigens. Non-limiting examples of cancer or tumour antigens include antigens from a cancer or tumour selected from ABL1 proto-oncogene, AIDS related cancers, acoustic neuroma, acute lymphocytic leukemia, acute myeloid leukemia, adenoid cystic carcinoma (ACC), adrenocortical cancer, agnogenic myeloid metaplasia, alopecia, alveolar soft-part sarcoma, anal cancer, angiosarcoma, aplastic anemia, astrocytoma, ataxia-telangiectasia, basal cell carcinoma (skin), bladder cancer, bone cancers, bowel cancer, brain stem glioma, brain and CNS tumours, breast cancer, CNS tumours, carcinoid tumours, cervical cancer, childhood brain tumours, childhood cancer, childhood leukemia, childhood soft tissue sarcoma, chondrosarcoma, choriocarcinoma, chronic lymphocytic leukemia, chronic myeloid leukemia, colorectal cancers, cutaneous T-cell lymphoma, dermatofibrosarcoma protuberans, desmoplastic small round cell tumour, ductal carcinoma, endocrine cancers, endometrial cancer, ependymoma, oesophageal cancer, Ewing's Sarcoma, extra-hepatic bile duct cancer, eye cancer, melanoma, retinoblastoma, fallopian tube cancer, Fanconi anemia, fibrosarcoma, gall bladder cancer, gastric cancer, gastrointestinal cancers, gastrointestinal-carcinoid-tumour, genitourinary cancers, germ cell tumours, gestational-trophoblastic-disease, glioma, gynaecological cancers, haematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, intraocular melanoma, islet cell cancer, Kaposi's sarcoma, kidney cancer, Langerhans cell histiocytosis, laryngeal cancer, leiomyosarcoma, leukemia, Li-Fraumeni syndrome, lip cancer, liposarcoma, liver cancer, lung cancer, lymphedema, lymphoma, Hodgkin's lymphoma, non-Hodgkin's lymphoma, male breast cancer, malignant-rhabdoid tumour of kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, nasal cancer, nasopharyngeal cancer, nephroblastoma, neuroblastoma, neurofibromatosis, Nijmegen breakage syndrome, non-melanoma skin cancer, non-small-cell-lung-cancer (NSCLC), ocular cancers, esophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal tumours, pituitary cancer, polycythemia vera, prostate cancer, rare cancers and associated disorders, renal cell carcinoma, retinoblastoma, rhabdomyosarcoma, Rothmund-Thomson syndrome, salivary gland cancer, sarcoma, schwannoma, Sezary syndrome, skin cancer, small cell lung cancer (SCLC), small intestine cancer, soft tissue sarcoma, spinal cord tumours, squamous-cell-carcinoma-(skin), stomach cancer, synovial sarcoma, testicular cancer, thymus cancer, thyroid cancer, transitional-cell-cancer-(bladder), transitional-cell-cancer-(renal-pelvis-/-ureter), trophoblastic cancer, urethral cancer, urinary system cancer, uroplakins, uterine sarcoma, uterus cancer, vaginal cancer, vulva cancer, Waldenstrom's macroglobulinemia, Wilms' tumour. In certain embodiments, the cancer or tumour relates to nasopharyngeal cancer. Illustrative examples of nasopharyngeal cancer antigens include EBNA-1, LMP-1, LMP-2, or a combination thereof. Other tumour-specific antigens include, but are not limited to MAGE, MART-1/Melan-A, gplOO, Dipeptidyl peptidase IV (DPPIV), adenosine deaminase-binding protein (ABP), cyclophilin B, Colorectal associated antigen (CRC)-CO17-1A/GA733, Carcinoembryonic Antigen (CEA) and its immunogenic epitopes CAP-1 and CAP-2, etvó, amll, Prostate Specific Antigen (PSA) and its immunogenic epitopes PSA-1, PSA-2, and PSA-3, prostate-specific membrane antigen (PSMA), T-cell receptor/CD3ζ chain, MAGE-family of tumour antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumour antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gplOOPmdm, PRAME, NY-ESO-1, CDC27, adenomatous polyposis coli protein (APC), fodrin, Connexin 37, Ig-idiotype, gp75, GM2 and GD2 gangliosides, viral products such as human papilloma virus proteins, Smad family of tumour antigens, Imp-1, EBV-encoded nuclear antigen (EBNA)-1, brain glycogen phosphorylase, SSX-1, SSX-2 (HOM-MEL-40), SSX-3, SSX-4, SSX-5, SCP-1, CT-7, and c-erbB-2.

Foreign or exogenous antigens are suitably selected from antigens of pathogenic organisms. Exemplary pathogenic organisms include, but are not limited to, viruses, bacteria, fungi, parasites, algae and protozoa and amoebae. Illustrative viruses include viruses responsible for diseases including, but not limited to, severe acute respiratory syndrome (SARS), measles, mumps, rubella, poliomyelitis, hepatitis A, B (e.g., GenBank Accession No. E02707), and C (e.g., GenBank Accession No. E06890), as well as other hepatitis viruses, influenza, adenovirus (e.g., types 4 and 7), rabies (e.g., GenBank Accession No. M34678), yellow fever, Epstein-Barr virus and other herpesviruses such as papillomavirus, Ebola virus, Japanese encephalitis (e.g., GenBank Accession No. E07883), dengue (e.g., GenBank Accession No. M24444), hantavirus, Sendai virus, respiratory syncytial virus, othromyxoviruses, vesicular stomatitis virus, Visna virus, cytomegalovirus and human immunodeficiency virus (HFV) (e.g., GenBank Accession No. U18552).

Any suitable antigen derived from such viruses are useful in the practice of the present invention. For example, illustrative examples of coronavirus antigens include, but are not limited to, the spike protein (e.g., the RBD of the spike protein) and nucleocapsid. Illustrative retroviral antigens derived from HFV include, but are not limited to, antigens such as gene products of the gag, pol, and env genes, the Nef protein, reverse transcriptase, and other HIV components. Illustrative examples of hepatitis viral antigens include, but are not limited to, antigens such as the S, M, and L proteins of hepatitis B virus, the pre-S antigen of hepatitis B virus, and other hepatitis (e.g., hepatitis A, B, and C) viral components (such as hepatitis C viral RNA). Illustrative examples of influenza viral antigens include, but are not limited to, antigens such as hemagglutinin and neuraminidase and other influenza viral components. Illustrative examples of measles viral antigens include, but are not limited to, antigens such as the measles virus fusion protein and other measles virus components. Illustrative examples of rubella viral antigens include, but are not limited to, antigens such as proteins E1 and E2 and other rubella virus components; rotaviral antigens such as VP7sc and other rotaviral components. Illustrative examples of cytomegaloviral antigens include, but are not limited to, antigens such as envelope glycoprotein B and other cytomegaloviral antigen components. Non-limiting examples of respiratory syncytial viral antigens include antigens such as the RSV fusion protein, the M2 protein and other respiratory syncytial viral antigen components. Illustrative examples of herpes simplex viral antigens include, but are not limited to, antigens such as immediate early proteins, glycoprotein D, and other herpes simplex viral antigen components. Non-limiting examples of varicella zoster viral antigens include antigens such as gp1, gp2, and other varicella zoster viral antigen components. Non-limiting examples of Japanese encephalitis viral antigens include antigens such as proteins E, M-E, M-E-NS1, NS1, NS1-NS2A, and other Japanese encephalitis viral antigen components. Representative examples of rabies viral antigens include, but are not limited to, antigens such as rabies glycoprotein, rabies nucleoprotein and other rabies viral antigen components. Illustrative examples of papillomavirus antigens include, but are not limited to, the L1 and L2 capsid proteins as well as the E6/E7 antigens associated with cervical cancers (see, Fundamental Virology, Second Edition, eds. Fields, B. N. and Knipe, D. M., 1991, Raven Press, New York, for additional examples of viral antigens).

Illustrative examples of fungi include Acremonium spp., Aspergillus spp., Basidiobolus spp., Bipolaris spp., Blastomyces dermatidis, Candida spp., Cladophialophora carrionii, Coccoidiodes immitis, Conidiobolus spp., Cryptococcus spp., Curvularia spp., Epidermophyton spp., Exophiala jeanselmei, Exserohilum spp., Fonsecaea compacta, Fonsecaea pedrosoi, Fusarium oxysporum, Fusarium solani, Geotrichum candidum, Histoplasma capsulatum var. capsulatum, Histoplasma capsulatum var. duboisii, Hortaea werneckii, Lacazia loboi, Lasiodiplodia theobromae, Leptosphaeria senegalensis, Madurella grisea, Madurella mycetomatis, Malassezia furfur, Microsporum spp., Neotestudina rosatii, Onychocola canadensis, Paracoccidioides brasiliensis, Phialophora verrucosa, Piedraia hortae, Piedra iahortae, Pityriasis versicolor, Pseudallesheria boydii, Pyrenochaeta romeroi, Rhizopus arrhizus, Scopulariopsis brevicaulis, Scytalidium dimidiatum, Sporothrix schenckii, Trichophyton spp., Trichosporon spp., Zygomcete fungi, Absidia corymbifera, Rhizomucor pusillus and Rhizopus arrhizus. Thus, representative fungal antigens that can be used in the compositions and methods of the present invention include, but are not limited to, Candida fungal antigen components; Histoplasma fungal antigens such as heat shock protein 60 (HSP60) and other Histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components.

Illustrative examples of bacteria include bacteria that are responsible for diseases including, but not restricted to, diphtheria (e.g., Corynebacterium diphtheria), pertussis (e.g., Bordetella pertussis, GenBank Accession No. M35274), tetanus (e.g., Clostridium tetani, GenBank Accession No. M64353), tuberculosis (e.g., Mycobacterium tuberculosis), bacterial pneumonias (e.g., Haemophilus influenzae), cholera (e.g., Vibrio cholerae), anthrax (e.g., Bacillus anthracis), typhoid, plague, shigellosis (e.g., Shigella dysenteriae), botulism (e.g., Clostridium botulinum), salmonellosis (e.g., GenBank Accession No. L03833), peptic ulcers (e.g., Helicobacter pylori), Legionnaire's Disease, Lyme disease (e.g., GenBank Accession No. U59487), Other pathogenic bacteria include Escherichia coli, Clostridium perfringens, Pseudomonas aeruginosa, Staphylococcus aureus and Streptococcus pyogenes. Thus, bacterial antigens which can be used in the compositions and methods of the invention include, but are not limited to: pertussis bacterial antigens such as pertussis toxin, filamentous hemagglutinin, pertactin, FIM2, FIM3, adenylate cyclase and other pertussis bacterial antigen components; diphtheria bacterial antigens such as diphtheria toxin or toxoid and other diphtheria bacterial antigen components; tetanus bacterial antigens such as tetanus toxin or toxoid and other tetanus bacterial antigen components, streptococcal bacterial antigens such as M proteins and other streptococcal bacterial antigen components; gram-negative bacilli bacterial antigens such as lipopolysaccharides and other gram-negative bacterial antigen components; Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components, pneumococcal bacterial antigens such as pneumolysin, pneumococcal capsular polysaccharides and other pnermiococcal bacterial antigen components; Haemophilus influenza bacterial antigens such as capsular polysaccharides and other Haemophilus influenza bacterial antigen components; anthrax bacterial antigens such as anthrax protective antigen and other anthrax bacterial antigen components; rickettsiae bacterial antigens such as rompA and other rickettsiae bacterial antigen component. Also included with the bacterial antigens described herein are any other bacterial, mycobacterial, mycoplasmal, rickettsial, or chlamydial antigens.

Illustrative examples of protozoa include protozoa that are responsible for diseases including, but not limited to, malaria (e.g., GenBank Accession No. X53832), hookworm, onchocerciasis (e.g., GenBank Accession No. M27807), schistosomiasis (e.g., GenBank Accession No. L05198), toxoplasmosis, trypanosomiasis, leishmaniasis, giardiasis (GenBank Accession No. M33641), amoebiasis, filariasis (e.g., GenBank Accession No. J03266), borreliosis, and trichinosis. Thus, protozoal antigens which can be used in the compositions and methods of the invention include, but are not limited to: Plasmodium falciparum antigens such as merozoite surface antigens, sporozoite surface antigens, circumsporozoite antigens, gametocyte/gamete surface antigens, blood-stage antigen of 155/RESA and other plasmodial antigen components; toxoplasma antigens such as SAG-I, p30 and other toxoplasmal antigen components; Schistosomae antigens such as glutathione-S-transferase, paramyosin, and other schistosomal antigen components; Leishmania major and other leishmaniae antigens such as gp63, lipophosphoglycan and its associated protein and other leishmanial antigen components; and Trypanosoma cruzi antigens such as the 75-77 kDa antigen, the 56 kDa antigen, and other Trypanosoma/antigen components.

The present invention also contemplates toxin components as antigens. Illustrative examples of toxins include, but are not restricted to, staphylococcal enterotoxins, toxic shock syndrome toxin; retroviral antigens (e.g., antigens derived from HFV), streptococcal antigens, staphylococcal enterotoxin-A (SEA), staphylococcal enterotoxin-B (SEB), staphylococcal enterotoxin-C(SEC), staphylococcal enterotoxin-D (SED), staphylococcal enterotoxin-E (SEE), as well as toxins derived from mycoplasma, mycobacterium, and herpes viruses.

Peptide antigens may be of any suitable size that can be utilised to stimulate or inhibit an immune response to a target antigen of interest. A number of factors can influence the choice of peptide size. For example, the size of a peptide can be chosen such that it includes, or corresponds to the size of, T cell epitopes and/or B cell epitopes, and their processing requirements. Practitioners in the art will recognise that class I-restricted T cell epitopes are typically between 8 and 10 amino acid residues in length and if placed next to unnatural flanking residues, such epitopes can generally require 2 to 3 natural flanking amino acid residues to ensure that they are efficiently processed and presented. Class II-restricted T cell epitopes usually range between 12 and 25 amino acid residues in length and may not require natural flanking residues for efficient proteolytic processing although it is believed that natural flanking residues may play a role. Another important feature of class II-restricted epitopes is that they generally contain a core of 9-10 amino acid residues in the middle which bind specifically to class II MHC molecules with flanking sequences either side of this core stabilising binding by associating with conserved structures on either side of class II MHC antigens in a sequence independent manner. Thus, the functional region of class II-restricted epitopes is typically less than about 15 amino acid residues long.

Criteria for identifying and selecting effective antigenic peptides (e.g., minimal peptide sequences capable of eliciting an immune response) can be found in the art. For example, Apostolopoulos et al. (2000, Curr. Opin. Mol. Ther. 2:29-36) discusses the strategy for identifying minimal antigenic peptide sequences based on an understanding of the three-dimensional structure of an antigen-presenting molecule and its interaction with both an antigenic peptide and T cell receptor. Shastri (1996, Curr. Opin. Immunol. 8:271-277) discloses how to distinguish rare peptides that serve to activate T cells from the thousands of peptides normally bound to MHC molecules.

### Polynucleotide Immune Stimulators

In some embodiments, the second agent comprises a nucleic acid composition comprising a coding sequence for an immune stimulator, wherein the immune stimulator stimulates or otherwise enhances an immune response to a target antigen in a subject. The synthetic construct may also comprise a 3′ non-translated sequence. A 3′ non-translated sequence refers to that portion of a gene comprising a DNA segment that contains a polyadenylation signal and any other regulatory signals capable of effecting mRNA processing or gene expression. The polyadenylation signal is characterized by effecting the addition of polyadenylic acid tracts to the 3′ end of the mRNA precursor.

Polyadenylation signals are commonly recognized by the presence of homology to the canonical form 5′ AATAAA-3′ although variations are not uncommon. The 3′ non-translated regulatory DNA sequence preferably includes from about 50 to 1,000 nucleotide base pairs and may contain transcriptional and translational termination sequences in addition to a polyadenylation signal and any other regulatory signals capable of effecting mRNA processing or gene expression.

It will be understood, however, that expression of polypeptide-encoding polynucleotides is now well known, and the present invention is not directed to or dependent on any particular nucleic acid sequence, vector, transcriptional control sequence, or technique for its production. Rather, synthetic polynucleotides prepared according to the methods as set forth herein may be introduced into selected cells or tissues or into a precursors or progenitors thereof in any suitable manner in conjunction with any suitable synthetic construct or vector, and the synthetic polynucleotides may

In some other embodiments, in order to enhance the class I presentation of the antigen, the immune stimulator is modified to comprise an intracellular degradation signal or degron. The degron is suitably a ubiquitin-mediated degradation signal selected from a destabilizing amino acid at the amino-terminus of an antigen, a ubiquitin acceptor, a ubiquitin or combination thereof.

Thus, in one embodiment, the immune stimulator is modified to include a destabilizing amino acid at its amino-terminus so that the protein so modified is subject to the N-end rule pathway as disclosed, for example, by Bachmair et al., in U.S. Pat. No. 5,093,242 and by Varshavsky et al., in U.S. Pat. No. 5,122,463. In a preferred embodiment of this type, the destabilizing amino acid is selected from isoleucine and glutamic acid, more preferably from histidine tyrosine and glutamine, and even more preferably from aspartic acid, asparagine, phenylalanine, leucine, tryptophan and lysine. In an especially preferred embodiment, the destabilizing amino acid is arginine.

Modification or design of the amino-terminus of a protein can also be accomplished at the genetic level. Conventional techniques of site-directed mutagenesis for addition or substitution of appropriate codons to the 5′ end of an isolated or synthesized antigen-encoding polynucleotide can be employed to provide a desired amino-terminal structure for the encoded protein. For example, so that the protein expressed has the desired amino acid at its amino-terminus the appropriate codon for a destabilizing amino acid can be inserted or built into the amino-terminus of the protein-encoding sequence. Where necessary, a nucleic acid sequence encoding the amino-terminal region of a protein can be modified to introduce one or more lysine residues in an appropriate context, which act as a ubiquitin acceptor as described in more detail below. This can be achieved most conveniently by employing DNA constructs encoding “universal destabilizing segments”. A universal destabilizing segment comprises a nucleic acid construct which encodes a polypeptide structure, preferably segmentally mobile, containing one or more lysine residues, the codons for lysine residues being positioned within the construct such that when the construct is inserted into the coding sequence of the antigen-encoding polynucleotide, the lysine residues are sufficiently spatially proximate to the amino-terminus of the encoded protein to serve as the second determinant of the complete amino-terminal degradation signal. The insertion of such constructs into the 5′ portion of an antigen-encoding polynucleotide would provide the encoded protein with a lysine residue (or residues) in an appropriate context for destabilization.

The codon for the amino-terminal amino acid of the protein of interest can be made to encode the desired amino acid by, for example, site-directed mutagenesis techniques currently standard in the field. Suitable mutagenesis methods are described for example in the relevant sections of Ausubel, et al., supra; and of Sambrook, et al., supra. Alternatively, suitable methods for altering DNA are set forth, for example, in U.S. Pat. Nos. 4,184,917, 4,321,365 and 4,351,901, which are incorporated herein by reference. Instead of in vitro mutagenesis, the synthetic polynucleotide can be synthesized de novo using readily available machinery. Sequential synthesis of DNA is described, for example, in U.S. Pat. No. 4,293,652. However, it should be noted that the present invention is not dependent on, and not directed to, any one particular technique for constructing a polynucleotide encoding a modified antigen as described herein.

If the antigen-encoding polynucleotide is a synthetic or recombinant polynucleotide the appropriate 5′ codon can be built-in during the synthetic process. Alternatively, nucleotides for a specific codon can be added to the 5′ end of an isolated or synthesized polynucleotide by ligation of an appropriate nucleic acid sequence to the 5′ (amino-terminus-encoding) end of the polynucleotide. Nucleic acid inserts encoding appropriately located lysine residues (such as the “universal destabilizing segments” described above) can suitably be inserted into the 5′ region to provide for the second determinant of the complete amino-terminal degradation.

In a preferred embodiment, the modified antigen, which comprises a destabilizing amino acid at its amino terminus, is fused or otherwise conjugated to a masking entity, which masks said amino terminus so that when unmasked the antigen will exhibit the desired rate of intracellular proteolytic degradation. Suitably, the masking entity is a masking protein sequence. The fusion protein is designed so that the masking protein sequence fused to the amino-terminus of the protein of interest is susceptible to specific cleavage at the junction between the two. Removal of the protein sequence thus unmasks the amino-terminus of the protein of interest and the half-life of the released protein is thus governed by the predesigned amino-terminus. The fusion protein can be designed for specific cleavage in vivo, for example, by a host cell endoprotease or for specific cleavage in an in vitro system where it can be cleaved after isolation from a producer cell (which lacks the capability to cleave the fusion protein). Thus, in a preferred embodiment, the masking protein sequence is cleavable by an endoprotease, which is preferably an endogenous endoprotease of a mammalian cell. Suitable endoproteases include, but are not restricted to, serine endoproteases (e.g., subtilisins and furins) as described, for example, by Creemers, et al. (1998, Semin. Cell Dev. Biol. 9 (1): 3-10), proteasomal endopeptidases as described, for example, by Zwickl, et al. (2000, Curr. Opin. Struct. Biol. 10 (2): 242-250), proteases relating to the MHC class I processing pathway as described, for example, by Stolze et al. (2000, Nat. Immunol. 1 413-418) and signal peptidases as described, for example, by Dalbey, et al. (1997, Protein Sci. 6 (6): 1129-1138). In some preferred embodiments of this type, the masking protein sequence comprises a signal peptide sequence. Suitable signal peptides sequences are described, for example, by Nothwehr et al. (1990, BioEssays 12 (10): 479-484), Izard, et al. (1994, Mol. Microbiol. 13 (5): 765-773), Menne, et al. (2000, Bioinformatics. 16 (8): 741-742) and Ladunga (2000, Curr. Opin. Biotechnol. 11 (1): 13-18). Suitably, an endoprotease cleavage site is interposed between the masking protein sequence and the antigen.

A modified antigen with an attached masking sequence may be conveniently prepared by fusing a nucleic acid sequence encoding a masking protein sequence upstream of another nucleic acid sequence encoding an antigen, which corresponds to the target antigen of interest and which includes a destabilizing amino acid at its amino-terminus. The codon for the amino-terminal amino acid of the antigen of interest is suitably located immediately adjacent to the 3′ end of the masking protein-encoding nucleic acid sequence.

In another embodiment, the antigen is modified to include, or is otherwise associated with, a ubiquitin acceptor which is a molecule that preferably contains at least one residue appropriately positioned from the N-terminal of the antigen as to be able to be bound by ubiquitin molecules. Such residues preferentially have an epsilon amino group such as lysine. Physical analysis demonstrates that multiple lysine residues function as ubiquitin acceptor sites (King et al., 1996, Mol. Biol. Cell 7: 1343-1357; King et al., 1996, Science 274: 1652-1659).

Examples of other ubiquitin acceptors include Sindis virus RNA polymerase. Ubiquitination at the N-terminal of the protein specifically targets the protein for degradation via the ubiquitin-proteosome pathway.

Other protein processing signals that destabilize an antigen of interest and allow for enhanced intracellular degradation are contemplated in the present invention. These other methods may not necessarily be mediated by the ubiquitin pathway, but may otherwise permit degradation of proteins in the cytoplasm via proteasomes. For example, the present invention contemplates the use of other intracellular processing signals which govern the rate(s) of intracellular protein degradation including, but not limited to, those described by Bohley et al. (1996, Biol. Chem. Hoppe. Seyler 377: 425-435). Such processing signals include those that allow for phosphorylation of the target protein (Yaglom et al., 1996, Mol. Cell Biol. 16: 3679-3684; Yaglom et al., 1995, Mol. Cell Biol. 15: 731-741). Also contemplated by the present invention are modification of a parent antigen that allow for post-translational arginylation (Ferber et al. 1987, Nature 326: 808-811; Bohley et al., 1991, Biomed. Biochim. Acta 50: 343-346) of the protein which can enhance its rate(s) of intracellular degradation. The present invention also contemplates the use of certain structural features of proteins that can influence higher rates of intracellular protein turn-over, including protein surface hydrophobicity, clusters of hydrophobic residues within the protein (Sadis et al., Mol Cell Biol, 1995, 15: 4086-4094), certain hydrophobic pentapeptide motifs at the protein's carboxy-terminus (C-terminus) (e.g., ARINV), as found on the C-terminus of ornithine decarboxylase (Ghoda et al., Mol Cell Biol, 1992, 12: 2178-2185; Li, et al., Mol Cell Biol, 1994, 14: 87-92), or AANDENYALAA, as found in C-terminal tags of aberrant polypeptides (Keiler et al., Science, 1996, 271: 990-993) or PEST regions (regions rich in proline (P), glutamic acid (E), serine (S), and threonine (T), which are optionally flanked by amino acids comprising electropositive side chains (Rogers et al., Science, 1986, 234 (4774): 364-368; 1988, J. Biol. Chem. 263: 19833-19842). Moreover, certain motifs have been identified in proteins that appear necessary and possibly sufficient for achieving rapid intracellular degradation. Such motifs include RXALGXIXN region (where X=any amino acid) in cyclins (Glotzer et al., Nature, 1991, 349: 132-138) and the KTKRNYSARD motif in isocitrate lyase (Ordiz et al., FEBS Lett, 1996, 385: 43-46).

The present invention also contemplates enhanced cellular degradation of a parent antigen which may occur by the incorporation into that antigen of known protease cleavage sites. For example, amyloid beta-protein can be cleaved by beta- and gamma-secretase (Iizuka et al., 1996, Biochem. Biophys. Res. Commun. 218: 238-242) and the two-chain vitamin K-dependent coagulation factor X can be cleaved by calcium-dependent endoprotease(s) in liver (Wallin et al., 1994, Thromb. Res. 73: 395-403).

In yet another embodiment, the parent antigen is conjugated to a ubiquitin or a biologically active fragment thereof, to produce a modified antigen whose rate of intracellular proteolytic degradation is increased, enhanced or otherwise elevated relative to the parent antigen. In a preferred embodiment of this type, the ubiquitin or biologically active fragment is fused, or otherwise conjugated, to the antigen. Suitably, the ubiquitin is of mammalian origin, more preferably of human or other primate origin.

In one embodiment, the ubiquitin-antigen fusion protein is suitably produced by covalently attaching an antigen corresponding to the target antigen to a ubiquitin or a biologically active fragment thereof. Covalent attachment may be effected by any suitable means known to persons of skill in the art. For example, protein conjugates may be prepared by linking proteins together using bifunctional reagents. The bifunctional reagents can be homobifunctional or heterobifunctional.

Homobifunctional reagents are molecules with at least two identical functional groups. The functional groups of the reagent generally react with one of the functional groups on a protein, typically an amino group. Examples of homobifunctional reagents include glutaraldehyde and diimidates. An example of the use of glutaraldehyde as a cross-linking agent is described by Poznansky et al. (1984, Science, 223: 1304-1306). The use of diimidates as a cross-linking agent is described for example by Wang, et al. (1977, Biochemistry, 16: 2937-2941). Although it is possible to use homobifunctional reagents for the purpose of forming a modified antigen according to the invention, skilled practitioners in the art will appreciate that it is difficult to attach different proteins in an ordered fashion with these reagents. In this regard, in attempting to link a first protein with a second protein by means of a homobifunctional reagent, one cannot prevent the linking of the first protein to each other and of the second to each other. Heterobifunctional crosslinking reagents are, therefore, preferred because one can control the sequence of reactions, and combine proteins at will. Heterobifunctional reagents thus provide a more sophisticated method for linking two proteins. These reagents require one of the molecules to be joined, hereafter called Partner B, to possess a reactive group not found on the other, hereafter called Partner A, or else require that one of the two functional groups be blocked or otherwise greatly reduced in reactivity while the other group is reacted with Partner A. In a typical two-step process for forming heteroconjugates, Partner A is reacted with the heterobifunctional reagent to form a derivatized Partner A molecule. If the unreacted functional group of the cross linker is blocked, it is then deprotected. After deprotection, Partner B is coupled to derivatized Partner A to form the conjugate. Primary amino groups on Partner A are reacted with an activated carboxylate or imidate group on the cross linker in the derivatization step. A reactive thiol or a blocked and activated thiol at the other end of the cross linker is reacted with an electrophilic group or with a reactive thiol, respectively, on Partner B. When the cross linker possesses a reactive thiol, the electrophile on Partner B preferably will be a blocked and activated thiol, a maleimide, or a halomethylene carbonyl (e.g., bromoacetyl or iodoacetyl) group. Because biological macromolecules do not naturally contain such electrophiles, they must be added to Partner B by a separate derivatization reaction. When the cross linker possesses a blocked and activated thiol, the thiol on Partner B with which it reacts may be native to Partner B.

An example of a heterobifunctional reagent is N-succinimidyl 3-(2-pyridyldithio) propionate (SPDP) (see for example Carlsson et al., 1978, Biochem. J., 173: 723-737). Other heterobifunctional reagents for linking proteins include for example succinimidyl 4-(N-maleimidomethyl) cyclohexane-1-carboxylate (SMCC) (Yoshitake et al., 1979, Eur. J. Biochem, 101: 395-399), 2-iminothiolane (IT) (Jue et al., 1978, Biochemistry, 17: 5399-5406), and S-acetyl mercaptosuccinic anhydride (SAMSA) (Klotz and Heiney, 1962, Arch. Biochem. Biophys., 96: 605-612). All three react preferentially with primary amines (e.g., lysine side chains) to form an amide or amidine group which links a thiol to the derivatized molecule (e.g., a heterologous antigen) via a connecting short spacer arm, one to three carbon atoms long. Examples of heterobifunctional reagents comprising reactive groups having a double bond that reacts with a thiol group include SMCC mentioned above, succinimidyl m-maleimidobenzoate, succinimidyl 3-(maleimido)propionate, sulfosuccinimidyl 4-(p-maleimidophenyl)butyrate, sulfosuccinimidyl 4-(N-maleimidomethylcyclohexane-1-carboxylate and maleimidobenzoyl-N-hydroxysuccinimide ester (MBS). In a preferred embodiment, MBS is used to produce the conjugate. Other heterobifunctional reagents for forming conjugates of two proteins are described for example by Rodwell et al., in U.S. Pat. No. 4,671,958 and by Moreland et al. in U.S. Pat. No. 5,241,078.

In an alternate embodiment, a ubiquitin-antigen fusion protein is suitably expressed by a synthetic chimeric polynucleotide comprising a first nucleic acid sequence, which encodes an antigen corresponding to the target antigen, and which is linked downstream of, and in reading frame with, a second nucleic acid sequence encoding a ubiquitin or biologically active fragment thereof. In a preferred embodiment of this type, the second polynucleotide comprises a first nucleic acid sequence, which encodes an antigen corresponding to the target antigen, and which is linked immediately adjacent to, downstream of, and in reading frame with, a second nucleic acid sequence encoding a ubiquitin or biologically active fragment thereof. In another embodiment, the second polynucleotide comprises a first nucleic acid sequence, which encodes an antigen corresponding to the target antigen, and which is linked upstream of, and in reading frame with, a second nucleic acid sequence encoding a ubiquitin or biologically active fragment thereof. In yet another embodiment of this type, the second polynucleotide comprises a first nucleic acid sequence, which encodes an antigen corresponding to the target antigen, and which is linked immediately adjacent to, upstream of, and in reading frame with, a second nucleic acid sequence encoding a ubiquitin or biologically active fragment thereof.

### 2. Encapsulation/Complexation in Nanoparticles

In some preferred embodiments, the second agent is complexed, encapsulated, partially encapsulated, or associated with one or more lipids (e.g., cationic lipids and/or ionisable lipids and/or neutral lipids), thereby forming lipid-based carriers such as liposomes, lipid nanoparticles, lipoplexes and/or nanoliposomes. The second agent may be completely or partially located in the interior space of the lipid-based carrier. The incorporation of a nucleic acid into lipid-carriers is also referred to herein as “encapsulation” wherein the nucleic acid (e.g., the RNA is entirely contained within the interior space of the lipid-carrier). The purpose of incorporating the second agent into the lipid-carrier is to protect the second agent, preferably RNA from an environment which may contain enzymes or chemicals or conditions that degrade nucleic acid and/or systems or receptors that cause the rapid excretion of the nucleic acid. Moreover, incorporating nucleic acid, preferably RNA, into lipid-based carriers may promote the uptake of the nucleic acid, and hence, may enhance the therapeutic effect of the nucleic acid. Accordingly, incorporating a nucleic acid (e.g., RNA or DNA) into liposomes, lipid nanoparticles (LNPs), lipoplexes, and/or nanoliposomes may be particularly suitable for the immunostimulatory molecules of the present invention (e.g., for intramuscular, intravenous, and/or intradermal administration).

In this context, the terms “complexed” or “associated” refer to the essentially stable combination of nucleic acid with one or more lipids into larger complexes or assemblies without covalent binding.

### 2.1 Lipid Nanoparticles (LNPs)

The LNPs of the invention are suitably characterised as microscopic vesicles having an interior aqua space sequestered from an outer medium by a membrane or one or more bilayers. Bilayer membranes of LNPs are typically formed by amphiphilic molecules, such as lipids of synthetic or natural origin that comprise spatially separated hydrophilic and hydrophobic domains. Bilayer membranes of the liposomes can also be formed by amphophilic polymers and surfactants (e.g., polymerosomes, niosomes, etc.). In the context of the present invention, an LNP typically serves to transport the second agent (preferably a polynucleotide that encodes the immune stimulator) to a target tissue.

In some embodiments, a lipid nanoparticle (LNP) may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as a “cationic lipid”. In certain embodiments, an ionizable lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and tertiary amines), ammonium groups, pyridinium group, guanidine groups, and imidazolium groups. In some embodiments, the charged moieties comprise amine groups. Examples of negatively-charged groups or precursors thereof, include carboxylate groups, sulphonate groups, sulphate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired. Ionizable lipids can also be the compounds disclosed in International Publication Nos. WO2017/075531, WO2015/199952, WO2013/086354, or WO2013/116126, or selected from formulae CLI-CLXXXXII of U.S. Pat. No. 7,404,969 (each of which is hereby incorporated by reference in its entirety for this purpose).

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid, sometimes referred to in the art as an “ionizable cationic lipid”. In one embodiment, the ionizable amino lipid may have a positively charged hydrophilic head and a hydrophobic tail that are connected via a linker structure. In addition to these, an ionizable lipid may also be a lipid including a cyclic amine group.

Vaccines of the present disclosure are typically formulated into lipid nanoparticles. In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises a molar ratio of 20-60% ionizable amino lipid. For example, the lipid nanoparticle may comprise a molar ratio of 20-50%, 20-40%, 20-30%, 30-60%, 30-50%, 30-40%, 40-60%, 40-50%, or 50-60% ionizable amino lipid. In some embodiments, the lipid nanoparticle comprises a molar ratio of 20%, 30%, 40%, 50, or 60% ionizable amino lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or 25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable amino lipid, 5-25% non-cationic lipid, 25-55% sterol, and 0.5-15% PEG-modified lipid.

In some embodiments, an ionizable amino lipid of the disclosure comprises a compound of Formula (X):

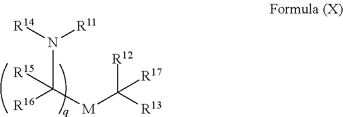

or a pharmaceutically acceptable salt or isomer thereof, wherein:
R¹¹ is selected from the group consisting of C₅₋₃₀ alkyl, C₅₋₂₀ alkenyl, —R*Y′R″, —Y′R″, and —R″M′R′;
R¹² and R¹³ are independently selected from the group consisting of H, C₁₋₁₄ alkyl, C₂₋₁₄ alkenyl, —R*Y′R″, —Y′R″, and —R*OR″, or R¹² and R¹³, together with the atom to which they are attached, form a heterocycle or carbocycle;
R¹⁴ is selected from the group consisting of a C₃₋₆ carbocycle, (CH₂)ₙQ, (CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX′₃, —CX′₂H, —CX′H₂, —CN, —N(R)₂, —C(O)N(R)₂, —N(R)C(O)R, —N(R)S(O)₂R, —N(R)C(O)N(R)₂, —N(R)C(S)N(R)₂, —N(R)RS, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R¹⁵ is independently selected from the group consisting of C₁₋₃ alkyl, C₂₋₃ alkenyl, and H;
each R¹⁵ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R¹⁷ is selected from the group consisting of C₁₋₃ alkyl, C₂₋₃ alkenyl, and H;
R¹⁸ is selected from the group consisting of C₃₋₅ carbocycle and heterocycle;
R¹⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle; each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*Y′R″, —Y′R″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y′ is independently a C₃₋₆ carbocycle; and
each X′ is independently selected from the group consisting of F, Cl, Br, and I; and q is selected from 5, 6, 7, 8, 9, 10, 11, 12, and 13.

In some embodiments, a subset of compounds of Formula (X) includes those in which when R¹⁴ is —(CH₂)ₙQ, —(CH₂)ₙCHQR, —CHQR, or -CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (X) includes those in which
R″ is selected from the group consisting of C₅₋₃₀ alkyl, C₅₋₂₀ alkenyl, —R*Y′R″, —Y′R″, and —R″M′R′;
R¹² and R¹³ are independently selected from the group consisting of H, C₁₋₁₄ alkyl, C₂₋₁₄ alkenyl, —R*Y′R″, —Y′R″, and —R*OR″, or R¹² and R¹³, together with the atom to which they are attached, form a heterocycle or carbocycle;
R¹⁴ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX′, —CX′₂H, —CX′H₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)RS, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R¹⁵ is independently selected from the group consisting of C₁₋₃ alkyl, C₂₋₃ alkenyl, and H;
each R¹⁶ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R¹⁷ is selected from the group consisting of C₁₋₃ alkyl, C₂₋₃ alkenyl, and H;
R¹⁸ is selected from the group consisting of C₃₋₆ carbocycle and heterocycle;
R¹⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*Y′R″, —Y′R″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y′ is independently a C₃₋₆ carbocycle;
each X′ is independently selected from the group consisting of F, Cl, Br, and I; and q is selected from 5, 6, 7, 8, 9, 10, 11, 12, and 13, or salts or isomers thereof.

In some embodiments, another subset of compounds of Formula (I) includes those in which
R″ is selected from the group consisting of C₅₋₃₀ alkyl, C₅₋₂₀ alkenyl, —R*Y′R″, —Y′R″, and —R″M′R′;
R¹² and R¹³ are independently selected from the group consisting of H, C₁₋₁₄ alkyl, C₂₋₁₄ alkenyl, —R*Y′R″, —Y′R″, and —R*OR″, or R₂ and R₃, together with the atom to which they are attached, form a heterocycle or carbocycle;
R¹⁴ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX′3, —CX′₂H, —CX′H₂, —CN, —C(O)N(R)₂, —N(R)C(O)R, —N(R)S(O)₂R, —N(R)C(O)N(R)₂, —N(R)C(S)N(R)₂, —CRN(R₂C(O)OR, —N(R)RS, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and -CQ(R)₂, then Q is either a 5- to 14-membered heteroaryl or 8- to 14-membered heterocycloalkyl;
each R¹⁵ is independently selected from the group consisting of C₁₋₃ alkyl, C₂₋₃ alkenyl, and H;
each R¹⁵ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R¹⁷ is selected from the group consisting of C₁₋₃ alkyl, C₂₋₃ alkenyl, and H;
R¹⁸ is selected from the group consisting of C₃₋₆ carbocycle and heterocycle;
R¹⁹ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*Y′R″, —Y′R″ and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y′ is independently a C₃₋₆ carbocycle;
each X′ is independently selected from the group consisting of F, Cl, Br, and I; and q is selected from 5, 6, 7, 8, 9, 10, 11, 12, and 13,
   or pharmaceutically acceptable salts or isomers thereof.

In some embodiments, another subset of compounds of Formula (X) includes those in which
R¹¹ is selected from the group consisting of C₅₋₃₀ alkyl, C₅₋₂₀ alkenyl, —R*Y′R″, —Y′R″, and —R″M′R′;
R¹² and R¹³ are independently selected from the group consisting of H, C₁₋₁₄ alkyl, C₂₋₁₄ alkenyl, —R*Y′R″, —Y′R″, and —R*OR″, or R₂ and R₃, together with the atom to which they are attached, form a heterocycle or carbocycle;
R¹⁴ is selected from the group consisting of a C₃₋₆ carbocycle, —(CH₂)ₙQ, —(CH₂)ₙCHQR, —CHQR, —CQI₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX′, —CX′₂H, —CX′H₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)RS, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and each n is independently selected from 1, 2, 3, 4, and 5;
each R¹⁵ is independently selected from the group consisting of C₁₋₃ alkyl, C₂₋₃ alkenyl, and H;
each R¹⁶ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R¹⁷ is selected from the group consisting of C₁₋₃ alkyl, C₂₋₃ alkenyl, and H;
R¹⁸ is selected from the group consisting of C₃₋₈ carbocycle and heterocycle;
R¹⁹ is selected from I group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₅ alkenyl, —R*Y′R″, —Y′R′, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y′ is independently a C3-6 carbocycle;
   each X′ is independently selected from the group consisting of F, Cl, Br, and I; and m is selected from 5, 6, 7, 8, 9, 10, 11, 12, and 13, or pharmaceutically acceptable salts or isomers thereof.

In some embodiments, another subset of compounds of Formula (X) includes those in which
R″ is selected from the group consisting of C₅₋₃₀ alkyl, C₅₋₂₀ alkenyl, —R*Y′R″, —Y′R″, and —R″M′R′;
R¹² and R¹³ are independently selected from the group consisting of H, C₂₋₁₄ alkyl, C₂₋₁₄ alkenyl, —R*Y′R″, —Y′R″, and —R*OR″, or R¹² and R¹³, together with the atom to which they are attached, form a heterocycle or carbocycle;
R¹⁴ is —(CH₂)ₙQ or —(CH₂)ₙCHQR, where Q is —N(R)₂, and n is selected from 3, 4, and 5; each R₅ is independently selected from the group consisting of C₁₋₃ alkyl, C₂₋₃ alkenyl, and H;
each R¹⁶ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R¹⁷ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₅ alkenyl, —R*Y′R″, —Y′R″, and H;
each R″ is independently selected from the group consisting of C₃₋₁₄ alkyl and C₃₋₁₄ alkenyl;
each R* is independently selected from the group consisting of C₁₋₁₂ alkyl and CM₂ alkenyl;
each Y′ is independently a C₃₋₆ carbocycle;
each X′ is independently selected from the group consisting of F, Cl, Br, and I; and q is selected from 5, 6, 7, 8, 9, 10, 11, 12, and 13,
or pharmaceutically acceptable salts or isomers thereof.

In some embodiments, another subset of compounds of Formula (X) includes those in which
R″ is selected from the group consisting of C₅₋₃₀ alkyl, C₅₋₂₀ alkenyl, —R*Y′R″, —Y′R″, and —R″M′R′;
R¹² and R¹³ are independently selected from the group consisting of C₁₋₁₄ alkyl, C₂₋₁₄ alkenyl, —R*Y′R″, —Y′R″, and —R*OR″, or R₂ and R₃, together with the atom to which they are attached, form a heterocycle or carbocycle;
R¹⁴ is selected from the group consisting of —(CH₂)ₙQ, —(CH₂)ₙCHQR, —CHQR, and -CQ(R)₂, where Q is —N(R)₂, and n is selected from 1, 2, 3, 4, and 5;
each R¹⁵ is independently selected from the group consisting of C₁₋₃ alkyl, C₂₋₃ alkenyl, and H;
each R¹⁶ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R¹⁷ is selected from the group consisting of C₁₋₃ alkyl, C₂₋₃ alkenyl, and H; each R is independently selected from the group consisting of C₁₋₃ alkyl, C₂₋₃ alkenyl, and H;
each R′ is independently selected from the group consisting of CMS alkyl, C₂₋₁₈ alkenyl, —R*Y′R″, —Y′R″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
   each Y′ is independently a C₃₋₆ carbocycle;
   each X′ is independently selected from the group consisting of F, Cl, Br, and I; and q is selected from 5, 6, 7, 8, 9, 10, 11, 12, and 13,
   or pharmaceutically acceptable salts or isomers thereof.

In some embodiments, a subset of compounds of Formula (X) includes those of Formula (XA):

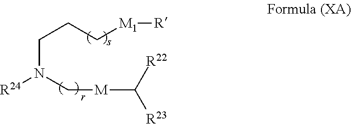

or a pharmaceutically acceptable salt or isomer thereof, wherein s is selected from 1, 2, 3, 4, and 5; r is selected from 5, 6, 7, 8, and 9; M₁ is a bond or M′; R²⁴ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)RS, NHC(═NR⁹)N(R)₂, —NHC(═CHR⁹)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R²² and R²³ are independently selected from the group consisting of H, C₁₋₁₄ alkyl, and C₂₋₁₄alkenyl.

In some embodiments, a subset of compounds of Formula (X) includes those of Formula (XB):

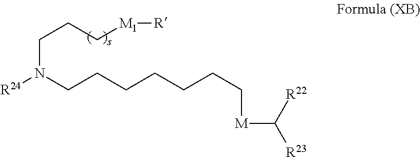

or a pharmaceutically acceptable salt or isomer thereof, wherein s is selected from 1, 2, 3, 4, and 5; M₁ is a bond or M′; R²⁴ is unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R⁸, —NHC(═NR⁹)N(R)₂, —NHC(═CHR⁹)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R²² and R²³ are independently selected from the group consisting of H, C₁₋₁₄ alkyl, and C₂₋₁₄ alkenyl.

In some embodiments, a subset of compounds of Formula (X) includes those of Formula (XC), (XD), (XE), or (XF):

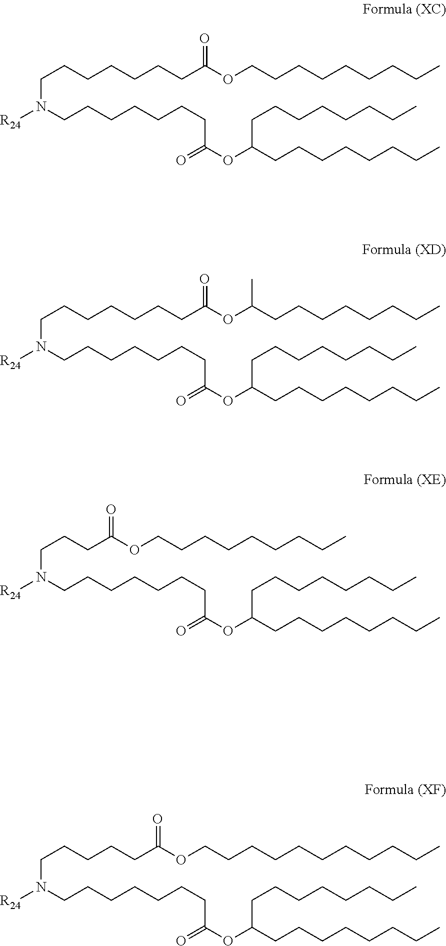

or a pharmaceutically acceptable salt or isomer thereof, wherein R²⁴ is as described herein.

In some embodiments, a subset of compounds of Formula (X) includes those of Formula (XG):

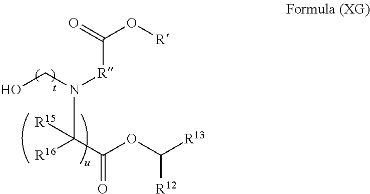

or a pharmaceutically acceptable salt or isomer thereof, wherein t is 2, 3, or 4; and m, R′, R″, and R¹², R¹³, R¹⁵, R¹⁶ are as described herein. For example, each of R¹² and R¹³ may be independently selected from the group consisting of C₅₋₁₄ alkyl and C₅₋₁₄ alkenyl.

In some embodiments, an ionizable cationic lipid of the disclosure comprises a compound (100) having structure:

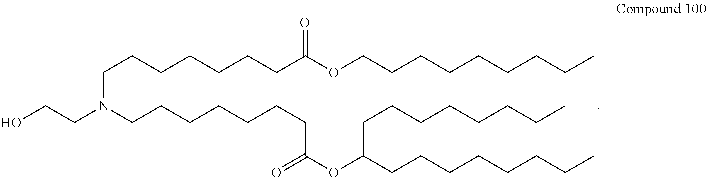 [SMILES: CCCCCCCCCOC(=O)CCCCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, brassicasterol, tomatidine, ursolic acid, alpha-tocopherol, and mixtures thereof.

In some embodiments, a LNP of the disclosure comprises an ionizable amino lipid of Compound 1, wherein the non-cationic lipid is DSPC, the structural lipid is cholesterol, and the PEG lipid is PEG-DMG.

In some embodiments, a LNP of the disclosure comprises an N:P ratio of from about 2:1 to about 30:1. In some embodiments, a LNP of the disclosure comprises an N:P ratio of about 9:1. In some embodiments, a LNP of the disclosure comprises an N:P ratio of about 6:1. In some embodiments, a LNP of the disclosure comprises an N:P ratio of about 4:1. In some embodiments, a LNP of the disclosure comprises an N:P ratio of about 3:1.

In some embodiments, a LNP of the disclosure comprises a wt/wt ratio of the ionizable cationic lipid component to the RNA of from about 10:1 to about 100:1.

In some embodiments, a LNP of the disclosure comprises a wt/wt ratio of the ionizable cationic lipid component to the RNA of about 20:1.

In some embodiments, a LNP of the disclosure comprises a wt/wt ratio of the ionizable cationic lipid component to the RNA of about 10:1.

In various embodiments, the LNPs have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In some embodiments, the lipid may be a cleavable lipid such as those described in International Publication No. WO 2012/170889, herein incorporated by reference in its entirety. In some embodiments, the lipid may be synthesized by methods known in the art and/or as described in International Publication No. WO 2013/086354; the contents of which is herein incorporated by reference in its entirety.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide. As used herein, “size” or “mean size” in the context of nanoparticle compositions refers to the mean diameter of a nanoparticle composition.

### Pharmaceutical Compositions

In some embodiments, the immunostimulatory composition is formulated as a pharmaceutical composition. By way of an example the pharmaceutical composition may comprise an effective amount of the immunostimulatory composition as described above and/or elsewhere herein, and a pharmaceutically acceptable carrier, excipient, or diluent.

In some preferred embodiments of this type, the pharmaceutical composition is formulated as a vaccine. Typically, the vaccine also comprises a pharmaceutically acceptable carrier, excipient, or diluent.

Relative amounts of the active ingredient (e.g., the immune stimulator or nucleic acid encoding the immune stimulator), the pharmaceutically acceptable excipient, and/or any additional ingredients in a vaccine composition may vary, depending upon the identity, size, and/or condition of the subject being treated and further depending upon the route by which the composition is to be administered.

For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the package containing the pharmaceutical product contains 0.1 mg to 1 mg of nucleic acid (e.g., mRNA). In some embodiments, the package containing the pharmaceutical product contains 0.35 mg of nucleic acid (e.g., mRNA). In some embodiments, the concentration of the nucleic acid (e.g., mRNA) is 1 mg/mL.

In some embodiments, the nucleic acid (e.g., mRNA) vaccine compositions may be administered at dosage levels sufficient to deliver 0.1 μg/kg to 100 mg/kg, 1 μg/kg to 50 μg/kg, 5 μg/kg to 50 μg/kg, 1 μg/kg to 5 μg/kg, 50 μg/kg to 0.5 mg/kg, 10 μg/kg to 50 mg/kg, 0.1 mg/kg to 40 mg/kg, 0.5 mg/kg to 30 mg/kg, 10 μg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see e.g., the range of unit doses described in International Publication No. WO 2013/078199, herein incorporated by reference in its entirety). In some embodiments, the nucleic acid (e.g., mRNA) vaccine is administered at a dosage level sufficient to deliver 10 μg, 25 μg, 40 μg, 50 μg, 75 μg, 100 μg, 125 μg, 130 μg, 150 μg, 175 μg, 200 μg, 225 μg, 250 μg, 275 μg, 300 μg, 325 μg, 350 μg, 375 μg, 390 μg, 400 μg, 425 μg, 450 μg, 475 μg, 500 μg, 525 μg, 550 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, or 1000 μg.

In some embodiments, the nucleic acid (e.g., mRNA) vaccine is administered at a dosage level sufficient to deliver between 10 μg and 400 μg of the mRNA vaccine to the subject. In some embodiments, the nucleic acid (e.g., mRNA) vaccine is administered at a dosage level sufficient to deliver at least 0.033 mg, at least 0.040 mg, at least 0.1 mg, at least 0.13 mg, at least 0.2 mg, at least 0.39 mg, at least 0.4 mg, or at least 1.0 mg to the subject.

In some embodiments, the nucleic acid (e.g., mRNA) vaccine is administered at a dosage level sufficient to deliver at least 1.0 mg, at least 1.2 mg, at least 1.4 mg, at least 1.6 mg, at least 1.8 mg, or at least 2.0 mg, at least to the subject. In some embodiments, the nucleic acid (e.g., mRNA) vaccine is administered at a dosage level sufficient to deliver at least 2.0 mg, at least 2.2 mg, at least 2.4 mg, at least 2.6 mg, at least 2.8 mg, or at least 3.0 mg, at least to the subject. In some embodiments, the nucleic acid (e.g., mRNA) vaccine is administered at a dosage level sufficient to deliver at least 3.0 mg, at least 3.2 mg, at least 3.4 mg, at least 3.6 mg, at least 3.8 mg, or at least 4.0 mg, at least to the subject. In some embodiments, the nucleic acid (e.g., mRNA) vaccine is administered at a dosage level sufficient to deliver at least 4.0 mg, at least 4.2 mg, at least 4.4 mg, at least 4.6 mg, at least 42.8 mg, or at least 5.0 mg, at least to the subject.

The desired dosage may be delivered three times a day, two times a day, once a day, every other day, every third day, every week, every two weeks, every three weeks, every four weeks, every 2 months, every three months, every 6 months, etc. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In some embodiments, the nucleic acid (e.g., mRNA) vaccine compositions may be administered at dosage levels sufficient to deliver 0.5 μg/kg to 10 μg/kg, e.g., about 0.5 μg/kg to about 7.5 μg/kg, e.g., about 0.5 μg/kg, about 1 μg/kg, about 2 μg/kg, about 3 μg/kg, about 0.004 mg/kg or about 5 μg/kg. In some embodiments, the nucleic acid (e.g., mRNA) vaccine compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, the nucleic acid (e.g., mRNA) vaccine compositions may be administered twice (e.g., day 0 and day 7, day 0 and day 14, day 0 and day 21, day 0 and day 28, day 0 and day 60, day 0 and day 90, day 0 and day 120, day 0 and day 150, day 0 and day 180, day 0 and 3 months later, day 0 and 6 months later, day 0 and 9 months later, day 0 and 12 months later, day 0 and 18 months later, day 0 and 2 years later, day 0 and 5 years later, or day 0 and 10 years later) at a total dose of or at dosage levels sufficient to deliver a total dose of 0.0100 mg, 0.025 mg, 0.040 mg, 0.050 mg, 0.075 mg, 0.100 mg, 0.125 mg, 0.130 mg, 0.150 mg, 0.175 mg, 0.200 mg, 0.225 mg, 0.250 mg, 0.275 mg, 0.300 mg, 0.325 mg, 0.350 mg, 0.375 mg, 0.390 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg.

Higher and lower dosages and frequency of administration are encompassed by the present disclosure. For example, a nucleic acid (e.g., mRNA) vaccine composition may be administered three or four times, or more. In some embodiments, the mRNA vaccine composition is administered once a day every three weeks.

In some embodiments, the nucleic acid (e.g., mRNA) vaccine compositions may be administered twice (e.g., day 0 and day 7, day 0 and day 14, day 0 and day 21, day 0 and day 28, day 0 and day 60, day 0 and day 90, day 0 and day 120, day 0 and day 150, day 0 and day 180, day 0 and 3 months later, day 0 and 6 months later, day 0 and 9 months later, day 0 and 12 months later, day 0 and 18 months later, day 0 and 2 years later, day 0 and 5 years later, or day 0 and 10 years later) at a total dose of or at dosage levels sufficient to deliver a total dose of 0.010 mg, 0.025 mg, 0.100 mg or 0.400 mg.

In some embodiments the nucleic acid (e.g., mRNA) vaccine for use in a method of vaccinating a subject is administered the subject a single dosage of between 10 μg/kg and 400 μg/kg of the nucleic acid vaccine in an effective amount to vaccinate the subject. In some embodiments the RNA vaccine for use in a method of vaccinating a subject is administered the subject a single dosage of between 10 μg and 400 μg of the nucleic acid vaccine in an effective amount to vaccinate the subject.

### 5. Methods for Stimulating an Immune Response

The compositions of the invention may be used for stimulating an immune response to a target antigen in a subject that is immunologically native to the target antigen or that has previously raised an immune response to that antigen. Thus, the present invention also extends to methods for enhancing an immune response in a subject by administering to the subject the compositions or vaccines of the invention. Advantageously, the immune response is a cell-mediated immune response (e.g., a T cell mediated response, which desirably includes CD8⁺ IFN-γ-producing T cells).

Also encapsulated by the present invention is a method for treatment and/or prophylaxis of a disease or condition, comprising administering to a patient in need of such treatment an effective amount of an iNKT cell agonist that activates or induces the expansion of TRM cells, together with an effective amount of an immune stimulator, as broadly described above. In certain embodiments, the target antigen is associated with or responsible for a disease or condition which is suitably selected from infectious diseases, cancers, and diseases characterized by immunodeficiency.

In the context of infectious diseases, the composition is suitable for the treatment or prophylaxis of a viral, bacterial, or parasitic infection. Viral infections contemplated by the present invention include, but are not restricted to, infections caused by, for example, HIV, hepatitis, influenza, coronavirus, Japanese encephalitis virus, Epstein-Barr virus, respiratory syncytial virus.

Bacterial infections include, but are not restricted to, those caused by Neisseria species, Meningococcal species, Haemophilus species, Salmonella species, Streptococcal species, Legionella species, and Mycobacterium species (e.g., M. tuberculosis).

Parasitic infections encompassed by the invention include, but are not restricted to, those caused by Plasmodium species, Schistosoma species, Leishmania species, Trypanosoma species, Toxoplasma species, and Giardia species.

Examples of cancer include but are not limited to ABL1 proto-oncogene, AIDS related cancers (e.g., Kaposi's sarcoma), acoustic neuroma, acute lymphocytic leukaemia, acute myeloid leukaemia, adenocystic carcinoma, adrenocortical cancer, agnogenic myeloid metaplasia, alopecia, alveolar soft-part sarcoma, anal cancer, angiosarcoma, aplastic anemia, astrocytoma, ataxia telangiectasia, basal cell carcinoma (skin), bladder cancer, bone cancers, bowel cancer, brain stem glioma, brain and CNS tumours, breast cancer, CNS tumours, carcinoid tumours, cervical cancer, childhood brain tumours, childhood cancer, childhood leukaemia, childhood soft tissue sarcoma, chondrosarcoma, choriocarcinoma, chronic lymphocytic leukaemia, chronic myeloid leukaemia, colorectal cancers, cutaneous T-cell lymphoma, dermatofibrosarcoma protuberans, desmoplastic small round cell tumour, ductal carcinoma, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anemia, fibrosarcoma, gall bladder cancer, gastric cancer, gastrointestinal cancers, gastrointestinal carcinoid tumour, genitourinary cancers, germ cell tumours, gestational-trophoblastic disease, glioma, gynaecological cancers, hematological malignancies, hairy cell leukaemia, head and neck cancer, hepatocellular cancer, hereditary breast cancer, histiocytosis, Hodgkin's disease, human papillomavirus, hydatidiform mole, hypercalcemia, hypopharynx cancer, intraocular melanoma, islet cell cancer, Kaposi's sarcoma, kidney cancer, Langerhans' cell histiocytosis, laryngeal cancer, leiomyosarcoma, leukaemia, Li-Fraumeni syndrome, lip cancer, liposarcoma, liver cancer, lung cancer, lymphedema, lymphoma, Hodgkin's lymphoma, non-Hodgkin's lymphoma, male breast cancer, malignant-rhabdoid tumour of kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, nasal cancer, nasopharyngeal cancer, nephroblastoma, neuroblastoma, neurofibromatosis, Nijmegen breakage syndrome, non-melanoma skin cancer, non-small cell lung cancer (NSCLC), ocular cancers, esophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal tumours, pituitary cancer, polycythemia vera, prostate cancer, rare-cancers-and-associated-disorders, renal cell carcinoma, retinoblastoma, rhabdomyosarcoma, Rothmund Thomson syndrome, salivary gland cancer, sarcoma, schwannoma, Sezary syndrome, skin cancer, small cell lung cancer (SCLC), small intestine cancer, soft tissue sarcoma, spinal cord tumours, squamous-cell-carcinoma-(skin), stomach cancer, synovial sarcoma, testicular cancer, thymus cancer, thyroid cancer, transitional-cell-cancer-(bladder), transitional-cell-cancer-(renal-pelvis-/-ureter), trophoblastic cancer, urethral cancer, urinary system cancer, uroplakins, uterine sarcoma, uterus cancer, vaginal cancer, vulva cancer, Waldenstrom's-macroglobulinemia, Wilms' tumour.

In other embodiments, the composition of the invention could also be used for generating large numbers of TRM cells, for adoptive transfer to immunodeficient individuals who are unable to mount normal immune responses and/or generate TRM cells. For example, TRM cells can be adoptively transferred for therapeutic purposes in individuals afflicted with, for example, cancers (e.g., malignant tumours such as melanoma (see, Beumer-Chuwonpad et al., 2021, Cells 10: 2234; Van der Brogen et al., 1991, Science 254: 1643-1647; and Young and Steinman 1990, J. Exp. Med., 171: 1315-1332).

The effectiveness of an immunization may be assessed using any suitable technique known in the art. For example, a CTL lysis assays may be employed using stimulated splenocytes or peripheral blood mononuclear cells (PBMC) on peptide-coated or recombinant virus-infected cells using ⁵¹Cr or Alamar Blue™ labelled target cells. Such assays can be performed using for example primate, mouse or human cells (as described, for example, in Allen et al., J Immunol, 2000, 164(9): 4968-4978; and Woodberry et al., infra). Alternatively, the efficacy of the immunization may be monitored using one or more techniques including, but not limited to, HLA class I tetramer staining both fresh and stimulated PBMCs (see, for example, Allen et al., supra), proliferation assays (Allen et al., supra), ELISPOT assays, and intracellular IFN-γ staining (Allen et al., supra); and Western blots of cell sample expressing the synthetic polynucleotides.

### 6. Kits

The present invention also provides kits comprising an immunostimulatory compositions as broadly described above and elsewhere herein. Such kits may additionally comprise alternative immunogenic agents for concurrent use with the immunostimulatory compositions of the invention.

In some embodiments, in addition to the immunostimulatory compositions of the present invention the kit may include suitable components for performing an administration regimen as described above. For example, the kit may include a priming dose of the immune stimulator.

The kit may comprise additional components to assist in performing the methods of the present invention such as, for example, administration device(s), buffer(s), and/or diluent(s). The kits may include containers for housing the various components and instructions for using the kit components in the methods of the present invention.

In order that the invention may be readily understood and put into practical effect, particular preferred embodiments will now be described by way of the following non-limiting examples.

### EXAMPLES

### Example 1

### mRNA Vaccines Containing a Modified α-GalCer Protect Against Sporozoite Challenge

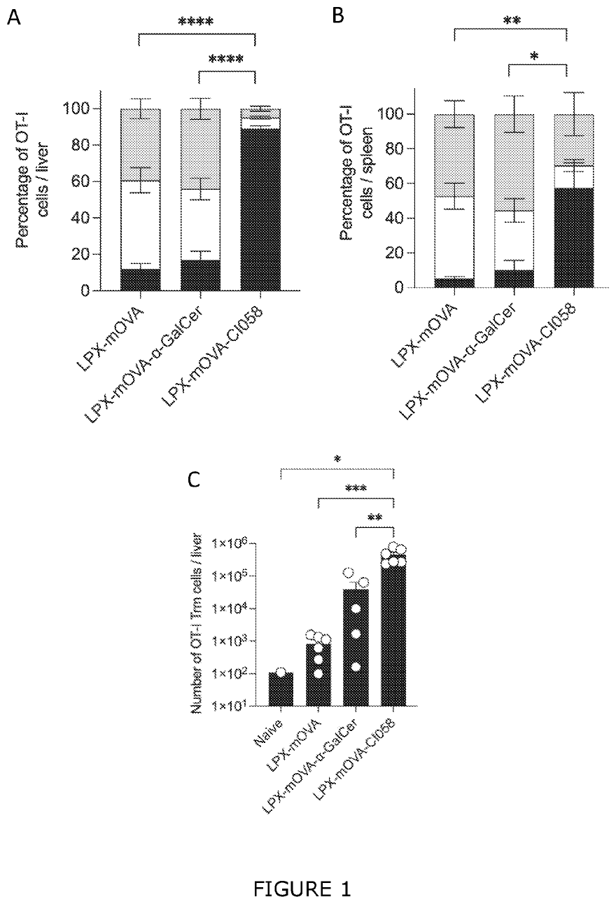

A lipoplex (LPX) vaccine was prepared using mRNA encoding a defined antigen with a known CD8⁺ T cell epitope complexed with a standard liposome prepared from DOTMA and DOPE lipids via a thin film hydration method. Intravenous (i.v.) administration of this vaccine (LPX-mOVA) can induce detectable antigen-specific T cell responses in liver and spleen of some animals, featuring a small proportion of T_{RM} cells, and larger populations of effector memory T cells (T_{EM}) cells and central memory T (T_{CM}) cells. (FIG. 1A, B).

When α-GalCer is included in the LPX vaccine, which binds CD1d and specifically stimulates NKT cells, antigen-specific T cell responses are again detected in some animals, and again with low proportions of T_{RM} cells in liver and spleen. However, when CI058³ (a modified α-GalCer structure with BODIPY attached via a thioether at the 6′ position of the galactose) is included the proportion of liver T_{RM} cells is enhanced in both tissues, although is consistently more pronounced in liver (in repeated experiments).

Vaccines with CI058 induce higher overall levels of T cell accumulation in liver when compared to vaccines without CI058. When when combined with higher T_{RM} cell proportion, this results in accumulation of significantly larger populations of T_{RM} cells in the liver tissue (FIG. 1C).

These data show that LPX vaccines that include CI058 induce antigen-specific CD8⁺ T cells responses that are significantly biased towards T_{RM} cells. This effect is more pronounced in liver, where there was an associated increase in antigen-specific CD8⁺ T cell numbers, giving significantly enhanced populations of liver T_{RM} cells compared to vaccines with α-GalCer, or without any NKT cell agonist.

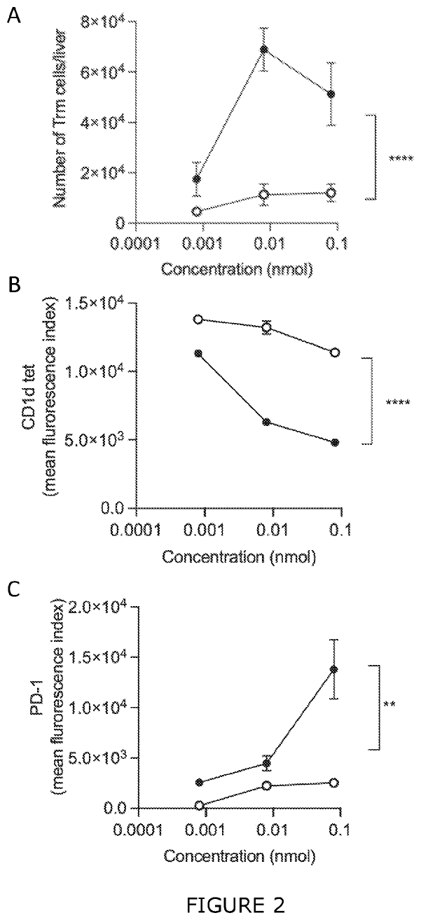

In a dose response analysis, vaccines with CI058 are more potent at inducing liver T_{RM} than vaccines with α-GalCer, achieving parity in liver T_{RM} cells revels with a 10-fold lower dose (FIG. 2A). In addition, while the dose response to both vaccines is saturated at similar concentrations, the peak level of liver T_{RM} cells reached with vaccines containing CI058 is significantly higher than achieved with vaccines with α-GalCer, implying differences in mechanism to drive T_{RM} cell accumulation. The superior liver T_{RM} response induced with vaccines containing CI058 is correlated with a change in NKT cell phenotype in the liver, featuring significantly reduced expression of the NKT cell-associated “invariant” TCR (detected by flow cytometry with CD1d/α-GalCer tetramers), and significantly increased expression of PD-1; both changes correlate with dose (FIG. 2B, C).

FIG. 2 clearly shows that LPX vaccines that include CI058 are more potent at inducing liver T_{RM} cells than vaccines with α-GalCer, achieving similar liver T_{RM} cell levels with a 10-fold lower dose. The T_{RM} cell response induced with vaccines containing CI058 is correlated with a change in NKT cell phenotype in the liver, featuring significantly reduced expression of the TCR, and significantly increased expression of PD-1.

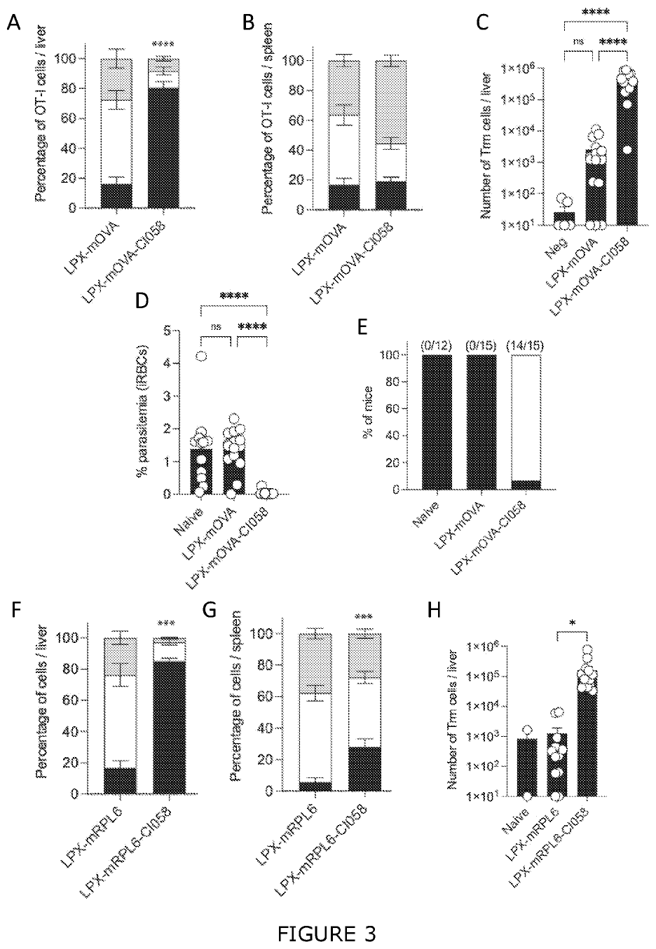

To examine the functionality of the T_{RM} cell response induced by vaccines with CI058, protection was examined in a mouse model of malaria infection, which is known to be correlated with malaria-specific T_{RM} numbers in liver. Vaccines encompassing mRNA for a model antigen expressed in Plasmodium berghei induce significant changes in proportion of antigen-specific T_{RM} cells, which is more pronounced in liver compared to spleen (FIG. 3A-B). The accumulation of T_{RM} cell in liver is also enhanced (FIG. 3C). Importantly, these vaccines also provide protection against challenge with P. berghei sporozoites expressing the target antigen, and significantly limit blood parasitemia, whereas vaccines without the agonist did not provide any protection. A similar profile of T cell response, with CI058 enhancing liver T_{RM} cell induction, is seen using vaccines encoding a known malaria antigen (rather than model antigen) (FIG. 3F-H). These data show that LPX vaccines with CI058 induce functional antigen-specific CD8⁺ T_{RM} cells that provide protection from infection in the liver, and that this vaccine platform can be used to generate T_{RM} cells to genuine parasite antigens

Overall, these data show that the NKT cell agonist CI058 is superior to α-GalCer in facilitating mRNA vaccine-induced accumulation of liver T_{RM} cells, and that the T_{RM} cells induced have capacity to protect against infection of the liver.

### Example 2

### LPX-mRNA Vaccines Containing CI058 Induce Liver T_{RM} Cells

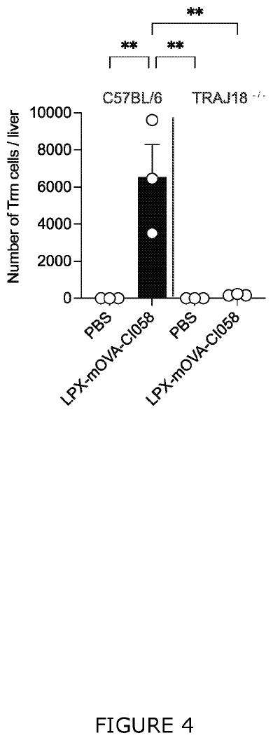

As CI058 is a NKT cell agonist, the mechanism of activity is likely dependent on the activity of these cells. Indeed, no liver T_{RM} cell accumulation is induced with LPX vaccines incorporating CI058 when administered to TRAJ18-deficient mice, which do not harbour any NKT cells (FIG. 4). These data show that NKT cells are critically involved in the mechanism by which LPX vaccines that include CI058 have an enhanced capacity for induction of liver T_{RM} cells.

Given the mechanistic role of NKT cells in T_{RM} cell induction, the superior activity of CI058 compared to α-GalCer could be attributed to the way in which the agonist is presented via CD1d, perhaps requiring the bulky BODIPY moiety to be cleaved before the agonist can bind efficiently to CD1d to stimulate NKT cells. The CI058 structure may therefore be acting as a “prodrug”, affecting the level and kinetics of presentation by CD1d, which could affect the quality of NKT cell stimulation and alter functional activity.

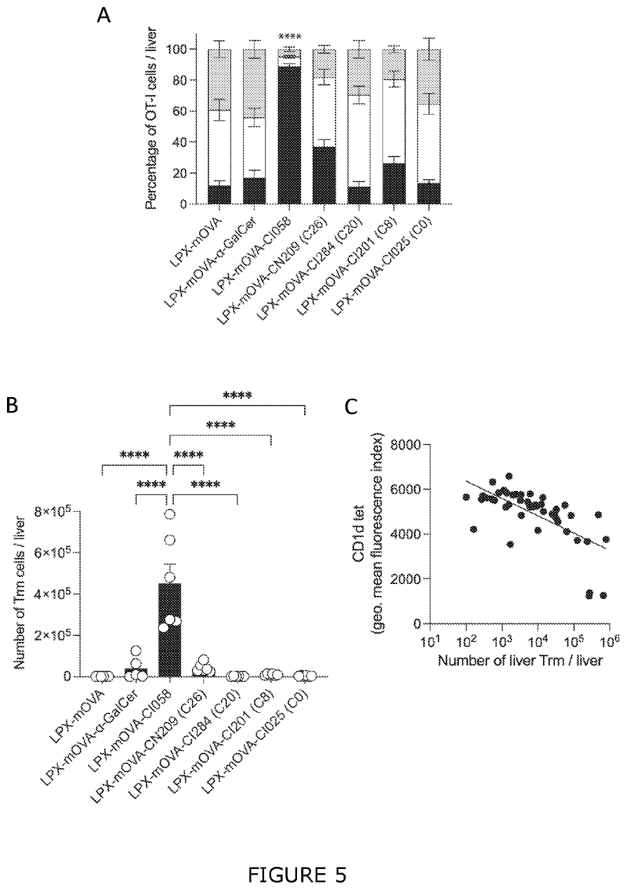

To examine whether such a prodrug mechanism can work as a general mechanism to enhance T_{RM} cell induction, prodrugs based on α-GalCer were used where the acyl chain is mis-orientated to form an inactive ligand until in vivo enzymatic activity releases the active agonist. A series of prodrugs were used with different acyl chains, as these have different binding capacities for CD1d and may therefore also alter the quality of presentation to NKT cells (CN209, C26 acyl chain; CI284, C20; CI201, C8; CI025, C0). Having established that enhanced proportion of liver T_{RM} cells among all vaccine-induced antigen-specific T cells is a feature of CI058, this measure was used as an indicator of T_{RM} cell-inducing capability of the different prodrugs. By this analysis, CI058 is a superior liver T_{RM} cell-inducing agonist, with the other prodrugs having limited activity that was similar to α-GalCer (FIG. 5A). Vaccines containing CI058 were also significantly better at inducing accumulation of T_{RM} cells (FIG. 5B). Capacity to induce liver T_{RM} cell is therefore not an attribute of all prodrug agonists. It is also noteworthy that overall T_{RM} cell-inducing capacity of the vaccines used in this experiment is significantly correlated with an NKT cell phenotype in the liver defined by down-regulation of the TCR (FIG. 5C), as had been shown earlier in FIG. 2B.

These data show that the capacity to induce liver T_{RM} cells is not an attribute of all prodrug agonists. The experiment also provides further support for the observation that T_{RM} cell-inducing capacity of the vaccines is correlated to level of down-regulation of the TCR on NKT cells in the liver.

### Example 3

### α-GalCer Derivatives with 6′ Substitutions Increase Proportion of Liver T_{RM}

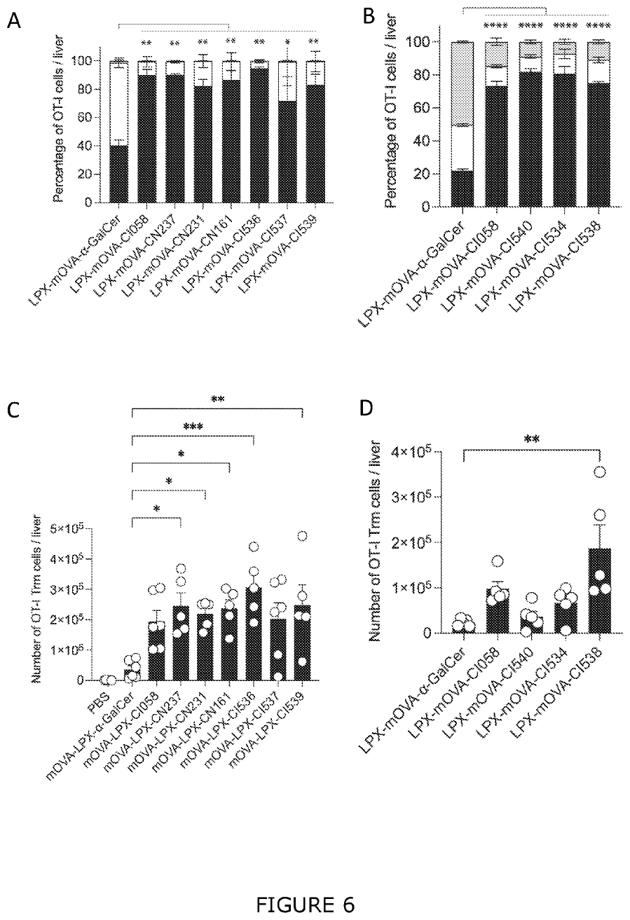

The T_{RM} cell-inducing capability of CI058 in LPX vaccines was examined further through structure-activity relationship studies of the NKT cell agonist, with readout being capacity to enhance proportion of liver T_{RM} among all vaccine-induced antigen-specific T cells. As CI058 has BODIPY attached via a thioether at the 6′position of the galactose, vaccines containing compounds featuring a 6'S-substitution alone (CN237),⁴ or further modifications via this group (CN161, CI534, CI536-40) were tested. All vaccines with such 6'S-substitutions have T_{RM} cell-inducing capability, resulting in a bias towards T_{RM} over other T cell subsets, that is significantly enhanced relative to vaccines with α-GalCer (FIG. 6A, B). All but two of the 6'S-substituted agonists (CI534, CI540) also induced significantly higher levels of overall T_{RM} cell accumulation in the liver (FIG. 6C, D). These data show that α-GalCer-based agonists with modifications that include a 6'S-substitution of the galactose are T_{RM} cell-enhancing when incorporated into LPX-mRNA vaccines.

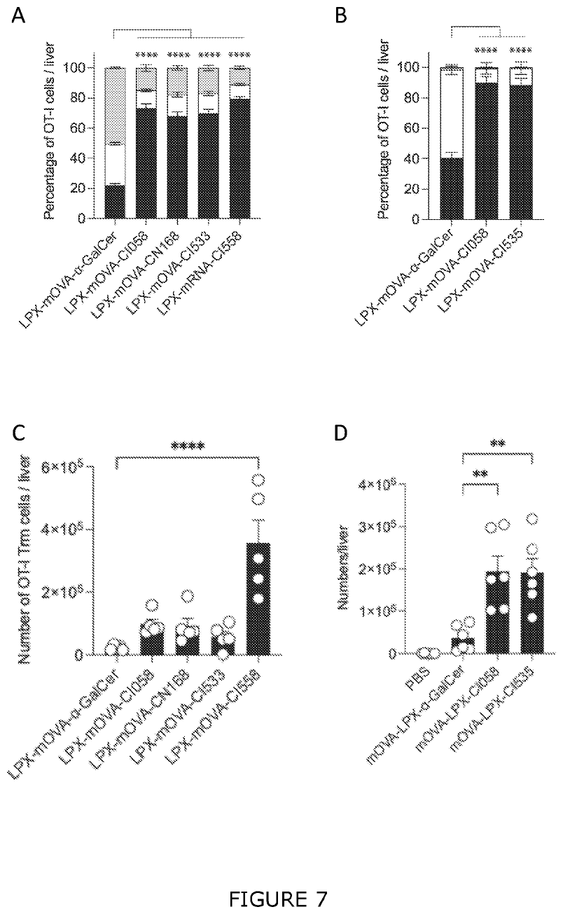

Having established a role for 6'S-substitutions in enhanced T_{RM} cell-inducing capability of NKT cell agonists, the possibility of utilising a different substitution was considered. A series of compounds was therefore generated to evaluate 6-amino substitution through structure-activity relationship studies. Focusing firstly on capacity to enhance proportion of liver T_{RM} among all vaccine-induced antigen-specific T cells, vaccines containing compounds featuring a 6-amino substitution alone (CN168), or further modifications including an amido (CI533), tetrzole (CI535) or carbamate (CI558), all have T_{RM}-inducing capability that is significantly enhanced relative to vaccines with α-GalCer (FIG. 7A, B). Some (i.e., CI535 and CI558), but not all, of the 6-nitrogen substituted agonists also induced significantly higher levels of liver T_{RM} cell accumulation in addition to the bias in proportion (FIG. 7C, D). These data demonstrate that α-GalCer-based agonists with modifications that include an amino substitution of the galactose 6-hydroxyl with an amine are T_{RM}-enhancing when incorporated into an LPX-mRNA vaccine.

In sum, these data demonstrate that while all of the agonists modified via 6′-N or 6′-S are uniquely liver T_{RM} cell inducing, resulting in an antigen-specific T cell profile that is dominated by T_{RM} cells over T_{EM} cells and T_{CM} cells, some selected compounds also have a heightened capacity to enhance overall T cell accumulation in the liver, resulting in significantly larger pools of liver T_{RM} cells.

### Example 4

### Intramuscular Administration of Lipid Nanoparticles Containing CI058

LNPs are well regarding as being effective delivery vehicles for mRNA and have been described in several vaccine applications. The capacity of LNP-mRNA vaccines containing CI058 to stimulate liver T_{RM} was therefore evaluated.

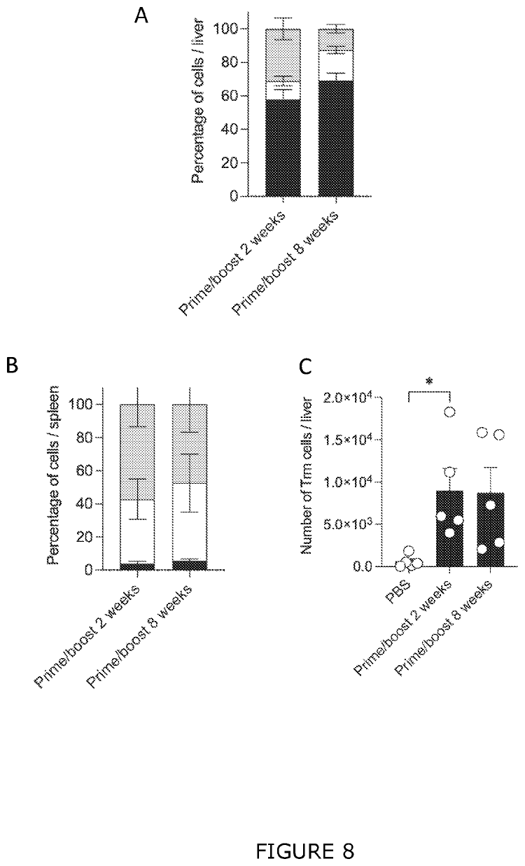

As the lipid composition used to form LNP has yet to be optimised and an intramuscular (i.m.) administration protocol adopted, a prime and boost regimen was used in order to maximise chances of detecting induced T cell responses. Using a two-week interval between doses, or an 8-week interval, LNP-mOVA-CI058 vaccines are able to induce responses with a high proportion of T_{RM} cells in the liver (FIG. 8A), which is not observed in spleen (FIG. 8B). While there is a trend toward increased overall liver T_{RM} cell accumulation when compared to vehicle-treated controls with both dosing regimens, this is only statistically significant with the two-week interval between doses (FIG. 8C).

These data show that the capacity of CI058 to induce liver TRM cells is not just a feature of a liposomal dosage form delivered i.v., but instead, this activity can be exploited in other delivery vehicles and administration routes.

### Example 5

### Intramuscular Administration of CI536/Ionisable Lipid Nanoparticles Increase Liver T_{RM}

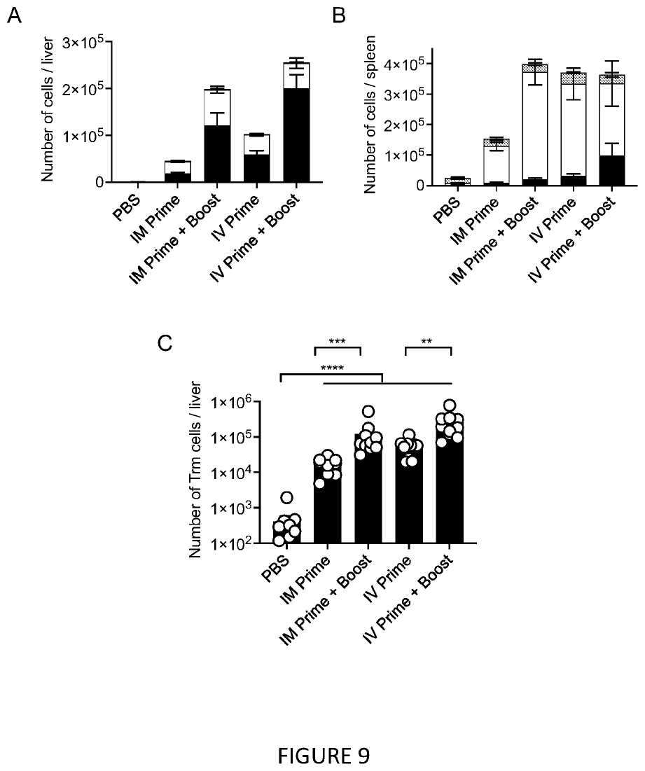

LNP-mRNA vaccines comprising an ionizable lipid are an established formulation strategy to deliver mRNA vaccines safely and effectively. Therefore, the capacity of LNP-mRNA vaccines containing CI536 and the ionizable lipid DLin-MC3-DMA to stimulate liver T_{RM} was evaluated. Both intramuscular (i.m.) and intravenous (i.v.) prime and boost administration protocols were adopted using a two-week interval between doses. To better model the intended use there was no prior transfer of antigen specific T cells. The memory T cell responses were assessed 28 days after the prime and boost so the impact of the second dose could be assessed. Memory T cell responses were evident in all four test groups (FIGS. 9A and 9B) with an increased proportion of liver T_{RM} in the liver as compared to the spleen. In the spleen a higher proportion of T_{EM} were evident.

Both intramuscular and intravenous administration route boosted groups gave the highest number of liver T_{RM} compared to PBS control group (FIG. 9C). These data demonstrate that LNP-mRNA vaccines comprising CI536 and DLin-MC3-DMA lipid nanoparticles can produce high numbers of liver T_{RM} following a homologous prime boost protocol. These data also show this vaccine approach is capable of generating endogenous liver T_{RM} responses that do not require the prior transfer of antigen-specific T cells.

### Materials & Methods

### 1. Synthesis of α-GalCer Derivatives

Anhydrous solvents are obtained commercially. Air sensitive reactions are carried out under Ar. Thin layer chromatography (TLC) is performed on aluminium sheets coated with 60 F₂₅₄ silica. Flash column chromatography is performed on Merck or SiliCycle silica gel (40-63 μm) or SiliCycle reversed phase (C18) silica gel (40-63 μm). NMR spectra are recorded on a Bruker 500 MHz spectrometer. 1H NMR spectra are referenced to tetramethylsilane at 0 ppm (internal standard) or to residual solvent peak (CHCl₃ 7.26 ppm, CHD₂OD 3.31 ppm). ¹³C NMR spectra are referenced to tetramethylsilane at 0 ppm (internal standard) or to the deuterated solvent peak (CDCl₃ 77.0 ppm, CD₃OD 49.0 ppm). CDCl₃-CD₃OD solvent mixtures are always referenced to the methanol peak. High resolution electrospray ionization mass spectra are recorded on a Q-Tof Premier mass spectrometer.

### 1.1 Synthesis of (2S,3S,4R)-1-(6-Deoxy-6-((2-((4-(5,5-difluoro-1,3,7,9-tetramethyl-5H-4λ4,5λ4-dipyrrolo[1,2-c:2′,1′-f][1,3,2]diazaborinin-10-yl)phenyl)amino)-2-oxoethyl)thio)-α-D-galactopyranosyloxy)-2-hexacosanoylamino-3,4-octadecandiol (CI058)1

To a solution of CN237² (34 mg, 0.039 mmol) in MeOH/CH₂C₂ (1:1, 10 mL) is added N-(4-(5,5-difluoro-1,3,7,9-tetramethyl-5H-4λ⁴,5λ⁴-dipyrrolo[1,2-c:2′,1′-f][1,3,2]diazaborinin-10-yl)phenyl)-2-iodoacetamide² (75 mg, 0.041 mmol, 35 mass %) in MeOH/CH₂Cl₂ (1:1, 6 mL) followed by DIPEA (15 μL, 0.086 mmol) and the reaction is stirred at RT under Ar. The mixture is diluted with CH₂Cl₂ (10 mL), concentrated, and the residue is purified by silica gel chromatography (CHCl₃ changing to 15% MeOH/CHCl₃) to yield the target compound CI058 (40 mg, 82%) as a red solid. NMR data was consistent with that reported.¹

### 1.2 Synthesis of (2S,3S,4R)-1-(6-Deoxy-6-((2-amino-2-oxoethyl)thio)-α-D-galactopyranosyloxy)-2-hexacosanoylamino-3,4-octadecandiol (CI536)

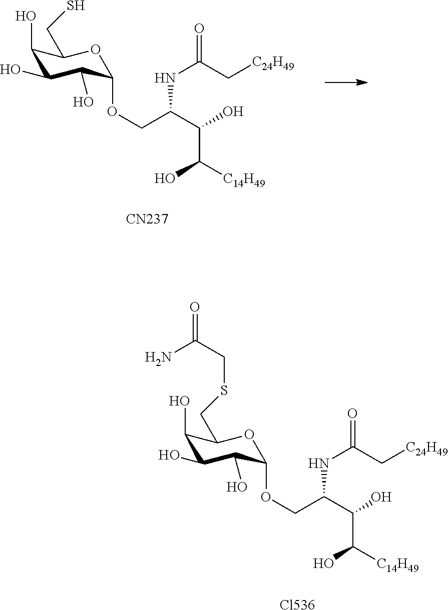

To a mixture of thiol CN237³ (5.1 mg, 5.8 μmol) and iodoacetamide (1.7 mg, 8.9 μmol) in 1:1 deoxygenated DMF/CHCl₃ (0.5 mL) is added i-Pr₂NEt (2.0 μL, 11 μmol). The vessel is briefly warmed to 50° C. to solubilize the mixture and stirred at rt overnight. After 24 h, the volatiles are concentrated (rotary evaporator) and the residue is triturated twice with MeOH. The remaining solid is dried under vacuum to afford the title compound as a white solid (3.7 mg, 68%). 1H NMR (500 MHz, 3:1 DMSO-d₆/CDCl₃) δ 7.54 (d, J=8.9 Hz, 1H), 7.36 (s, 1H), 6.95 (s, 1H), 4.70 (d, J=3.2 Hz, 1H), 4.56 (d, J=5.8 Hz, 1H), 4.52-4.51 (m, 2H), 4.34 (d, J=7.3 Hz, 1H), 4.22 (d, J=6.8 Hz, 1H), 4.05-4.00 (m, 1H), 3.77 (app t, J=6.9 Hz, 1H), 3.74-3.68 (m, 2H), 3.60-3.52 (m, 2H), 3.48 (dd, J=10.5, 4.2 Hz, 1H), 3.45-3.39 (m, 2H), 3.08 (s, 2H), 2.75-2.67 (m, 2H), 2.09 (t, J=7.5 Hz, 2H), 1.54-1.41 (m, 4H), 1.36-1.11 (m, 68H), 0.86 (t, J=6.7 Hz, 6H); ¹³C NMR (126 MHz, 3:1 DMSO-d₆/CDCl₃) δ 171.7, 171.1, 99.2, 73.8, 70.6, 69.9, 69.7, 69.6, 68.3, 66.4, 49.6, 35.5, 35.0, 32.5, 31.6, 31.25, 31.23, 29.3, 29.2, 29.13, 29.10, 29.05, 28.97, 28.9, 28.70, 28.67, 28.63, 25.4, 25.3, 22.0, 13.8. HRMS (ESI): m/z calcd for C₅₂H₁₀₃N₂O₉S [M+H]⁺ 931.7384, found 931.7372.

### 1.3 Synthesis of (2S,3S,4R)-1-(6-Deoxy-6-((2-oxo-2-(phenylamino)ethyl)thio)-α-D-galactopyranosyloxy)-2-hexacosanoylamino-3,4-octadecandiol (CI537)

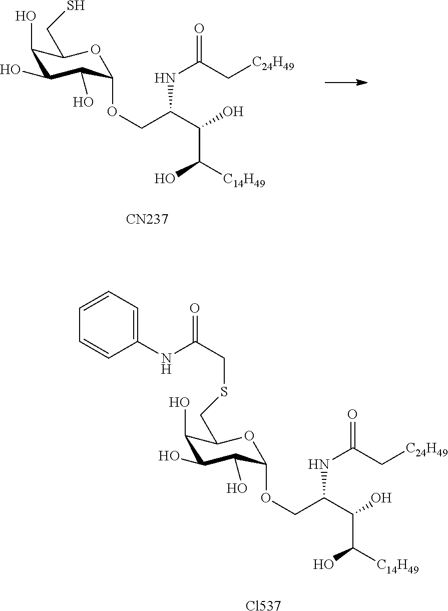

2-Iodo-N-phenylacetamide⁴ (2.3 mg, 0.0085 mmol) and i-Pr₂NEt (3 μL, 0.017 mmol) are added to thiol CN237³ (6.1 mg, 0.0070 mmol) in 1:1 CH₂Cl₂/MeOH (1 mL). After 2 hours stirring at room temperature, the reaction is concentrated and the solid triturated with ice-cold i-PrOH (2×4 mL) and dried under vacuum to yield the target compound CI537 (5.5 mg, 82%) as a white solid. 1H NMR (500 MHz, 3:1 CDCl₃/CD₃OD) δ 7.56 (d, J=8.0 Hz, 2H), 7.33 (t, J=7.8 Hz, 2H), 7.12 (t, J=7.8 Hz, 1H), 4.87 (d, J=3.8 Hz, 1H), 4.21 (q, J=4.6 Hz, 1H), 3.99-3.91 (m, 3H), 3.80-3.63 (m, 4H), 3.61-3.53 (m, 2H), 3.44 (d, J=14.8 Hz, 1H), 3.40-3.32 (obscured by solvent, obsvd hsqc, 1H), 2.93 (dd, J=13.9, 7.9 Hz, 1H), 2.83 (dd, J=13.9, 5.8 Hz, 1H), 2.20 (t, J=7.6 Hz, 2H), 1.72-1.44 (m, 4H), 1.45-1.20 (m, 68H), 0.88 (t, J=6.8 Hz, 6H). ¹³C NMR (126 MHz, 3:1 CDCl₃/CD₃OD) δ 174.4, 168.8, 137.8, 128.8, 124.5, 120.1, 99.5, 74.6, 72.0, 70.5, 70.2, 70.0, 68.7, 67.3, 50.2, 36.5, 32.8, 32.5, 31.8, 29.7, 29.6, 29.6, 29.5, 29.3, 29.3, 29.2, 25.8, 25.8, 22.6, 13.8. HRMS (ESI): m/z calcd for C₅₈H₁₀₆N₂O₉SNa [M+Na]⁺ 1029.7517, found 1029.7517.

### 1.4 (2S,3S,4R)-1-(6-Deoxy-6-phenylthio)-α-D-galactopyranosyloxy)-2-hexacosanoylamino-3,4-octadecandiol (CN231)⁵

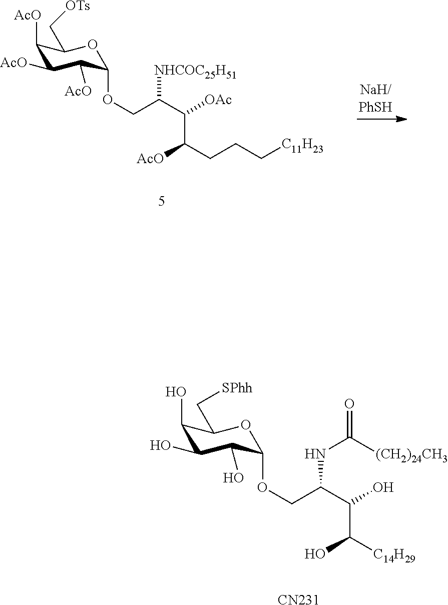

Sodium hydride (60% dispersion in mineral oil, 1.5 mg, 0.037 mmol) is added to a stirred solution of 5⁴ (6.2 mg, 0.0051 mmol) and thiophenol (5.0 μL, 0.048 mmol) in dry DMF (50 μL) under argon. After 1 h at 65° C. the cooled reaction mixture is partitioned between ethyl acetate (1 mL) and sat aq sodium bicarbonate (1 mL). The aqueous phase is thoroughly extracted with ethyl acetate and the combined organic extracts are dried (MgSO₄) and concentrated at reduced pressure to afford a solid (9.2 mg). The crude material is dissolved in 2:3 CH₂Cl₂/MeOH (0.25 mL), treated with NaOMe (0.5 M in MeOH, 20 μL, 0.01 mmol) and stirred at RT for 1 h. The reaction mixture is quenched with the addition of formic acid (2 μL, 0.053 mmol), and purified by silica gel chromatography (2% MeOH/CH₂Cl₂ changing to 6% MeOH/CH₂Cl₂) to yield the target compound CN231 (3.0 mg, 62%) as a white solid. 1H NMR (500 MHz, CDCl₃/CD₃OD 2:3) δ 0.89 (t, J=7.1 Hz, 6H), 1.22-1.45 (m, 68H), 1.50-1.68 (m, 4H), 2.16 (t, J=7.7 Hz, 2H), 3.14-3.24 (m, 2H), 3.52-3.61 (m, 2H), 3.69 (dd, J=10.6, 3.8 Hz, 1H), 3.72 (dd, J=10.0, 3.4 Hz, 1H), 3.79 (dd, J=10.0, 3.9 Hz, 1H), 3.84 (dd, J=10.7, 4.4 Hz, 1H), 3.89-3.92 (m, 1H), 3.95-3.96 (m, 1H), 4.16-4.19 (m, 1H), 4.88 (d, J=3.9 Hz) 7.15-7.18 (m, 1H), 7.26-7.30 (m, 2H), 7.33-7.36 (m, 2H); ¹³C NMR (126 MHz, CDCl₃/CD₃OD 2:3) δ 14.2, 23.0, 26.3, 29.7, 29.8, 30.0, 30.1, 30.2, 32.3, 32.7, 34.2, 36.9, 50.5, 68.1, 69.2, 70.27, 70.33, 70.8, 72.5, 74.9, 100.2, 126.4, 129.1, 129.4, 136.7, 174.6; HRMS (ESI): m/z calcd for C₅₆H₁₀₃NO₈SNa [M+Na]⁺ 972.7302, found 972.7294.

### 1.5 CI533

### 1.5.1 Dioxopyrrolidin-1-yl 6-((4-(5,5-difluoro-1,3,7,9-tetramethyl-5H-4λ4,5λ4-dipyrrolo[1,2-c:2′,1′-f][1,3,2]diazaborinin-10-yl)phenyl)amino)-6-oxohexanoate

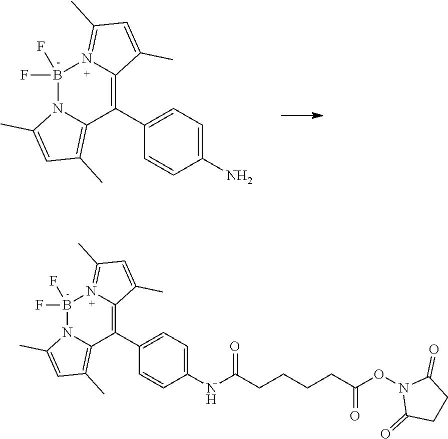

A mixture of 4-(5,5-difluoro-1,3,7,9-tetramethyl-5H-4λ⁴,5λ⁴-dipyrrolo[1,2-c:2′,1′-f][1,3,2]diazaborinin-10-yl)aniline⁶ (30 mg, 0.088 mmol) and bis(2,5-dioxopyrrolidin-1-yl) adipate⁷ (137 mg, 0.403 mmol) in DMF (1 mL) is warmed to 50° C. to solubilize the mixture, before adding i-Pr₂NEt (60 μL, 0.34 mmol) and stirring at 50° C. for 20 h. A second portion of i-Pr₂NEt (60 μL, 0.34 mmol) is added and stirring is continued at 70° C. for a further 19 h. The mixture is concentrated and purified by silica gel chromatography (100% CHCl₃ to 35% EtOAc/CHCl₃) to yield the target compound (16.8 mg, 34%) as a red solid. 1H NMR (500 MHz, CDCl₃) δ 7.82 (s, 1H), 7.72 (d, J=8.4 Hz, 2H), 7.21 (d, J=8.4 Hz, 2H), 5.97 (s, 2H), 2.87 (br, 4H), 2.70-2.67 (m, 2H), 2.55 (s, 6H), 2.46-2.43 (m, 2H), 1.92-1.87 (m, 4H), 1.43 (s, 6H); ¹³C NMR (101 MHz, CDCl₃) δ 171.0, 169.5, 168.7, 155.6, 143.3, 141.6, 139.2, 131.8, 130.5, 128.8, 121.3, 119.9, 37.0, 30.7, 25.8, 24.6, 24.1, 14.8, 14.7; HRMS (ESI): m/z calcd for C₂₉H₃₁¹¹BF₂N₄NaO₅ [M+Na]⁺ 587.2253, found 587.2267.

### 1.5.2 CI533

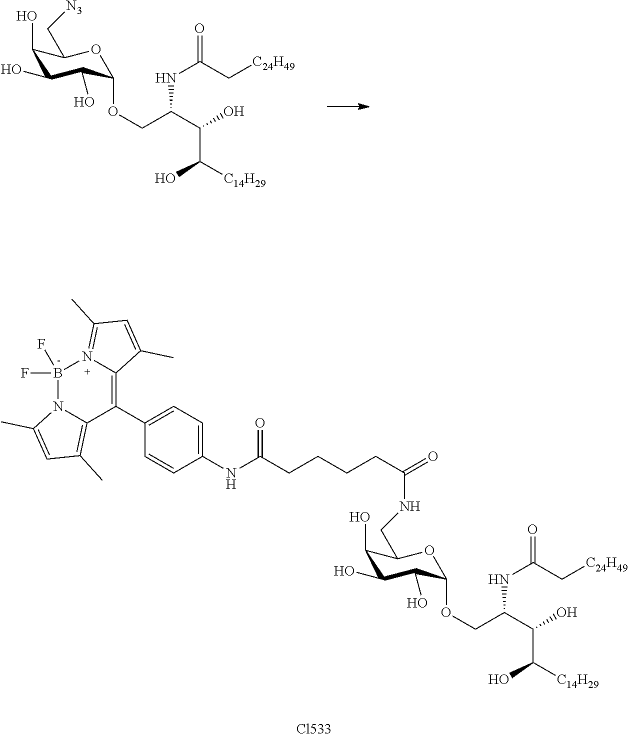

To a solution of 6-deoxy-6-azido-α-galactosylceramide⁸ (21 mg, 0.024 mmol) in CH₂Cl₂/MeOH (1:1, 10 mL) under argon is added 10% Pd/C (5 mg, 0.005 mmol), the atmosphere swapped for H₂ and reaction mixture is stirred at room temperature (2 h). The solution is filtered through glass fibre, the catalyst washed with CHCl₃/MeOH (1:1, 50 mL) then hot EtOH (50 mL) and the filtrate is concentrated. To the resulting residue is added a solution of 2,5-dioxopyrrolidin-1-yl 6-((4-(5,5-difluoro-1,3,7,9-tetramethyl-5H-4λ⁴,5λ⁴-dipyrrolo[1,2-c:2′,1′-f][1,3,2]diazabor-inin-10-yl)phenyl)amino)-6-oxohexanoate (15 mg, 0.027 mmol) in dry pyridine (5 mL) followed by i-Pr₂NEt (50 μL) and the mixture is stirred at room temperature (18 h). The reaction is quenched by the addition of methanol (10 mL), concentrated, and the residue is purified by silica gel chromatography (CHCl₃ changing to 15% MeOH/CHCl₃) to yield CI533 (21 mg, 68%, 2 steps) as a red solid. 1H NMR (500 MHz, CDCl₃/CD₃OD 1:1) δ 7.79 (d, J=8.2 Hz, 2H), 7.23 (d, J=8.2 Hz, 2H), 6.02 (s, 2H), 4.89 (d, J=3.7 Hz, 1H), 4.24-4.18 (m, 1H), 3.91-3.77 (m, 4H), 3.76-3.72 (m, 1H), 3.71-3.65 (m, 1H), 3.63-3.52 (m, 3H), 3.27 (dd, J=13.8, 7.8 Hz, 1H), 2.52 (s, 6H), 2.44 (t, J=7.0 Hz, 2H), 2.28 (t, J=7.0 Hz, 2H), 2.22 (t, J=7.6 Hz, 2H), 1.86-1.52 (m, 8H), 1.46 (s, 6H), 1.36-1.25 (m, 68H), 0.89 (t, J=6.8 Hz, 6H); ¹³C NMR (126 MHz, CDCl₃/CD₃OD 1:1) δ 174.98, 174.47, 172.91, 155.20, 143.31, 141.81, 139.64, 131.57, 129.93, 128.42, 121.08, 120.05, 99.59, 74.32, 71.84, 69.94, 69.60, 69.10, 68.77, 66.93, 50.39, 48.80, 48.75, 48.62, 48.58, 48.45, 48.40, 48.28, 48.23, 48.12, 48.07, 47.95, 47.90, 47.77, 47.72, 39.82, 36.57, 36.26, 35.58, 32.16, 31.77, 29.68, 29.63, 29.55, 29.50, 29.42, 29.30, 29.25, 29.19, 25.82, 25.76, 25.24, 25.04, 22.48, 14.16, 14.12, 13.90, 13.59, 13.56; ¹⁹F NMR (470 MHz, CDCl₃/CD₃OD 1:1) δ −146.2-146.5 (m); HRMS (ESI): m/z calcd for C₇₅H₁₂₆¹¹BN₅O₁₀F₂Na [M+Na]⁺ 1328.9464, found 1328.9463.

### 1.6 CI535

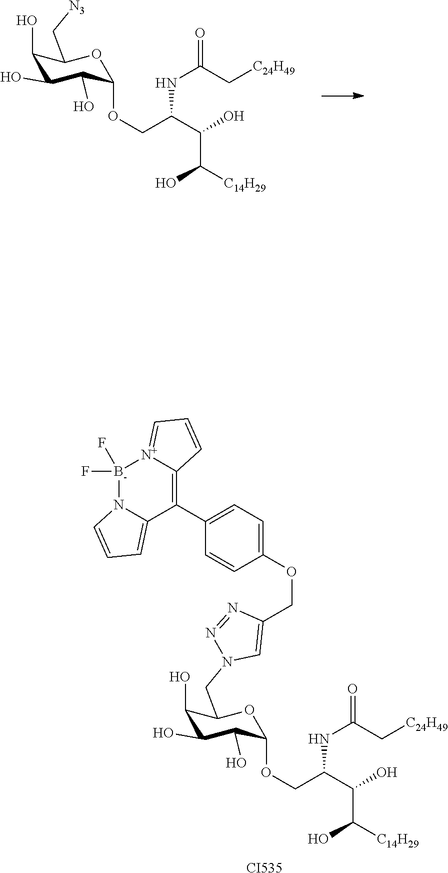

To a solution of 6-deoxy-6-azido-α-galactosylceramide⁸ (8.5 mg, 0.0096 mmol), 5,5-difluoro-10-(4-(prop-2-yn-1-yloxy)phenyl)-5H-4λ⁴,5λ⁴-dipyrrolo[1,2-c:2′,1′-f][1,3,2]diazaborinine⁹ (4.5 mg, 0.011 mmol) and TBTA (1.0 mg, 0.0018 mmol) in (2:1) MeOH/CHCl₃ (2.5 mL) is added a piece of copper foil (3 mm×5 mm) and 2.5 mM CuSO₄ (320 μL). The reaction is stirred at 30° C. for 24 h, then the solution is concentrated, and the residue is triturated with 50 mM EDTA (2×5 mL). The crude solid is purified by silica gel chromatography (CHCl₃ changing to 25% MeOH/CHCl₃ to yield the target compound CI535 (9.1 mg, 79%). 1H NMR (500 MHz, 3:1 CDCl₃/CD₃OD) δ 7.95 (s, 1H), 7.92 (s, 2H), 7.59 (d, J=8.7 Hz, 2H), 7.19 (d, J=8.7 Hz, 2H), 7.01 (d, J=4.2 Hz, 2H), 6.59 (dd, J=4.3, 1.9 Hz, 2H), 5.29 (s, 2H), 4.91 (d, J=3.8 Hz, 1H), 4.76-4.48 (m, 2H), 4.24 (dd, J=8.5, 5.0 Hz, 1H), 4.18-4.11 (m, obscured by water, obsvd hsqc, 1H), 3.87 (d, J=3.3 Hz, 1H), 3.83 (dd, J=10.0, 3.8 Hz, 1H), 3.78-3.72 (m, 2H), 3.58 (dd, J=10.5, 5.0 Hz, 1H), 3.52-3.43 (m, 3H), 2.23-2.08 (m, 2H), 1.70-1.45 (m, 4H), 1.42-1.17 (m, 68H), 0.91-0.85 (m, 6H); ¹³C NMR (126 MHz, 3:1 CDCl₃/CD₃OD) δ 174.2, 160.8, 147.3,143.4, 143.0, 134.8, 132.41, 131.5, 126.7, 124.9, 118.3, 114.8, 99.5, 74.6, 71.9, 69.8, 69.4, 69.3, 68.6, 67.2, 61.7, 51.0, 50.0, 36.4, 32.6, 31.8, 29.7, 29.6, 29.6, 29.5, 29.5, 29.3, 29.3, 29.2, 25.8, 25.7, 22.6, 13.82; HRMS (ESI): m/z calcd for C₅₈H₁₁₁¹¹BF₂N₆O₉Na [M+Na]⁺ 1227.8371, found 1227.8380.

### 1.7 CI534

### 1.7.1 Step A N-(5-Bromopentyl)-3-(5,5-difluoro-1,3,7,9-tetramethyl-5H-4λ⁴,5λ⁴-dipyrrolo[1,2-c:2′,1′-f][1,3,2]diazaborinin-10-yl)propenamide

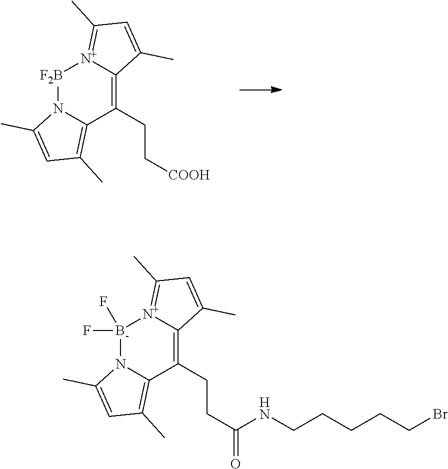

To a mixture of 5-bromopentan-1-amine hydrobromide (14 mg, 0.06 mmol) and 3-(5,5-difluoro-1,3,7,9-tetramethyl-5H-4λ⁴,5λ⁴-dipyrrolo[1,2-c:2′,1′-f][1,3,2]diazaborinin-10-yl)propanoic acid (15 mg, 0.055 mmol) in dichloromethane (3 mL) is added N-(3-dimethylaminopropyl)-N′-ethylcarbodiimide hydrochloride (20 mg, 0.10 mmol) then N,N-diisopropylethylamine (12 μL, 0.070 mmol) and the reaction is stirred at room temperature (18 h). The reaction is concentrated and resulting orange solid is purified by silica gel chromatography (toluene changing to 40% EtOAc/toluene) to yield N-(5-bromopentyl)-3-(5,5-difluoro-1,3,7,9-tetramethyl-5H-4λ⁴,5λ⁴-dipyrrolo[1,2-c:2′,1′-f][1,3,2]diazaborinin-10-yl)propanamide (17 mg, 77%) as an orange solid. ¹H NMR (500 MHz, CDCl₃) δ 6.05 (s, 2H), 5.54 (br t, J=6.0 Hz, 1H), 3.40 (t, J=6.7 Hz, 2H), 3.36-3.28 (m, 2H), 3.24 (q, J=6.5 Hz, 2H), 2.50 (s, 6H), 2.43 (s, 6H), 2.42-2.37 (m, 2H), 1.86 (p, J=6.8 Hz, 2H), 1.54-1.39 (m, 4H); ¹³C NMR (126 MHz, CDCl₃) δ 170.44, 154.46, 144.46, 140.56, 131.30, 121.89, 39.46, 39.43, 37.59, 33.51, 32.16, 28.64, 25.31, 23.84, 16.49, 14.45; ¹⁹F NMR (470 MHz, CDCl₃) δ −145.8, −146.7; HRMS (ESI): m/z calcd for C₂₁H₂₉¹¹BBrN₃OF₂Na [M+Na]⁺ 490.1453, found 490.1460.

### 1.7.2 Step B: N-(5-Iodopentyl)-3-(5,5-difluoro-1,3,7,9-tetramethyl-5H-4λ⁴,5λ⁴-dipyrrolo[1,2-c:2′,1′-f][1,3,2]diazaborinin-10-yl)propenamide

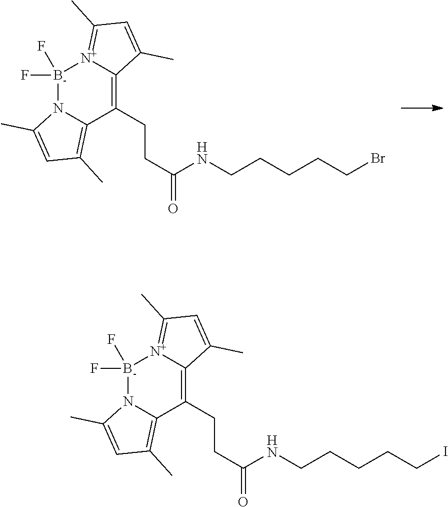

To a solution of N-(5-bromopentyl)-3-(5,5-difluoro-1,3,7,9-tetramethyl-5H-4λ⁴,5λ⁴-dipyrrolo[1,2-c:2′,1′-f][1,3,2]diazaborinin-10-yl)propanamide (17 mg, 0.04 mmol) in acetone (5 mL) is added sodium iodide (110 mg, 0.73 mmol) and the reaction mixture is stirred at 45° C. (18 h). The cooled solution is concentrated and the residue is diluted with EtOAc (20 mL) and washed with water (20 mL), brine (20 mL), dried (MgSO₄) then concentrated. The residue is purified by silica gel chromatography (toluene changing to 40% EtOAc/toluene) to yield N-(5-iodopentyl)-3-(5,5-difluoro-1,3,7,9-tetramethyl-5H-4λ⁴,5λ4-dipyrrolo[1,2-c:2′,1′-f][1,3,2]diazaborinin-10-yl)propanamide (15 mg, 80%) as an orange solid. 1H NMR (500 MHz, CDCl₃) δ 6.05 (s, 2H), 5.49 (br t, J=6.0 Hz, 1H), 3.37-3.30 (m, 2H), 3.24 (td, J=7.1, 5.8 Hz, 2H), 3.18 (t, J=6.9 Hz, 2H), 2.51 (s, 6H), 2.44 (s, 6H), 2.43-2.41 (m, 2H), 1.82 (p, J=6.9 Hz, 2H), 1.52-1.44 (m, 2H), 1.44-1.38 (m, 2H); ¹³C NMR (126 MHz, CDCl₃) δ 170.42, 154.48, 144.43, 140.54, 131.31, 121.89, 39.42, 37.67, 37.58, 32.80, 29.69, 28.42, 27.63, 23.85, 23.82, 16.54, 14.47, 6.64; ¹⁹F NMR (470 MHz, CDCl₃) δ −145.93, −146.79; HRMS (ESI): m/z calcd for C₂₁H₂₉BN₃OF₂NaI [M+Na]⁺ 538.1314, found 538.1307.

### 1.7.3 Step C: CI534

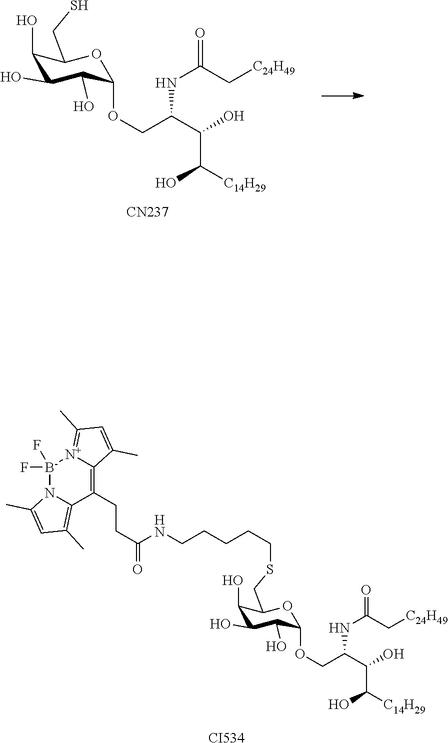

To a mixture of thiol CN2373 (12.7 mg, 0.015 mmol), N-(5-iodopentyl)-3-(5,5-difluoro-1,3,7,9-tetramethyl-5H-4λ4,5λ4-dipyrrolo[1,2-c:2′,1′-f][1,3,2]diazaborinin-10-yl)propanamide (15 mg, 0.03 mmol) and solid K2CO3 (7.8 mg, 0.06 mmol) is added dry DMF (500 μL) and the reaction is stirred under Ar at 50° C. (200 min). The reaction mixture is diluted with chloroform/MeOH (1:1, 20 mL) then concentrated. The residue is purified by silica gel chromatography (chloroform changing to 20% MeOH/chloroform) to yield CI534 (10 mg, 55%) as an orange-red solid. 1H NMR (500 MHz, 2:1 CDCl3/MeOD) δ 6.09 (s, 2H), 4.87 (d, J=3.7 Hz, 1H), 4.19 (q, J=4.5 Hz, 1H), 3.95-3.88 (m, 2H), 3.84 (t, J=6.9 Hz, 1H), 3.78 (dd, J=9.9, 3.8 Hz, 1H), 3.73 (dd, J=10.0, 3.2 Hz, 1H), 3.69 (dd, J=10.6, 3.8 Hz, 1H), 3.58-3.53 (m, 2H), 3.34-3.30 (m, 2H), 3.25-3.15 (m, 2H), 2.78 (dd, J=13.6, 7.3 Hz, 1H), 2.72 (dd, J=13.6, 6.5 Hz, 1H), 2.58 (t, J=7.3 Hz, 2H), 2.53-2.47 (m, 2H), 2.50 (s, 6H), 2.48 (s, 6H), 2.20 (t, J=7.6 Hz, 2H), 1.66-1.58 (m, 5H), 1.57-1.46 (m, 3H), 1.44-1.37 (m, 2H), 1.37-1.08 (m, 68H), 0.89 (t, J=6.8 Hz, 6H); 13C NMR (126 MHz, 2:1 CDCl3/MeOD) δ 174.98, 174.47, 172.91, 155.20, 143.31, 141.81, 139.64, 131.57, 129.93, 128.42, 121.08, 120.05, 99.59, 74.32, 71.84, 69.94, 69.60, 69.10, 68.97, 68.77, 66.93, 50.39, 39.82, 36.57, 36.26, 35.58, 32.16, 31.77, 29.68, 29.63, 29.55, 29.50, 29.42, 29.30, 29.25, 29.19, 25.82, 25.76, 25.24, 25.11, 25.04, 22.48, 14.16, 14.12, 13.90, 13.59, 13.56; 19F NMR (470 MHz, 2:1 CDCl3/MeOD) δ −146.1, −146.7; HRMS (ESI): m/z calcd for C71H127BN4O9F2NaS [M+Na]+ 1283.9283, found 1283.9287.

### 1.8 CI538

### 1.8.1 N-(5-Bromopentyl)-3-(5,5-difluoro-7,9-dimethyl-5H-4λ⁴,5λ⁴-dipyrrolo[1,2-c:2′,1′-f][1,3,2]diazaborinin-3-yl)propenamide

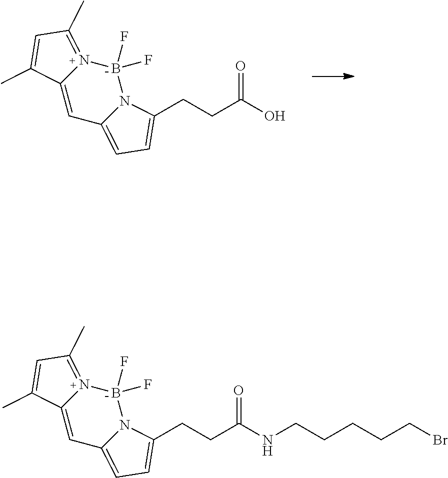

To an ice-cold solution of 3-(5,5-difluoro-7,9-dimethyl-5H-4λ⁴,5λ⁴-dipyrrolo[1,2-c:2′,1′-f][1,3,2]diazaborinin-3-yl)propanoic acid (20 mg, 0.068 mmol) and 5-bromopentan-1-amine hydrobromide (24 mg, 0.097 mmol) in CH₂Cl₂ (2 mL) is added N-(3-dimethylaminopropyl)-N′-ethylcarbodiimide hydrochloride (28 mg, 0.14 mmol) and Et₃N (14 μL, 0.10 mmol). The reaction is stirred at 0° C. for 3 h, before allowing the reaction to warm to room temperature overnight. The reaction is diluted with CH2Cl2 (20 mL) and washed with water, brine, dried (MgSO₄) and concentrated to give a crude red oil. The residue is purified by silica gel chromatography (25% EtOAc/PE changing to 40% EtOAc/PE) to yield N-(5-bromopentyl)-3-(5,5-difluoro-7,9-dimethyl-5H-4λ⁴,5λ⁴-dipyrrolo[1,2-c:2′,1′-f][1,3,2]diazaborinin-3-yl)propenamide (21 mg, 73%) as a red oil. 1H NMR (500 MHz, CDCl₃) δ 7.09 (s, 1H), 6.88 (d, J=4.0 Hz, 1H), 6.29 (d, J=4.0 Hz, 1H), 6.13 (s, 1H), 5.77 (d, J=6.4 Hz, 1H), 3.33 (t, J=6.8 Hz, 2H), 3.26 (t, J=7.4 Hz, 2H), 3.19 (q, J=6.7 Hz, 2H), 2.63 (t, J=7.5 Hz, 2H), 2.57 (s, 3H), 2.26 (s, 3H), 1.80 (p, J=7.0 Hz, 2H), 1.48-1.38 (m, 2H), 1.40-1.30 (m, 2H); ¹³C NMR (126 MHz, CDCl3) δ 171.6, 160.3, 157.3, 144.0, 135.1, 133.4, 128.3, 123.8, 120.5, 117.6, 39.1, 36.0, 33.5, 32.4, 28.6, 25.3, 24.9, 14.9, 11.3; HRMS (ESI): m/z calcd for C₁₉H₂₅¹¹BBrF₂N₃ONa [M+Na]⁺ 462.1140, found 462.1147.

### 1.8.2 3-(5,5-Difluoro-7,9-dimethyl-5H-4λ4,5λ4-dipyrrolo[1,2-c:2′,1′-f][1,3,2]diazaborinin-3-yl)-N-(5-iodopentyl)propenamide

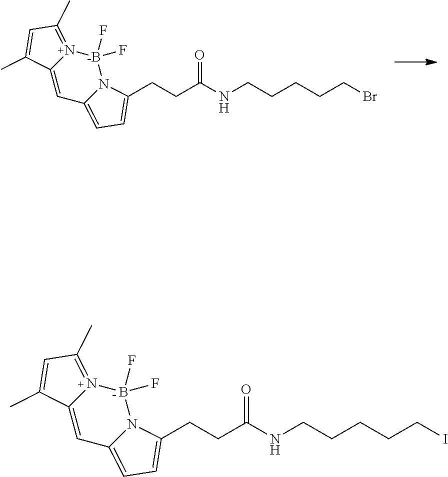

NaI (130 mg, 0.87 mmol) is added to a solution of N-(5-bromopentyl)-3-(5,5-difluoro-7,9-dimethyl-5H-4λ⁴,5λ⁴-dipyrrolo[1,2-c:2′,1′-f][1,3,2]diazaborinin-3-yl)propanamide (19 mg, 0.043 mmol) in acetone (2 mL). The reaction is heated at 45° C. for 16 h, then cooled to room temperature and concentrated to afford a crude red oil. The residue is partitioned between CH₂Cl₂/water (40 mL) and the organic layer is washed with brine, dried (MgSO4) to yield 3-(5,5-difluoro-7,9-dimethyl-5H-4λ⁴,5λ⁴-dipyrrolo[1,2-c:2′,1′-f][1,3,2]diazaborinin-3-yl)-N-(5-iodopentyl)propanamide (21 mg, 100%) as a red oil. ¹H NMR (500 MHz, CDCl₃) δ 7.10 (s, 1H), 6.89 (d, J=4.0 Hz, 1H), 6.30 (d, J=3.9 Hz, 1H), 6.13 (s, 1H), 5.74 (s, 1H), 3.26 (t, J=7.4 Hz, 2H), 3.19 (q, 2H), 3.11 (t, J=7.0 Hz, 2H), 2.64 (t, J=7.4 Hz, 2H), 2.57 (s, 3H), 2.27 (s, 3H), 1.77 (p, J=7.1 Hz, 2H), 1.47-1.38 (m, 2H), 1.35-1.24 (m, 2H); ¹³C NMR (126 MHz, CDCl₃) δ 171.6, 160.3, 157.3, 144.0, 135.2, 133.4, 128.3, 123.8, 120.5, 117.6, 39.1, 36.0, 33.1, 28.4, 27.6, 25.0, 15.0, 11.4, 6.5; HRMS (ESI): m/z calcd for C₁₉H₂₅¹¹BF₂IN₃ONa [M+Na]⁺ 510.1001, found 510.1011.

### 1.8.3 CI538

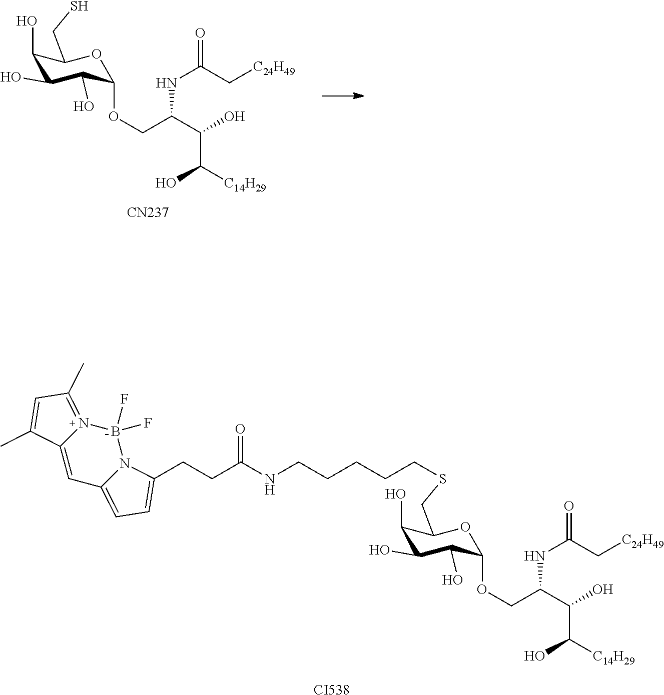

A mixture of thiol CN237³ (7.4 mg, 8.5 μmol), 3-(5,5-difluoro-7,9-dimethyl-5H-5λ⁴,6λ⁴-dipyrrolo[1,2-c:2′,1′-f][1,3,2]diazaborinin-3-yl)-N-(5-iodopentyl)propanamide (7.5 mg, 15 μmol) and potassium carbonate (3.8 mg, 27 μmol) in anhydrous deoxygenated DMF (0.36 mL) is stirred under Ar at 50° C. for 6.5 h. After cooling, the mixture is diluted with 9:1 CH₂Cl₂-MeOH, filtered and concentrated. The residue is purified by silica gel chromatography (gradient: 100% CHCl₃ through to 10% MeOH/CHCl₃) to yield the target compound CI538 as a red solid (5.7 mg, 57%). ¹H NMR (500 MHz, CDCl₃) δ 7.09 (s, 1H), 6.88 (d, J=4.0 Hz, 1H), 6.78 (d, J=8.7 Hz, 1H), 6.28 (d, J=4.0 Hz, 1H), 6.12-6.09 (m, 2H), 4.94 (d, J=3.6 Hz, 1H), 4.36-4.25 (m, 2H), 4.19 (d, J=6.5 Hz, 1H), 4.07 (br s, 1H), 4.02-4.00 (m, 1H), 3.98 (dd, J=10.4, 4.6 Hz, 1H), 3.91-3.80 (m, 3H), 3.73 (dd, J=10.4, 3.6 Hz, 1H), 3.69-3.62 (m, 2H), 3.54 (br s, 1H), 3.43 (br s, 1H), 3.25 (t, J=7.5 Hz, 2H), 3.20-3.13 (m, 2H), 2.75 (d, J=6.8 Hz, 2H), 2.64 (t, J=7.5 Hz, 2H), 2.56-2.52 (m, 5H), 2.26 (s, 3H), 2.18 (t, J=7.4 Hz, 2H), 1.66-1.50 (m, 6H), 1.45-1.39 (m, 4H), 1.35-1.11 (m, 68H), 0.88 (t, J=6.8 Hz, 6H); ¹³C NMR (126 MHz, CDCl₃) δ 173.6, 172.5, 160.6, 157.1, 144.3, 135.4, 133.5, 128.4, 124.0, 120.7, 117.4, 99.7, 75.4, 72.8, 71.2, 71.0, 70.1, 69.2, 68.7, 50.1, 39.5, 36.9, 36.0, 32.8, 32.4, 32.1, 29.94, 29.87, 29.8, 29.7, 29.61, 29.55, 29.51, 29.1, 29.0, 26.2, 26.0, 25.8, 25.0, 22.8, 15.1, 14.3, 11.5; ¹⁹F NMR (470 MHz, CDCl₃) δ −144.1 (q, J=33 Hz). HRMS (ESI): m/z calcd for C₆₉H₁₂₃¹¹BF₂N₄O₉SNa [M+Na]⁺ 1255.8970, found 1255.8960.

### 1.9 CI540

### 1.9.1 Anthracen-9-ylmethyl (5-bromopentyl)carbamate

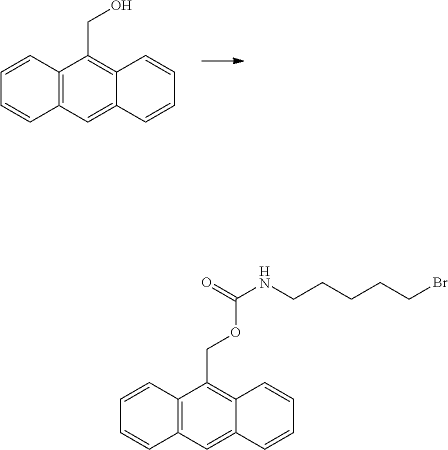 [SMILES: OCc1c2ccccc2cc2ccccc12 ; O=C(NCCCCCBr)OCc1c2ccccc2cc2ccccc12]

To a suspension of 9-anthracenemethanol (200 mg, 0.96 mmol) in CH₂Cl₂ (5 mL) is added bis-(4-nitrophenyl) carbonate (300 mg, 0.98 mmol), followed by i-Pr₂NEt (0.55 mL, 3.1 mmol). The mixture is stirred under Ar at rt, during which time a clear solution is obtained. After 5 h, 5-bromopentan-1-amine hydrobromide (221 mg, 0.89 mmol) is added and stirring is continued for 2.5 d. The reaction is subsequently diluted with CH₂Cl₂, washed with water (×2) and dried (MgSO₄) to give a crude product. Purification by silica gel chromatography (gradient: 30% CH₂Cl₂/hexane to 85% CH₂Cl₂/hexane) afforded an amorphous white solid (173 mg) consisting of a mixture of anthracen-9-ylmethyl (5-bromopentyl)carbamate (˜60%, 27% yield) and anthracen-9-ylmethyl piperidine-1-carboxylate (˜40%). 1H NMR (500 MHz, CDCl₃) δ 8.47-8.46 (m, 1H), 8.40-8.36 (m, 2H), 8.01-7.98 (m, 2H), 7.56-7.53 (m, 2H), 7.49-7.45 (m, 2H), 6.12 (s, 2H), 4.73-4.53 (m, 1H), 3.36 (t, J=6.6 Hz, 2H), 3.22-3.18 (m, 2H), 1.86-1.81 (m, 2H), 1.53-1.40 (m, 4H); HRMS (ESI): m/z calcd for C₂₁H₂₂BrNNaO₂ [M+Na]⁺ 422.0732, found 422.0734.

### 1.9.2 Anthracen-9-ylmethyl (5-iodopentyl)carbamate

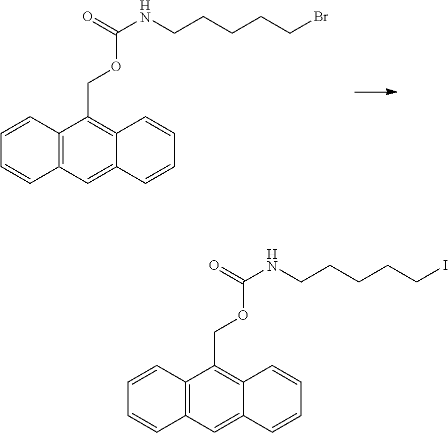 [SMILES: O=C(NCCCCCBr)OCc1c2ccccc2cc2ccccc12 ; O=C(NCCCCCI)OCc1c2ccccc2cc2ccccc12]

To a solution of anthracen-9-ylmethyl (5-bromopentyl)carbamate (˜60% pure, 154 mg, 0.23 mmol) in acetone (20 mL) is added potassium iodide (1.3 g, 7.8 mmol). The mixture is stirred under Ar at 50° C. for 16 h. The reaction is subsequently diluted with CH₂Cl₂, filtered, and concentrated under vacuum. The residue is taken up in CHCl₃, washed with water and dried (MgSO₄) to give a pale yellow solid (165 mg) which consisted of a mixture of anthracen-9-ylmethyl (5-iodopentyl)carbamate (˜60%, 96% yield) and anthracen-9-ylmethyl piperidine-1-carboxylate (˜40%). 1H NMR (500 MHz, CDCl₃) δ 8.47-8.46 (m, 1H), 8.40-8.36 (m, 2H), 8.01-7.98 (m, 2H), 7.56-7.53 (m, 2H), 7.49-7.45 (m, 2H), 6.12 (s, 2H), 4.73-4.53 (m, 1H), 3.21-3.17 (m, 2H), 3.13 (t, J=6.8 Hz, 2H), 1.82-1.77 (m, 2H), 1.53-1.35 (m, 4H); HRMS (ESI): m/z calcd for C₂₁H₂₂INNaO₂ [M+Na]⁺ 470.0593, found 470.0600.

### 1.9.3 CI540

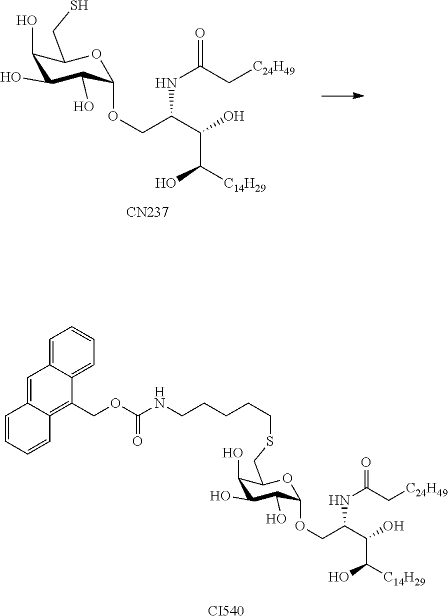

A mixture of thiol CN237³ (6.2 mg, 7.1 μmol), anthracen-9-ylmethyl (5-iodopentyl)carbamate (˜60% pure, 14.4 mg, 19.3 μmol) and potassium carbonate (3.2 mg, 23 μmol) in anhydrous deoxygenated DMF (0.3 mL) is stirred under Ar at 50° C. for 6.5 h, then left at rt overnight. The mixture is diluted with CH₂Cl₂, filtered and concentrated. The residue is purified by silica gel chromatography (gradient: 100% CHCl₃ through to 10% MeOH/CHCl₃) to yield CI540 as a white solid (5.8 mg, 68%). 1H NMR (400 MHz, 3:1 CDCl₃/CD₃OD) δ 8.47 (s, 1H), 8.34 (d, J=8.9 Hz, 2H), 7.99 (d, J=8.4 Hz, 2H), 7.55-7.51 (m, 2H), 7.47-7.43 (m, 2H), 6.09 (s, 2H), 4.82 (d, J=3.7 Hz, 1H), 4.14 (obscured by water, obsvd hsqc, 1H), 3.90-3.83 (m, 2H), 3.79-3.71 (m, 2H), 3.67 (dd, J=10.0, 3.1 Hz, 1H), 3.62 (dd, J=10.6, 3.8 Hz, 1H), 3.53-3.46 (m, 2H), 3.13 (t, J=6.8 Hz, 2H), 2.74-2.64 (m, 2H), 2.51 (t, J=7.2 Hz, 2H), 2.13 (t, J=7.5 Hz, 2H), 1.62-1.43 (m, 8H), 1.39-1.04 (m, 70H), 0.85-0.81 (m, 6H); ¹³C NMR (101 MHz, 3:1 CDCl₃/CD₃OD) δ 174.4, 131.7, 131.3, 129.3, 129.2, 126.8, 125.3, 124.3, 99.9, 74.9, 72.3, 71.1, 70.6, 70.2, 69.0, 67.8, 59.4, 50.3, 41.0, 36.8, 32.9, 32.7, 32.5, 32.1, 30.0, 29.92, 29.89, 29.8, 29.64, 29.57, 29.5, 26.1, 22.9, 14.2; HRMS (ESI): m/z calcd for C₇₁H₁₂₀N₂NaO₁₀S [M+Na]⁺ 1215.8561, found 1215.8567.

### 1.10 CI539

### 1.10.1 N-(4-(Anthracen-9-yl)phenyl)-2-chloroacetamide

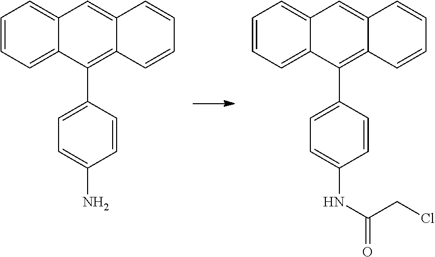 [SMILES: Nc1ccc(-c2c3ccccc3cc3ccccc23)cc1 ; O=C(CCl)Nc1ccc(-c2c3ccccc3cc3ccccc23)cc1]

Chloroacetyl chloride (125 μL, 1.57 mmol) is added to an ice-cold solution of 4-(anthracen-9-yl)aniline¹⁰ (270 mg, 1.00 mmol) in CH₂Cl₂ (2 mL). i-Pr₂NEt (250 μL, 1.36 mmol) is added dropwise to the suspension, and the resulting solution is stirred at 0° C. for 1 h, then allowed to warm to RT overnight. The reaction is diluted with CH₂Cl₂ (20 ml), washed with 0.5 M HCl (10 mL), sat. NaHCO₃ (20 mL), brine (20 mL) and dried (MgSO₄) to give a crude tan solid. The solid is recrystallised from CH₂Cl₂/EtOH to give N-(4-(anthracen-9-yl)phenyl)-2-chloroacetamide (79%) as a tan solid. 1H NMR (500 MHz, d6-DMSO) δ 10.53 (s, 1H), 8.67 (s, 1H), 8.15 (d, J=8.5 Hz, 2H), 7.85 (d, J=8.5 Hz, 2H), 7.58 (d, J=8.7 Hz, 2H), 7.52 (ddd, J=8.2, 6.5, 1.2 Hz, 2H), 7.43 (ddd, J=8.8, 6.5, 1.3 Hz, 2H), 7.38 (d, J=8.4 Hz, 2H), 4.34 (s, 2H); ¹³C NMR (126 MHz, d6-DMSO) δ 165.4, 138.5, 136.5, 133.7, 131.9, 131.4, 130.2, 128.9, 126.9, 126.5, 126.3, 125.8, 119.9, 44.1; HRMS (ESI): m/z calcd for C₂₂H₁₆ClNONa [M+Na]⁺ 368.0818, found 368.0815.

### 1.10.2 N-(4-(Anthracen-9-yl)phenyl)-2-iodoacetamide

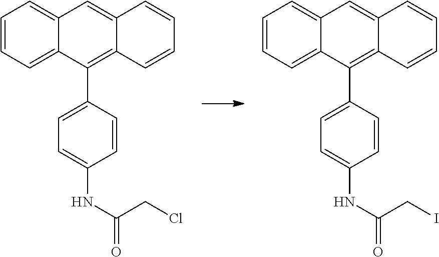 [SMILES: O=C(CCl)Nc1ccc(-c2c3ccccc3cc3ccccc23)cc1 ; O=C(CI)Nc1ccc(-c2c3ccccc3cc3ccccc23)cc1]

NaI (200 mg, 1.33 mmol) is added to a suspension of N-(4-(anthracen-9-yl)phenyl)-2-chloroacetamide (150 mg, 0.43 mmol) in acetone (3 mL), and the reaction is heated at 60° C. for 36 h. The reaction is concentrated, and the resulting material is partitioned between EtOAc/water (50 mL), the phases are separated and the aqueous layer is extracted with EtOAc. The combined organic extracts are washed with brine, dried (MgSO₄) and concentrated to give N-(4-(anthracen-9-yl)phenyl)-2-iodoacetamide (170 mg, 90%), as a tan solid. 1H NMR (500 MHz, d6-DMSO) δ 10.56 (s, 1H), 8.66 (s, 1H), 8.15 (d, J=8.4 Hz, 2H), 7.83 (d, J=8.4 Hz, 2H), 7.59 (dd, J=8.9, 1.1 Hz, 2H), 7.52 (ddd, J=8.0, 6.5, 1.1 Hz, 2H), 7.42 (ddd, J=8.1, 6.5, 1.3 Hz, 2H), 7.36 (d, J=8.4 Hz, 2H), 3.92 (s, 2H); ¹³C NMR (101 MHz, d6-DMSO) δ 167.3, 138.9, 136.5, 133.5, 131.9, 131.4, 130.1, 128.9, 126.9, 126.6, 126.3, 125.8, 119.6, 2.04; HRMS (ESI): m/z calcd for C₂₂H₁₆INONa [M+Na]⁺ 460.0174, found 460.0182.

### 1.10.3 CI539

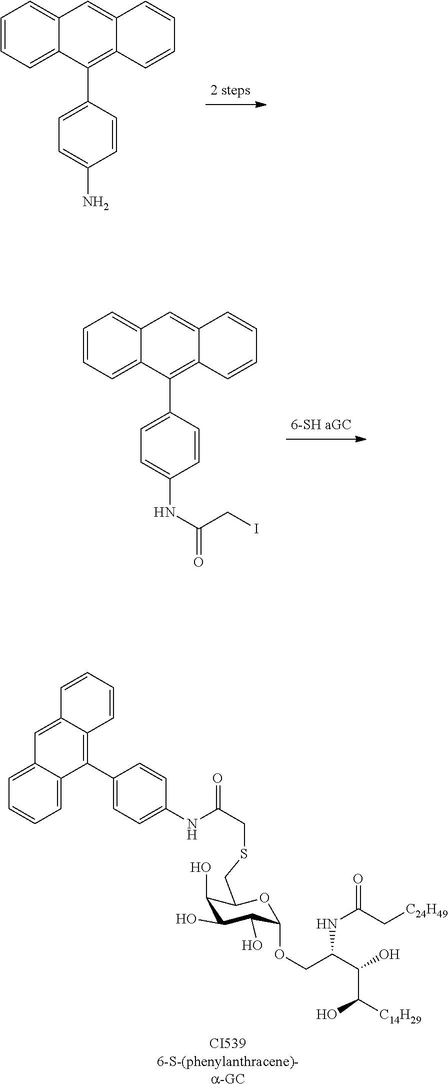

i-Pr₂NEt (4 μL, 0.023 mmol) is added to a solution of N-(4-(anthracen-9-yl)phenyl)-2-iodoacetamide (6.2 mg, 0.014 mmol) and thiol CN237² in 1:1 CH₂Cl₂/MeOH (1 mL). After overnight reaction, the reaction is concentrated. The residue is purified by silica gel chromatography (CHCl₃ changing to 25% MeOH/CHCl₃) to yield CI539 (8.09 mg, 59%) as a white solid. 1H NMR (500 MHz, 3:1 CDCl₃/CD₃OD) δ 8.50 (s, 1H), 8.05 (d, J=8.7 Hz, 2H), 7.82 (d, J=8.5 Hz, 2H), 7.68 (dd, J=8.8, 1.0 Hz, 2H), 7.49-7.42 (m, obscured by CHCl₃, obsvd hsqc, 3H), 7.40 (d, J=8.4 Hz, 2H), 7.35 (ddd, J=8.8, 6.5, 1.3 Hz, 2H), 4.92 (d, J=3.8 Hz, 1H), 4.24 (q, J=4.6 Hz, 1H), 4.03-3.95 (m, 3H), 3.83-3.69 (m, 3H), 3.62-3.56 (m, 2H), 3.55-3.38 (m, 2H), 3.01 (dd, J=13.9, 7.9 Hz, 1H), 2.90 (dd, J=13.9, 5.8 Hz, 1H), 2.31-2.15 (m, 2H), 1.78-1.45 (m, 4H), 1.42-1.19 (m, 68H), 0.91-0.86 (m, 6H). ¹³C NMR 174.5, 169.0, 137.4, 136.4, 134.9, 131.8, 131.4, 130.3, 128.4, 126.7, 126.6, 125.4, 125.1, 120.0, 99.5, 74.5, 72.1, 70.6, 70.3, 70.2, 67.3, 54.6, 50.3, 42.8, 36.9, 36.6, 32.8, 32.5, 31.9, 29.8, 29.7, 29.7, 29.6, 29.4, 29.4, 29.4, 25.9, 25.9, 22.7, 14.0. HRMS (ESI): m/z calcd for C₇₂H₁₁₄N₂O₉SNa [M+Na]⁺ 1205.8143, found 1205.8125.

### 1.11 CI558

### 1.11.1 1-(Aminooxy)-2,17-dioxo-6,9,12,15,21,24,27,30-octaoxa-3,18-diazadotriacontan-32-oic acid

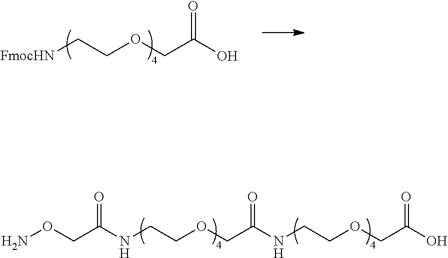

14-((((9H-Fluoren-9-yl)methoxy)carbonyl)amino)-3,6,9,12-tetraoxatetradecanoic acid (0.23 g, 0.5 mmol) is loaded on to 2-chlorotrityl chloride polystyrene resin (1.5 mmol/g, 100-200 mesh, 1% DVB) using i-Pr₂NEt (0.32 g, 2.5 mmol) in CH₂Cl₂ (5 mL). After agitating for 1 h, the resin is capped with MeOH (0.25 mL, 5 min). Fmoc deprotection and coupling of a second 14-((((9H-fluoren-9-yl)methoxy)carbonyl)amino)-3,6,9,12-tetraoxatetradecanoic acid residue is carried out on a Biotage® Initiator+Alstra™ automated peptide synthesiser as follows. The Fmoc group is removed with 20% piperidine/DMF (5 mL), agitating for 10 min. The resin is rinsed with DMF and 14-((((9H-fluoren-9-yl)methoxy)carbonyl)amino)-3,6,9,12-tetraoxatetradecanoic acid (0.45 g, 1 mmol) coupled to the resin in DMF (4 mL) with N,N′-diisopropylcarbodiimide (0.3 g, 2.5 mmol) and ethyl (hydroxyimino)cyanoacetate (0.35 g, 2.5 mmol) at 75° C. (microwave, 50 W) with agitation for 5 min. After rinsing the resin, the Fmoc group is removed with 20% piperidine/DMF, agitating for 10 min. 2,5-Dioxopyrrolidin-1-yl 2-(((tert-butoxycarbonyl)amino)oxy)acetate (0.1 g, 0.6 mmol) and collidine (92 μL, 0.75 mmol) in DMF (5 mL) are added to the resin and agitated for 20 min before rinsing with 15 mL CH₂Cl₂ and drying under vacuum. The product is cleaved from the resin in 5 mL of cleavage cocktail (93:5:2 TFA/water/triisopropylsilane) for 10 min, rinsing the resin with a further 5 mL of the same cocktail, and left to stand for 2 h to cleave the Boc group. The mixture is concentrated to give 1-(aminooxy)-2,17-dioxo-6,9,12,15,21,24,27,30-octaoxa-3,18-diazadotriacontan-32-oic acid as an oil which is used without purification.

### 1.11.2 4-(N-(8-Oxononanoyl)-L-valinyl-L-citrullinamido)benzyl alcohol

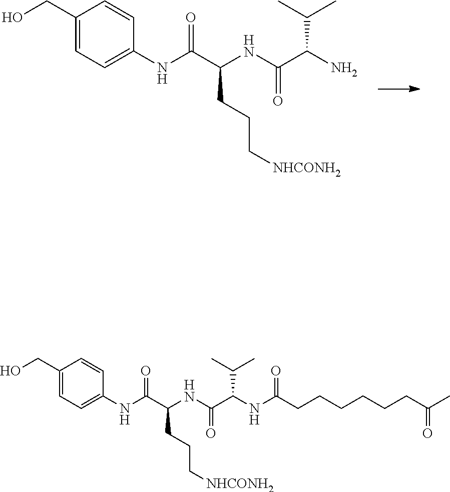

To an ice-cooled solution of 8-oxononanoic acid (280 mg, 1.63 mmol) in dry CH₂Cl₂ (8 mL) is added N-methylmorpholine (0.17 mL, 1.5 mmol) followed by isobutyl chloroformate (0.19 mL, 1.5 mmol). The ice bath is removed and the mixture is stirred at rt for 40 min, before adding a suspension of amine L-valinyl-L-citrullinamido)benzyl alcohol¹¹ (500 mg, 1.32 mmol) in MeOH (8 mL). The mixture is allowed to stir at rt overnight and the solvents are concentrated under vacuum. The crude material is triturated with MeOH and the solid residue kept aside. The MeOH-soluble fraction is concentrated and triturated again using 3:2 EtOH/diethyl ether. The solid residue from this treatment is combined with that obtained earlier and dried under vacuum to afford the amide product as a light tan solid (619 mg, 88%). 1H NMR (500 MHz, d6-DMSO) δ 0.83 (d, J=6.8 Hz, 3H), 0.86 (d, J=6.8 Hz, 3H), 1.17-1.26 (m, 4H), 1.31-1.51 (m, 6H), 1.55-1.62 (m, 1H), 1.67-1.74 (m, 1H), 1.94-2.01 (m, 1H), 2.05 (s, 3H), 2.10-2.21 (m, 2H), 2.38 (t, J=7.3 Hz, 2H) 2.90-2.97 (m, 1H), 2.98-3.05 (m, 1H), 4.17 (dd, J=7.0, 8.3 Hz, 1H), 4.35-4.39 (m, 1H), 4.42 (d, J=5.6 Hz, 2H), 5.10 (t, J=5.6 Hz, 1H), 5.39 (br s, 2H), 5.97 (br t, J=5.5 Hz, 1H), 7.23 (d, J=8.3 Hz, 2H), 7.53 (d, J=8.3 Hz, 2H), 7.79 (d, J=8.5 Hz, 1H), 8.01 (d, J=7.5 Hz, 1H), 9.86 (s, 1H); ¹³C NMR (126 MHz, d6-DMSO) δ 18.2, 19.3, 23.2, 25.3, 26.8, 28.3, 28.4, 29.4, 29.7, 30.3, 35.2, 38.7, 42.7, 53.1, 57.8, 62.6, 118.9, 127.0, 137.46, 137.50, 159.0, 170.4, 171.3, 172.6, 208.7; HRMS-ESI: m/z calcd for C₃₄H₄₇N₆O₁₀ [M+H]⁺ 699.3348, found 699.3360.

### 1.11.3 4-Nitrophenyl 4-(N-(8-oxononanoyl)-L-valinyl-L-citrullinamido)benzyl carbonate

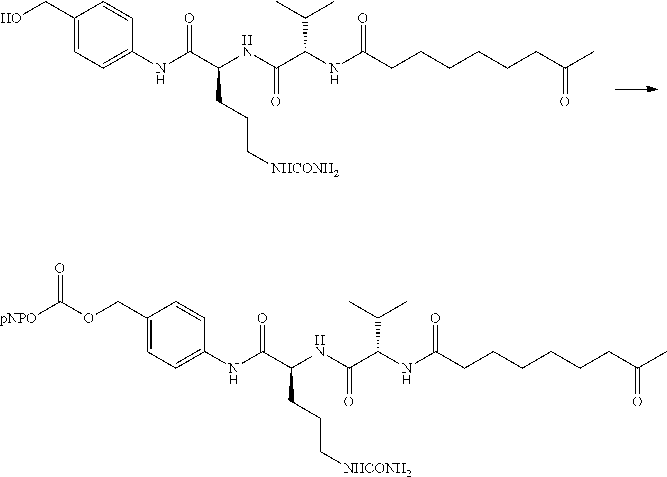

To a mixture of 4-(N-(8-oxononanoyl)-L-valinyl-L-citrullinamido)benzyl alcohol (265 mg, 0.496 mmol) in DMF (2.5 mL) is added bis(4-nitrophenyl) carbonate (165 mg, 0.537 mmol) and NEt₃ (0.08 mL, 0.6 mmol). After stirring under Ar at rt for 24 h, the reaction mixture is concentrated under vacuum onto silica gel and purified by flash chromatography on silica gel (gradient elution, MeOH/CH₂Cl₂=5:95 to 13:87) to give the title compound as an off-white solid (271 mg, 78%). 1H NMR (500 MHz, d6-DMSO) δ 0.84 (d, J=6.8 Hz, 3H), 0.86 (d, J=6.8 Hz, 3H), 1.17-1.26 (m, 4H), 1.32-1.51 (m, 6H), 1.56-1.64 (m, 1H), 1.68-1.75 (m, 1H), 1.94-2.01 (m, 1H), 2.05 (s, 3H), 2.10-2.21 (m, 2H), 2.38 (t, J=7.3 Hz, 2H) 2.91-2.98 (m, 1H), 2.99-3.06 (m, 1H), 4.18 (dd, J=6.9, 8.5 Hz, 1H), 4.36-4.40 (m, 1H), 5.24 (s, 2H), 5.40 (br s, 2H), 5.98 (br t, J=5.7 Hz, 1H), 7.40 (d, J=8.6 Hz, 2H), 7.54-7.57 (m, 2H), 7.64 (d, J=8.6 Hz, 2H), 7.79 (d, J=8.6 Hz, 1H), 8.05 (d, J=7.5 Hz, 1H), 8.29-8.32 (m, 2H), 10.02 (s, 1H); ¹³C NMR (126 MHz, d₆-DMSO) b 18.2, 19.2, 23.1, 25.2, 26.8, 28.2, 28.4, 29.2, 29.6, 30.3, 35.1, 38.5, 42.7, 53.1, 57.6, 70.2, 119.0, 122.5, 125.3, 129.3, 129.4, 139.3, 145.1, 151.9, 155.3, 158.8, 170.7, 171.3, 172.4, 208.4; HRMS-ESI: m/z calcd for C₂₇H₄₄N₅O₆ [M+H]⁺ 534.3286, found 534.3294.

### 1.1.1 CI282

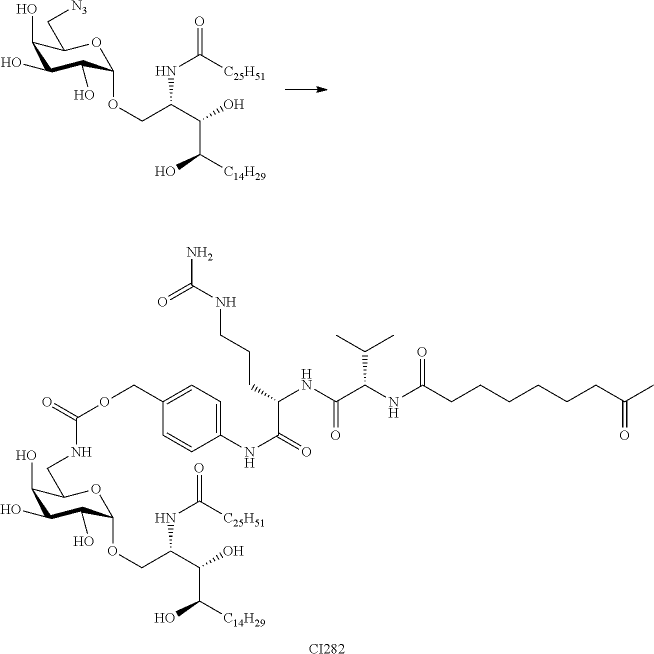

To a solution of 6-deoxy-6-azido-α-galactosylceramide⁸ (40 mg, 0.045 mmol) in CH₂Cl₂/MeOH (1:1, 12 mL) is added Pd/C (10%, 30 mg, 0.038 mmol) then aq HCl (100 μL, 1 M). The atmosphere is replaced with H₂ and the reaction is stirred at RT (18 h). The catalyst is filtered through glass fibre, washed with hot EtOH (3×50 mL) and the filtrate concentrated to give an off-white solid (63 mg). To the crude amine is added 4-nitrophenyl 4-(N-(8-oxononanoyl)-L-valinyl-L-citrullinamido)benzyl carbonate (45 mg, 0.064 mmol) and the two solids are thoroughly dried under high vacuum then placed under Ar before the addition of dry pyridine (5 mL) then dry Et₃N (100 μL). The reaction mixture is briefly sonicated before stirring at rt (18 h). MeOH (10 mL) is added, the reaction mixture is concentrated under vacuum and the residue is purified by automated flash column chromatography (MeOH/CHCl₃, 0:100 to 30:70) to give CI282 as a white solid (44 mg, 69%); HRMS(ESI) m/z calcd for C₇₈H₁₄₂N₇O₁₅ [M+H]⁺: 1417.0564, found 1417.0570.

### 1.1.2 CI558

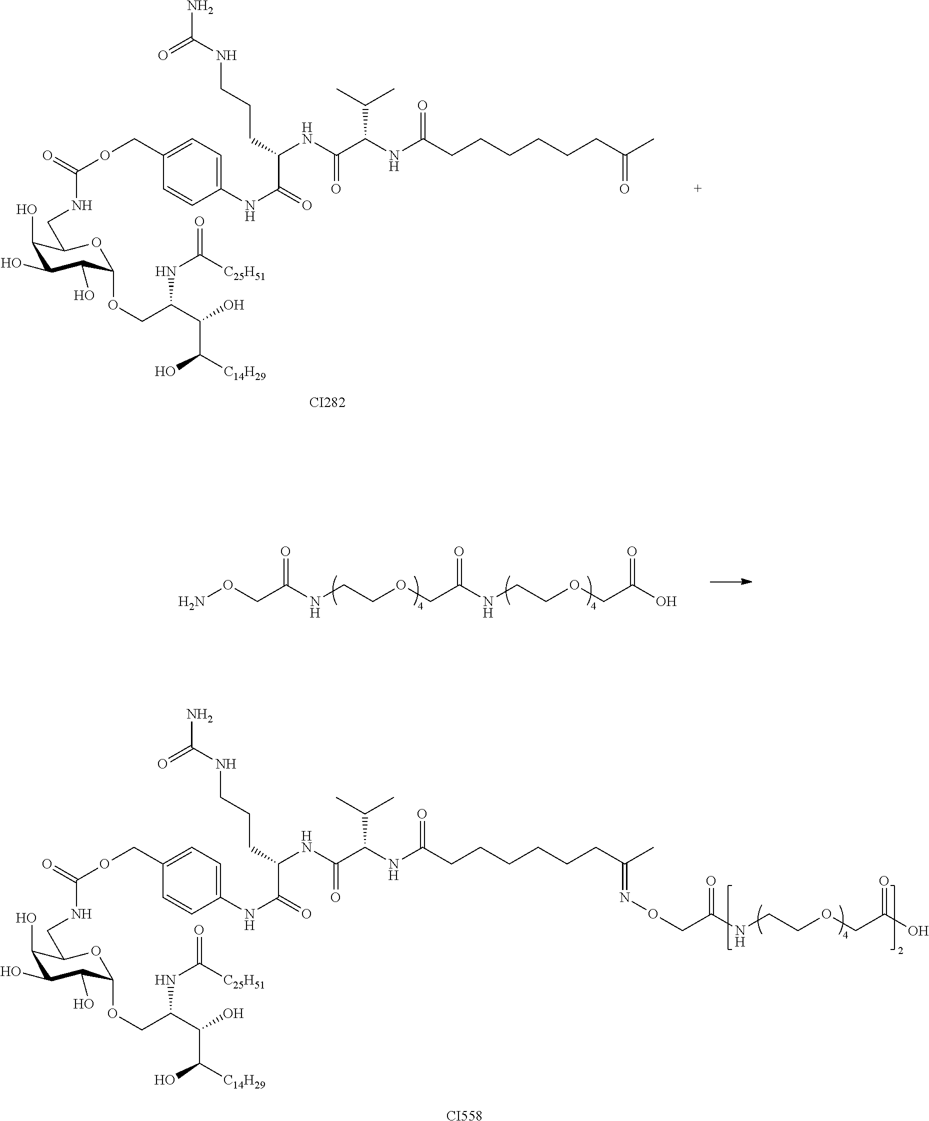

A solution of aniline in hexafluoroisopropanol (100 mM) is adjusted to pH 4 with TFA. 200 μL of this mixture is added to a vial containing 1-(aminooxy)-2,17-dioxo-6,9,12,15,21,24,27,30-octaoxa-3,18-diazadotriacontan-32-oic acid (6.42 mg, 11.5 μmol) and ketone CI282 (1.1 mg, 0.78 μmol) and the reaction is heated at 30° C. for 16 h. After concentration of the volatiles, the reaction is purified by silica gel chromatography (90:1.5:8.5 changing to 70:4.5:25.5 CHCl₃/water/MeOH) to yield CI558 (0.32 mg, 21%) as an oil. HRMS (ESI): m/z calcd for C₇₂H₁₁₄N₂O₉SNa [M+Na]⁺ 1205.8143, found 1205.8125.

### 1.12 CI585

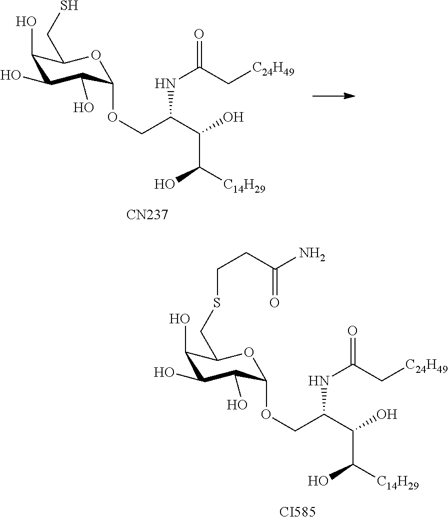

A mixture of thiol CN237 (7.0 mg, 0.008 mmol), acrylamide (1.4 mg, 0.02 mmol) and N,N′-diisopropylethylamine (5 μL, 0.03 mmol) in deoxygenated DMF (0.35 mL) is stirred at 55° C. under Ar. After 18 h, potassium carbonate (4.1 mg, 0.03 mmol) and acrylamide (2.8 mg, 0.04 mmol) are added and heating is continued for 5 h. The solvent is concentrated under vacuum and the residue is triturated with water (×3). Flash chromatography on silica gel (CHCl₃ to 85:15 CHCl₃/CH₃OH) affords the product CI585 as a white solid (3.9 mg, 52%). 1H NMR (500 MHz, 2:1 CDCl₃/CD₃OD) δ 4.87 (d, J=3.7 Hz, 1H), 4.21 (q, J=4.4 Hz, 1H), 3.95-3.90 (m, 2H), 3.87 (t, J=6.9 Hz, 1H), 3.77 (dd, J=10.0, 3.7 Hz, 1H), 3.73 (dd, J=10.0, 3.1 Hz, 1H), 3.66 (dd, J=10.5, 4.3 Hz, 1H), 3.59-3.55 (m, 2H), 2.90-2.79 (m, 3H), 2.73 (dd, J=13.8, 6.3 Hz, 1H), 2.52 (t, J=7.2 Hz, 2H), 2.23-2.20 (m, 2H), 1.70-1.52 (m, 4H), 1.43-1.21 (m, 68H), 0.89 (t, J=6.9 Hz, 6H); ¹³C NMR (126 MHz, 2:1 CDCl₃/CD₃OD) δ 174.8, 99.9, 75.0, 72.4, 71.4, 70.8, 70.4, 69.2, 67.6, 50.6, 36.9, 36.2, 32.8, 32.7, 32.3, 30.1, 30.05, 30.01, 29.9, 29.8, 29.72, 29.69, 28.7, 26.3, 26.2, 23.0, 14.2; HRMS (ESI): m/z calcd for C₅₃H₁₀₄N₂O₉NaS [M+Na]⁺ 967.7360, found 967.7361.

### 1.13 CI586

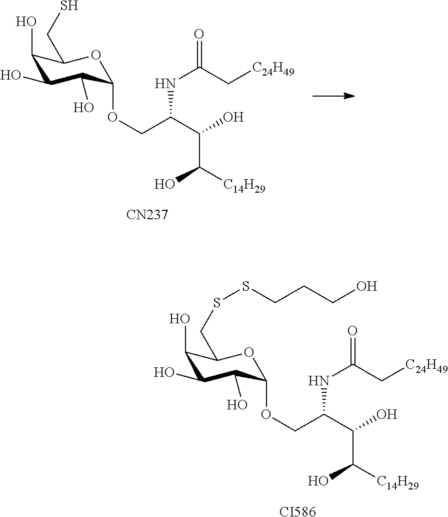

To a mixture of thiol CN237 (7.1 mg, 0.0081 mmol) and 3-(2-pyridyldisulfanyl)propan-1-ol (2.4 mg, 0.012 mmol) in 1:1 CH₂Cl₂—CH₃OH (0.6 mL) is added N,N-diisopropylethylamine (0.5 μL, 0.003 mmol) under a blanket of Ar. The vessel is capped and heated at 50° C. for 5 min to solubilise the reactants, then stirred at rt for 50 h. The solvents are concentrated under vacuum and the residue is purified by flash chromatography on silica gel (CHCl₃ to 85:15 CHCl₃/CH₃OH) to afford the product CI586 as a white solid (3.4 mg, 43%). ¹H NMR (500 MHz, 2:1 CDCl₃/CD₃OD) δ 4.90 (d, J=3.4 Hz, 1H), 4.21 (q, J=4.5 Hz, 1H), 4.01 (t, J=6.8 Hz, 1H), 3.96-3.95 (m, 1H), 3.92 (dd, J=10.7, 4.5 Hz, 1H), 3.79 (dd, J=10.0, 3.4 Hz, 1H), 3.76 (dd, J=10.0, 2.9 Hz, 1H), 3.71-3.66 (m, 3H), 3.59-3.55 (m, 2H), 2.99-2.91 (m, 2H), 2.81 (t, J=7.2 Hz, 2H), 2.24-2.21 (m, 2H), 1.96-1.90 (m, 2H), 1.69-1.52 (m, 4H), 1.43-1.22 (m, 68H), 0.89 (t, J=6.9 Hz, 6H); ¹³C NMR (126 MHz, 2:1 CDCl₃/CD₃OD) δ 174.8, 100.1, 74.9, 72.4, 70.7, 70.2, 70.0, 69.2, 67.9, 60.5, 50.5, 39.7, 36.9, 35.4, 32.7, 32.28, 32.276, 32.1, 30.2, 30.10, 30.07, 30.06, 30.04, 30.02, 30.01, 30.0, 29.9, 29.8, 29.74, 29.71, 29.69, 26.3, 26.2, 23.0, 14.2; HRMS (ESI): m/z calcd for C₅₃H₁₀₅NO₉NaS₂ [M+Na]⁺ 986.7128, found 986.7128.

### 1.14 CI587

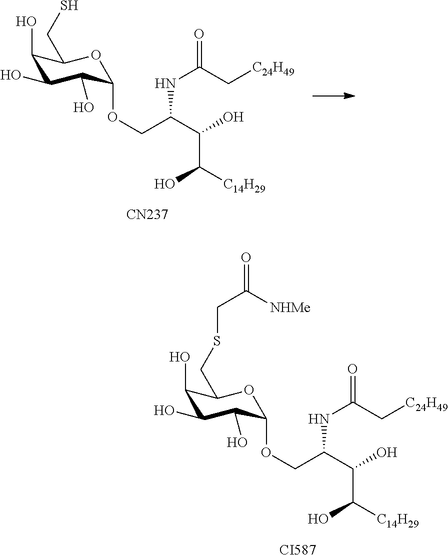

To a mixture of thiol CN237 (7.0 mg, 0.0080 mmol) and 2-iodo-N-methylacetamide (1.9 mg, 0.0095 mmol) in 1:1 CH₂Cl₂—CH₃OH (0.8 mL) is added N,N-diisopropylethylamine (2.0 μL, 0.011 mmol) under a blanket of Ar. The vessel is capped and heated at 50° C. briefly to solubilise the reactants, then stirred at rt. After 3 h, further 2-iodo-N-methylacetamide (0.85 mg, 0.0043 mmol) and N,N-diisopropylethylamine (1.0 uL, 0.057 mmol) are added. After a further 3 h, the solvents are concentrated under vacuum and the residue is triturated with acetonitrile (×3) to afford the product CI587 as a white solid (5.9 mg, 78%). ¹H NMR (500 MHz, 2:1 CDCl₃/CD₃OD) δ 4.88 (d, J=3.7 Hz, 1H), 4.22 (q, J=4.6 Hz, 1H), 3.95-3.89 (m, 3H), 3.77 (dd, J=10.0, 3.7 Hz, 1H), 3.73 (dd, J=10.0, 3.2 Hz, 1H), 3.66 (dd, J=10.5, 4.4 Hz, 1H), 3.59-3.55 (m, 2H), 3.29 (d, J=15.2 Hz, 1H), 3.20 (d, J=15.2 Hz, 1H), 2.84 (dd, J=13.7, 7.7 Hz, 1H), 2.80 (s, 3H), 2.75 (dd, J=13.7, 6.1 Hz, 1H), 2.22 (t, J=7.6 Hz, 2H), 1.70-1.51 (m, 4H), 1.43-1.21 (m, 68H), 0.89 (t, J=6.9 Hz, 6H); ¹³C NMR (126 MHz, 2:1 CDCl₃/CD₃OD) δ 174.9, 171.6, 100.0, 75.0, 72.4, 71.0, 70.7, 70.5, 69.2, 67.6, 50.8, 36.9, 36.4, 33.3, 32.9, 32.3, 30.2, 30.14, 30.09, 30.05, 30.02, 29.95, 29.83, 29.77, 29.74, 26.6, 26.32, 26.26, 23.0, 14.2; HRMS (ESI): m/z calcd for C₅₃H₁₀₄N₂O₉NaS [M+Na]⁺ 967.7360, found 967.7364.

### 1.15 CI588

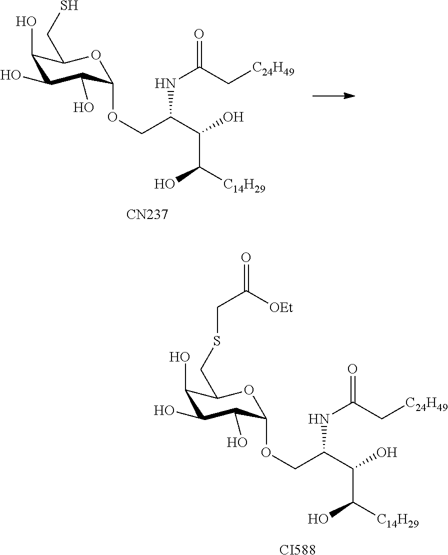

To a mixture of thiol CN237 (7.1 mg, 0.0081 mmol) in 1:1 CH₂Cl₂—CH₃OH (0.8 mL) is added ethyl bromoacetate (1.4 μL, 0.013 mmol) and N-methylmorpholine (1.4 uL, 0.013 mmol) under a blanket of Ar. The vessel is capped and heated at 50° C. briefly to solubilise the reactants, then stirred at rt. Further identical portions of ethyl bromoacetate and N-methylmorpholine are added after 24 h and 52 h. After 3 days (total reaction time), the volatiles are concentrated under vacuum and the residue is purified by flash chromatography on silica gel (100:0 to 90:10 CHCl₃/CH₃OH) to afford the product CI588 as a white solid (6.8 mg, 87%). ¹H NMR (500 MHz, 2:1 CDCl₃/CD₃OD) δ 4.88 (d, J=3.7 Hz, 1H), 4.23-4.17 (m, 3H), 3.94-3.90 (m, 3H), 3.78 (dd, J=10.0, 3.7 Hz, 1H), 3.73 (dd, J=10.0, 3.1 Hz, 1H), 3.69 (dd, J=10.6, 4.0 Hz, 1H), 3.59-3.54 (m, 2H), 3.38-3.35 (m, obscured by solvent), 3.29 (d, J=14.9 Hz, 1H), 2.94 (dd, J=13.8, 7.8 Hz, 1H), 2.82 (dd, J=13.8, 6.1 Hz, 1H), 2.26-2.17 (m, 2H), 1.70-1.52 (m, 4H), 1.43-1.21 (m, 68H), 0.89 (t, J=6.9 Hz, 6H); ¹³C NMR (126 MHz, 2:1 CDCl₃/CD₃OD) δ 174.7, 171.6, 100.0, 75.0, 72.4, 70.9, 70.7, 70.4, 69.1, 67.8, 62.0, 50.5, 36.9, 34.6, 33.2, 32.8, 32.3, 30.14, 30.09, 30.05, 30.01, 29.98, 29.9, 29.8, 29.73, 29.70, 29.68, 26.3, 26.2, 23.0, 14.3, 14.2; HRMS (ESI): m/z calcd for C₅₄H₁₀₅NO₁₀NaS [M+Na]⁺ 982.7357, found 982.7365.

### 1.16 CI589

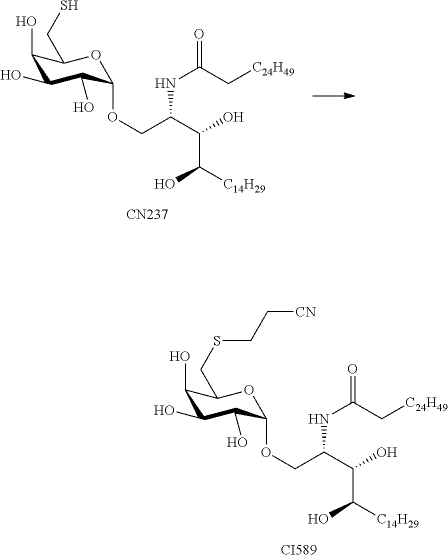

To a mixture of thiol CN237 (7.1 mg, 0.0081 mmol) and acrylonitrile (1.0 μL, 0.015 mmol) in 1:1 CH₂Cl₂—CH₃OH (0.6 mL) is added N,N′-diisopropylethylamine (2.0 μL, 0.011 mmol) under a blanket of Ar. The vessel is capped and heated at 50° C. briefly to solubilise the reactants, then stirred at rt. After 24 h, the solvents are concentrated under vacuum and the residue is purified by flash chromatography on silica gel (100:0 to 89:11 CHCl₃/CH₃OH) to afford the product CI589 as a white solid (6.2 mg, 83%). ¹H NMR (500 MHz, 2:1 CDCl₃/CD₃OD) δ 4.90 (d, J=3.6 Hz, 1H), 4.22 (q, J=4.6 Hz, 1H), 3.95 (dd, J=10.6, 4.8 Hz, 1H), 3.90-3.87 (m, 2H), 3.78 (dd, J=10.0, 3.6 Hz, 1H), 3.74 (dd, J=10.0, 3.1 Hz, 1H), 3.67 (dd, J=10.6, 4.1 Hz, 1H), 3.59-3.54 (m, 2H), 2.96-2.82 (m, 3H), 2.79-2.73 (m, 3H), 2.26-2.17 (m, 2H), 1.70-1.50 (m, 4H), 1.43-1.22 (m, 68H), 0.89 (t, J=6.9 Hz, 6H); ¹³C NMR (126 MHz, 2:1 CDCl₃/CD₃OD) δ 174.8, 119.1, 99.9, 75.0, 72.4, 71.8, 70.7, 70.6, 69.1, 67.6, 50.6, 36.9, 32.8, 32.3, 30.15, 30.11, 30.07, 30.02, 29.99, 29.93, 29.8, 29.73, 29.71, 29.70, 28.9, 26.3, 26.2, 23.0, 19.0, 14.2; HRMS (ESI): m/z calcd for C₅₃H₁₀₂N₂O₈NaS [M+Na]⁺ 949.7255, found 949.7250.

### 1.17 CI590

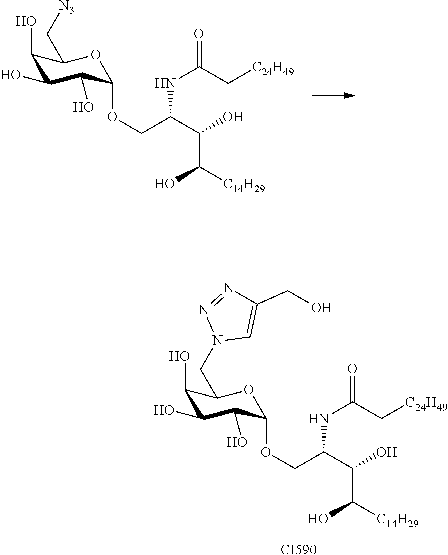

To a mixture of 6″-deoxy-6″-azido-α-galactosylceramide (7.2 mg, 0.0082 mmol) and tris(benzyltriazolylmethyl)amine (1.3 mg, 0.0024 mmol) in 1:1 CH₂Cl₂—CH₃OH (0.4 mL) is added propargyl alcohol (1.0 μL, 0.017 mmol) and a small piece of Cu foil (1×10 mm) under a blanket of Ar. The vessel is capped and the mixture is heated at 50° C. with stirring for 12 h. After cooling, the Cu foil is removed and solvents are concentrated. The residue is purified by flash chromatography on silica gel (100:0 to 85:15 CHCl₃/CH₃OH) to afford the product CI590 as a white solid (6.1 mg, 80%). ¹H NMR (500 MHz, 2:1 CDCl₃/CD₃OD) δ 7.81 (s, 1H), 4.87 (d, J=3.8 Hz, 1H), 4.72 (d, J=13.3 Hz, 1H), 4.70 (d, J=13.3 Hz, 1H), 4.64-4.56 (m, 2H), 4.13 (dd, J=7.7, 5.2 Hz, 1H), 4.08 (q, J=4.8 Hz, 1H), 3.87 (br d, J=3.2 Hz, 1H), 3.82 (dd, J=10.0, 3.8 Hz, 1H), 3.73 (dd, J=10.0, 3.2 Hz, 1H), 3.51-3.45 (m, 3H), 3.41 (dd, J=10.5, 4.8 Hz, 1H), 2.17-2.13 (m, 2H), 1.65-1.50 (m, 4H), 1.41-1.22 (m, 68H), 0.89 (t, J=6.9 Hz, 6H); ¹³C NMR (126 MHz, 2:1 CDCl₃/CD₃OD) δ 175.0, 148.4, 124.0, 99.8, 74.9, 72.3, 70.3, 70.1, 70.0, 69.0, 67.4, 56.1, 51.6, 50.6, 36.8, 32.9, 32.3, 30.2, 30.11, 30.07, 30.03, 30.00, 29.9, 29.8, 29.7, 26.25, 26.17, 23.0, 14.2; HRMS (ESI): m/z calcd for C₅₃H₁₀₂N₄O₉Na [M+Na]⁺ 961.7545, found 961.7542.

### 1.18 CI591

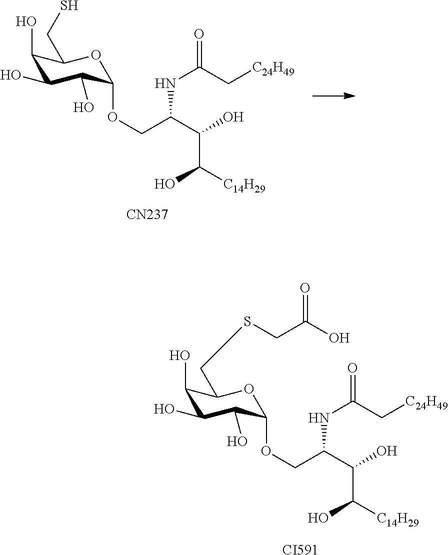

To a mixture of thiol CN237 (7.2 mg, 0.0083 mmol) and iodoacetic acid (5.4 mg, 0.029 mmol) in 1:1 CH₂Cl₂—CH₃OH (0.6 mL) is added N,N-diisopropylethylamine (7.7 μL, 0.044 mmol) under a blanket of Ar. The vessel is capped and heated at 50° C. briefly to solubilise the reactants, then stirred at rt. After 28 h, the solvents are concentrated under vacuum and the residue is triturated with acetonitrile (×2). The solid residue is purified by flash chromatography on silica gel (100:0 to 90:10 CH₂Cl₂/CH₃OH, then 90:9.5:0.5 to 85:14.25:0.75 CH₂Cl₂/CH₃OH/HOAc) to afford the product CI591 as a white solid (3.5 mg, 45%). 1H NMR (400 MHz, 2:1 CDCl₃/CD₃OD) δ 4.87 (d, J=3.5 Hz, 1H), 4.21 (q, J=4.5 Hz, 1H), 3.95-3.90 (m, 3H), 3.77 (dd, J=10.0, 3.5 Hz, 1H), 3.73 (dd, J=10.0, 2.9 Hz, 1H), 3.67 (dd, J=10.6, 4.3 Hz, 1H), 3.60-3.55 (m, 2H), 3.30 (d, J=14.8 Hz, 1H), 3.26 (d, J=14.8 Hz, 1H), 2.92 (dd, J=13.8, 7.4 Hz, 1H), 2.83 (dd, J=13.8, 6.5 Hz, 1H), 2.24-2.20 (m, 2H), 1.70-1.51 (m, 4H), 1.44-1.19 (m, 68H), 0.89 (t, J=6.9 Hz, 6H); ¹³C NMR (101 MHz, 2:1 CDCl₃/CD₃OD) δ 175.0, 173.9, 100.1, 75.0, 72.4, 70.9, 70.7, 70.3, 69.2, 67.8, 50.7, 36.9, 35.3, 33.4, 32.8, 32.3, 30.16, 30.12, 30.07, 30.03, 30.00, 29.9, 29.81, 29.75, 29.72, 26.3, 26.2, 23.0, 14.2; HRMS (ESI): m/z calcd for C₅₂H₁₀NO₁₀S [M−H]⁻ 930.7068, found 930.7072.

### 1.19 CI592

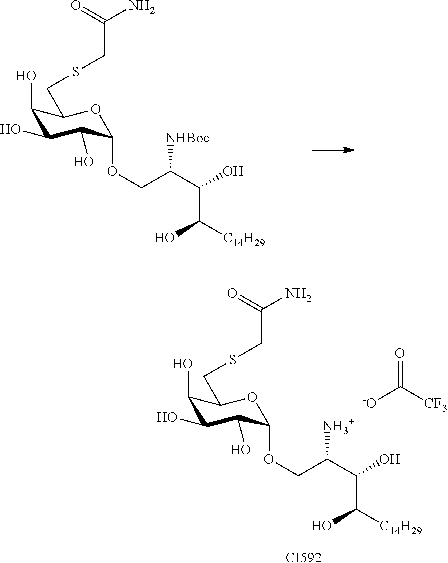

To a solution of (2S,3S,4R)-1-(6-Deoxy-6-thioacetimido)-α-D-galactopyranosyloxy)-2-(N-tert-butoxycarbonyl)amino-3,4-octadecandiol (190 mg, 0.291 mmol) in trifluoroacetic acid/CH₂Cl₂ (1:1, 15 mL) is added anisole (320 μL, 2.90 mmol) and the reaction is stirred at RT (1.5 h). The reaction is diluted with MeOH (30 mL) and concentrated under vacuum and the residue is purified by automated C18 reversed phase column chromatography (MeOH/H₂O 40:60 to 60:40) to give the trifluoroacetate salt of CI592 as a white solid (148 mg, 76%). 1H NMR (500 MHz, CD₃OD) δ 4.88-4.79 (m, obscured by water, obsvd hsqc, 1H), 4.24 (dd, J=10.7, 3.2 Hz, 1H), 3.95-3.90 (m, 2H), 3.84 (dd, J=10.3, 3.8 Hz, 1H), 3.77-3.70 (m, 2H), 3.58-3.49 (m, 3H), 3.29-3.17 (m, 2H), 2.88 (dd, J=14.2, 7.7 Hz, 1H), 2.79 (dd, J=14.2, 6.1 Hz, 1H), 1.86-1.76 (m, 1H), 1.61-1.50 (m, 1H), 1.43-1.23 (m, 24H), 0.90 (t, J=7.1 Hz, 3H); ¹³C NMR (126 MHz, CD₃OD) δ 175.6, 100.8, 73.8, 73.0, 72.0, 71.5, 71.3, 70.0, 54.9, 36.8, 35.4, 34.2, 33.1, 30.8, 30.7, 30.5, 26.2, 23.7, 14.4; HRMS(ESI) m/z calcd for C₂₆H₅₃N₂O₈S [M+H]⁺: 553.3517, found 553.3526.

### 1.20 CI593

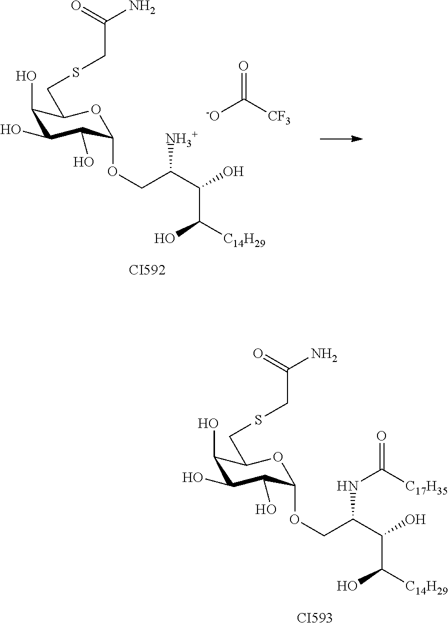

To a suspension of stearic acid (11 mg, 0.037 mmol) in dry CH₂Cl₂ (3 mL) is added Et₃N (42 μL, 0.295 mmol) and isobutyl chloroformate (5 μL, 0.037 mmol). After 50 minutes of stirring at room temperature, the reaction is added to a solution of amine CI592 (20 mg, 0.030 mmol) in dry DMF (1 mL) and the resulting reaction is stirred at room temperature for 23 h. MeOH (3 mL) and diethyl amine (30 μL) are added to the reaction and after stirring for 10 minutes at room temperature the reaction is concentrated under vacuum and purified by automated silica gel flash chromatography (MeOH/CHCl₃ 0:100 to 10:90) and triturated with water to yield the target compound CI593 (5 mg, 20%) as a white solid. 1H NMR (500 MHz, DMSO-d₆) δ 7.59 (d, J=8.9 Hz, 1H), 7.38 (br s, 1H), 6.98 (br s, 1H), 4.67 (d, J=3.3 Hz, 1H), 4.61-4.53 (m, 3H), 4.35 (d, J=7.4 Hz, 1H), 4.26 (d, J=6.9 Hz, 1H), 4.03-3.96 (m, 1H), 3.74 (t, J=7.1 Hz, 1H), 3.70-3.64 (m, 2H), 3.58-3.49 (m, 2H), 3.48-3.36 (m, 3H), 3.06 (s, 2H), 2.69 (m, 2H), 2.07 (td, J=7.3, 2.3 Hz, 2H), 1.54-1.39 (m, 4H), 1.32-1.14 (m, 52H), 0.85 (t, J=7.1 Hz, 6H); ¹³C NMR (126 MHz, DMSO-d₆) δ 171.7, 171.1, 99.3, 73.7, 70.6, 70.0, 69.7, 69.7, 68.3, 66.4, 49.6, 35.5, 34.9, 32.5, 31.4, 31.3, 29.2, 29.2, 29.1, 29.1, 29.1, 29.1, 29.0, 29.0, 29.0, 28.9, 28.7, 28.7, 25.4, 25.4, 22.1, 13.9; HRMS(ESI) m/z calcd for C₄₄H₈₆N₂O₉SNa [M+Na]⁺: 841.5952, found 553.3526.

### 1.21 CI594

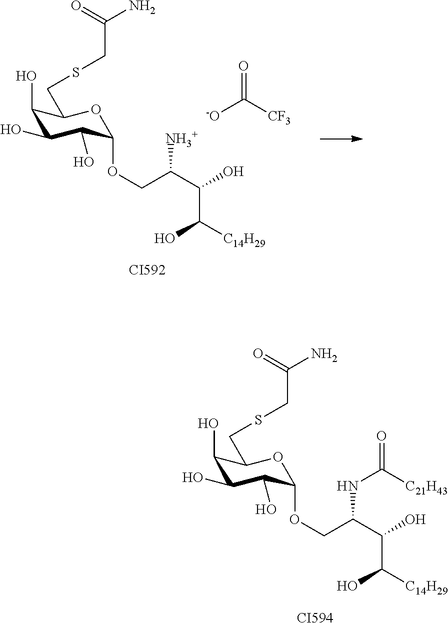

To a suspension of behenic acid (7.4 mg, 0.022 mmol) in dry CH₂Cl₂ (2 mL) is added Et₃N (23 μL, 0.162 mmol) and isobutyl chloroformate (2.6 μL, 0.019 mmol). After 50 minutes of stirring at room temperature, the reaction is added to a solution of amine CI592 (11 mg, 0.017 mmol) in dry DMF (1 mL) and the resulting reaction is stirred at room temperature for 1.5 h. MeOH (2 mL) and diethyl amine (20 μL) are added to the reaction and after stirring for 10 minutes at room temperature the reaction is concentrated under vacuum and purified by automated silica gel flash chromatography (MeOH/CHCl₃ 0:100 to 10:90) and triturated with water to yield the target compound CI594 (2.7 mg, 19%) as a white solid. 1H NMR (500 MHz, DMSO-d₆) δ 7.59 (d, J=8.9 Hz, 1H), 7.38 (br s, 1H), 6.98 (br s, 1H), 4.67 (d, J=3.3 Hz, 1H), 4.60-4.52 (m, 3H), 4.34 (d, J=7.4 Hz, 1H), 4.25 (d, J=6.9 Hz, 1H), 4.02-3.95 (m, 1H), 3.74 (t, J=7.1 Hz, 1H), 3.70-3.64 (m, 2H), 3.58-3.49 (m, 2H), 3.48-3.35 (m, 3H), 3.06 (s, 2H), 2.73-2.65 (m, 2H), 2.07 (td, J=7.3, 2.8 Hz, 1H), 1.55-1.34 (m, 5H), 1.31-1.14 (m, 59H), 0.85 (t, J=7.1 Hz, 6H); ¹³C NMR (126 MHz, DMSO-d₆) δ 171.7, 171.1, 99.3, 73.7, 70.6, 70.0, 69.7, 69.7, 68.3, 66.4, 49.6, 35.5, 34.9, 32.6, 31.4, 31.3, 31.3, 29.3, 29.2, 29.1, 29.1, 29.1, 29.0, 29.0, 28.9, 28.7, 28.7, 25.4, 25.4, 22.1, 13.9; HRMS(ESI) m/z calcd for C₄₈H₉₄N₂O₉SNa [M+Na]⁺: 897.6578, found 897.6575.

### 1.22 CI595

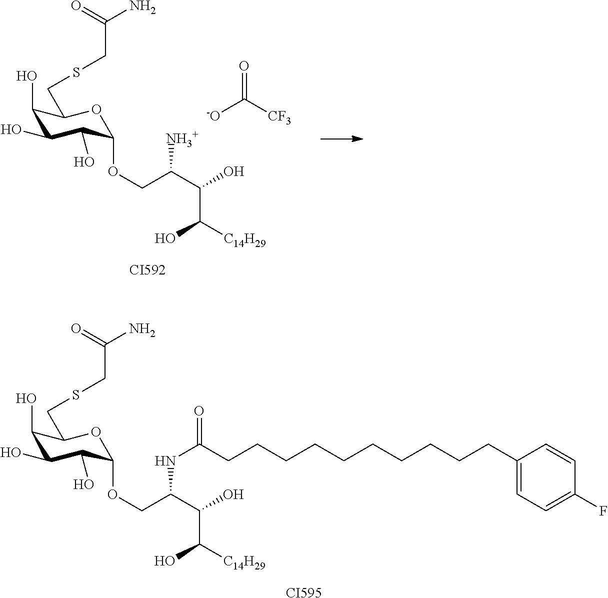

To a solution of 11-(4-fluorophenyl)undecanoic acid (6.0 mg, 0.020 mmol) in dry CH₂Cl₂ (2 mL) is added Et₃N (25 μL, 0.176 mmol) and isobutyl chloroformate (2.8 μL, 0.021 mmol). After 50 minutes of stirring at room temperature, the reaction is added to a solution of amine CI592 (12 mg, 0.018 mmol) in dry DMF (1 mL) and the resulting reaction is stirred at room temperature for 1.5 h. MeOH (2 mL) and diethyl amine (20 μL) are added to the reaction and after stirring for 10 minutes at room temperature the reaction is concentrated under vacuum and purified by automated silica gel flash chromatography (MeOH/CHCl₃ 0:100 to 8:92) and triturated with water to yield the target compound CI595 (7.0 mg, 48%) as a white solid. 1H NMR (500 MHz, 1:1 CDCl₃/CD₃OD) δ 7.12-7.04 (m, 2H), 6.91-6.84 (m, 2H), 4.81 (d, J=3.7 Hz, 1H), 4.16 (c, J=4.8 Hz, 1H), 3.90-3.83 (m, 3H), 3.71 (dd, J=9.9, 3.7 Hz, 1H), 3.67 (dd, J=9.9, 3.2 Hz, 1H), 3.60 (dd, J=10.6, 4.6 Hz, 1H), 3.54-3.48 (m, 2H), 3.25-3.12 (m, 2H), 2.82 (dd, J=13.6, 7.8 Hz, 1H), 2.72 (dd, J=13.6, 6.0 Hz, 1H), 2.51 (t, J=7.7 Hz, 2H), 2.16 (t, J=7.5 Hz, 2H), 1.65-1.45 (m, 6H), 1.37-1.15 (m, 36H), 0.82 (t, J=7.1 Hz, 3H); ¹³C NMR (126 MHz, 1:1 CDCl₃/CD₃OD) δ 175.2, 174.4, 162.8, 160.9, 139.1, 130.3, 130.2, 115.4, 115.2, 100.2, 75.1, 72.5, 71.3, 70.9, 70.8, 69.4, 67.7, 51.0, 37.0, 36.3, 33.5, 32.9, 32.5, 32.2, 30.4, 30.3, 30.3, 30.2, 30.1, 30.1, 30.0, 30.0, 29.9, 29.7, 26.5, 26.4, 23.2, 14.3; HRMS(ESI) m/z calcd for C₄₃H₇₆FN₂O₉S [M+H]⁺: 815.5250, found 815.5259.

### 1.23 CI596

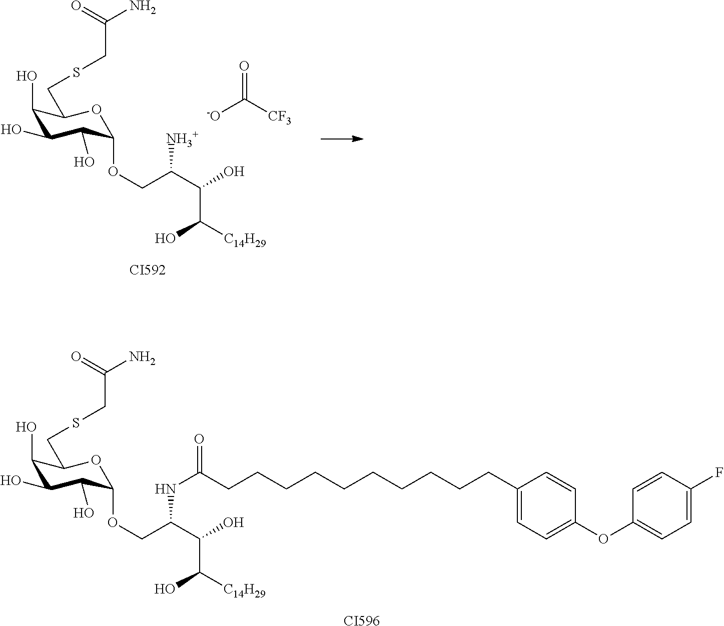

To a solution of 11-(4-(4-fluorophenoxy)phenyl)undecanoic acid (8.0 mg, 0.020 mmol) in dry CH₂Cl₂ (2 mL) is added Et₃N (25 μL, 0.176 mmol) and isobutyl chloroformate (2.8 μL, 0.021 mmol). After 50 minutes of stirring at room temperature, the reaction is added to a solution of amine CI592 (12 mg, 0.018 mmol) in dry DMF (1 mL) and the resulting reaction is stirred at room temperature for 1.5 h. MeOH (2 mL) and diethyl amine (20 μL) are added to the reaction and after stirring for 10 minutes at room temperature the reaction is concentrated under vacuum and purified by automated silica gel flash chromatography (MeOH/CHCl₃ 0:100 to 7:93) and triturated with water to yield the target compound CI596 (12 mg, 75%) as a white solid. ¹H NMR (500 MHz, 1:1 CDCl₃/CD₃OD) δ 7.07 (d, J=8.5 Hz, 2H), 6.99-6.93 (m, 2H), 6.92-6.87 (m, 2H), 6.81 (d, J=8.5 Hz, 2H), 4.81 (d, J=3.7 Hz, 1H), 4.16 (q, J=4.8 Hz, 1H), 3.90-3.82 (m, 3H), 3.71 (dd, J=9.9, 3.7 Hz, 1H), 3.67 (dd, J=9.9, 3.2 Hz, 1H), 3.60 (dd, J=10.6, 4.6 Hz, 1H), 3.54-3.48 (m, 2H), 3.25-3.11 (m, 2H), 2.82 (dd, J=13.6, 7.8 Hz, 1H), 2.72 (dd, J=13.6, 6.0 Hz, 1H), 2.52 (t, J=7.7 Hz, 2H), 2.16 (t, J=7.5 Hz, 2H), 1.66-1.44 (m, 6H), 1.40-1.14 (m, 36H), 0.82 (t, J=7.1 Hz, 3H); ¹³C NMR (126 MHz, 1:1 CDCl₃/CD₃OD) δ 174.2, 174.4, 160.2, 158.3, 156.0, 154.2, 138.6, 130.2, 120.6, 120.5, 118.9, 116.7, 116.5, 100.2, 75.1, 72.5, 72.3, 70.9, 70.8, 69.4, 67.6, 51.0, 37.0, 36.3, 35.7, 33.6, 32.5, 32.2, 30.4, 30.3, 30.3, 30.3, 30.2, 30.2, 30.1, 30.0, 30.0, 29.9, 29.9, 29.8, 26.5, 26.4, 23.2, 14.3; HRMS(ESI) m/z calcd for C₄₉H₇₉FN₂O₉SNa [M+Na]⁺: 929.5337, found 929.5345.

### 2. Generation of mRNA Construct

### 2.1 Preparation of Plasmid (pVAX1) Containing Ovalbumin (Gallus Gallus)

The entire protein sequence derived from the chicken egg ovalbumin gene (OVA) (GenBank AUD54526.1; set forth in SEQ ID NO: 1) for the species Gallus gallus was used. The gene sequence designed with the sequence set forth in SEQ ID NO: 2. In brief, kozak consensus mammalian ribosome binding site (RBS), GCCACCATGG was included as the methionine start codon and first nucleotide (G) of the codon for glycine, as part of the gene. The stop codon TGA was used at the end of the gene. The nucleotide sequence was codon optimized for mammalian expression by choosing codons that align with the most abundant tRNAs in mammalian cells and synthesised as a double stranded DNA product by Twist Biosciences LTD (San Francisco, CA, USA) with the solid media

Micro agar was added to liquid Luria Broth (LB) prepared using 8 g of LB and 6 g of micro agar and added to 400 mL of water with a final concentration of 1.5% (w/v) and autoclave sterilized. Once the media had cooled <50° C. the appropriate antibiotic was added. 20 mL of agar was poured into a 150 mm plate and swirled to cover plate surface and let cool to form a solid medium in a sterile hood. Plates were store at 4° C. for future use.

### 2.2 DNA Vaccine Purification

pDNA was purified using an EndoFree Plasmid Maxi Kit (Qiagen: Hilden, Germany). 1 μL of the −80° C. glycerol stock of the EC100 E. coli stock containing pDNA was diluted in 1 mL of LB and streaked on a Luria Broth (LB) agar plate containing kanamycin prepared by method in section 2.1, above, from an 8 hr culture with 10 mL LB media containing 50 μg/ml kanamycin was inoculated with a single colony and grown at 37° C., 200 rpm until OD600 ≥2 was reached. An overnight culture containing 500 mL of LB, high salt media was inoculated with a 1:100 dilution from the day culture and grown for 16 hr at 37° C., 200 rpm. The cultures were put on ice for 20 min prior to centrifugation at 6,000× for 15 min at 4° C. Plasmid DNA was extracted and purified from cultured cells as per the manufacturer's instructions on an anion-exchange column in a laminar flow hood. Plasmid DNA was precipitated from the 15 mL elution by adding 0.7 volume (10.5 mL) of isopropanol and centrifuged at 25,000×g for 30 min at 4° C. The resulting pellet was washed with ice-cold 70% ethanol, resuspended in endotoxin-free TE buffer and stored at 4° C. A 10 μL aliquot was diluted 10-fold and concentration was determined using a Spectrophotometer® NP80 (Implen: Westlake Village, CA, USA). This yielded 1 mg of plasmid DNA, stored at 4° C. for future use.

### 2.3 Vector Linearization

10 μg of pDNA prepared from EndoFree Plasmid Mega Kit was linearized with 10 μL of NotI restriction enzyme using FastDigest buffer (ThermoFisher: Waltham, MA, USA) in a total volume of 100 μL at 37° C. for 16 hr and purified using a DNA Clean and Concentrator-5 column (Zymo: Tustin, CA, USA) and eluted in 20 μl of elution buffer (10 mM Tris-Cl, pH 8.5) with a concentration of 250 ng/μL (50% yield).

### 2.4 In Vitro Transcription (IVT)

In vitro transcription was performed using the HiScribe™ T7 Quick High Yield RNA Synthesis Kit (New England Biolabs; Ipswich, MA, USA). 1 μg of linearized plasmid DNA was diluted to total volume of 8 μl in MQ. 2 μL of 10× T7 RNA polymerase reaction buffer was adding followed by 8 μL of NTP® mix (100 mM total, 25 mM each NTP) and finally 2 μL of T7 RNA polymerase (50 IU/μL). The reaction was incubated at 37° C. for 2 hr. The reaction mixture is incubated for 2 hr at 37° C. *dNTP (nucleoside triphosphates) can be guanine triphosphate, uridine triphosphate, pseudouridine triphosphate or any other modified nucleoside triphosphate.

The IVT reaction was diluted with 70 μL MQ water & treated with 1 μL DNase I (2 IU/μL) following the addition of 10 μL of 10×DNAse I buffer and incubation at 37° C. for 15 min to remove the DNA template. RNA was purified by adding 50 μL (50% volume) of 6 M LiCl to afford a final concentration of 2 M LiCl, incubating at −20° C. for 30 min and centrifuging at 4° C., 16,000×g for 20 min. The resulting pellet was washed with 500 μL of ice-cold 70% ethanol and resuspended in 100 μL of RNase-free water to be stored at −20° C. with a yield of 200 μg (2 mg/mL). This is defined as RNA_{OVA}.

### 2.5 5′ RNA Capping

Addition of the 5′-N7-methylguanosine triphosphate cap to mOVA IVT products was performed using the Vaccinia Capping System (New England Biolabs; Ipswich, MA, USA). RNA in water is denatured by incubating at 65° C. for 5 min then placed on ice for 5 min. 10 μg of RNA was diluted to 15 μL with Type 1 water and incubated for 5 min at 65° C. followed by a 5 min incubation on ice. 2 μL of each GTP (10 mM) & S-adenosylmethionine (SAM, 2 mM) was added and 1 μL of Vaccinia Capping Enzyme (10 IU/μL) to the reaction and incubated at 37° C. for 30 min.

RNA was precipitated using by adding 10 μL of 6 M LiCl, incubating at −20° C. overnight and centrifugation at 4° C. for 20 min at 16,000×g and stored at −20° C. until further use. This is defined as Cap0RNA_{OVA}.

### 2.6 PolvA Adenylation

5′-capped RNA was polyadenylated with the E. coli Poly(A) Polymerase (New England Biolabs; Ipswich, MA, USA). To 10 μL of RNA in 15 μL of MQ was added 2 μL of 10× reaction buffer & ATP (10 mM) followed by 1 μL of E. coli PolyA polymerase (5 IU/μL). The reaction was incubated for 30 min at 37° C. for an average polyA tail length of 200 bp. RNA was precipitated using by adding 10 μL of 6 M LiCl, incubating at −20° C. overnight and centrifugation at 4° C. for 20 min at 16,000×g and stored at −20° C. until further use. This is defined as mRNA.

### 3. Preparation of Liposomal mRNA Vaccines

DOTMA-DOPE-adjuvant liposomes were prepared either by thin-film preparation or by microfluidic mixing.

### 3.1 DOTMA-DOPE-Adjuvant Liposomes (Thin-Film Preparation)

Stock chloroform solutions of DOTMA (10 mg/mL, 302 μL, 4.5 μmol), DOPE (10 mg/mL, 117 μL, 1.5 μmol) and glycolipid adjuvant (where applicable) (1 mg/mL, 4.4 μL, 35.1 nmol) are added to a 25 mL round bottom flask. EtOH (3 mL) is added and the solvents are removed by rotary evaporation at 40° C., 100 rpm, 80 mBar for 30 min to afford a lipid film. The lipid film is hydrated with Type 1 water (1 mL) to bring the final lipid concentration to 6 mM and adjuvant concentration to 3.51 μM. The lipid dispersion is incubated at 4° C. overnight to equilibrate before 2 min sonication at 40 kHz, room temperature (Ultrasonic Cleaner). The transparent lipid dispersion is extruded using an Avanti® Mini-Extruder fitted with a 200 nm polycarbonate membrane and 1 mL gastight syringes attached at either end (11 passes) (Avanti Polar Lipids, Inc.: Alabaster, AL, USA).

### 3.2 DOTMA-DOPE-Adjuvant Liposomes (Microfluidic Mixing Preparation)

DOTMA, DOPE and glycolipid adjuvant were solubilized in EtOH (10 mg/mL total lipid concentration) at a molar ratio of 75:25:0.059. A NanoAssemblr Ignite microfluidics mixer with staggered herringbone cartridge was used to mix the ethanolic lipid solution with Type 1 water at a ratio of 1:3 (v/v) with a total flow rate of 5 mL/min. The EtOH was then removed by iterative dilution with water and concentration using a centrifugal filter with Mw cut off 30,000 Da. Lipid concentration was determined by HPLC, and the required volume of water was added to give a total lipid concentration of 6 mM. Liposome size is determined by dynamic light scattering using a Malvern Zetasizer Nano ZS (Malvern, UK). Representative size, PDI and zeta potential is 102 nm, 0.25 and 55.7 mV respectively.

### 4. RNA-Lipoplex Formulation

### 4.1 mRNA-DOTMA-DOPE-Adjuvant liposomes (LPX-mRNA-Adjuvant)

mRNA in Type 1 water was diluted to 1 mg/mL, with 1 M HEPES buffer (pH 7.4) and water to final HEPES buffer 100 mM. 30 μL of this was further mixed with 60 μL of aqueous NaCl (1.5 M) to give a 1:2 volume ratio (i.e., of mOVA-HEPES/NaCl). 136.6 μL of 6 mM liposome preparation was mixed with 373 μL of water and added to the mRNA/salt mixture to afford mRNA-lipoplexes with a 9:1 lipid:nucleotide molar ratio and a final RNA concentration of 0.1 mg/mL. The RNA-LPX formulation was diluted to 0.05 mg/mL using a HEPES-buffered solution (5 mM, 150 mM NaCl). Lipoplex size is determined by dynamic light scattering using a Malvern Zetasizer Nano ZS (Malvern, UK). Lipoplexes varied in size from 130-280 nm with PDI of 0.3-0.8 with zeta potentials in the 50-60 mV range.

### 5. mOVA-DOTMA Cationic Lipid-CI058 Nanoparticles (LNP-mOVA-CI058)

DOTMA, DSPC, cholesterol, DSPE-PEG-Mal and the CI058 adjuvant were solubilized in EtOH (10 mg/mL total lipid concentration) at a molar ratio of 50:10:38.5:1.5:0.15. Lipid nanoparticles were prepared at a basic lipid amine (i.e., DOTMA) to nucleotide ratio of approximately 9:1. Briefly, the mRNA was diluted to 0.1 mg/mL in 100 mM acetate buffer, pH 5. A NanoAssemblr Ignite microfluidics mixer with staggered herringbone cartridge was used to mix the ethanolic lipid solution with the mRNA aqueous solution at a ratio of 1:3 (v/v) with total flow rate 12 mL/min. The EtOH was then removed and the external buffer replaced with PBS by 40-fold dilution and concentration using a centrifugal filter with Mw cut off of 30,000 Da. Lipid nanoparticle size is determined by dynamic light scattering using a Malvern Zetasizer Nano ZS (Malvern, UK). Size (108 nm), PDI (0.28) and Zeta potential (17.0 mV).

### 6. Biological Methods

### 6.1 Mice

C57BL/6 (B6), OT-I¹, and TRAJ18^{-/-} mice were bred and maintained at the Department of Microbiology and Immunology, The University of Melbourne, or the BRU, Malaghan Institute of Medical Research, New Zealand. All mice used were 6-12 weeks of age and littermates of the same sex were randomly assigned to experimental groups. Animals used for the generation of the sporozoites were 4-5 week-old male Swiss Webster mice purchased from the Monash Animal Services (Melbourne, Victoria, Australia) and housed at 22° C. to 25° C. on a 12 hr light/dark cycle at the School of Biosciences, The University of Melbourne, Australia.

### 6.2 Transfer of Naïve OVA-Specific CD8⁺ T Cells from OT-I Mouse Strain

Naïve OT-I CD8′ T cells were isolated by negative selection from the spleen of OT-I mice. The tissue from each spleen was disrupted by passing through 70 mm cell strainers, pelleted by centrifugation (1600×g, 4° C., 5 min), washed by adding RPMI supplemented with 2.5% FBS followed by centrifugation, and then red cells were lysed by incubation of the single cell suspension in 2 mL red blood cells lysis solution (Sigma Aldrich) for 1 min at room temperature. For FIGS. 1, 2A-E, 5, 6B, 6D, 7A, and 7C, transgenic OT-I cells were enriched by negative selection of cells that were not CD8⁺ T cells. To accomplish this, the cells were harvested by centrifugation, washed and resuspended in RPMI/2.5% FBS solution (10 μL for every 10⁶ cells) containing a cocktail of rat monoclonal antibodies specific for: mouse CD4 (clone GK1.5; 1/400 dilution), MHC class II (M5/114; 1/100), macrophages (F4/80; 1/50) and neutrophils (Gr1, RB6-8C55; 1/100); incubation was for 30 min at 4° C. The cells were washed and resuspended in 0.2 mL RPMI/2.5% FBS, and then a 0.2 mL solution containing BioMag goat anti-rat IgG beads (10 beads per cell) (Qiagen, Chadstone, VIC, Australia) was added for 20 min, and the antibody-bound cells sequestered to the side of the tube using a magnet. Enriched naïve CD8+ T cells in the supernatant were collected, harvested by centrifugation, washed and resuspended in RPMI/2.5% FBS. The cells were counted and their purity analyzed by staining with anti-CD8a and anti-Va2 TCR antibodies (expressed by OT-I cells). Cell counts were adjusted to 2.5×10⁵/mL in PBS and mice were injected with 200 mL intravenously one day prior to vaccination with mRNA vaccines. For FIGS. 6A, 6C, 7B and 7D, there was no negative selection, and volume was adjusted according to flow cytometry results to give 50,000 OT-I CD8⁺ T cells injected per mouse.

### 6.3 Plasmodium Infection

Anopheles stephensi mosquitoes (STE2, MRA-128, from BEI Resources) were reared in an Australian Biosecurity (Department of Agriculture and Water Resources) approved insectary. The conditions were maintained at 27° C. and 75-80% humidity with a 12 h light and dark photo-period in filtered drinking water (Frantelle beverages, Australia) and fed with Sera vipan baby fish food (Sera). The larvae were bred in plastic food trays (P.O.S.M Pty Ltd, Australia) containing 300 larvae, each with regular water changes every 3 days. Upon ecloding, the adult mosquitoes were transferred to aluminium cages (BioQuip Products, Inc. St.Rancho Dominguez, CA, USA) and kept in a secure incubator (Conviron), in the insectary at the same temperature and humidity and maintained on 10% sucrose.

P. berghei ANKA expressing OVA under the HSP70 promoter were used to challenge vaccinated mice². Infections of naïve Swiss mice were carried out by i.p inoculation of PbA infected RBCs obtained from a donor mouse between the first and fourth passages from a cryopreserved stock. Parasitemia was monitored by Giemsa smear and exflagellation quantified 3 days post infection. 1 mL of tail prick blood was mixed with 100 mL of exflagellation media (RPMI [Invitrogen]supplemented with 10% v/v foetal bovine serum, pH 8.4), incubated for 15 min at 20° C., and exflagellation events per 1×10⁴ red blood cells were counted. A. stephensi mosquitoes were allowed to feed on anaesthetised mice once the exflagellation rate was assessed between 12-15 exflagellation events per 1×10⁴ red blood cells. 22 days after biting, salivary glands were dissected and checked for the presence of sporozoites.

Sporozoites were dissected from mosquito salivary glands3, and resuspended in cold PBS. For challenge experiments, 200 freshly dissected PbA-OVA sporozoites were injected i.v. as indicated. Mice were assessed for parasitemia at day 6, 7, 8, 10 and 12 using flow cytometry. Briefly, a drop of blood was collected from the mice and stained with Hoechst 33258 dye (ThermoFisher, Scoresby, Victoria, Australia) for 1 hr at 37° C. Samples were analyzed on a LSR Fortessa (BD Biosciences, San Jose, USA) using a violet laser (405 nm) to excite the dye. After gating on RBCs the percentage of Hoechst positive cells were measured. Values were compared to uninfected controls and typically values of >0.1% were considered positive for parasites. Mice positive for parasites on two consecutive days were euthanized. Mice were considered sterilely protected if they remained parasitemia-negative on day 12 after challenge.

### 6.4 Lymphocyte Isolation from Organs

Tissues were harvested from mice at different time points after immunization. For spleen cell preparations, the organ was passed through 70 mm mesh and red blood cells were lysed. Liver cell suspensions were passed through 70 mm mesh and resuspended in 35% isotonic Percoll (Sigma). Cells were then centrifuged at 500 g for 20 min at RT, the pellet harvested and then red cells lysed before further analysis.

### 6.5 Flow Cytometry

Lymphocytes were stained with monoclonal antibodies for: CD8a (53-6.7), Ly5.1 (A20), CD44 (IM7), CXCR6 (SA051D1), CX3CR1 (SA011F11), from BioLegend (Australian Biosearch, Karrinyup, WA. Australia), and CD62L (MEL-14) and CD69 (H1.2F3), from eBioscience (Jomar Life Research, Scoresby, VIC, Australia). Dead cells were excluded by propidium iodide staining or far red live/dead fixable dye (ThermoFisher). In some experiments cells were stained with an α-GalCer (PBS-44—a gift from Prof. Paul Savage, Brigham-Young University, UT, USA)—loaded CD1d tetramer produced in-house at 4° C. for 30 min, washed, and further antibody staining was conducted for 30 min at 4° C. Antibodies used were: CD3 (17A2), CD19 (1D3), NK1.1 (PK136), CD69 (H1.2F3), all from BioLegend (CA, USA). The viability dye used was DAPI (Invitrogen, NZ). To track the endogenous T cell response to RPL6 expressing vaccines, cells were stained with H2-K^{b}-PbRPL6₁₂₀₋₁₂₇ tetramers (made in house) for 1 h at room temp prior to staining with surface antibodies. Single-color positive control samples were used to adjust compensation and cells were analyzed by flow cytometry on a LSR Fortessa (BD Biosciences), or LSRII SORP using Flowjo software (Tree Star Inc.).

### 6.6 Statistical Analysis

Figures were generated using GraphPad Prism 7. Data are shown as mean values±S.E.M as indicated in the figure legends. Data was log transformed, assessed for normality then a one-way ANOVA with Tukey's multiple comparison test was performed. To compare survival after challenge, groups were compared using Fisher's exact test. The statistical tests performed on the data are indicated in the figure legends and results, along with sample size indicating the number of animals used. P-values <0.05 (*), <0.01 (**), 0.001 (***) or 0.0001 (****) were considered statistically significant.

### 6.7 CI536-MC3-mOVA Ionisable Lipid-Nanoparticles (CI536-MC3-mOVA-LNP)

MC3, DSPC, cholesterol, DMG-PEG-2000 and the CI536 adjuvant are solubilized in EtOH (10 mg/mL total lipid concentration) at a molar ratio of 50:10:38.5:1.5:0.065, respectively. Preformed vesicles are prepared by mixing the lipids mixture in ethanol with 100 mM sodium acetate buffer pH 5.3 using a NanoAssemblr Ignite microfluidics mixer with staggered herringbone cartridge with total flow rate 12 mL/min and flow rate ratio of ethanolic to aqueous solution 1:3 (v/v). 0.6 mL of preformed vesicle per 100 μg mRNA was added to the mRNA at 1 mg/mL in 100 mM sodium acetate buffer pH 5.5 to achieve an ionisable lipid amine to nucleotide ratio of 4:1. The buffer was replaced by PBS 40-fold dilution and concentration using a centrifugal filter with Mw cut off of 30 k Da. Lipid nanoparticle size is determined by dynamic light scattering using a Malvern Zetasizer Nano ZS (Malvern, UK). Size 142 nm, PDI 0.22. Encapsulation efficiency and concentration of mRNA was measured using the RiboGreen reagent kit, the mRNA concentration is adjusted to 50 μg/mL with PBS. Encapsulation efficiency 75%.

### REFERENCES

1. Guevara, M. L.; Jilesen, Z.; Stojdl, D.; Persano, S., Codelivery of mRNA with alpha-Galactosylceramide Using a New Lipopolyplex Formulation Induces a Strong Antitumor Response upon Intravenous Administration. ACS Omega 2019, 4 (8), 13015-13026.
2. Verbeke, R.; Lentacker, I.; Breckpot, K.; Janssens, J.; Van Calenbergh, S.; De Smedt, S. C.; Dewitte, H., Broadening the Message: A Nanovaccine Co-loaded with Messenger RNA and alpha-GalCer Induces Antitumor Immunity through Conventional and Natural Killer T Cells. ACS Nano 2019, 13 (2), 1655-1669.
3. Compton, B. J.; Farrand, K. J.; Tang, C. W.; Osmond, T. L.; Speir, M.; Authier-Hall, A.; Wang, J.; Ferguson, P. M.; Chan, S. T. S.; Anderson, R. J.; Cooney, T. R.; Hayman, C. M.; Williams, G. M.; Brimble, M. A.; Brooks, C. R.; Yong, L. K.; Metelitsa, L. S.; Zajonc, D. M.; Godfrey, D. I.; Gasser, O.; Weinkove, R.; Painter, G. F.; Hermans, I. F., Enhancing T cell responses and tumour immunity by vaccination with peptides conjugated to a weak NKT cell agonist. Org. Biomol. Chem. 2019, 17 (5), 1225-1237.
4. Compton, B. J.; Tang, C. W.; Johnston, K. A.; Osmond, T. L.; Hayman, C. M.; Larsen, D. S.; Hermans, I. F.; Painter, G. F., Synthesis and Activity of 6″-Deoxy-6″-thio-α-GalCer and Peptide Conjugates. Org. Lett. 2015, 17 (24), 5954-5957.
5. Holz, L. E.; Chua, Y. C.; de Menezes, M. N.; Anderson, R. J.; Draper, S. L.; Compton, B. J.; Chan, S. T. S.; Mathew, J.; Li, J.; Kedzierski, L.; Wang, Z.; Beattie, L.; Enders, M. H.; Ghilas, S.; May, R.; Steiner, T. M.; Lange, J.; Fernandez-Ruiz, D.; Valencia-Hernandez, A. M.; Osmond, T. L.; Farrand, K. J.; Seneviratna, R.; Almeida, C. F.; Tullett, K. M.; Bertolino, P.; Bowen, D. G.; Cozijnsen, A.; Mollard, V.; McFadden, G. I.; Caminschi, I.; Lahoud, M. H.; Kedzierska, K.; Turner, S. J.; Godfrey, D. I.; Hermans, I. F.; Painter, G. F.; Heath, W. R., Glycolipid-peptide vaccination induces liver-resident memory CD8(+) T cells that protect against rodent malaria. Sci Immunol 2020, 5 (48).

The claims defining the invention are as follows:

1. An immunomodulatory composition comprising a first agent that comprises an invariant NKT cell (iNKT cell) agonist that activates or induces the expansion of tissue-resident memory T cells (T_{RM} cells), together with a second agent comprising an immune stimulator that stimulates or otherwise enhances an immune response to a target antigen in a subject or a polynucleotide sequence encoding an immune stimulator that stimulates or otherwise enhances an immune response to the target antigen in a subject.

2. The composition of claim 1, wherein the first agent modifies a phenotype of an iNKT cell.

3. The composition of claim 2, wherein the modified phenotype comprises a downregulation of TCR expression.

4. The composition of claim 2 or claim 3, wherein the modified phenotype comprises an enhanced expression of PD1 on the surface of the iNKT cell.

5. The composition of any one of claims 1 to 4, wherein the iNKT cell is in the liver of the subject.

6. The composition of any one of claims 1 to 3, wherein the iNKT cell agonist induces T_{RM} cell accumulation in the liver of the subject.

7. The composition of any one of claims 1 to 6, wherein upon administration to a subject the ratio of T_{RM} cells to central memory T (T_{CM}) cells and/or effector memory (T_{EM}) cells present in the liver is increased.

8. The composition of any claim 7, wherein the ratio of T_{RM} cells to T_{CM} and T_{EM} cells (i.e., T_{RM} cells:T_{CM}+T_{EM} cells) is greater than about 1:1.

9. The composition of claim 6 or claim 7, wherein the ratio of T_{RM} cells to T_{CM} and T_{EM} cells (i.e., T_{RM} cells:T_{CM}±T_{EM} cells) is greater than about 3:2.

10. The composition of any one of claims 7 to 9, wherein the ratio of T_{RM} cells to T_{CM} and T_{EM} cells (i.e., T_{RM} cells:T_{CM}±T_{EM} cells) is greater than about 2.3:1

11. The composition of any one of claims 7 to 10, wherein at least 30%, 35%, 40%, 45%, 50%, 55%, 60%, 65%, 66%, 70%, 75% of the memory T cell population in the liver are T_{RM} cells.

12. The composition of claim 7, wherein greater than about 70% of the memory T cell population in the liver are T_{RM} cells.

13. The composition of any one of claims 1 to 12, wherein the iNKT cell agonist is a derivative of α-galactosylceramide, wherein the derivation is at the 6 position of the galactose ring.

14. The composition of any one of claims 1 to 9, wherein the iNKT cell agonist comprises a Compound of Formula (I): 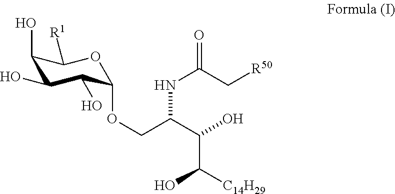
or a pharmaceutically acceptable salt thereof, wherein:
R¹ is selected from hydrogen, halo, alkyl, alkenyl, alkynyl, aryl, acyl, carbocyclyl, heterocyclyl, heteroaryl, alkyloxy, alkenyloxy, alkynyloxy, aryloxy, acyloxy, carbocyclyloxy, heterocyclyloxy, heteroaryloxy, alkylthio, alkenylthio, alkynylthio, arylthio, acylthio, carbocyclylthio, heterocyclylthio, heteroarylthio, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkyloxyacylalkyl, alkylcarbocyclyloxy, alkylheterocyclyloxy, alkylheteroaryloxy, alkylthioalkyl, alkenylthioalkyl, alkynylthioalkyl, alkenylthioalkenyl, alkenylthioalkynyl, alkynylthioalkynyl, arylthioalkyl, alkylacylthio, alkylcarbocyclylthio, alkylheterocyclylalkyl, alkenylheterocyclylalkyl, alkynylheterocyclylalkyl, alkenylheterocyclylalkenyl, alkenylheterocyclylalkynyl, alkynylheterocyclylalkynyl, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, arylacyloxy, arylcarbocyclyloxy, arylheterocyclyloxy, arylheteroaryloxy, alkylthioaryl, alkenylthioaryl, alkynylthioaryl, arylthioaryl, arylacylthio, arylcarbocyclylthio, arylheterocyclylthio, arylheteroarylthio, alkylamino, alkenylamino, alkynylamino, arylamino, acylamino, carbocyclylamino, heterocyclylamino, heteroarylamino, alkylaminoalkyl, alkenylaminoalkyl, alkynylaminoalkyl, alkenylaminoalkenyl, alkenylaminoalkynyl, alkynylaminoalkynyl, arylaminoalkyl, alkylacylamino, alkylcarbocyclylamino, alkylheterocyclylamino, alkylheteroarylamino, alkylaminoaryl, alkenylaminoaryl, alkynylaminoaryl, arylaminoaryl, arylacylamino, arylcarbocyclylamino, arylheterocyclylamino, arylheteroarylamino, alkylamido, alkenylamido, alkynylamido, arylamido, acylamido, carbocyclylamido, heterocyclylamido, heteroarylamido, alkylamidoalkyl, alkenylamidoalkyl, alkynylamidoalkyl, alkenylamidoalkenyl, alkenylamidoalkynyl, alkynylamidoalkynyl, arylamidoalkyl, alkylacylamido, alkylcarbocyclylamido, alkylheterocyclylamido, alkylheteroarylamido, alkylamidoaryl, alkenylamidoaryl, alkynylamidoaryl, arylamidoaryl, arylacylamido, arylcarbocyclylamido, arylheterocyclylamido, arylheteroarylamido, alkylcarbamyl, alkenylcarbamyl, alkynylcarbamyl, arylcarbamyl, acylcarbamyl, carbocyclylcarbamyl, heterocyclylcarbamyl, heteroarylcarbamyl, alkylcarbamylalkyl, alkenylcarbamylalkyl, alkynylcarbamylalkyl, alkenylcarbamylalkenyl, alkenylcarbamylalkynyl, alkynylcarbamylalkynyl, arylcarbamylalkyl, alkylacylcarbamyl, alkylcarbocyclylcarbamyl, alkylheterocyclylcarbamyl, alkylheteroarylcarbamyl, alkylcarbamylaryl, alkenylcarbamylaryl, alkynylcarbamylaryl, arylcarbamylaryl, arylacylcarbamyl, arylcarbocyclylcarbamyl, arylheterocyclylcarbamyl, arylheteroarylcarbamyl, alkyldisulfidyl, alkenyldisulfidyl, alkynyldisulfidyl, aryldisulfidyl, acyldisulfidyl, carbocyclyldisulfidyl, heterocyclyldisulfidyl, heteroaryldisulfidyl, alkyldisulfidylalkyl, alkenyldisulfidylalkyl, alkynyldisulfidylalkyl, alkenyldisulfidylalkenyl, alkenyldisulfidylalkynyl, alkynyldisulfidylalkynyl, aryldisulfidylalkyl, alkylacyldisulfidyl, alkylcarbocyclyldisulfidyl, alkylheterocyclyldisulfidyl, alkylheteroaryldisulfidyl, alkyldisulfidylaryl, alkenyldisulfidylaryl, alkynyldisulfidylaryl, aryldisulfidylaryl, arylacyldisulfidyl, arylcarbocyclyldisulfidyl, arylheterocyclyldisulfidyl, and arylheteroaryldisulfidyl, and wherein R¹ is optionally substituted;
and wherein R¹ is not —CH₂OH;
R⁵⁰ is selected from optionally substituted alkyl, alkenyl, alkynyl.

15. The composition of claim 14, wherein R¹ is selected from C₁-C₁₈ alkyl, C₂-C₁₈ alkenyl, C₂-C₁₈ alkynyl, C₆-C₁₈ aryl, C₁-C₁₈ acyl, C₃-C₁₈ carbocyclyl, C₂-C₁₈ heterocyclyl, C₃-C₁₈ heteroaryl, C₁-C₁₈ alkyloxy, C₂-C₁₈ alkenyloxy, C₂-C₁₈ alkynyloxy, C₆-C₁₈ aryloxy, C₁-C₁₈ acyloxy, C₃-C₁₈ carbocyclyloxy, C₂-C₁₈ heterocyclyloxy, C₃-C₁₈ heteroaryloxy, C₁-C₈₅ alkylthio, C₂-C₁₈ alkenylthio, C₂-C₁₈ alkynylthio, C₅-C₁₈ arylthio, C₁-C₁₈ acylthio, C₃-C₁₈ carbocyclylthio, C₂-C₁₈ heterocyclylthio, C₃-C₁₉ heteroarylthio, C₃-C₁₉ alkylalkenyl, C₃-C₁₉ alkylalkynyl C₇-C₂₄ alkylaryl, C₂-C₁₈ alkylacyl, C₄-C₁₈ alkylcarbocyclyl, C₃-C₁₈ alkylheterocyclyl, C₄-C₁₈ alkylheteroaryl, C₂-C₁₈ alkyloxyalkyl, C₃-C₁₈ alkenyloxyalkyl, C₃-C₁₈ alkynyloxyalkyl, C₇-C₂₄ aryloxyalkyl, C₂-C₁₈ alkylacyloxy, C₂₋₁₈ alkyloxyacyl C₂₋₁₈ alkyl, C₄-C₁₈ alkylcarbocyclyloxy, C₃-C₁₈ alkylheterocyclyloxy, C₄-C₁₈ alkylheteroaryloxy, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₉ alkylcarbocyclylthio, C₄-C₁₈ alkylheterocyclylalkyl, C₄-C₁₉ alkenylheterocyclylalkyl, C₄-C₁₉ alkynylheterocyclylalkyl, C₅-C₁₈ alkenylheterocyclylalkenyl, C₅-C₁₈ alkenylheterocyclylalkynyl, C₅-C₁₈ alkynylheterocyclylalkynyl, C₃-C₁₈ alkylheterocyclylthio, C₄-C₁₈ alkylheteroarylthio, C₄-C₁₈ alkylalkenylalkyl, C₄-C₁₈ alkylalkynylalkyl, C₈-C₂₄ alkylarylalkyl, C₃-C₁₈ alkylacylalkyl, C₁₃-C₂₄ arylalkylaryl, C₁₄-C₂₄ arylalkenylaryl, C₁₄-C₂₄ arylalkynylaryl, C₁₃-C₂₄ arylacylaryl, C₇-C₁₈ arylacyl, C₉-C₁₈ arylcarbocyclyl, C₈-C₁₈ arylheterocyclyl, C₉-C₁₈ arylheteroaryl, C₈-C₁₈ alkenyloxyaryl, C₈-C₁₈ alkynyloxyaryl, C₁₂-C₂₄ aryloxyaryl, C₇-C₁₈ arylacyloxy, C₉-C₁₈ arylcarbocyclyloxy, C₈-C₁₈ arylheterocyclyloxy, C₉-C₁₈ arylheteroaryloxy, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylthio, C₂-C₁₈ alkenylthio, C₂-C₁₈ alkynylthio, C₅-C₁₈ arylthio, C₁-C₁₈ acylthio, C₃-C₁₈ carbocyclylthio, C₂-C₁₈ heterocyclylthio, C₃-C₁₈ heteroarylthio, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₉ alkylcarbocyclylthio, C₃-C₁₈ alkylheterocyclylthio, C₄-C₁₈ alkylheteroarylthio, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₅ arylcarbocyclylthio, C₈-C₁₈ arylheterocyclylthio, C₉-C₁₈ arylheteroarylthio, C₁-C₁₉ alkylamino, C₂-C₁₈ alkenylamino, C₂-C₁₈ alkynylamino, C₆-C₁₈ arylamino, C₁-C₁₈ acylamino, C₃-C₁₈ carbocyclylamino, C₂-C₁₈ heterocyclylamino, C₃-C₁₈ heteroarylamino, C₂-C₁₈ alkylaminoalkyl, C₃-C₁₈ alkenylaminoalkyl, C₃-C₁₈ alkynylaminoalkyl, C₄-C₁₈ alkenylaminoalkenyl, C₄-C₁₈ alkenylaminoalkynyl, C₄-C₁₈ alkynylaminoalkynyl, C₇-C₂₄ arylaminoalkyl, C₂-C₁₈ alkylacylamino, C₄—C₁₈ alkylcarbocyclylamino, C₃-C₁₃ alkylheterocyclylamino, C₄-C₁₈ alkylheteroarylamino, C₈-C₁₈ alkylaminoaryl, C₈-C₁₈ alkenylaminoaryl, C₈-C₁₈ alkynylaminoaryl, C₁₂-C₂₄ arylaminoaryl, C₇-C₁₈ arylacylamino, C₉-C₁₈ arylcarbocyclylamino, C₈-C₁₈ arylheterocyclylamino, C₉-C₁₈ arylheteroarylamino, C₁-C₁₈ alkylamido, C₂-C₁₈ alkenylamido, C₂-C₁₈ alkynylamido, C₆-C₁₈ arylamido, C₁-C₁₈ acylamido, C₃-C₁₈ carbocyclylamido, C₂-C₁₈ heterocyclylamido, C₃-C₁₈ heteroarylamido, C₂-C₁₈ alkylamidoalkyl, C₃-C₁₈ alkenylamidoalkyl, C₃-C₁₈ alkynylamidoalkyl, C₄-C₁₈ alkenylamidoalkenyl, C₄-C₁₈ alkenylamidoalkynyl, C₄-C₁₈ alkynylamidoalkynyl, C₇-C₂₄ arylamidoalkyl, C₂-C₁₈ alkylacylamido, C₄-C₁₈ alkylcarbocyclylamido, C₃-C₁₈ alkylheterocyclylamido, C₄-C₁₈ alkylheteroarylamido, C₈-C₁₈ alkylamidoaryl, C₈-C₁₈ alkenylamidoaryl, C₈-C₁₈ alkynylamidoaryl, C₁₂-C₂₄ arylamidoaryl, C₇-C₁₈ arylacylamido, C₉-C₁₈ arylcarbocyclylamido, C₈-C₁₈ arylheterocyclylamido, C₉-C₁₈ arylheteroarylamido, C₁-C₁₈ alkylcarbamyl, C₂-C₁₈ alkenylcarbamyl, C₂-C₁₈ alkynylcarbamyl, C₆-C₁₈ arylcarbamyl, C₁-C₁₈ acylcarbamyl, C₃-C₁₈ carbocyclylcarbamyl, C₂-C₁₈ heterocyclylcarbamyl, C₃-C₁₈ heteroarylcarbamyl, C₂-C₁₈ alkylcarbamylalkyl, C₃-C₁₈ alkenylcarbamylalkyl, C₃-C₁₈ alkynylcarbamylalkyl, C₄-C₁₈ alkenylcarbamylalkenyl, C₄-C₁₈ alkenylcarbamylalkynyl, C₄-C₁₈ alkynylcarbamylalkynyl, C₇-C₂₄ arylcarbamylalkyl, C₂-C₁₈ alkylacylcarbamyl, C₄-C₁₈ alkylcarbocyclylcarbamyl, C₃-C₁₈ alkylheterocyclylcarbamyl, C₄-C₁₈ alkylheteroarylcarbamyl, C₈-C₁₈ alkylcarbamylaryl, C₈-C₁₈ alkenylcarbamylaryl, C₈-C₁₈ alkynylcarbamylaryl, C₁₂-C₂₄ arylcarbamylaryl, C₇-C₁₈ arylacylcarbamyl, C₉-C₁₈ arylcarbocyclylcarbamyl, C₈-C₁₈ arylheterocyclylcarbamyl, C₉-C₁₈ arylheteroarylcarbamyl, C₁-C₁₈ alkyldisulfidyl, C₂-C₁₈ alkenyldisulfidyl, C₂-C₁₈ alkynyldisulfidyl, C₆-C₁₈ aryldisulfidyl, C₁-C₁₈ acyldisulfidyl, C₃-C₁₈ carbocyclyldisulfidyl, C₂-C₁₈ heterocyclyldisulfidyl, C₃-C₁₈ heteroaryldisulfidyl, C₂-C₁₈ alkyldisulfidylalkyl, C₃-C₁₈ alkenyldisulfidylalkyl, C₃-C₁₈ alkynyldisulfidylalkyl, C₄-C₁₈ alkenyldisulfidylalkenyl, C₄-C₁₈ alkenyldisulfidylalkynyl, C₄-C₁₈ alkynyldisulfidylalkynyl, C₇-C₂₄ aryldisulfidylalkyl, C₂-C₁₈ alkylacyldisulfidyl, C₄-C₁₈ alkylcarbocyclyldisulfidyl, C₃-C₁₈ alkylheterocyclyldisulfidyl, C₄-C₁₈ alkylheteroaryldisulfidyl, C₈-C₁₈ alkyldisulfidylaryl, C₈-C₁₈ alkenyldisulfidylaryl, C₈-C₁₈ alkynyldisulfidylaryl, C₁₂-C₂₄ aryldisulfidylaryl, C₇-C₁₈ arylacyldisulfidyl, C₉-C₁₈ arylcarbocyclyldisulfidyl, C₈-C₁₈ arylheterocyclyldisulfidyl, and C₉-C₁₈ arylheteroaryldisulfidyl, and wherein R¹ is optionally substituted;
and wherein R is not —CH₂OH.

16. The composition of claim 14 or claim 15, wherein R′ is selected from alkyl (e.g., C₁-C₁₈, C₁-C₆, C₁-C₅, C₈-C₁₈, or C₉-C₁₈), aryl (e.g., C₆-C₁₈), heteroaryl (e.g., C₃-C₁), carbocyclyl (e.g., C₃-C₁₈), heterocyclyl (e.g., C₂-C₁₈), alkylaryl (e.g., C₇-C₂₄), alkylheteroaryl (e.g., C₄-C₁₈), alkylcarbocyclyl (e.g., C₄-C₁₈), alkylthioalkyl (e.g., wherein each alkyl is C₁-C₁₈), arylthioalkyl (e.g., wherein each alkyl is C₇-C₁₈), alkylaminoalkyl (e.g., wherein each alkyl is C₁-C₁₈), alkylamidoalkyl (e.g., wherein each alkyl is C₁-C₁₈), alkylheterocyclylalkyl (e.g., C₃-C₁₈), alkylthio (e.g., C₁-C₁₉; such as thiomethyl) and alkylheterocyclyl (e.g., C₃-C₁₈);
and wherein R¹ is not —CH₂OH.

17. The method of any one of claims 1 to 15, wherein the iNKT agonist comprises a Compound of Formula (II): 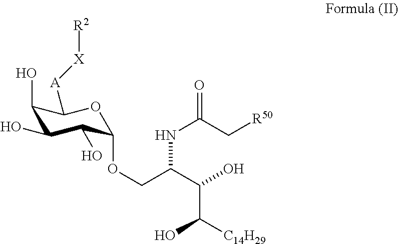
or a pharmaceutically acceptable salt thereof, wherein:
A is selected from alkyl, alkenyl, and alkynyl;
X is selected from —CR¹⁰R¹¹—, —O—, —S—, —NR^{a}—, —NR^{a}C(O)—, —NR^{a}C(O)O—, —S—S—, heterocyclyl, heteroaryl, wherein R¹⁰ and R¹¹ where present are independently selected from H, halo, C₁₋₂alkyl, and wherein R^{a} may be selected from hydrogen, alkyl, alkenyl, alkynyl, aryl, carbocyclyl, heteroaryl, heterocyclyl, arylalkyl, and acyl;
R² is selected from hydrogen, halo, alkyl, alkenyl, alkynyl, aryl, acyl, carbocyclyl, heterocyclyl, heteroaryl, alkyloxy, alkenyloxy, alkynyloxy, aryloxy, acyloxy, carbocyclyloxy, heterocyclyloxy, heteroaryloxy, alkylthio, alkenylthio, alkynylthio, arylthio, acylthio, carbocyclylthio, heterocyclylthio, heteroarylthio, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkyloxyacylalkyl, alkylcarbocyclyloxy, alkylheterocyclyloxy, alkylheteroaryloxy, alkylthioalkyl, alkenylthioalkyl, alkynylthioalkyl, alkenylthioalkenyl, alkenylthioalkynyl, alkynylthioalkynyl, arylthioalkyl, alkylacylthio, alkylcarbocyclylthio, alkylheterocyclylalkyl, alkenylheterocyclylalkyl, alkynylheterocyclylalkyl, alkenylheterocyclylalkenyl, alkenylheterocyclylalkynyl, alkynylheterocyclylalkynyl, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, arylacyloxy, arylcarbocyclyloxy, arylheterocyclyloxy, arylheteroaryloxy, alkylthioaryl, alkenylthioaryl, alkynylthioaryl, arylthioaryl, arylacylthio, arylcarbocyclylthio, arylheterocyclylthio, arylheteroarylthio, alkylamino, alkenylamino, alkynylamino, arylamino, acylamino, carbocyclylamino, heterocyclylamino, heteroarylamino, alkylaminoalkyl, alkenylaminoalkyl, alkynylaminoalkyl, alkenylaminoalkenyl, alkenylaminoalkynyl, alkynylaminoalkynyl, arylaminoalkyl, alkylacylamino, alkylcarbocyclylamino, alkylheterocyclylamino, alkylheteroarylamino, alkylaminoaryl, alkenylaminoaryl, alkynylaminoaryl, arylaminoaryl, arylacylamino, arylcarbocyclylamino, arylheterocyclylamino, arylheteroarylamino, alkylamido, alkenylamido, alkynylamido, arylamido, acylamido, carbocyclylamido, heterocyclylamido, heteroarylamido, alkylamidoalkyl, alkenylamidoalkyl, alkynylamidoalkyl, alkenylamidoalkenyl, alkenylamidoalkynyl, alkynylamidoalkynyl, arylamidoalkyl, alkylacylamido, alkylcarbocyclylamido, alkylheterocyclylamido, alkylheteroarylamido, alkylamidoaryl, alkenylamidoaryl, alkynylamidoaryl, arylamidoaryl, arylacylamido, arylcarbocyclylamido, arylheterocyclylamido, arylheteroarylamido, alkylcarbamyl, alkenylcarbamyl, alkynylcarbamyl, arylcarbamyl, acylcarbamyl, carbocyclylcarbamyl, heterocyclylcarbamyl, heteroarylcarbamyl, alkylcarbamylalkyl, alkenylcarbamylalkyl, alkynylcarbamylalkyl, alkenylcarbamylalkenyl, alkenylcarbamylalkynyl, alkynylcarbamylalkynyl, arylcarbamylalkyl, alkylacylcarbamyl, alkylcarbocyclylcarbamyl, alkylheterocyclylcarbamyl, alkylheteroarylcarbamyl, alkylcarbamylaryl, alkenylcarbamylaryl, alkynylcarbamylaryl, arylcarbamylaryl, arylacylcarbamyl, arylcarbocyclylcarbamyl, arylheterocyclylcarbamyl, arylheteroarylcarbamyl, alkyldisulfidyl, alkenyldisulfidyl, alkynyldisulfidyl, aryldisulfidyl, acyldisulfidyl, carbocyclyldisulfidyl, heterocyclyldisulfidyl, heteroaryldisulfidyl, alkyldisulfidylalkyl, alkenyldisulfidylalkyl, alkynyldisulfidylalkyl, alkenyldisulfidylalkenyl, alkenyldisulfidylalkynyl, alkynyldisulfidylalkynyl, aryldisulfidylalkyl, alkylacyldisulfidyl, alkylcarbocyclyldisulfidyl, alkylheterocyclyldisulfidyl, alkylheteroaryldisulfidyl, alkyldisulfidylaryl, alkenyldisulfidylaryl, alkynyldisulfidylaryl, aryldisulfidylaryl, arylacyldisulfidyl, arylcarbocyclyldisulfidyl, arylheterocyclyldisulfidyl, and arylheteroaryldisulfidyl, and wherein R² is optionally substituted;
and wherein -A-X—R² is not —CH₂OH;
R⁵⁰ is selected from optionally substituted alkyl, alkenyl, alkynyl.

18. The method of claim 17, wherein R^{a} is selected from hydrogen, C₁-C₈ alkyl, C₂-C₈ alkenyl, C₂-C₈ alkynyl, C₆-C₁₈ aryl, C₃-C₁₈ carbocyclyl, C₃-C₁₈ heteroaryl, C₂-C₁₈ heterocyclyl, C₇-C₂₄ arylalkyl, and C₁-C₈ acyl.

19. The method of claim 17 or claim 18, wherein X is S or —NR^{a}—, and wherein R^{a} is selected from hydrogen, alkyl, alkenyl, alkynyl, aryl, carbocyclyl, heteroaryl, heterocyclyl, arylalkyl, and acyl.

20. The method of claim 19, wherein R^{a} is selected from hydrogen, C₁-C₁₈ alkyl, C₂-C₈ alkenyl, C₂-C₈ alkynyl, C₆-C₁₈ aryl, C₃-C₁₈ carbocyclyl, C₃-C₁₈ heteroaryl, C₂-C₁₈ heterocyclyl, C₇-C₂₄ arylalkyl, and C₁-C₈ acyl.

21. The method of any one of claims 14 to 20, wherein A is —CR¹²R¹³— and R¹² and R¹³ are independently selected from H, halo, C₁₋₂alkyl.

22. The method of any one of claims 14 to 20, wherein A is —CH₂—.

23. The method of any one of claims 14 to 22, wherein R² is selected from C₁-C₁₈ alkyl, C₂-C₁₈ alkenyl, C₂-C₁₈ alkynyl, C₆-C₁₈ aryl, C₁-C₁₈ acyl, C₃-C₁₈ carbocyclyl, C₂-C₁₈ heterocyclyl, C₃-C₁₈ heteroaryl, C₁-C₁₈ alkyloxy, C₂-C₁₈ alkenyloxy, C₂-C₁₈ alkynyloxy, C₆-C₁₈ aryloxy, C₁-C₁₈ acyloxy, C₃-C₁₈ carbocyclyloxy, C₂-C₁₈ heterocyclyloxy, C₃-C₁₈ heteroaryloxy, C₁-C₁₈ alkylthio, C₂-C₁₈ alkenylthio, C₂-C₁₈ alkynylthio, C₅-C₁₈ arylthio, C₁-C₁₈ acylthio, C₃-C₁₈ carbocyclylthio, C₂-C₁₈ heterocyclylthio, C₃-C₁₈ heteroarylthio, C₃-C₁₈ alkylalkenyl, C₃-C₁₈ alkylalkynyl C₇-C₂₄ alkylaryl, C₂-C₁₈ alkylacyl, C₄-C₁₈ alkylcarbocyclyl, C₃-C₁₈ alkylheterocyclyl, C₄-C₁₈ alkylheteroaryl, C₂-C₁₈ alkyloxyalkyl, C₃-C₁₈ alkenyloxyalkyl, C₃-C₁₈ alkynyloxyalkyl, C₇-C₂₄ aryloxyalkyl, C₂-C₁₈ alkylacyloxy, C₂₋₁₈ alkyloxyacyl C₂₋₁₈ alkyl, C₄-C₁₈ alkylcarbocyclyloxy, C₃-C₁₈ alkylheterocyclyloxy, C₄-C₁₈ alkylheteroaryloxy, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₄-C₁₈ alkylheterocyclylalkyl, C₄-C₁₈ alkenylheterocyclylalkyl, C₄-C₁₈ alkynylheterocyclylalkyl, C₅—C₁₈ alkenylheterocyclylalkenyl, C₅-C₁₈ alkenylheterocyclylalkynyl, C₅-C₁₈ alkynylheterocyclylalkynyl, C₃-C₁₈ alkylheterocyclylthio, C₄-C₁₈ alkylheteroarylthio, C₄-C₁₈ alkylalkenylalkyl, C₄-C₁₈ alkylalkynylalkyl, C₈-C₂₄ alkylarylalkyl, C₃-C₁₈ alkylacylalkyl, C₁₃-C₂₄ arylalkylaryl, C₁₄-C₂₄ arylalkenylaryl, C₁₄-C₂₄ arylalkynylaryl, C₁₃-C₂₄ arylacylaryl, C₇-C₁₈ arylacyl, C₉-C₁₈ arylcarbocyclyl, C₈-C₁₈ arylheterocyclyl, C₉-C₁₈ arylheteroaryl, C₈-C₁₈ alkenyloxyaryl, C₈-C₁₈ alkynyloxyaryl, C₁₂-C₂₄ aryloxyaryl, C₇-C₁₈ arylacyloxy, C₉-C₁₈ arylcarbocyclyloxy, C₈-C₁₈ arylheterocyclyloxy, C₉-C₁₈ arylheteroaryloxy, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylthio, C₂-C₁₈ alkenylthio, C₂-C₁₈ alkynylthio, C₅-C₁₈ arylthio, C₁-C₁₈ acylthio, C₃-C₁₈ carbocyclylthio, C₂-C₁₈ heterocyclylthio, C₃-C₁₉ heteroarylthio, C₂-C₁₉ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₃-C₁₈ alkylheterocyclylthio, C₄-C₁₈ alkylheteroarylthio, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylamino, C₂-C₁₈ alkenylamino, C₂-C₁₈ alkynylamino, C₆-C₁₈ arylamino, C₁-C₁₈ acylamino, C₃-C₁₈ carbocyclylamino, C₂-C₁₈ heterocyclylamino, C₃-C₁₈ heteroarylamino, C₂-C₁₈ alkylaminoalkyl, C₃-C₁₉ alkenylaminoalkyl, C₃-C₁₈ alkynylaminoalkyl, C₄-C₁₈ alkenylaminoalkenyl, C₄-C₁₈ alkenylaminoalkynyl, C₄-C₁₈ alkynylaminoalkynyl, C₇-C₂₄ arylaminoalkyl, C₂-C₁₈ alkylacylamino, C₄-C₁₈ alkylcarbocyclylamino, C₃-C₁₈ alkylheterocyclylamino, C₄-C₁₈ alkylheteroarylamino, C₈-C₁₈ alkylaminoaryl, C₈-C₁₈ alkenylaminoaryl, C₈-C₁₈ alkynylaminoaryl, C₁₂-C₂₄ arylaminoaryl, C₇-C₁₈ arylacylamino, C₈-C₁₈ arylcarbocyclylamino, C₈-C₁₈ arylheterocyclylamino, C₉-C₁₈ arylheteroarylamino, C₁-C₁₈ alkylamido, C₂-C₁₈ alkenylamido, C₂-C₁₈ alkynylamido, C₆-C₁₈ arylamido, C₁-C₁₈ acylamido, C₃-C₁₉ carbocyclylamido, C₂-C₁₉ heterocyclylamido, C₃-C₁₈ heteroarylamido, C₂-C₁₈ alkylamidoalkyl, C₃-C₁₈ alkenylamidoalkyl, C₃-C₁₈ alkynylamidoalkyl, C₄-C₁₈ alkenylamidoalkenyl, C₄-C₁₈ alkenylamidoalkynyl, C₄-C₁₈ alkynylamidoalkynyl, C₇-C₂₄ arylamidoalkyl, C₂-C₁₈ alkylacylamido, C₄-C₁₈ alkylcarbocyclylamido, C₃-C₁₈ alkylheterocyclylamido, C₄-C₁₈ alkylheteroarylamido, C₈-C₁₈ alkylamidoaryl, C₈-C₁₈ alkenylamidoaryl, C₈-C₁₈ alkynylamidoaryl, C₁₂-C₂₄ arylamidoaryl, C₇-C₁₈ arylacylamido, C₉-C₁₈ arylcarbocyclylamido, C₈-C₁₈ arylheterocyclylamido, C₉-C₁₈ arylheteroarylamido, C₁-C₁₈ alkylcarbamyl, C₂-C₁₉ alkenylcarbamyl, C₂-C₁₈ alkynylcarbamyl, C₈-C₁₈ arylcarbamyl, C₁-C₁₈ acylcarbamyl, C₃-C₁₈ carbocyclylcarbamyl, C₂-C₁₈ heterocyclylcarbamyl, C₃-C₁₈ heteroarylcarbamyl, C₂-C₁₈ alkylcarbamylalkyl, C₃-C₁₈ alkenylcarbamylalkyl, C₃-C₁₈ alkynylcarbamylalkyl, C₄-C₁₈ alkenylcarbamylalkenyl, C₄-C₁₈ alkenylcarbamylalkynyl, C₄-C₁₈ alkynylcarbamylalkynyl, C₇-C₂₄ arylcarbamylalkyl, C₂-C₁₈ alkylacylcarbamyl, C₄-C₁₈ alkylcarbocyclylcarbamyl, C₃-C₁₈ alkylheterocyclylcarbamyl, C₄-C₁₈ alkylheteroarylcarbamyl, C₈-C₁₈ alkylcarbamylaryl, C₈-C₁₈ alkenylcarbamylaryl, C₈-C₁₈ alkynylcarbamylaryl, C₁₂-C₂₄ arylcarbamylaryl, C₇-C₁₉ arylacylcarbamyl, C₉-C₁₉ arylcarbocyclylcarbamyl, C₈-C₁₈ arylheterocyclylcarbamyl, C₉-C₁₈ arylheteroarylcarbamyl, C₁-C₁₈ alkyldisulfidyl, C₂-C₁₈ alkenyldisulfidyl, C₂-C₁₈ alkynyldisulfidyl, C₆-C₁₈ aryldisulfidyl, C₁-C₁₈ acyldisulfidyl, C₃-C₁₈ carbocyclyldisulfidyl, C₂-C₁₈ heterocyclyldisulfidyl, C₃-C₁₈ heteroaryldisulfidyl, C₂-C₁₈ alkyldisulfidylalkyl, C₃-C₁₈ alkenyldisulfidylalkyl, C₃-C₁₈ alkynyldisulfidylalkyl, C₄-C₁₈ alkenyldisulfidylalkenyl, C₄-C₁₈ alkenyldisulfidylalkynyl, C₄-C₁₈ alkynyldisulfidylalkynyl, C₇—C₂₄ aryldisulfidylalkyl, C₂-C₁₈ alkylacyldisulfidyl, C₄-C₁₈ alkylcarbocyclyldisulfidyl, C₃-C₁₈ alkylheterocyclyldisulfidyl, C₄-C₁₈ alkylheteroaryldisulfidyl, C₈-C₁₈ alkyldisulfidylaryl, C₈-C₁₈ alkenyldisulfidylaryl, C₈-C₁₈ alkynyldisulfidylaryl, C₁₂-C₂₄ aryldisulfidylaryl, C₇-C₁₈ arylacyldisulfidyl, C₉-C₁₈ arylcarbocyclyldisulfidyl, C₈-C₁₈ arylheterocyclyldisulfidyl, and C₉-C₁₈ arylheteroaryldisulfidyl, and wherein R² is optionally substituted;
and wherein -A-X—R² is not —CH₂OH.

24. The method of any one of claims 14 to 23, wherein R² is selected from alkyl (e.g., C₁-C₁₈, C₁-C₆, C₁-C₅, C₈-C₁₈, or C₉-C₁₈), aryl (e.g., C₆-C₁₈), heteroaryl (e.g., C₃-C₁₈), carbocyclyl (e.g., C₃-C₁₈), heterocyclyl (e.g., C₂-C₁₈), alkylaryl (e.g., C₇-C₂₄), alkylheteroaryl (e.g., C₄-C_{1S}), alkylcarbocyclyl (e.g., C₄-C₁₉), alkylthioalkyl (e.g., wherein each alkyl is C₁-C₁₈), arylthioalkyl (e.g., wherein each alkyl is C₇-C₁₈), alkylaminoalkyl (e.g., wherein each alkyl is C₁-C₁₈), alkylamidoalkyl (e.g., wherein each alkyl is C₁-C₁₈), alkylheterocyclylalkyl (e.g., C₃-C₁₈), alkylthio (e.g., C₁-C₁₈; such as thiomethyl) and alkylheterocyclyl (e.g., C₃-C₁₈).

25. The method of any one of claims 1 to 18, wherein the iNKT cell agonist is a Compound of Formula (III): 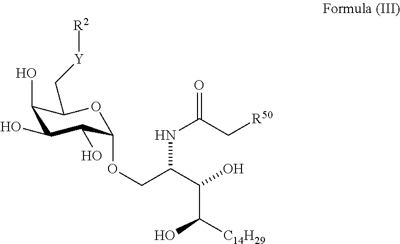
or a pharmaceutically acceptable salt thereof, wherein:
Y is selected from —S—, —NR^{a}—, —NR^{a}C(O)—, —NR^{a}C(O)O—, —S—S—, heterocyclyl, heteroaryl, wherein R^{a} may be selected from hydrogen, alkyl, alkenyl, alkynyl, aryl, carbocyclyl, heteroaryl, heterocyclyl, arylalkyl, and acyl;
R² is selected from hydrogen, halo, alkyl, alkenyl, alkynyl, aryl, acyl, carbocyclyl, heterocyclyl, heteroaryl, alkyloxy, alkenyloxy, alkynyloxy, aryloxy, acyloxy, carbocyclyloxy, heterocyclyloxy, heteroaryloxy, alkylthio, alkenylthio, alkynylthio, arylthio, acylthio, carbocyclylthio, heterocyclylthio, heteroarylthio, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkyloxyacylalkyl, alkylcarbocyclyloxy, alkylheterocyclyloxy, alkylheteroaryloxy, alkylthioalkyl, alkenylthioalkyl, alkynylthioalkyl, alkenylthioalkenyl, alkenylthioalkynyl, alkynylthioalkynyl, arylthioalkyl, alkylacylthio, alkylcarbocyclylthio, alkylheterocyclylalkyl, alkenylheterocyclylalkyl, alkynylheterocyclylalkyl, alkenylheterocyclylalkenyl, alkenylheterocyclylalkynyl, alkynylheterocyclylalkynyl, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, arylacyloxy, arylcarbocyclyloxy, arylheterocyclyloxy, arylheteroaryloxy, alkylthioaryl, alkenylthioaryl, alkynylthioaryl, arylthioaryl, arylacylthio, arylcarbocyclylthio, arylheterocyclylthio, arylheteroarylthio, alkylamino, alkenylamino, alkynylamino, arylamino, acylamino, carbocyclylamino, heterocyclylamino, heteroarylamino, alkylaminoalkyl, alkenylaminoalkyl, alkynylaminoalkyl, alkenylaminoalkenyl, alkenylaminoalkynyl, alkynylaminoalkynyl, arylaminoalkyl, alkylacylamino, alkylcarbocyclylamino, alkylheterocyclylamino, alkylheteroarylamino, alkylaminoaryl, alkenylaminoaryl, alkynylaminoaryl, arylaminoaryl, arylacylamino, arylcarbocyclylamino, arylheterocyclylamino, arylheteroarylamino, alkylamido, alkenylamido, alkynylamido, arylamido, acylamido, carbocyclylamido, heterocyclylamido, heteroarylamido, alkylamidoalkyl, alkenylamidoalkyl, alkynylamidoalkyl, alkenylamidoalkenyl, alkenylamidoalkynyl, alkynylamidoalkynyl, arylamidoalkyl, alkylacylamido, alkylcarbocyclylamido, alkylheterocyclylamido, alkylheteroarylamido, alkylamidoaryl, alkenylamidoaryl, alkynylamidoaryl, arylamidoaryl, arylacylamido, arylcarbocyclylamido, arylheterocyclylamido, arylheteroarylamido, alkylcarbamyl, alkenylcarbamyl, alkynylcarbamyl, arylcarbamyl, acylcarbamyl, carbocyclylcarbamyl, heterocyclylcarbamyl, heteroarylcarbamyl, alkylcarbamylalkyl, alkenylcarbamylalkyl, alkynylcarbamylalkyl, alkenylcarbamylalkenyl, alkenylcarbamylalkynyl, alkynylcarbamylalkynyl, arylcarbamylalkyl, alkylacylcarbamyl, alkylcarbocyclylcarbamyl, alkylheterocyclylcarbamyl, alkylheteroarylcarbamyl, alkylcarbamylaryl, alkenylcarbamylaryl, alkynylcarbamylaryl, arylcarbamylaryl, arylacylcarbamyl, arylcarbocyclylcarbamyl, arylheterocyclylcarbamyl, arylheteroarylcarbamyl, alkyldisulfidyl, alkenyldisulfidyl, alkynyldisulfidyl, aryldisulfidyl, acyldisulfidyl, carbocyclyldisulfidyl, heterocyclyldisulfidyl, heteroaryldisulfidyl, alkyldisulfidylalkyl, alkenyldisulfidylalkyl, alkynyldisulfidylalkyl, alkenyldisulfidylalkenyl, alkenyldisulfidylalkynyl, alkynyldisulfidylalkynyl, aryldisulfidylalkyl, alkylacyldisulfidyl, alkylcarbocyclyldisulfidyl, alkylheterocyclyldisulfidyl, alkylheteroaryldisulfidyl, alkyldisulfidylaryl, alkenyldisulfidylaryl, alkynyldisulfidylaryl, aryldisulfidylaryl, arylacyldisulfidyl, arylcarbocyclyldisulfidyl, arylheterocyclyldisulfidyl, and arylheteroaryldisulfidyl, and wherein R² is optionally substituted;
R⁵⁰ is selected from optionally substituted alkyl, alkenyl, alkynyl.

26. The composition of claim 25, wherein R^{a} is hydrogen.

27. The composition of claim 25 or claim 26, wherein Y is selected from —S—, —NH—, —NHC(O)—, —NHC(O)O—, and —S—S—, triazolyl.

28. The composition of any one of claims 25 to 27, wherein A is —CR¹²R¹³—, wherein R¹² and R¹³ where present are independently selected from H, halo, C₁₋₂alkyl. More preferably A is —CH₂—.

29. The composition of any one of claims 25 to 28, wherein R² is selected from C₁-C₁₈ alkyl, C₂-C₁₈ alkenyl, C₂-C₁₈ alkynyl, C₆-C₁₈ aryl, C₁-C₁₈ acyl, C₃-C₁₈ carbocyclyl, C₂-C₁₈ heterocyclyl, C₃-C₁₈ heteroaryl, C₁-C₁₈ alkyloxy, C₂-C₁₈ alkenyloxy, C₂-C₁₈ alkynyloxy, C₆-C₁₈ aryloxy, C₁-C₁₈ acyloxy, C₃-C₁₈ carbocyclyloxy, C₂-C₁₈ heterocyclyloxy, C₃-C₁₈ heteroaryloxy, C₁-C₁₈ alkylthio, C₂-C₁₈ alkenylthio, C₂-C₁₈ alkynylthio, C₅-C₁₈ arylthio, C₁-C₁₈ acylthio, C₃-C₁₈ carbocyclylthio, C₂-C₁₈ heterocyclylthio, C₃-C₁₈ heteroarylthio, C₃-C₁₈ alkylalkenyl, C₃-C₁₈ alkylalkynyl C₇-C₂₄ alkylaryl, C₂-C₁₈ alkylacyl, C₄-C₁₈ alkylcarbocyclyl, C₃-C₁₈ alkylheterocyclyl, C₄-C₁₈ alkylheteroaryl, C₂—C₁₈ alkyloxyalkyl, C₃-C₁₈ alkenyloxyalkyl, C₃-C₁₈ alkynyloxyalkyl, C₇-C₂₄ aryloxyalkyl, C₂-C₁₈ alkylacyloxy, C₂₋₁₈ alkyloxyacyl C₂₋₁₈ alkyl, C₄-C₁₈ alkylcarbocyclyloxy, C₃-C₁₈ alkylheterocyclyloxy, C₄-C₁₈ alkylheteroaryloxy, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₄-C₁₈ alkylheterocyclylalkyl, C₄-C₁₈ alkenylheterocyclylalkyl, C₄-C₁₈ alkynylheterocyclylalkyl, C₅-C₁₈ alkenylheterocyclylalkenyl, C₅-C₁₈ alkenylheterocyclylalkynyl, C₅-C₁₈ alkynylheterocyclylalkynyl, C₃-C₁₈ alkylheterocyclylthio, C₄-C₁₈ alkylheteroarylthio, C₄-C₁₈ alkylalkenylalkyl, C₄-C₁₈ alkylalkynylalkyl, C₈-C₂₄ alkylarylalkyl, C₃-C₁₈ alkylacylalkyl, C₁₃-C₂₄ arylalkylaryl, C₁₄-C₂₄ arylalkenylaryl, C₁₄-C₂₄ arylalkynylaryl, C₁₃-C₂₄ arylacylaryl, C₇-C₁₈ arylacyl, C₉-C₁₈ arylcarbocyclyl, C₈-C₁₈ arylheterocyclyl, C₉-C₁₈ arylheteroaryl, C₈-C₁₈ alkenyloxyaryl, C₈-C₁₈ alkynyloxyaryl, C₁₂-C₂₄ aryloxyaryl, C₇-C₁₈ arylacyloxy, C₉-C₁₈ arylcarbocyclyloxy, C₈-C₁₈ arylheterocyclyloxy, C₉-C₁₈ arylheteroaryloxy, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylthio, C₂-C₁₈ alkenylthio, C₂-C₁₈ alkynylthio, C₆-C₁₈ arylthio, C₁-C₁₈ acylthio, C₃-C₁₈ carbocyclylthio, C₂-C₁₈ heterocyclylthio, C₃-C₁₈ heteroarylthio, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₃-C₁₈ alkylheterocyclylthio, C₄-C₁₈ alkylheteroarylthio, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₈-C₁₈ arylcarbocyclylthio, C₈-C₁₈ arylheterocyclylthio, C₉-C₁₈ arylheteroarylthio, C₁-C₁₈ alkylamino, C₂-C₁₈ alkenylamino, C₂-C₁₈ alkynylamino, C₆-C₁₈ arylamino, C₁-C₁₈ acylamino, C₃-C₁₈ carbocyclylamino, C₂-C₁₈ heterocyclylamino, C₃-C₁₈ heteroarylamino, C₂-C₁₈ alkylaminoalkyl, C₃-C₁₈ alkenylaminoalkyl, C₃-C₁₈ alkynylaminoalkyl, C₄-C₁₈ alkenylaminoalkenyl, C₄-C₁₈ alkenylaminoalkynyl, C₄-C₁₈ alkynylaminoalkynyl, C₇-C₂₄ arylaminoalkyl, C₂-C₁₈ alkylacylamino, C₄-C₁₈ alkylcarbocyclylamino, C₃-C₁₈ alkylheterocyclylamino, C₄-C₁₈ alkylheteroarylamino, C₈-C₁₈ alkylaminoaryl, C₈-C₁₈ alkenylaminoaryl, C₈-C₁₈ alkynylaminoaryl, C₁₂-C₂₄ arylaminoaryl, C₇-C₁₈ arylacylamino, C₉-C₁₈ arylcarbocyclylamino, C₈-C₁₈ arylheterocyclylamino, C₉-C₁₈ arylheteroarylamino, C₁-C₁₈ alkylamido, C₂-C₁₈ alkenylamido, C₂-C₁₈ alkynylamido, C₆-C₁₈ arylamido, C₁-C₁₈ acylamido, C₃-C₁₉ carbocyclylamido, C₂-C₁₈ heterocyclylamido, C₃-C₁₈ heteroarylamido, C₂-C₁₈ alkylamidoalkyl, C₃-C₁₈ alkenylamidoalkyl, C₃-C₁₈ alkynylamidoalkyl, C₄-C₁₈ alkenylamidoalkenyl, C₄-C₁₈ alkenylamidoalkynyl, C₄-C₁₈ alkynylamidoalkynyl, C₇-C₂₄ arylamidoalkyl, C₂-C₁₈ alkylacylamido, C₄-C₁₉ alkylcarbocyclylamido, C₃-C₁₈ alkylheterocyclylamido, C₄-C₁₈ alkylheteroarylamido, C₈-C₁₈ alkylamidoaryl, C₈-C₁₈ alkenylamidoaryl, C₈-C₁₈ alkynylamidoaryl, C₁₂-C₂₄ arylamidoaryl, C₇-C₁₈ arylacylamido, C₉-C₁₈ arylcarbocyclylamido, C₈-C₁₈ arylheterocyclylamido, C₉-C₁₈ arylheteroarylamido, C₁-C₁₈ alkylcarbamyl, C₂-C₁₈ alkenylcarbamyl, C₂-C₁₈ alkynylcarbamyl, C₅-C₁₈ arylcarbamyl, C₁-C₁₈ acylcarbamyl, C₃-C₁₈ carbocyclylcarbamyl, C₂-C₁₈ heterocyclylcarbamyl, C₃-C₁₈ heteroarylcarbamyl, C₂-C₁₈ alkylcarbamylalkyl, C₃-C₁₈ alkenylcarbamylalkyl, C₃-C₁₈ alkynylcarbamylalkyl, C₄-C₁₈ alkenylcarbamylalkenyl, C₄-C₁₈ alkenylcarbamylalkynyl, C₄-C₁₈ alkynylcarbamylalkynyl, C₇-C₂₄ arylcarbamylalkyl, C₂-C₁₈ alkylacylcarbamyl, C₄-C₁₈ alkylcarbocyclylcarbamyl, C₃-C₁₉ alkylheterocyclylcarbamyl, C₄-C₁₈ alkylheteroarylcarbamyl, C₈-C₁₈ alkylcarbamylaryl, C₈-C₁₈ alkenylcarbamylaryl, C₈-C₁₈ alkynylcarbamylaryl, C₁₂—C₂₄ arylcarbamylaryl, C₇-C₁₈ arylacylcarbamyl, C₉-C₁₈ arylcarbocyclylcarbamyl, C₈-C₁₈ arylheterocyclylcarbamyl, C₉-C₁₈ arylheteroarylcarbamyl, C₁-C₁₈ alkyldisulfidyl, C₂-C₁₈ alkenyldisulfidyl, C₂-C₁₈ alkynyldisulfidyl, C₆-C₁₈ aryldisulfidyl, C₁-C₁₈ acyldisulfidyl, C₃-C₁₈ carbocyclyldisulfidyl, C₂-C₁₉ heterocyclyldisulfidyl, C₃-C₁₈ heteroaryldisulfidyl, C₂-C₁₈ alkyldisulfidylalkyl, C₃-C₁₈ alkenyldisulfidylalkyl, C₃-C₁₈ alkynyldisulfidylalkyl, C₄-C₁₈ alkenyldisulfidylalkenyl, C₄-C₁₈ alkenyldisulfidylalkynyl, C₄-C₁₈ alkynyldisulfidylalkynyl, C₇-C₂₄ aryldisulfidylalkyl, C₂-C₁₈ alkylacyldisulfidyl, C₄-C₁₈ alkylcarbocyclyldisulfidyl, C₃-C₁₈ alkylheterocyclyldisulfidyl, C₄-C₁₈ alkylheteroaryldisulfidyl, C₈-C₁₈ alkyldisulfidylaryl, C₈-C₁₈ alkenyldisulfidylaryl, C₈-C₁₈ alkynyldisulfidylaryl, C₁₂-C₂₄ aryldisulfidylaryl, C₇-C₁₈ arylacyldisulfidyl, C₉-C₁₈ arylcarbocyclyldisulfidyl, C₈-C₁₈ arylheterocyclyldisulfidyl, and C₉-C₁₈ arylheteroaryldisulfidyl, and wherein R² is optionally substituted.

30. The composition of ay one of any one of claims 27 to 29, wherein R² in Formula (III) is selected from alkyl (e.g., C₁-C₁₉, C₁-C₆, C₁-C₅, C₈-C₁₈, or C₉-C₁₈), aryl (e.g., C₈-C₁), heteroaryl (e.g., C₃-C₁₈), carbocyclyl (e.g., C₃-C₁₈), heterocyclyl (e.g., C₂-C₁₈), alkylaryl (e.g., C₇-C₂₄), alkylheteroaryl (e.g., C₄-C₁₉), alkylcarbocyclyl (e.g., C₄-C₁₉), alkylthioalkyl (e.g., wherein each alkyl is C₁-C₁₈), arylthioalkyl (e.g., wherein each alkyl is C₇-C₁₈), alkylaminoalkyl (e.g., wherein each alkyl is C₁-C₁₈), alkylamidoalkyl (e.g., wherein each alkyl is C₁-C₁₈), alkylheterocyclylalkyl (e.g., C₃-C₁₈), alkylthio (e.g., C₁-C₁₈; such as thiomethyl) and alkylheterocyclyl (e.g., C₃-C₁₈).

31. The composition of any one of claims 1 to 14, wherein the iNKT cell agonists is a compound selected from the following GROUP I compounds:

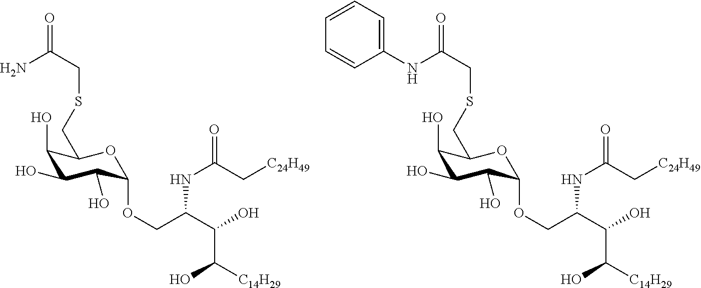

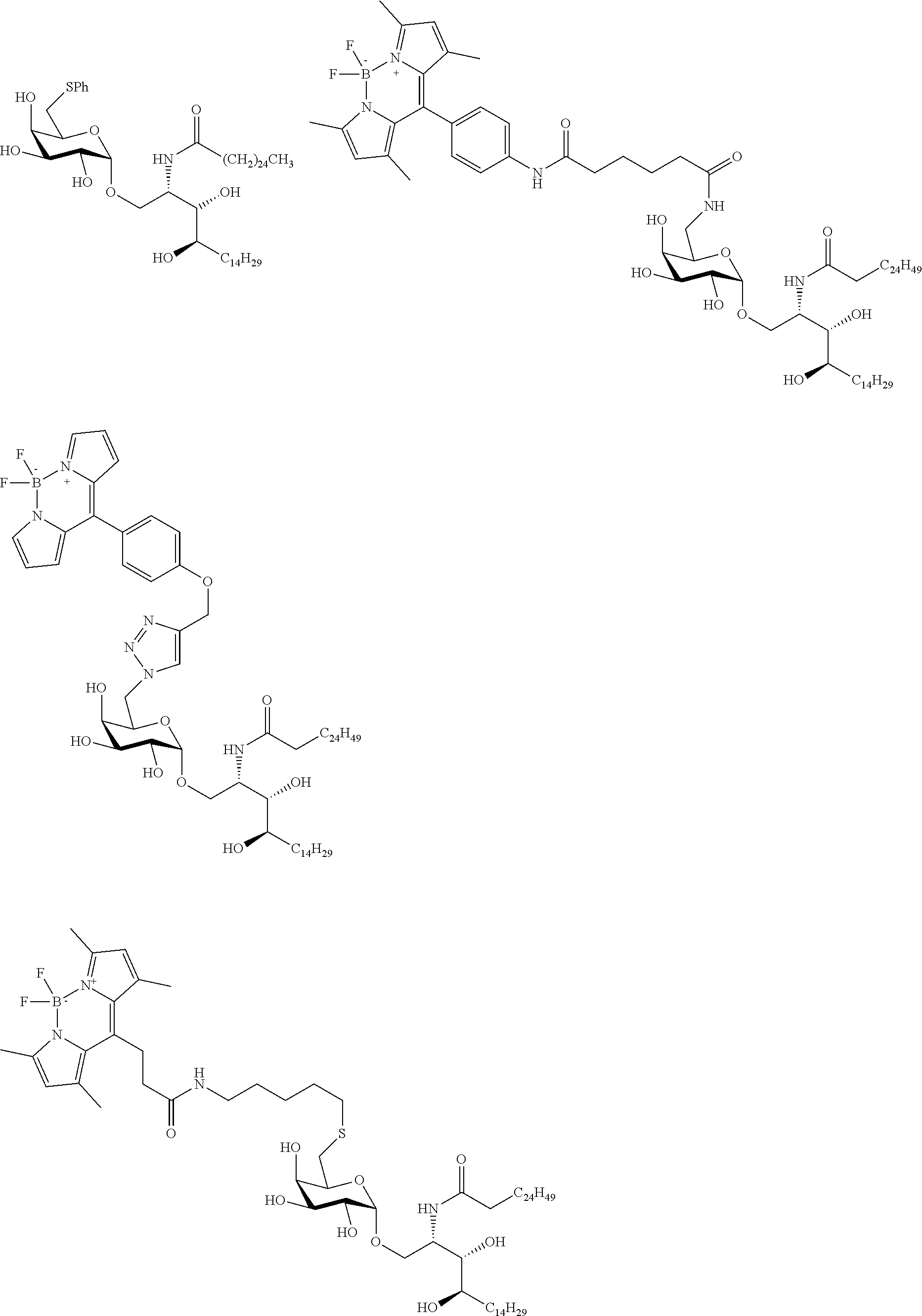

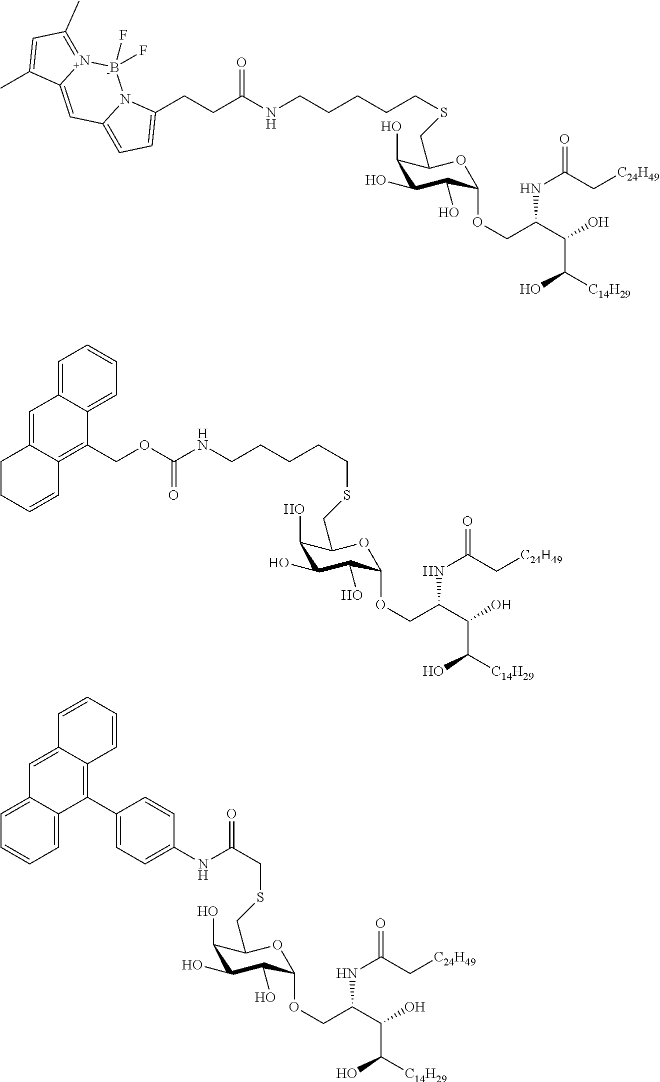

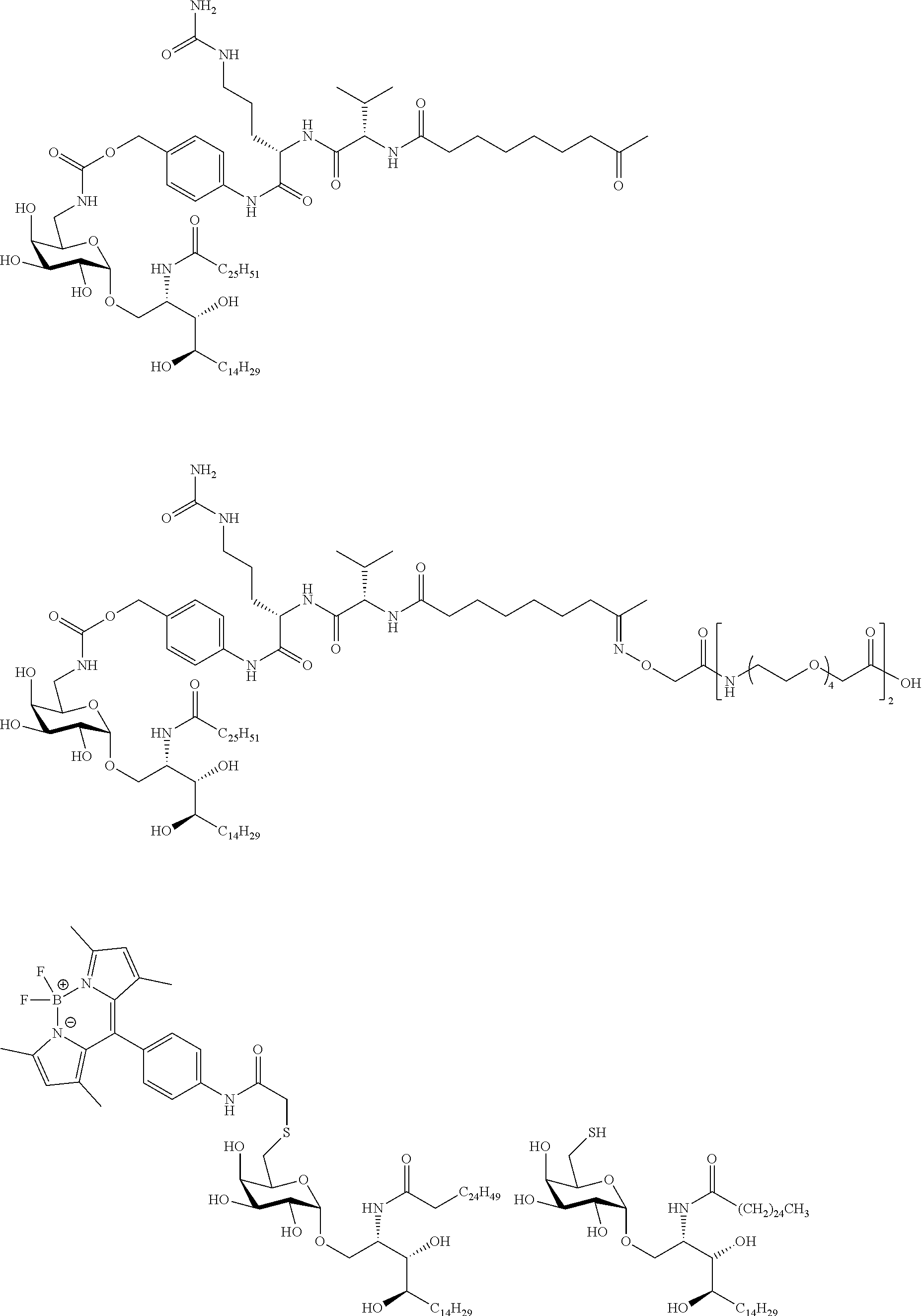

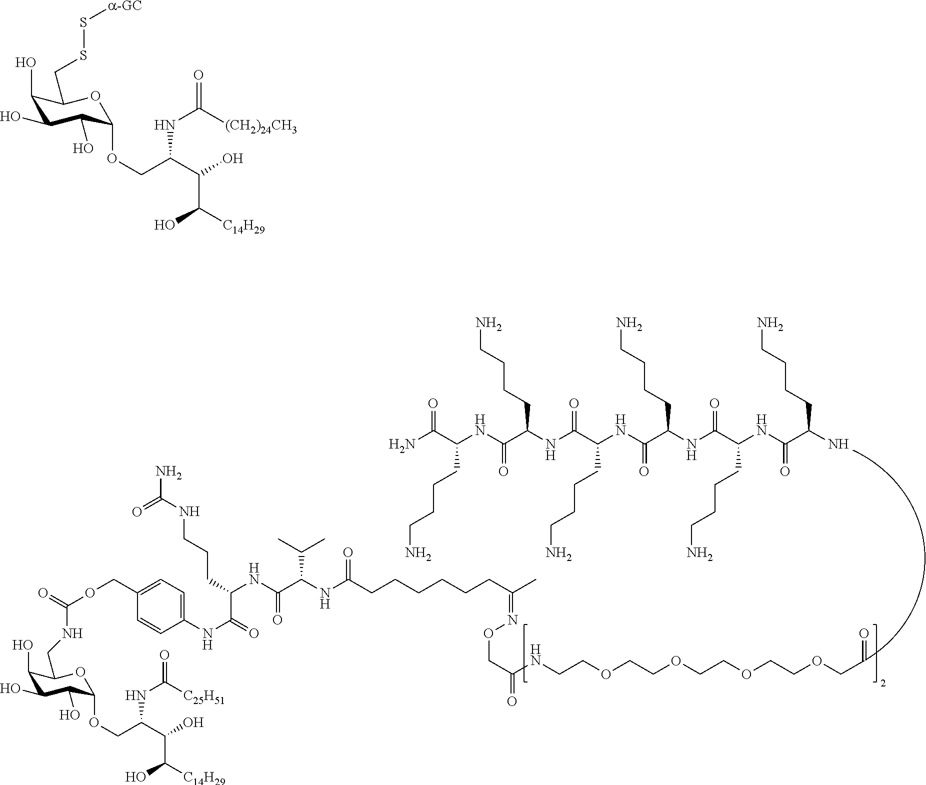

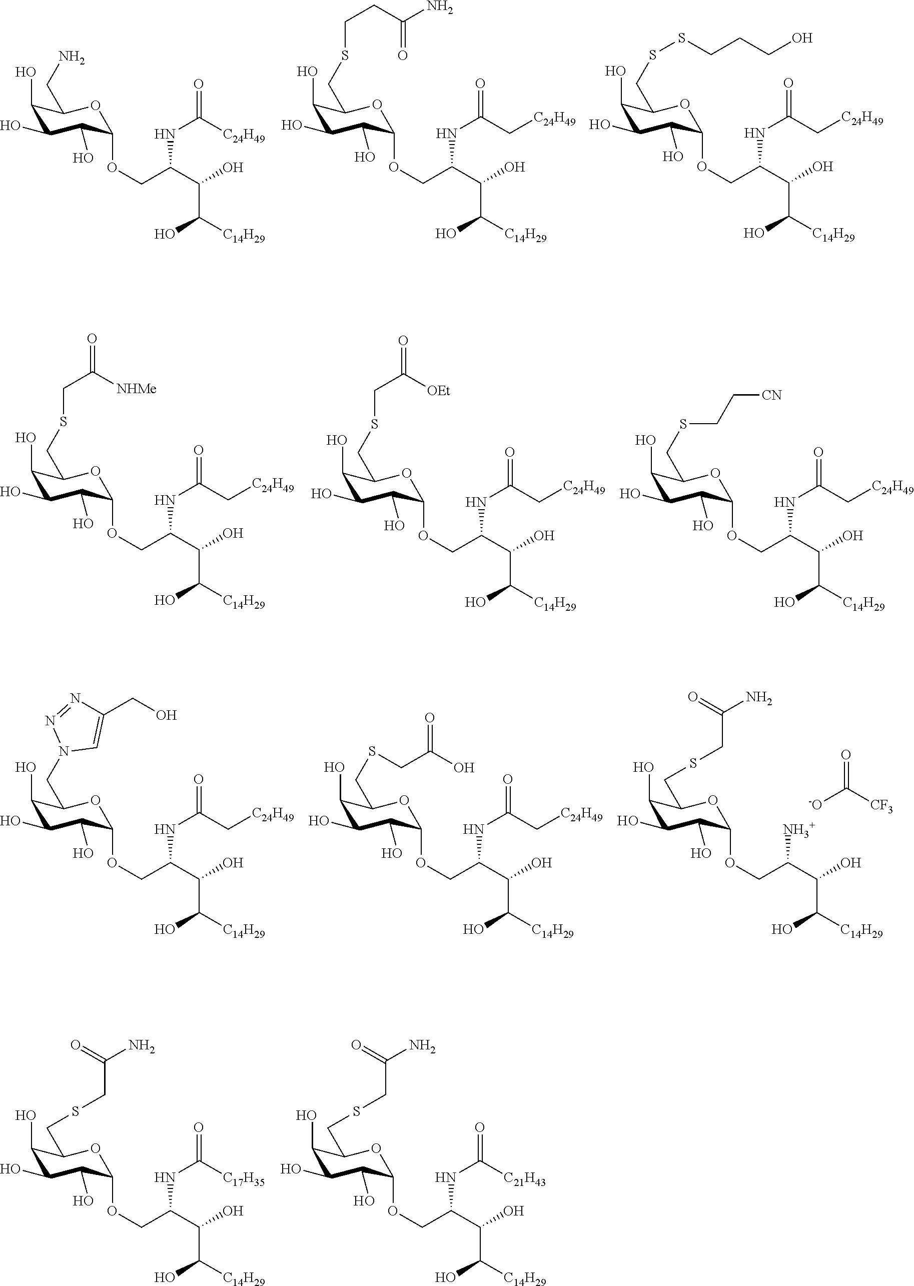

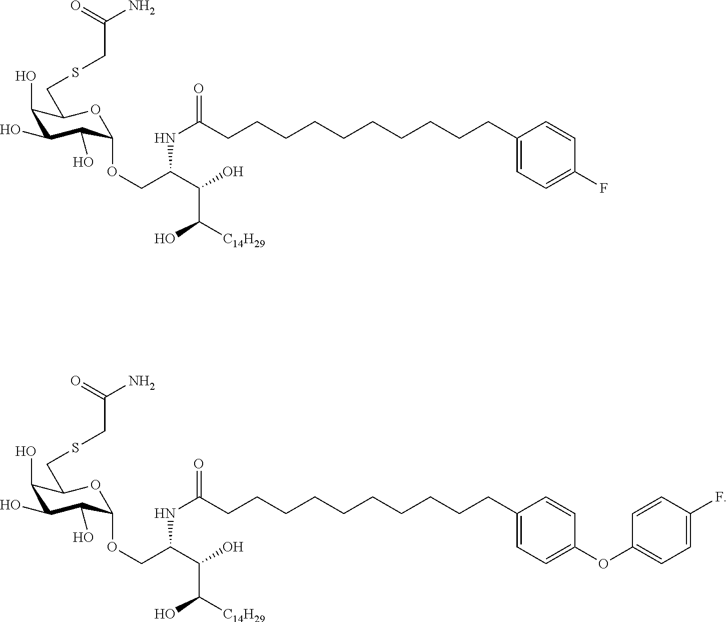

32. The composition of any one of claims 1 to 31, wherein the immune stimulator is a CD8⁺ T cell epitope.

33. The composition of any one of claims 1 to 321, wherein the target antigen is selected from a virus antigen, a bacterial antigen, a parasite antigen, and a cancer antigen.

34. The composition of claim 33, wherein the parasite antigen is derived from a parasite selected from Plasmodium falciparum, P. vivax, P. malariae, P. ovale, and P. knowlesi.

35. The composition of claim 33, wherein the virus antigen is a hepatitis (e.g., hepatitis A, hepatitis B and hepatitis C) antigen.

36. The composition of claim 33, wherein the cancer antigen is a liver cancer antigen.

37. The composition of any one of claims 1 to 36, wherein the second agent is a polynucleotide sequence, and wherein the polynucleotide is a ribonucleic acid (RNA) molecule.

38. The composition of any one of claims 1 to 37, wherein the polynucleotide is codon-optimised, and wherein the codon optimisation is selected to enhance translation in the subject and/or to reduce innate immunity against the polynucleotide molecule.

39. The composition of any one of claims 1 to 38, wherein the polynucleotide comprises at least one chemical modification.

40. The composition of claim 39, wherein the at least one chemical modification is selected from the group consisting of pseudouridine, N1-methylpseudouridine, N1-ethylpseudouridine, 2thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2thio-dihydrouridine, 2-thio-pseudouridine, 4-methoxy-2-thio-pseudouridine, 4methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

41. The composition of claim 39 or claim 40, wherein the polynucleotide is fully modified.

42. The composition of any one of claims 1 to 41, wherein the composition further comprises a nanoparticle and the second agent is encapsulated within the nanoparticle or complexed to the surface of the nanoparticle.

43. The composition of claim 42, wherein the nanoparticle is a lipid nanoparticle (e.g., a liposome, or a lipoplex).

44. The composition of claim 42 or claim 43, wherein the first agent is contained at least partially within the surface of the lipid nanoparticle.

45. The composition of any one of claims 42 to 44, wherein the lipid nanoparticle has a diameter of between about 50 nm and 500 nm.

46. The composition of any one of claims 42 to 45, wherein the nanoparticle comprises a cationic lipid, a PEG-modified lipid, a sterol, and/or a non-cationic lipid.

47. The composition of claim 46, wherein the cationic lipid is an ionizable cationic lipid.

48. The composition of claim 46 or claim 47, wherein the ionizable cationic lipid is selected from the group comprising or consisting of 1,2-di-O-octadecenyl-3-trimethylammonium propane (DOTMA), N-[1-(2,3-dioleyloxy)propyl]-N,N,N-trimethylammonium chloride (DOTMA), 1,2-Dioleoyl-3-Trimethylammonium-Propane (DOTAP), 5-carboxyspermylglycinedioctadecylamide (DOGS), 2,3-dioleyloxy-N-[2(spermine-carboxamido)ethyl]-N,N-dimethyl-I-propanaminium (DOSPA), 1,2-Dioleoyl-3-Dimethylammonium-Propane (DODAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), heptatriaconta-6,9,28,31-tetraen19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dilinolenyloxy-N,N-dimethyl-3-aminopropane (DLenDMA), N-dioleyl-N,N-dimethyl ammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis, cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminoethyl-[1,3]-dioxolane (DLin-K-XTC2-DMA), and C12-200.

49. The composition of any one of claims 42 to 48, wherein the nanoparticle comprises a dioleoylphosphatidylethanolamine (DOPE) lipid.

50. A pharmaceutical composition comprising the composition of any one of claims 1 to 49, and a pharmaceutically acceptable carrier, diluent, or excipient.

51. A vaccine that comprises a composition of any one of claims 1 to 50 and a pharmaceutically acceptable carrier, diluent, or excipient.

52. A composition for inducing an immune response to a target antigen in a subject, the composition comprising at least one isolated RNA molecule encoding an immune stimulator that stimulates or otherwise enhances an immune response to the target antigen in a subject, and a lipid nanoparticle, wherein the lipid nanoparticle comprises an iNKT agonist, wherein the iNKT agonist induces proliferation or accumulation of tissue-resident memory T (T_{RM}) cells in the subject.

53. The composition of claim 52, wherein the first agent modifies a phenotype of an iNKT cell.

54. The composition of claim 53, wherein the modified phenotype comprises a downregulation of TCR expression.

55. The composition of claim 53 or claim 54, wherein the modified phenotype comprises an enhanced expression of PD1 on the surface of the iNKT cell.

56. The composition of any one of claims 52 to 55, wherein the iNKT cell is in the liver of the subject.

57. The composition of any one of claims 52 to 56, wherein the iNKT cell agonist induces T_{RM} cell accumulation in the liver of the subject.

58. The composition of any one of claims 52 to 57, wherein upon administration to a subject the ratio of T_{RM} cells to central memory T (T_{CM}) cells and/or effector memory (T_{EM}) cells present in the liver is increased.

59. The composition of any claim 58, wherein the ratio of T_{RM} cells to T_{CM} and T_{EM} cells (i.e., T_{RM} cells:T_{CM}+T_{EM} cells) is greater than about 1:1.

60. The composition of claim 58 or claim 59, wherein the ratio of T_{RM} cells to T_{CM} and T_{EM} cells (i.e., T_{RM} cells:T_{CM}+T_{EM} cells) is greater than about 3:2.

61. The composition of any one of claims 58 to 60, wherein the ratio of T_{RM} cells to T_{CM} and T_{EM} cells (i.e., T_{RM} cells:T_{CM}+T_{EM} cells) is greater than about 2.3:1

62. The composition of any one of claims 57 to 61, wherein at least 30%, 35%, 40%, 45%, 50%, 55%, 60%, 65%, 66%, 70%, 75% of the memory T cell population in the liver are T_{RM} cells.

63. The composition of claim 62, wherein greater than about 70% of the memory T cell population in the liver are T_{RM} cells.

64. The composition of any one of claims 52 to 63, wherein the antigen is selected from the group comprising a virus antigen, a bacterial antigen, a parasite antigen, and a cancer or tumour antigen.

65. The composition of any one of claims 1 to 12, wherein the iNKT cell agonist is a derivative of α-galactosylceramide, wherein the derivation is at the 6 position of the galactose ring.

66. The composition of any one of claims 52 to 65, wherein the iNKT cell agonist comprises a Compound of Formula (I), or a pharmaceutically acceptable salt thereof, wherein:
R¹ is selected from hydrogen, halo, alkyl, alkenyl, alkynyl, aryl, acyl, carbocyclyl, heterocyclyl, heteroaryl, alkyloxy, alkenyloxy, alkynyloxy, aryloxy, acyloxy, carbocyclyloxy, heterocyclyloxy, heteroaryloxy, alkylthio, alkenylthio, alkynylthio, arylthio, acylthio, carbocyclylthio, heterocyclylthio, heteroarylthio, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkyloxyacylalkyl, alkylcarbocyclyloxy, alkylheterocyclyloxy, alkylheteroaryloxy, alkylthioalkyl, alkenylthioalkyl, alkynylthioalkyl, alkenylthioalkenyl, alkenylthioalkynyl, alkynylthioalkynyl, arylthioalkyl, alkylacylthio, alkylcarbocyclylthio, alkylheterocyclylalkyl, alkenylheterocyclylalkyl, alkynylheterocyclylalkyl, alkenylheterocyclylalkenyl, alkenylheterocyclylalkynyl, alkynylheterocyclylalkynyl, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, arylacyloxy, arylcarbocyclyloxy, arylheterocyclyloxy, arylheteroaryloxy, alkylthioaryl, alkenylthioaryl, alkynylthioaryl, arylthioaryl, arylacylthio, arylcarbocyclylthio, arylheterocyclylthio, arylheteroarylthio, alkylamino, alkenylamino, alkynylamino, arylamino, acylamino, carbocyclylamino, heterocyclylamino, heteroarylamino, alkylaminoalkyl, alkenylaminoalkyl, alkynylaminoalkyl, alkenylaminoalkenyl, alkenylaminoalkynyl, alkynylaminoalkynyl, arylaminoalkyl, alkylacylamino, alkylcarbocyclylamino, alkylheterocyclylamino, alkylheteroarylamino, alkylaminoaryl, alkenylaminoaryl, alkynylaminoaryl, arylaminoaryl, arylacylamino, arylcarbocyclylamino, arylheterocyclylamino, arylheteroarylamino, alkylamido, alkenylamido, alkynylamido, arylamido, acylamido, carbocyclylamido, heterocyclylamido, heteroarylamido, alkylamidoalkyl, alkenylamidoalkyl, alkynylamidoalkyl, alkenylamidoalkenyl, alkenylamidoalkynyl, alkynylamidoalkynyl, arylamidoalkyl, alkylacylamido, alkylcarbocyclylamido, alkylheterocyclylamido, alkylheteroarylamido, alkylamidoaryl, alkenylamidoaryl, alkynylamidoaryl, arylamidoaryl, arylacylamido, arylcarbocyclylamido, arylheterocyclylamido, arylheteroarylamido, alkylcarbamyl, alkenylcarbamyl, alkynylcarbamyl, arylcarbamyl, acylcarbamyl, carbocyclylcarbamyl, heterocyclylcarbamyl, heteroarylcarbamyl, alkylcarbamylalkyl, alkenylcarbamylalkyl, alkynylcarbamylalkyl, alkenylcarbamylalkenyl, alkenylcarbamylalkynyl, alkynylcarbamylalkynyl, arylcarbamylalkyl, alkylacylcarbamyl, alkylcarbocyclylcarbamyl, alkylheterocyclylcarbamyl, alkylheteroarylcarbamyl, alkylcarbamylaryl, alkenylcarbamylaryl, alkynylcarbamylaryl, arylcarbamylaryl, arylacylcarbamyl, arylcarbocyclylcarbamyl, arylheterocyclylcarbamyl, arylheteroarylcarbamyl, alkyldisulfidyl, alkenyldisulfidyl, alkynyldisulfidyl, aryldisulfidyl, acyldisulfidyl, carbocyclyldisulfidyl, heterocyclyldisulfidyl, heteroaryldisulfidyl, alkyldisulfidylalkyl, alkenyldisulfidylalkyl, alkynyldisulfidylalkyl, alkenyldisulfidylalkenyl, alkenyldisulfidylalkynyl, alkynyldisulfidylalkynyl, aryldisulfidylalkyl, alkylacyldisulfidyl, alkylcarbocyclyldisulfidyl, alkylheterocyclyldisulfidyl, alkylheteroaryldisulfidyl, alkyldisulfidylaryl, alkenyldisulfidylaryl, alkynyldisulfidylaryl, aryldisulfidylaryl, arylacyldisulfidyl, arylcarbocyclyldisulfidyl, arylheterocyclyldisulfidyl, and arylheteroaryldisulfidyl, and wherein R¹ is optionally substituted;
and wherein R¹ is not —CH₂OH;
R⁵⁰ is selected from optionally substituted alkyl, alkenyl, alkynyl.

67. The composition of claim 66, wherein R¹ is selected from C₁-C₁₈ alkyl, C₂-C₁₈ alkenyl, C₂-C₁₈ alkynyl, C₆-C₁₈ aryl, C₁-C₁₈ acyl, C₃-C₁₈ carbocyclyl, C₂-C₁₈ heterocyclyl, C₃-C₁₈ heteroaryl, C₁-C₁₈ alkyloxy, C₂-C₁₈ alkenyloxy, C₂-C₁₈ alkynyloxy, C₆-C₁₈ aryloxy, C₁-C₁₈ acyloxy, C₃-C₁₈ carbocyclyloxy, C₂-C₁₈ heterocyclyloxy, C₃-C₁₈ heteroaryloxy, C₁-C₁₈ alkylthio, C₂-C₁₈ alkenylthio, C₂-C₁₈ alkynylthio, C₆-C₁₈ arylthio, C₁-C₁₈ acylthio, C₃-C₁₈ carbocyclylthio, C₂-C₁₈ heterocyclylthio, C₃-C₁₈ heteroarylthio, C₃-C₁₈ alkylalkenyl, C₃-C₁₈ alkylalkynyl C₇-C₂₄ alkylaryl, C₂-C₁₈ alkylacyl, C₄-C₁₈ alkylcarbocyclyl, C₃-C₁₈ alkylheterocyclyl, C₄-C₁₈ alkylheteroaryl, C₂-C₁₈ alkyloxyalkyl, C₃-C₁₈ alkenyloxyalkyl, C₃-C₁₈ alkynyloxyalkyl, C₇-C₂₄ aryloxyalkyl, C₂-C₁₈ alkylacyloxy, C₂₋₁₈ alkyloxyacyl C₂₋₁₈ alkyl, C₄-C₁₈ alkylcarbocyclyloxy, C₃-C₁₈ alkylheterocyclyloxy, C₄-C₁₈ alkylheteroaryloxy, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₄-C₁₈ alkylheterocyclylalkyl, C₄-C₁₈ alkenylheterocyclylalkyl, C₄-C₁₈ alkynylheterocyclylalkyl, C₅-C₁₈ alkenylheterocyclylalkenyl, C₅-C₁₈ alkenylheterocyclylalkynyl, C₅-C₁₈ alkynylheterocyclylalkynyl, C₃-C₁₈ alkylheterocyclylthio, C₄-C₁₈ alkylheteroarylthio, C₄-C₁₈ alkylalkenylalkyl, C₄-C₁₈ alkylalkynylalkyl, C₈-C₂₄ alkylarylalkyl, C₃-C₁₈ alkylacylalkyl, C₁₃-C₂₄ arylalkylaryl, C₁₄-C₂₄ arylalkenylaryl, C₁₄-C₂₄ arylalkynylaryl, C₁₃-C₂₄ arylacylaryl, C₇-C₁₈ arylacyl, C₉-C₁₈ arylcarbocyclyl, C₈-C₁₈ arylheterocyclyl, C₉-C₁₈ arylheteroaryl, C₈—C₁₈ alkenyloxyaryl, C₈-C₁₈ alkynyloxyaryl, C₁₂-C₂₄ aryloxyaryl, C₇-C₁₈ arylacyloxy, C₉-C₁₈ arylcarbocyclyloxy, C₈-C₁₈ arylheterocyclyloxy, C₉-C₁₈ arylheteroaryloxy, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylthio, C₂-C₁₈ alkenylthio, C₂-C₁₈ alkynylthio, C₆-C₁₈ arylthio, C₁-C₁₈ acylthio, C₃-C₁₈ carbocyclylthio, C₂-C₁₈ heterocyclylthio, C₃-C₁₈ heteroarylthio, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₉ alkylcarbocyclylthio, C₃-C₁₈ alkylheterocyclylthio, C₄-C₁₈ alkylheteroarylthio, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylamino, C₂-C₁₈ alkenylamino, C₂-C₁₈ alkynylamino, C₆-C₁₈ arylamino, C₁-C₁₈ acylamino, C₃-C₁₈ carbocyclylamino, C₂-C₁₈ heterocyclylamino, C₃-C₁₈ heteroarylamino, C₂-C₁₈ alkylaminoalkyl, C₃-C₁₈ alkenylaminoalkyl, C₃-C₁₈ alkynylaminoalkyl, C₄-C₁₈ alkenylaminoalkenyl, C₄-C₁₈ alkenylaminoalkynyl, C₄-C₁₈ alkynylaminoalkynyl, C₇-C₂₄ arylaminoalkyl, C₂-C₁₈ alkylacylamino, C₄-C₁₈ alkylcarbocyclylamino, C₃-C₁₈ alkylheterocyclylamino, C₄-C₁₈ alkylheteroarylamino, C₈-C₁₈ alkylaminoaryl, C₈-C₁₈ alkenylaminoaryl, C₈-C₁₈ alkynylaminoaryl, C₁₂-C₂₄ arylaminoaryl, C₇-C₁₈ arylacylamino, C₉-C₁₈ arylcarbocyclylamino, C₈-C₁₈ arylheterocyclylamino, C₉-C₁₈ arylheteroarylamino, C₁-C₁₈ alkylamido, C₂-C₁₈ alkenylamido, C₂-C₁₈ alkynylamido, C₆-C₁₈ arylamido, C₁-C₁₈ acylamido, C₃-C₁₈ carbocyclylamido, C₂-C₁₈ heterocyclylamido, C₃-C₁₈ heteroarylamido, C₂-C₁₈ alkylamidoalkyl, C₃-C₁₈ alkenylamidoalkyl, C₃-C₁₈ alkynylamidoalkyl, C₄-C₁₈ alkenylamidoalkenyl, C₄-C₁₈ alkenylamidoalkynyl, C₄-C₁₈ alkynylamidoalkynyl, C₇-C₂₄ arylamidoalkyl, C₂-C₁₈ alkylacylamido, C₄-C₁₈ alkylcarbocyclylamido, C₃-C₁₈ alkylheterocyclylamido, C₄-C₁₈ alkylheteroarylamido, C₈-C₁₈ alkylamidoaryl, C₈-C₁₈ alkenylamidoaryl, C₈-C₁₈ alkynylamidoaryl, C₁₂-C₂₄ arylamidoaryl, C₇-C₁₈ arylacylamido, C₈-C₁₈ arylcarbocyclylamido, C₈-C₁₈ arylheterocyclylamido, C₉-C₁₈ arylheteroarylamido, C₁-C₁₈ alkylcarbamyl, C₂-C₁₈ alkenylcarbamyl, C₂-C₁₈ alkynylcarbamyl, C₈-C₁₈ arylcarbamyl, C₁-C₁₈ acylcarbamyl, C₃-C₁₈ carbocyclylcarbamyl, C₂-C₁₈ heterocyclylcarbamyl, C₃-C₁₈ heteroarylcarbamyl, C₂-C₁₈ alkylcarbamylalkyl, C₃-C₁₈ alkenylcarbamylalkyl, C₃-C₁₈ alkynylcarbamylalkyl, C₄-C₁₈ alkenylcarbamylalkenyl, C₄-C₁₈ alkenylcarbamylalkynyl, C₄-C₁₈ alkynylcarbamylalkynyl, C₇-C₂₄ arylcarbamylalkyl, C₂-C₁₈ alkylacylcarbamyl, C₄-C₁₈ alkylcarbocyclylcarbamyl, C₃-C₁₈ alkylheterocyclylcarbamyl, C₄-C₁₈ alkylheteroarylcarbamyl, C₈-C₁₈ alkylcarbamylaryl, C₅-C₁₈ alkenylcarbamylaryl, C₈-C₁₈ alkynylcarbamylaryl, C₁₂-C₂₄ arylcarbamylaryl, C₇-C₁₈ arylacylcarbamyl, C₉-C₁₈ arylcarbocyclylcarbamyl, C₈-C₁₈ arylheterocyclylcarbamyl, C₉-C₁₈ arylheteroarylcarbamyl, C₁-C₁₈ alkyldisulfidyl, C₂-C₁₈ alkenyldisulfidyl, C₂-C₁₈ alkynyldisulfidyl, C₆-C₁₈ aryldisulfidyl, C₁-C₁₈ acyldisulfidyl, C₃-C₁₈ carbocyclyldisulfidyl, C₂-C₁₈ heterocyclyldisulfidyl, C₃-C₁₈ heteroaryldisulfidyl, C₂-C₁₈ alkyldisulfidylalkyl, C₃-C₁₈ alkenyldisulfidylalkyl, C₃-C₁₈ alkynyldisulfidylalkyl, C₄-C₁₈ alkenyldisulfidylalkenyl, C₄-C₁₈ alkenyldisulfidylalkynyl, C₄-C₁₈ alkynyldisulfidylalkynyl, C₇-C₂₄ aryldisulfidylalkyl, C₂-C₁₈ alkylacyldisulfidyl, C₄-C₁₈ alkylcarbocyclyldisulfidyl, C₃-C₁₈ alkylheterocyclyldisulfidyl, C₄-C₁₈ alkylheteroaryldisulfidyl, C₈-C₁₈ alkyldisulfidylaryl, C₈-C₁₈ alkenyldisulfidylaryl, C₈-C₁₈ alkynyldisulfidylaryl, C₁₂-C₂₄ aryldisulfidylaryl, C₇-C₁₈ arylacyldisulfidyl, C₉-C₁₈ arylcarbocyclyldisulfidyl, C₈-C₁₈ arylheterocyclyldisulfidyl, and C₉—C₁₈ arylheteroaryldisulfidyl, and wherein R¹ is optionally substituted;
and wherein R¹ is not —CH₂OH.

68. The composition of claim 66 or claim 67, wherein R¹ is selected from alkyl (e.g., C₁-C₁₈, C₁-C₆, C₁-C₅, C₈-C₁₈, or C₉-C₁₈), aryl (e.g., C₆-C₁₈), heteroaryl (e.g., C₃-C₁), carbocyclyl (e.g., C₃-C₁₈), heterocyclyl (e.g., C₂-C₁₈), alkylaryl (e.g., C₇-C₂₄), alkylheteroaryl (e.g., C₄-C₁₈), alkylcarbocyclyl (e.g., C₄-C₁₈), alkylthioalkyl (e.g., wherein each alkyl is C₁-C₁₈), arylthioalkyl (e.g., wherein each alkyl is C₇-C₁₈), alkylaminoalkyl (e.g., wherein each alkyl is C₁-C₁₈), alkylamidoalkyl (e.g., wherein each alkyl is C₁-C₁₈), alkylheterocyclylalkyl (e.g., C₃-C₁₈), alkylthio (e.g., C₁-C₁₈; such as thiomethyl) and alkylheterocyclyl (e.g., C₃-C₁₈);
and wherein R¹ is not —CH₂OH.

69. The method of any one of claims 52 to 67, wherein the iNKT agonist comprises a Compound of Formula (II) or a pharmaceutically acceptable salt thereof, wherein:
A is selected from alkyl, alkenyl, and alkynyl;
X is selected from —CR¹⁰R¹¹—, —O—, —S—, —NR^{a}—, —NR^{a}C(O)—, —NR^{a}C(O)O—, —S—S—, heterocyclyl, heteroaryl, wherein R¹⁰ and R¹¹ where present are independently selected from H, halo, C₁₋₂alkyl, and wherein R^{a} may be selected from hydrogen, alkyl, alkenyl, alkynyl, aryl, carbocyclyl, heteroaryl, heterocyclyl, arylalkyl, and acyl;
R² is selected from hydrogen, halo, alkyl, alkenyl, alkynyl, aryl, acyl, carbocyclyl, heterocyclyl, heteroaryl, alkyloxy, alkenyloxy, alkynyloxy, aryloxy, acyloxy, carbocyclyloxy, heterocyclyloxy, heteroaryloxy, alkylthio, alkenylthio, alkynylthio, arylthio, acylthio, carbocyclylthio, heterocyclylthio, heteroarylthio, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkyloxyacylalkyl, alkylcarbocyclyloxy, alkylheterocyclyloxy, alkylheteroaryloxy, alkylthioalkyl, alkenylthioalkyl, alkynylthioalkyl, alkenylthioalkenyl, alkenylthioalkynyl, alkynylthioalkynyl, arylthioalkyl, alkylacylthio, alkylcarbocyclylthio, alkylheterocyclylalkyl, alkenylheterocyclylalkyl, alkynylheterocyclylalkyl, alkenylheterocyclylalkenyl, alkenylheterocyclylalkynyl, alkynylheterocyclylalkynyl, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, arylacyloxy, arylcarbocyclyloxy, arylheterocyclyloxy, arylheteroaryloxy, alkylthioaryl, alkenylthioaryl, alkynylthioaryl, arylthioaryl, arylacylthio, arylcarbocyclylthio, arylheterocyclylthio, arylheteroarylthio, alkylamino, alkenylamino, alkynylamino, arylamino, acylamino, carbocyclylamino, heterocyclylamino, heteroarylamino, alkylaminoalkyl, alkenylaminoalkyl, alkynylaminoalkyl, alkenylaminoalkenyl, alkenylaminoalkynyl, alkynylaminoalkynyl, arylaminoalkyl, alkylacylamino, alkylcarbocyclylamino, alkylheterocyclylamino, alkylheteroarylamino, alkylaminoaryl, alkenylaminoaryl, alkynylaminoaryl, arylaminoaryl, arylacylamino, arylcarbocyclylamino, arylheterocyclylamino, arylheteroarylamino, alkylamido, alkenylamido, alkynylamido, arylamido, acylamido, carbocyclylamido, heterocyclylamido, heteroarylamido, alkylamidoalkyl, alkenylamidoalkyl, alkynylamidoalkyl, alkenylamidoalkenyl, alkenylamidoalkynyl, alkynylamidoalkynyl, arylamidoalkyl, alkylacylamido, alkylcarbocyclylamido, alkylheterocyclylamido, alkylheteroarylamido, alkylamidoaryl, alkenylamidoaryl, alkynylamidoaryl, arylamidoaryl, arylacylamido, arylcarbocyclylamido, arylheterocyclylamido, arylheteroarylamido, alkylcarbamyl, alkenylcarbamyl, alkynylcarbamyl, arylcarbamyl, acylcarbamyl, carbocyclylcarbamyl, heterocyclylcarbamyl, heteroarylcarbamyl, alkylcarbamylalkyl, alkenylcarbamylalkyl, alkynylcarbamylalkyl, alkenylcarbamylalkenyl, alkenylcarbamylalkynyl, alkynylcarbamylalkynyl, arylcarbamylalkyl, alkylacylcarbamyl, alkylcarbocyclylcarbamyl, alkylheterocyclylcarbamyl, alkylheteroarylcarbamyl, alkylcarbamylaryl, alkenylcarbamylaryl, alkynylcarbamylaryl, arylcarbamylaryl, arylacylcarbamyl, arylcarbocyclylcarbamyl, arylheterocyclylcarbamyl, arylheteroarylcarbamyl, alkyldisulfidyl, alkenyldisulfidyl, alkynyldisulfidyl, aryldisulfidyl, acyldisulfidyl, carbocyclyldisulfidyl, heterocyclyldisulfidyl, heteroaryldisulfidyl, alkyldisulfidylalkyl, alkenyldisulfidylalkyl, alkynyldisulfidylalkyl, alkenyldisulfidylalkenyl, alkenyldisulfidylalkynyl, alkynyldisulfidylalkynyl, aryldisulfidylalkyl, alkylacyldisulfidyl, alkylcarbocyclyldisulfidyl, alkylheterocyclyldisulfidyl, alkylheteroaryldisulfidyl, alkyldisulfidylaryl, alkenyldisulfidylaryl, alkynyldisulfidylaryl, aryldisulfidylaryl, arylacyldisulfidyl, arylcarbocyclyldisulfidyl, arylheterocyclyldisulfidyl, and arylheteroaryldisulfidyl, and wherein R² is optionally substituted;
and wherein -A-X—R² is not —CH₂OH;
R⁵⁰ is selected from optionally substituted alkyl, alkenyl, alkynyl.

70. The method of claim 69, wherein R^{a} is selected from hydrogen, C₁-C₈ alkyl, C₂-C₈ alkenyl, C₂-C₈ alkynyl, C₆-C₁₈ aryl, C₃-C₁₈ carbocyclyl, C₃-C₁₈ heteroaryl, C₂-C₁₈ heterocyclyl, C₇-C₂₄ arylalkyl, and C₁-C₈ acyl.

71. The method of claim 69 or claim 70, wherein X is S or —NR^{a}—, and wherein R^{a} is selected from hydrogen, alkyl, alkenyl, alkynyl, aryl, carbocyclyl, heteroaryl, heterocyclyl, arylalkyl, and acyl.

72. The method of claim 71, wherein R^{a} is selected from hydrogen, C₁-C₁₈ alkyl, C₂-C₈ alkenyl, C₂-C₈ alkynyl, C₆-C₁₈ aryl, C₃-C₁₈ carbocyclyl, C₃-C₁₈ heteroaryl, C₂-C₁₈ heterocyclyl, C₇-C₂₄ arylalkyl, and C₁-C₈ acyl.

73. The method of any one of claims 66 to 72, wherein A is —CR¹²R¹³— and R¹² and R¹³ are independently selected from H, halo, C₁₋₂alkyl.

74. The method of any one of claims 66 to 73, wherein A is —CH₂—.

75. The method of any one of claims 66 to 74, wherein R² is selected from C₁-C₁₈ alkyl, C₂-C₁₈ alkenyl, C₂-C₁₈ alkynyl, C₆-C₁₈ aryl, C₁-C₁₈ acyl, C₃-C₁₈ carbocyclyl, C₂-C₁₈ heterocyclyl, C₃-C₁₈ heteroaryl, C₁-C₁₈ alkyloxy, C₂-C₁₈ alkenyloxy, C₂-C₁₈ alkynyloxy, C₆-C₁₈ aryloxy, C₁-C₁₈ acyloxy, C₃-C₁₈ carbocyclyloxy, C₂-C₁₈ heterocyclyloxy, C₃-C₁₈ heteroaryloxy, C₁-C₁₈ alkylthio, C₂-C₁₈ alkenylthio, C₂-C₁₈ alkynylthio, C₆-C₁₈ arylthio, C₁-C₁₈ acylthio, C₃-C₁₈ carbocyclylthio, C₂-C₁₈ heterocyclylthio, C₃-C₁₈ heteroarylthio, C₃-C₁₈ alkylalkenyl, C₃-C₁₈ alkylalkynyl C₇-C₂₄ alkylaryl, C₂-C₁₈ alkylacyl, C₄-C₁₈ alkylcarbocyclyl, C₃-C₁₈ alkylheterocyclyl, C₄-C₁₈ alkylheteroaryl, C₂-C₁₈ alkyloxyalkyl, C₃-C₁₈ alkenyloxyalkyl, C₃-C₁₈ alkynyloxyalkyl, C₇-C₂₄ aryloxyalkyl, C₂-C₁₈ alkylacyloxy, C₂₋₁₈ alkyloxyacyl C₂₋₁₈ alkyl, C₄-C₁₈ alkylcarbocyclyloxy, C₃-C₁₈ alkylheterocyclyloxy, C₄-C₁₈ alkylheteroaryloxy, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₄-C₁₈ alkylheterocyclylalkyl, C₄-C₁₈ alkenylheterocyclylalkyl, C₄-C₁₈ alkynylheterocyclylalkyl, C₅-C₁₈ alkenylheterocyclylalkenyl, C₅-C₁₈ alkenylheterocyclylalkynyl, C₅-C₁₈ alkynylheterocyclylalkynyl, C₃-C₁₈ alkylheterocyclylthio, C₄-C₁₈ alkylheteroarylthio, C₄-C₁₈ alkylalkenylalkyl, C₄-C₁₈ alkylalkynylalkyl, C₈-C₂₄ alkylarylalkyl, C₃-C₁₈ alkylacylalkyl, C₁₃-C₂₄ arylalkylaryl, C₁₄-C₂₄ arylalkenylaryl, C₁₄-C₂₄ arylalkynylaryl, C₁₃-C₂₄ arylacylaryl, C₇-C₁₈ arylacyl, C₉-C₁₈ arylcarbocyclyl, C₈-C₁₈ arylheterocyclyl, C₉-C₁₈ arylheteroaryl, C₈-C₁₈ alkenyloxyaryl, C₈-C₁₈ alkynyloxyaryl, C₁₂-C₂₄ aryloxyaryl, C₇-C₁₈ arylacyloxy, C₉-C₁₈ arylcarbocyclyloxy, C₈-C₁₈ arylheterocyclyloxy, C₉-C₁₈ arylheteroaryloxy, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylthio, C₂-C₁₈ alkenylthio, C₂-C₁₈ alkynylthio, C₈-C₁₈ arylthio, C₁-C₁₈ acylthio, C₃-C₁₈ carbocyclylthio, C₂-C₁₈ heterocyclylthio, C₃-C₁₈ heteroarylthio, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₃-C₁₈ alkylheterocyclylthio, C₄-C₁₈ alkylheteroarylthio, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylamino, C₂-C₁₈ alkenylamino, C₂-C₁₈ alkynylamino, C₆-C₁₈ arylamino, C₁-C₁₈ acylamino, C₃-C₁₉ carbocyclylamino, C₂-C₁₈ heterocyclylamino, C₃-C₁₈ heteroarylamino, C₂-C₁₈ alkylaminoalkyl, C₃-C₁₈ alkenylaminoalkyl, C₃-C₁₈ alkynylaminoalkyl, C₄-C₁₈ alkenylaminoalkenyl, C₄-C₁₈ alkenylaminoalkynyl, C₄-C₁₈ alkynylaminoalkynyl, C₇-C₂₄ arylaminoalkyl, C₂-C₁₈ alkylacylamino, C₄-C₁₈ alkylcarbocyclylamino, C₃-C₁₈ alkylheterocyclylamino, C₄-C₁₈ alkylheteroarylamino, C₈-C₁₈ alkylaminoaryl, C₈-C₁₈ alkenylaminoaryl, C₈-C₁₈ alkynylaminoaryl, C₁₂-C₂₄ arylaminoaryl, C₇-C₁₈ arylacylamino, C₉-C₁₈ arylcarbocyclylamino, C₈-C₁₈ arylheterocyclylamino, C₉-C₁₈ arylheteroarylamino, C₁-C₁₈ alkylamido, C₂-C₁₈ alkenylamido, C₂-C₁₈ alkynylamido, C₆-C₁₈ arylamido, C₁-C₁₈ acylamido, C₃-C₁₈ carbocyclylamido, C₂-C₁₈ heterocyclylamido, C₃-C₁₈ heteroarylamido, C₂-C₁₈ alkylamidoalkyl, C₃-C₁₈ alkenylamidoalkyl, C₃-C₁₈ alkynylamidoalkyl, C₄-C₁₈ alkenylamidoalkenyl, C₄-C₁₈ alkenylamidoalkynyl, C₄-C₁₈ alkynylamidoalkynyl, C₇-C₂₄ arylamidoalkyl, C₂-C₁₈ alkylacylamido, C₄-C₁₈ alkylcarbocyclylamido, C₃-C₁₈ alkylheterocyclylamido, C₄-C₁₈ alkylheteroarylamido, C₈-C₁₈ alkylamidoaryl, C₈-C₁₈ alkenylamidoaryl, C₈-C₁₈ alkynylamidoaryl, C₁₂-C₂₄ arylamidoaryl, C₇-C₁₈ arylacylamido, C₉-C₁₈ arylcarbocyclylamido, C₈-C₁₈ arylheterocyclylamido, C₉-C₁₈ arylheteroarylamido, C₁-C₁₈ alkylcarbamyl, C₂-C₁₈ alkenylcarbamyl, C₂-C₁₈ alkynylcarbamyl, C₆-C₁₈ arylcarbamyl, C₁-C₁₈ acylcarbamyl, C₃-C₁₈ carbocyclylcarbamyl, C₂-C₁₈ heterocyclylcarbamyl, C₃-C₁₈ heteroarylcarbamyl, C₂-C₁₈ alkylcarbamylalkyl, C₃-C₁₈ alkenylcarbamylalkyl, C₃-C₁₈ alkynylcarbamylalkyl, C₄-C₁₈ alkenylcarbamylalkenyl, C₄-C₁₈ alkenylcarbamylalkynyl, C₄-C₁₈ alkynylcarbamylalkynyl, C₇-C₂₄ arylcarbamylalkyl, C₂-C₁₈ alkylacylcarbamyl, C₄-C₁₈ alkylcarbocyclylcarbamyl, C₃-C₁₈ alkylheterocyclylcarbamyl, C₄-C₁₈ alkylheteroarylcarbamyl, C₈-C₁₈ alkylcarbamylaryl, C₈-C₁₈ alkenylcarbamylaryl, C₈-C₁₈ alkynylcarbamylaryl, C₁₂-C₂₄ arylcarbamylaryl, C₇-C₁₈ arylacylcarbamyl, C₉-C₁₈ arylcarbocyclylcarbamyl, C₈-C₁₈ arylheterocyclylcarbamyl, C₉-C₁₈ arylheteroarylcarbamyl, C₁-C₁₈ alkyldisulfidyl, C₂-C₁₈ alkenyldisulfidyl, C₂-C₁₈ alkynyldisulfidyl, C₆-C₁₈ aryldisulfidyl, C₁-C₁₈ acyldisulfidyl, C₃—C₁₈ carbocyclyldisulfidyl, C₂-C₁₈ heterocyclyldisulfidyl, C₃-C₁₈ heteroaryldisulfidyl, C₂-C₁₈ alkyldisulfidylalkyl, C₃-C₁₈ alkenyldisulfidylalkyl, C₃-C₁₈ alkynyldisulfidylalkyl, C₄-C₁₈ alkenyldisulfidylalkenyl, C₄-C₁₈ alkenyldisulfidylalkynyl, C₄-C₁₉ alkynyldisulfidylalkynyl, C₇-C₂₄ aryldisulfidylalkyl, C₂-C₁₈ alkylacyldisulfidyl, C₄-C₁₈ alkylcarbocyclyldisulfidyl, C₃-C₁₈ alkylheterocyclyldisulfidyl, C₄-C₁₈ alkylheteroaryldisulfidyl, C₈-C₁₈ alkyldisulfidylaryl, C₈-C₁₈ alkenyldisulfidylaryl, C₈-C₁₈ alkynyldisulfidylaryl, C₁₂-C₂₄ aryldisulfidylaryl, C₇-C₁₈ arylacyldisulfidyl, C₉-C₁₈ arylcarbocyclyldisulfidyl, C₈-C₁₈ arylheterocyclyldisulfidyl, and C₉-C₁₈ arylheteroaryldisulfidyl, and wherein R² is optionally substituted;
and wherein -A-X—R² is not —CH₂OH.

76. The method of any one of claims 66 to 75, wherein R² is selected from alkyl (e.g., C₁-C₁₈, C₁-C₆, C₁-C₅, C₈-C₁₈, or C₉-C₁₈), aryl (e.g., C₆-C₁₈), heteroaryl (e.g., C₃-C₁₈), carbocyclyl (e.g., C₃-C₁₈), heterocyclyl (e.g., C₂-C₁₈), alkylaryl (e.g., C₇-C₂₄), alkylheteroaryl (e.g., C₄-C₁₈), alkylcarbocyclyl (e.g., C₄-C₁₉), alkylthioalkyl (e.g., wherein each alkyl is C₁-C₁₈), arylthioalkyl (e.g., wherein each alkyl is C₇-C₁₈), alkylaminoalkyl (e.g., wherein each alkyl is C₁-C₁₈), alkylamidoalkyl (e.g., wherein each alkyl is C₁-C₁), alkylheterocyclylalkyl (e.g., C₃-C₁₈), alkylthio (e.g., C₁-C₁₉; such as thiomethyl) and alkylheterocyclyl (e.g., C₃-C₁₈).

77. The method of any one of claims 52 to 76, wherein the iNKT cell agonist is a Compound of Formula (III): 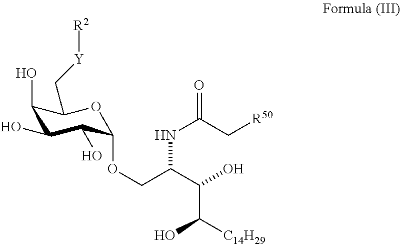
or a pharmaceutically acceptable salt thereof, wherein:
Y is selected from —S—, —NR^{a}—, —NR^{a}C(O)—, —NR^{a}C(O)O—, —S—S—, heterocyclyl, heteroaryl, wherein R^{a} may be selected from hydrogen, alkyl, alkenyl, alkynyl, aryl, carbocyclyl, heteroaryl, heterocyclyl, arylalkyl, and acyl;
R² is selected from hydrogen, halo, alkyl, alkenyl, alkynyl, aryl, acyl, carbocyclyl, heterocyclyl, heteroaryl, alkyloxy, alkenyloxy, alkynyloxy, aryloxy, acyloxy, carbocyclyloxy, heterocyclyloxy, heteroaryloxy, alkylthio, alkenylthio, alkynylthio, arylthio, acylthio, carbocyclylthio, heterocyclylthio, heteroarylthio, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkyloxyacylalkyl, alkylcarbocyclyloxy, alkylheterocyclyloxy, alkylheteroaryloxy, alkylthioalkyl, alkenylthioalkyl, alkynylthioalkyl, alkenylthioalkenyl, alkenylthioalkynyl, alkynylthioalkynyl, arylthioalkyl, alkylacylthio, alkylcarbocyclylthio, alkylheterocyclylalkyl, alkenylheterocyclylalkyl, alkynylheterocyclylalkyl, alkenylheterocyclylalkenyl, alkenylheterocyclylalkynyl, alkynylheterocyclylalkynyl, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, arylacyloxy, arylcarbocyclyloxy, arylheterocyclyloxy, arylheteroaryloxy, alkylthioaryl, alkenylthioaryl, alkynylthioaryl, arylthioaryl, arylacylthio, arylcarbocyclylthio, arylheterocyclylthio, arylheteroarylthio, alkylamino, alkenylamino, alkynylamino, arylamino, acylamino, carbocyclylamino, heterocyclylamino, heteroarylamino, alkylaminoalkyl, alkenylaminoalkyl, alkynylaminoalkyl, alkenylaminoalkenyl, alkenylaminoalkynyl, alkynylaminoalkynyl, arylaminoalkyl, alkylacylamino, alkylcarbocyclylamino, alkylheterocyclylamino, alkylheteroarylamino, alkylaminoaryl, alkenylaminoaryl, alkynylaminoaryl, arylaminoaryl, arylacylamino, arylcarbocyclylamino, arylheterocyclylamino, arylheteroarylamino, alkylamido, alkenylamido, alkynylamido, arylamido, acylamido, carbocyclylamido, heterocyclylamido, heteroarylamido, alkylamidoalkyl, alkenylamidoalkyl, alkynylamidoalkyl, alkenylamidoalkenyl, alkenylamidoalkynyl, alkynylamidoalkynyl, arylamidoalkyl, alkylacylamido, alkylcarbocyclylamido, alkylheterocyclylamido, alkylheteroarylamido, alkylamidoaryl, alkenylamidoaryl, alkynylamidoaryl, arylamidoaryl, arylacylamido, arylcarbocyclylamido, arylheterocyclylamido, arylheteroarylamido, alkylcarbamyl, alkenylcarbamyl, alkynylcarbamyl, arylcarbamyl, acylcarbamyl, carbocyclylcarbamyl, heterocyclylcarbamyl, heteroarylcarbamyl, alkylcarbamylalkyl, alkenylcarbamylalkyl, alkynylcarbamylalkyl, alkenylcarbamylalkenyl, alkenylcarbamylalkynyl, alkynylcarbamylalkynyl, arylcarbamylalkyl, alkylacylcarbamyl, alkylcarbocyclylcarbamyl, alkylheterocyclylcarbamyl, alkylheteroarylcarbamyl, alkylcarbamylaryl, alkenylcarbamylaryl, alkynylcarbamylaryl, arylcarbamylaryl, arylacylcarbamyl, arylcarbocyclylcarbamyl, arylheterocyclylcarbamyl, arylheteroarylcarbamyl, alkyldisulfidyl, alkenyldisulfidyl, alkynyldisulfidyl, aryldisulfidyl, acyldisulfidyl, carbocyclyldisulfidyl, heterocyclyldisulfidyl, heteroaryldisulfidyl, alkyldisulfidylalkyl, alkenyldisulfidylalkyl, alkynyldisulfidylalkyl, alkenyldisulfidylalkenyl, alkenyldisulfidylalkynyl, alkynyldisulfidylalkynyl, aryldisulfidylalkyl, alkylacyldisulfidyl, alkylcarbocyclyldisulfidyl, alkylheterocyclyldisulfidyl, alkylheteroaryldisulfidyl, alkyldisulfidylaryl, alkenyldisulfidylaryl, alkynyldisulfidylaryl, aryldisulfidylaryl, arylacyldisulfidyl, arylcarbocyclyldisulfidyl, arylheterocyclyldisulfidyl, and arylheteroaryldisulfidyl, and wherein R² is optionally substituted;
R⁵⁰ is selected from optionally substituted alkyl, alkenyl, alkynyl.

78. The composition of claim 77, wherein R^{a} is hydrogen.

79. The composition of claim 77 or claim 78, wherein Y is selected from —S—, —NH—, —NHC(O)—, —NHC(O)O—, and —S—S—, triazolyl.

80. The composition of any one of claims 77 to 79, wherein A is —CR¹²R¹³—, wherein R¹² and R¹³ where present are independently selected from H, halo, C₁₋₂alkyl. More preferably A is —CH₂—.

81. The composition of any one of claims 77 to 80, wherein R² is selected from C₁-C₁₈ alkyl, C₂-C₁₈ alkenyl, C₂-C₁₈ alkynyl, C₆-C₁₈ aryl, C₁-C₁₈ acyl, C₃-C₁₈ carbocyclyl, C₂-C₁₈ heterocyclyl, C₃-C₁₈ heteroaryl, C₁-C₁₈ alkyloxy, C₂-C₁₈ alkenyloxy, C₂-C₁₈ alkynyloxy, C₆-C₁₈ aryloxy, C₁-C₁₈ acyloxy, C₃-C₁₈ carbocyclyloxy, C₂-C₁₈ heterocyclyloxy, C₃-C₁₈ heteroaryloxy, C₁-C₁₈ alkylthio, C₂-C₁₈ alkenylthio, C₂-C₁₈ alkynylthio, C₅-C₁₈ arylthio, C₁-C₁₈ acylthio, C₃-C₁₈ carbocyclylthio, C₂-C₁₈ heterocyclylthio, C₃-C₁₈ heteroarylthio, C₃-C₁₈ alkylalkenyl, C₃-C₁₈ alkylalkynyl C₇-C₂₄ alkylaryl, C₂-C₁₈ alkylacyl, C₄-C₁₈ alkylcarbocyclyl, C₃-C₁₈ alkylheterocyclyl, C₄-C₁₈ alkylheteroaryl, C₂-C₁₈ alkyloxyalkyl, C₃-C₁₈ alkenyloxyalkyl, C₃-C₁₈ alkynyloxyalkyl, C₇-C₂₄ aryloxyalkyl, C₂-C₁₈ alkylacyloxy, C₂₋₁₈ alkyloxyacyl C₂₋₁₈ alkyl, C₄-C₁₈ alkylcarbocyclyloxy, C₃-C₁₈ alkylheterocyclyloxy, C₄-C₁₈ alkylheteroaryloxy, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₄-C₁₈ alkylheterocyclylalkyl, C₄-C₁₈ alkenylheterocyclylalkyl, C₄-C₁₈ alkynylheterocyclylalkyl, C₅-C₁₈ alkenylheterocyclylalkenyl, C₅-C₁₈ alkenylheterocyclylalkynyl, C₅-C₁₈ alkynylheterocyclylalkynyl, C₃-C₁₈ alkylheterocyclylthio, C₄-C₁₈ alkylheteroarylthio, C₄-C₁₈ alkylalkenylalkyl, C₄-C₁₈ alkylalkynylalkyl, C₈-C₂₄ alkylarylalkyl, C₃-C₁₈ alkylacylalkyl, C₁₃-C₂₄ arylalkylaryl, C₁₄-C₂₄ arylalkenylaryl, C₁₄-C₂₄ arylalkynylaryl, C₁₃-C₂₄ arylacylaryl, C₇-C₁₈ arylacyl, C₉-C₁₈ arylcarbocyclyl, C₈-C₁₈ arylheterocyclyl, C₉-C₁₈ arylheteroaryl, C₈-C₁₈ alkenyloxyaryl, C₈-C₁₈ alkynyloxyaryl, C₁₂-C₂₄ aryloxyaryl, C₇-C₁₈ arylacyloxy, C₉-C₁₈ arylcarbocyclyloxy, C₈-C₁₈ arylheterocyclyloxy, C₉-C₁₈ arylheteroaryloxy, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylthio, C₂-C₁₈ alkenylthio, C₂-C₁₈ alkynylthio, C₅-C₁₈ arylthio, C₁-C₁₈ acylthio, C₃-C₁₈ carbocyclylthio, C₂-C₁₈ heterocyclylthio, C₃-C₁₈ heteroarylthio, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₃-C₁₈ alkylheterocyclylthio, C₄-C₁₈ alkylheteroarylthio, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylamino, C₂-C₁₈ alkenylamino, C₂-C₁₈ alkynylamino, C₆-C₁₈ arylamino, C₁-C₁₈ acylamino, C₃-C₁₈ carbocyclylamino, C₂-C₁₈ heterocyclylamino, C₃-C₁₈ heteroarylamino, C₂-C₁₈ alkylaminoalkyl, C₃-C₁₈ alkenylaminoalkyl, C₃-C₁₈ alkynylaminoalkyl, C₄-C₁₈ alkenylaminoalkenyl, C₄-C₁₈ alkenylaminoalkynyl, C₄-C₁₈ alkynylaminoalkynyl, C₇-C₂₄ arylaminoalkyl, C₂-C₁₈ alkylacylamino, C₄-C₁₈ alkylcarbocyclylamino, C₃-C₁₈ alkylheterocyclylamino, C₄-C₁₈ alkylheteroarylamino, C₈-C₁₈ alkylaminoaryl, C₈-C₁₈ alkenylaminoaryl, C₈-C₁₈ alkynylaminoaryl, C₁₂-C₂₄ arylaminoaryl, C₇-C₁₈ arylacylamino, C₉-C₁₈ arylcarbocyclylamino, C₈-C₁₈ arylheterocyclylamino, C₉-C₁₈ arylheteroarylamino, C₁-C₁₈ alkylamido, C₂-C₁₈ alkenylamido, C₂-C₁₈ alkynylamido, C₆-C₁₈ arylamido, C₁-C₁₈ acylamido, C₃-C₁₈ carbocyclylamido, C₂-C₁₈ heterocyclylamido, C₃-C₁₈ heteroarylamido, C₂-C₁₈ alkylamidoalkyl, C₃-C₁₈ alkenylamidoalkyl, C₃-C₁₈ alkynylamidoalkyl, C₄-C₁₈ alkenylamidoalkenyl, C₄-C₁₈ alkenylamidoalkynyl, C₄-C₁₈ alkynylamidoalkynyl, C₇-C₂₄ arylamidoalkyl, C₂-C₁₈ alkylacylamido, C₄-C₁₈ alkylcarbocyclylamido, C₃-C₁₈ alkylheterocyclylamido, C₄-C₁₈ alkylheteroarylamido, C₈-C₁₈ alkylamidoaryl, C₈-C₁₈ alkenylamidoaryl, C₈-C₁₈ alkynylamidoaryl, C₁₂-C₂₄ arylamidoaryl, C₇-C₁₈ arylacylamido, C₈-C₁₈ arylcarbocyclylamido, C₈-C₁₈ arylheterocyclylamido, C₉-C₁₈ arylheteroarylamido, C₁-C₁₈ alkylcarbamyl, C₂-C₁₈ alkenylcarbamyl, C₂-C₁₈ alkynylcarbamyl, C₆-C₁₈ arylcarbamyl, C₁-C₁₈ acylcarbamyl, C₃-C₁₈ carbocyclylcarbamyl, C₂-C₁₈ heterocyclylcarbamyl, C₃-C₁₈ heteroarylcarbamyl, C₂-C₁₈ alkylcarbamylalkyl, C₃-C₁₈ alkenylcarbamylalkyl, C₃-C₁₈ alkynylcarbamylalkyl, C₄-C₁₈ alkenylcarbamylalkenyl, C₄-C₁₈ alkenylcarbamylalkynyl, C₄—C₁₈ alkynylcarbamylalkynyl, C₇-C₂₄ arylcarbamylalkyl, C₂-C₁₈ alkylacylcarbamyl, C₄-C₁₈ alkylcarbocyclylcarbamyl, C₃-C₁₈ alkylheterocyclylcarbamyl, C₄-C₁₈ alkylheteroarylcarbamyl, C₈-C₁₈ alkylcarbamylaryl, C₈-C₁₈ alkenylcarbamylaryl, C₈-C₁₈ alkynylcarbamylaryl, C₁₂-C₂₄ arylcarbamylaryl, C₇-C₁₈ arylacylcarbamyl, C₉-C₁₈ arylcarbocyclylcarbamyl, C₈-C₁₈ arylheterocyclylcarbamyl, C₉-C₁₈ arylheteroarylcarbamyl, C₁-C₁₈ alkyldisulfidyl, C₂-C₁₈ alkenyldisulfidyl, C₂-C₁₈ alkynyldisulfidyl, C₆-C₁₈ aryldisulfidyl, C₁-C₁₈ acyldisulfidyl, C₃-C₁₈ carbocyclyldisulfidyl, C₂-C₁₈ heterocyclyldisulfidyl, C₃-C₁₈ heteroaryldisulfidyl, C₂-C₁₈ alkyldisulfidylalkyl, C₃-C₁₈ alkenyldisulfidylalkyl, C₃-C₁₈ alkynyldisulfidylalkyl, C₄-C₁₈ alkenyldisulfidylalkenyl, C₄-C₁₈ alkenyldisulfidylalkynyl, C₄-C₁₈ alkynyldisulfidylalkynyl, C₇-C₂₄ aryldisulfidylalkyl, C₂-C₁₈ alkylacyldisulfidyl, C₄-C₁₉ alkylcarbocyclyldisulfidyl, C₃-C₁₈ alkylheterocyclyldisulfidyl, C₄-C₁₈ alkylheteroaryldisulfidyl, C₈-C₁₈ alkyldisulfidylaryl, C₈-C₁₈ alkenyldisulfidylaryl, C₈-C₁₈ alkynyldisulfidylaryl, C₁₂-C₂₄ aryldisulfidylaryl, C₇-C₁₈ arylacyldisulfidyl, C₉-C₁₈ arylcarbocyclyldisulfidyl, C₈-C₁₈ arylheterocyclyldisulfidyl, and C₉-C₁₈ arylheteroaryldisulfidyl, and wherein R² is optionally substituted.

82. The composition of ay one of any one of claims 77 to 819, wherein R² in Formula (III) is selected from alkyl (e.g., C₁-C₁₈, C₁-C₆, C₁-C₅, C₈-C₁₈, or C₉-C₁₈), aryl (e.g., C₆-C₁₈), heteroaryl (e.g., C₃-C₁₈), carbocyclyl (e.g., C₃-C₁₈), heterocyclyl (e.g., C₂-C₁₈), alkylaryl (e.g., C₇-C₂₄), alkylheteroaryl (e.g., C₄-C₁₉), alkylcarbocyclyl (e.g., C₄-C₁₈), alkylthioalkyl (e.g., wherein each alkyl is C₁-C₁₈), arylthioalkyl (e.g., wherein each alkyl is C₇-C₁₈), alkylaminoalkyl (e.g., wherein each alkyl is C₁-C₁₈), alkylamidoalkyl (e.g., wherein each alkyl is C₁-C₁₈), alkylheterocyclylalkyl (e.g., C₃-C₁₈), alkylthio (e.g., C₁-C₁₈; such as thiomethyl) and alkylheterocyclyl (e.g., C₃-C₁₈).

83. The composition of any one of claims 52 to 66, wherein the iNKT cell agonists is a compound selected from the following GROUP I compounds:

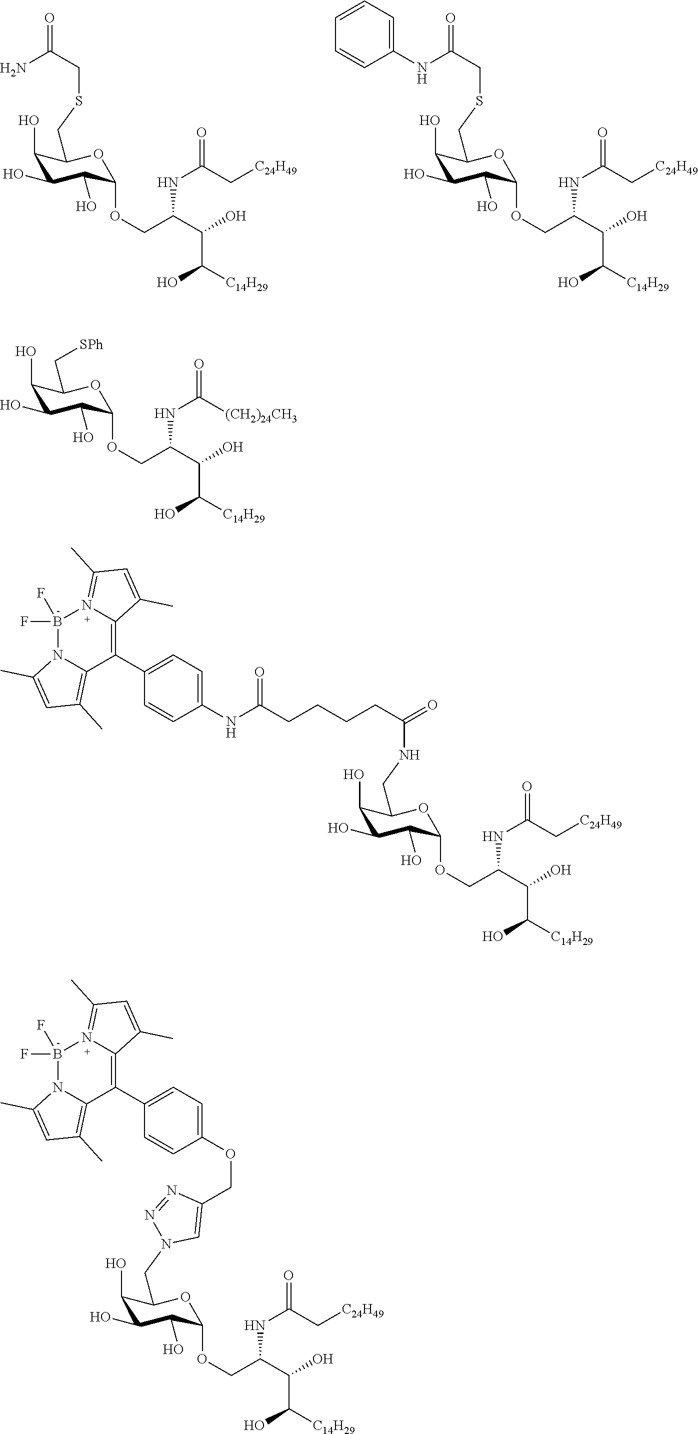

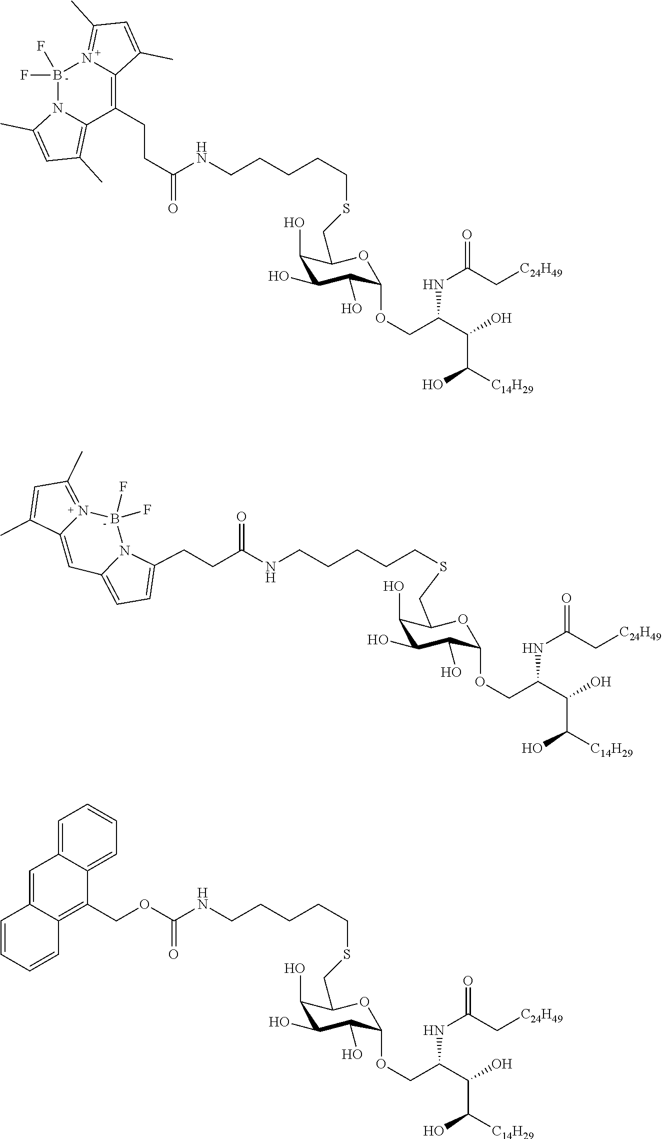

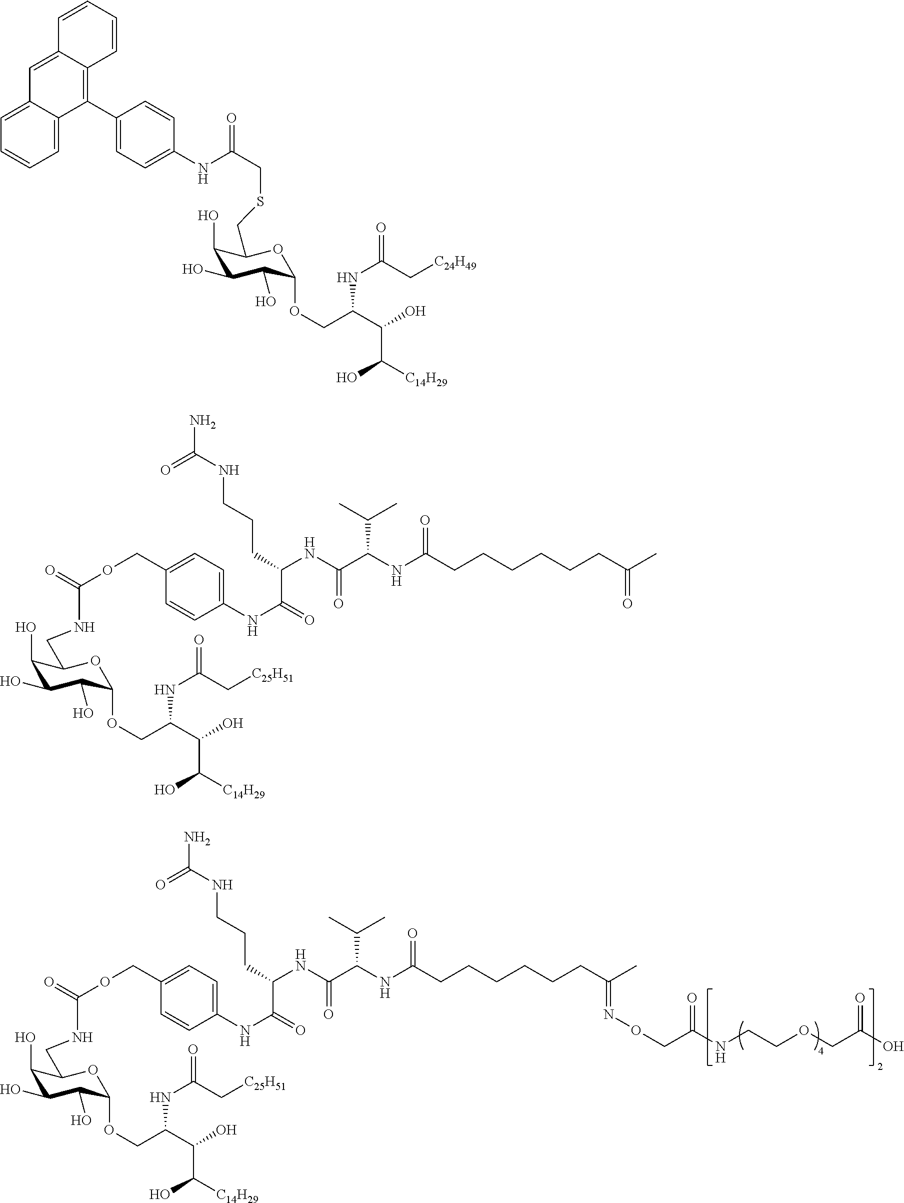

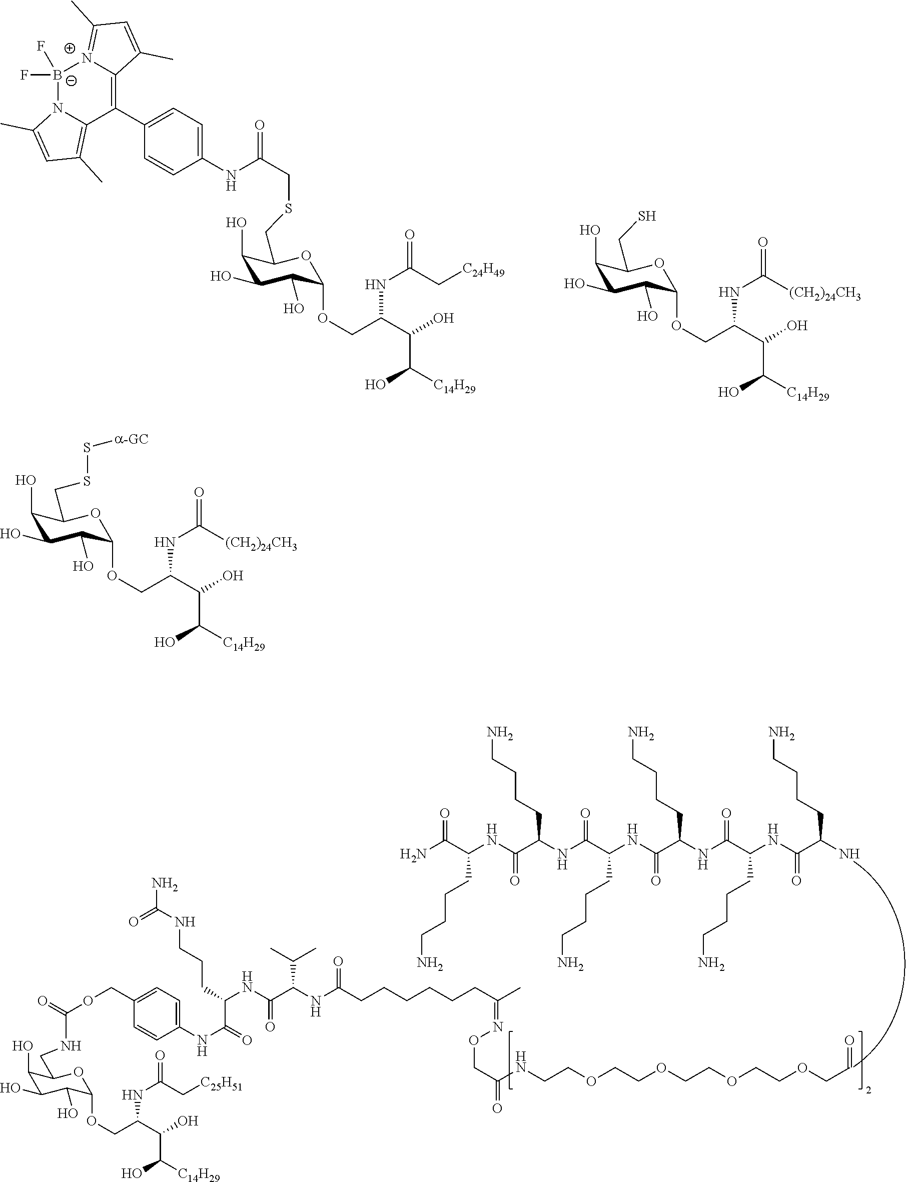

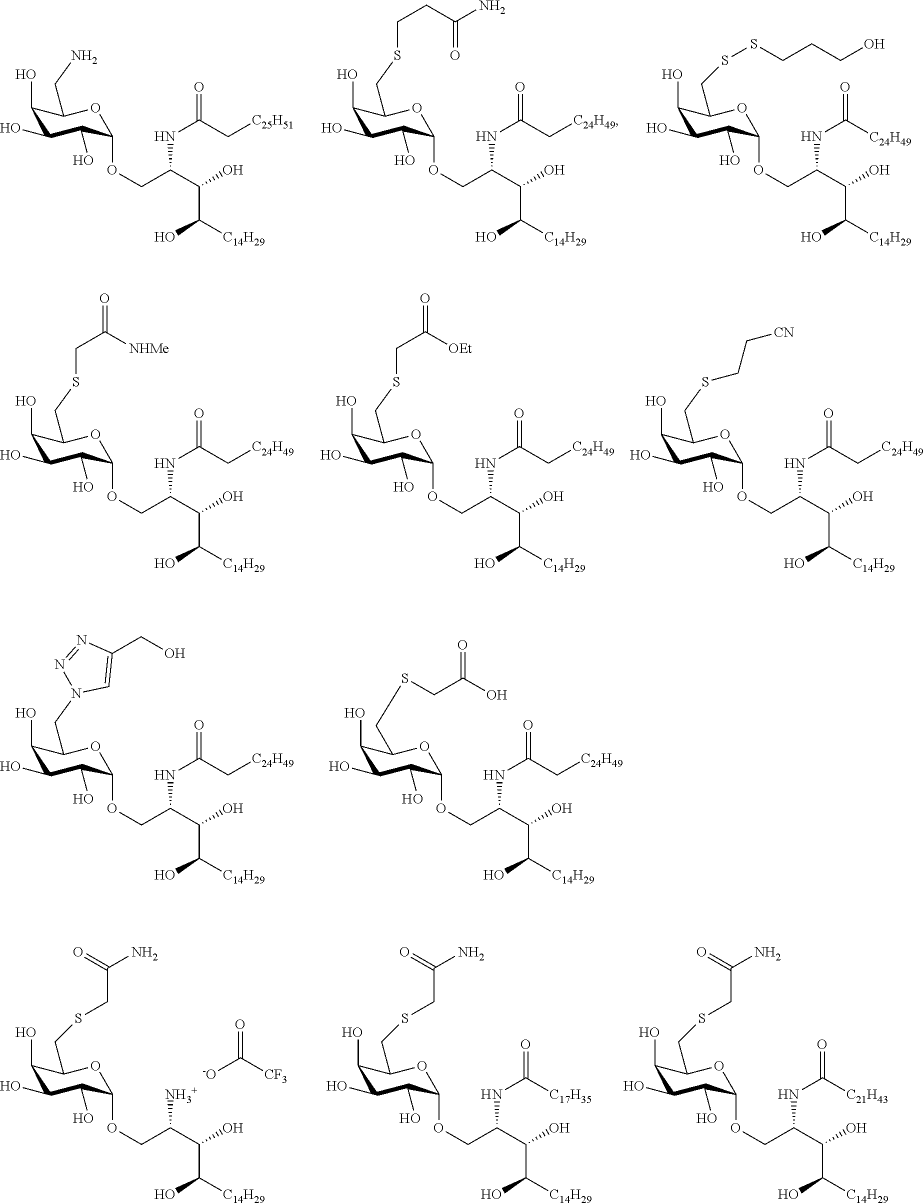

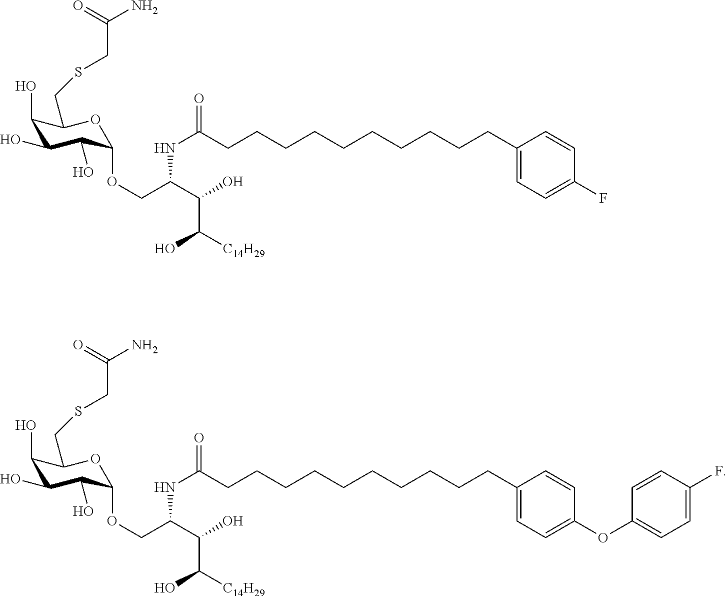

84. The composition of any one of claims 52 to 83, wherein the immune stimulator is a CD8⁺ T cell epitope.

85. The composition of any one of claims 52 to 84, wherein the target antigen is selected from a virus antigen, a bacterial antigen, a parasite antigen, and a cancer antigen.

86. The composition of claim 85, wherein the parasite antigen is derived from a parasite selected from Plasmodium falciparum, P. vivax, P. malariae, P. ovale, and P. knowlesi.

87. The composition of claim 85, wherein the virus antigen is a hepatitis (e.g., hepatitis A, hepatitis B and hepatitis C) antigen.

88. The composition of claim 85, wherein the cancer antigen is a liver cancer antigen.

89. The composition of any one of claims 52 to 88, wherein the polynucleotide is codon-optimised, and wherein the codon optimisation is selected to enhance translation in the subject and/or to reduce innate immunity against the polynucleotide molecule.

90. The composition of any one of claims 52 to 89, wherein the polynucleotide comprises at least one chemical modification.

91. The composition of claim 90, wherein the at least one chemical modification is selected from the group consisting of pseudouridine, N1-methylpseudouridine, N1-ethylpseudouridine, 2thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2thio-dihydrouridine, 2-thio-pseudouridine, 4-methoxy-2-thio-pseudouridine, 4methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

92. The composition of claim 90 or claim 91, wherein the polynucleotide is fully modified.

93. The composition of any one of claims 52 to 92, wherein the isolated RNA molecule is encapsulated within the nanoparticle or complexed to the surface of the nanoparticle.

94. The composition of claim 93, wherein the nanoparticle is a lipid nanoparticle (e.g., a liposome, or a lipoplex).

95. The composition of any one of claims 52 to 94, wherein the lipid nanoparticle has a diameter of between about 50 and 500 nm.

96. The composition of any one of claims 52 to 95, wherein the nanoparticle comprises a cationic lipid, a PEG-modified lipid, a sterol, and/or a non-cationic lipid.

97. The composition of claim 96, wherein the cationic lipid is an ionizable cationic lipid.

98. The composition of claim 96 or claim 97, wherein the ionizable cationic lipid is selected from the group comprising or consisting of 1,2-di-O-octadecenyl-3-trimethylammonium propane (DOTMA), N-[1-(2,3-dioleyloxy)propyl)-N,N,N-trimethylammonium chloride (DOTMA), 1,2-Dioleoyl-3-Trimethylammonium-Propane (DOTAP), 5-carboxyspermylglycinedioctadecylamide (DOGS), 2,3-dioleyloxy-N-[2(spermine-carboxamido)ethyl]-N,N-dimethyl-I-propanaminium (DOSPA), 1,2-Dioleoyl-3-Dimethylammonium-Propane (DODAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), heptatriaconta-6,9,28,31-tetraen19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dilinolenyloxy-N,N-dimethyl-3-aminopropane (DLenDMA), N-dioleyl-N,N-dimethyl ammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis, cis-9,12-octadecadienoxy) propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminoethyl-[1,3]-dioxolane (DLin-K-XTC2-DMA), and C12-200.

99. The composition of any one of claims 52 to 98, wherein the nanoparticle comprises a dioleoylphosphatidylethanolamine (DOPE) lipid.

100. A vaccine comprising the composition of any one of claims 52 to 99, and a pharmaceutically acceptable carrier, excipient, and/or diluent.

101. A compound selected from the following GROUP II compounds:

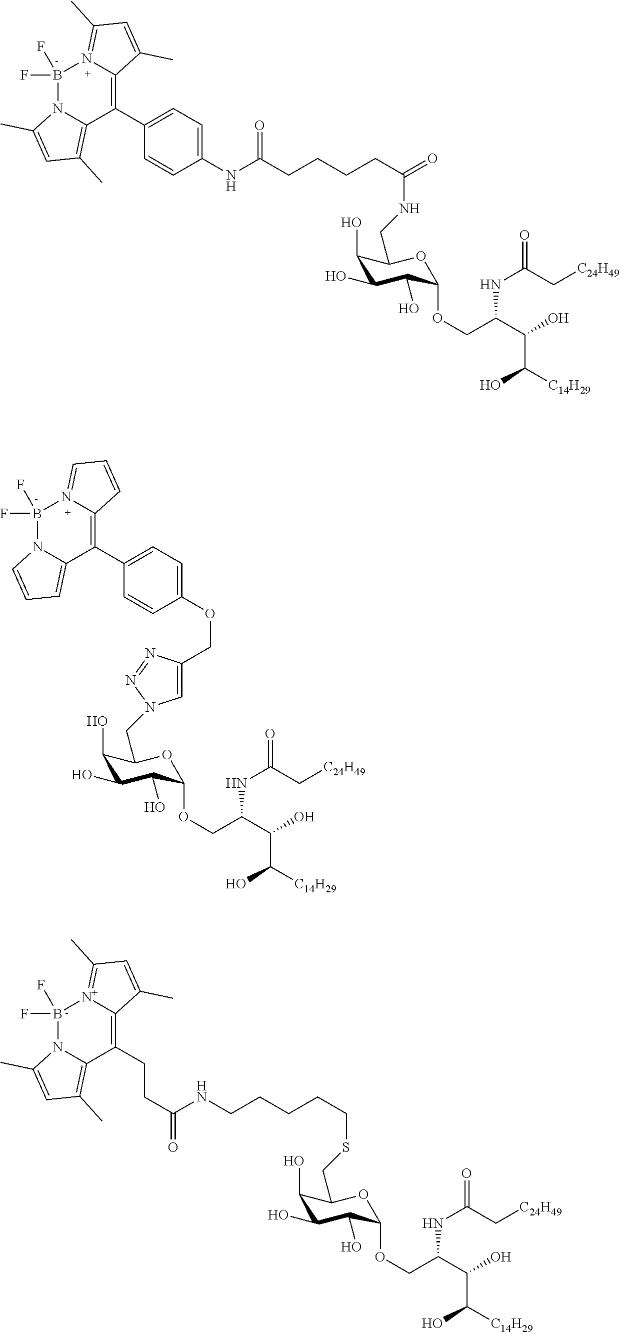

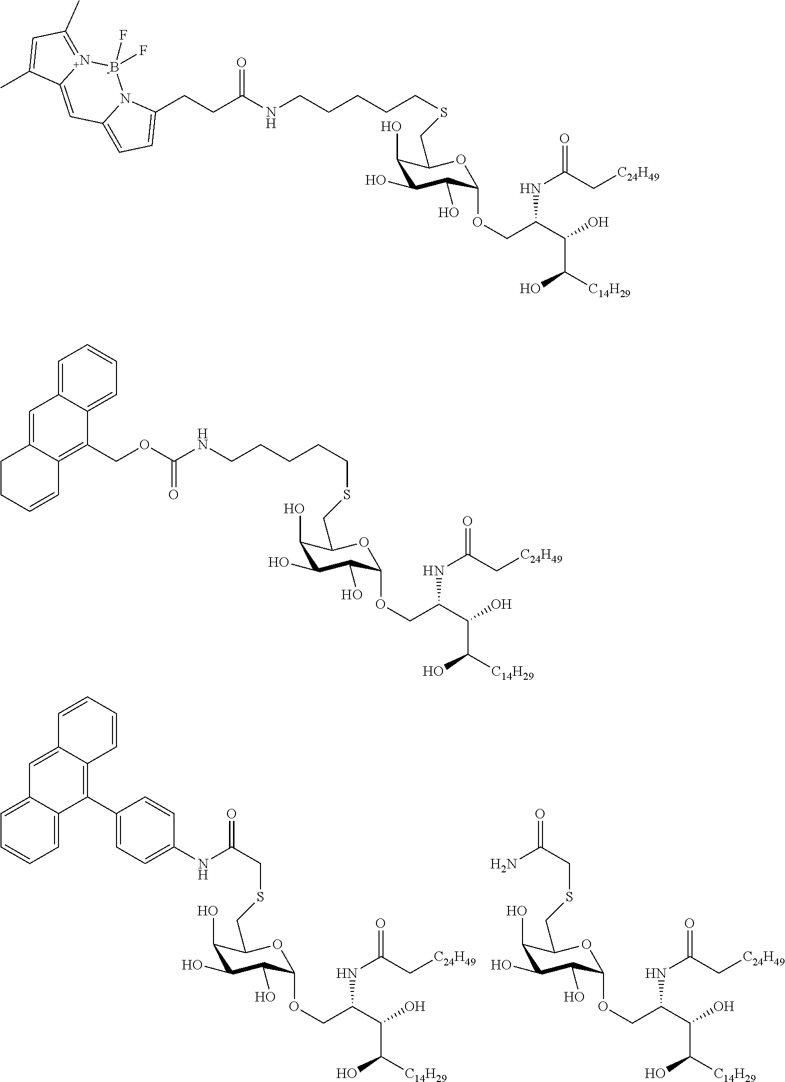

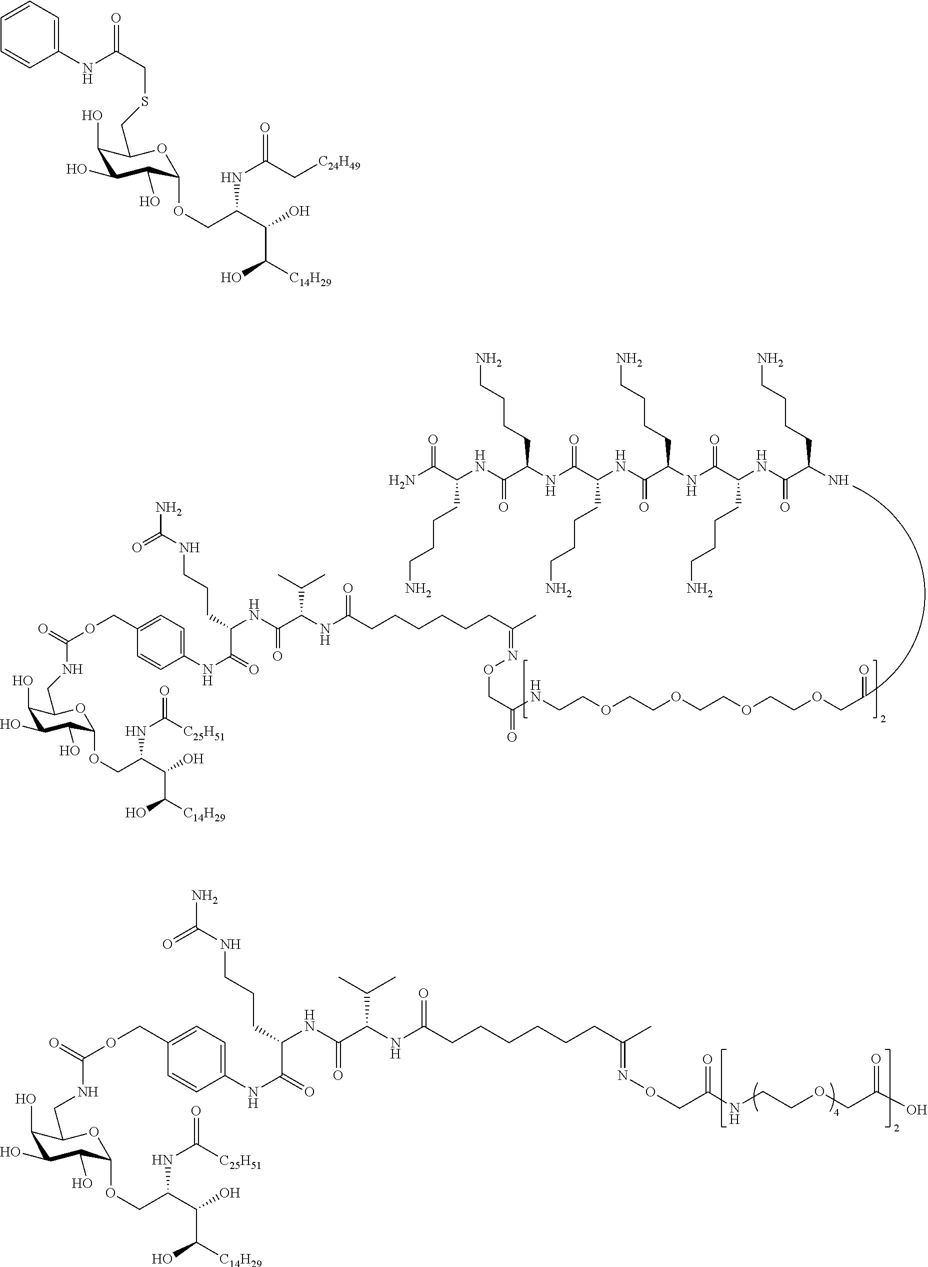

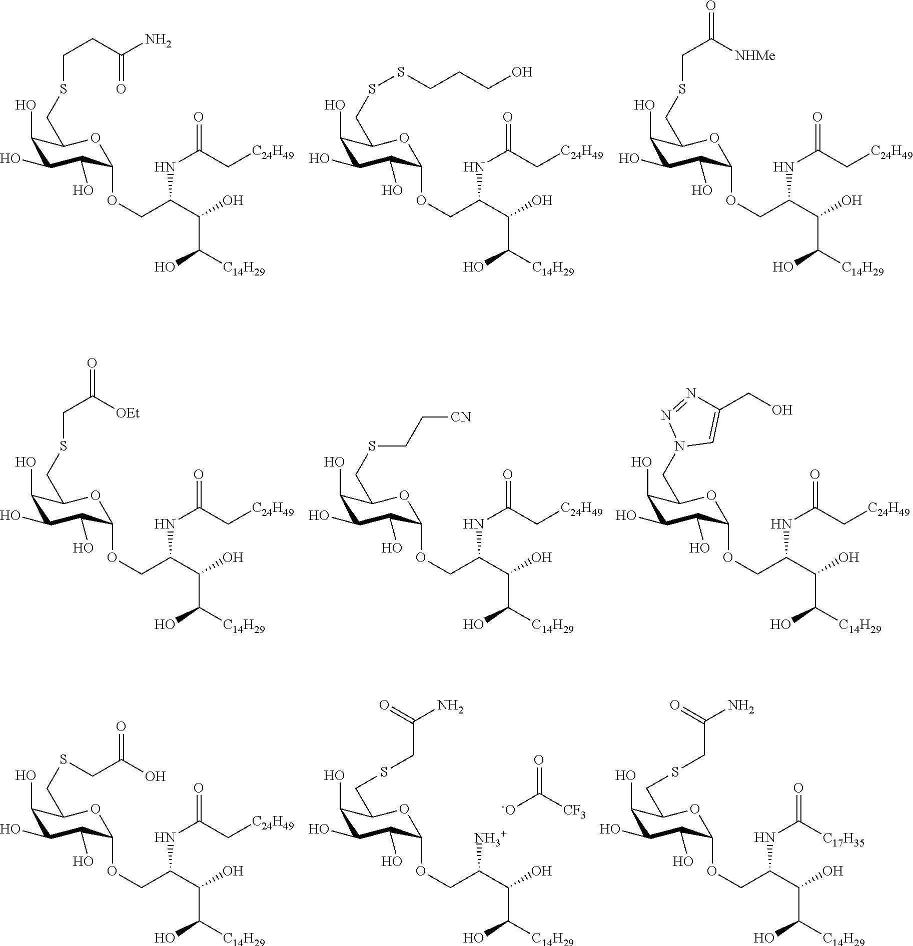

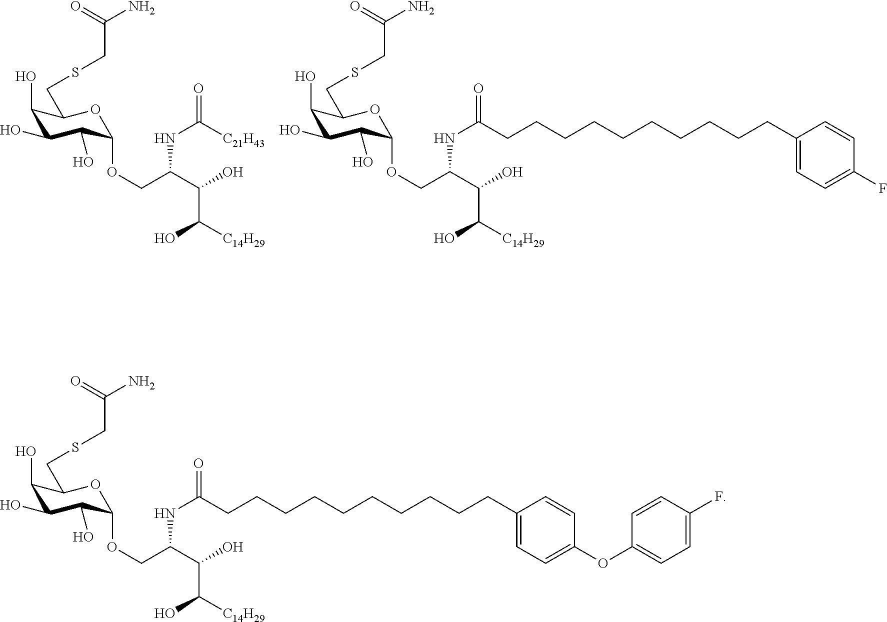

102. A pharmaceutical composition comprising a compound of claim 101 and a pharmaceutically acceptable excipient, diluent, or carrier.

103. A method of treating an infection in a subject, the method comprising administering to the subject a composition that comprises a first agent that comprises an iNKT cell agonist that activates or induces the production of tissue-resident memory T cells (T_{RM}), together with a second agent comprising an immune stimulator that stimulates or otherwise enhances an immune response to a target antigen in a subject or a polynucleotide sequence encoding an immune stimulator that stimulates or otherwise enhances an immune response to the target antigen in a subject, to thereby treat the infection in the subject.

104. A method of treating a cancer or tumour in a subject, the method comprising administering to the subject a composition that comprises a first agent that comprises an iNKT cell agonist that activates or induces the production of tissue-resident memory T cells (T_{RM}), together with a second agent comprising an immune stimulator that stimulates or otherwise enhances an immune response to a target antigen in a subject or a polynucleotide sequence encoding an immune stimulator that stimulates or otherwise enhances an immune response to the target antigen in a subject, to thereby treat the cancer or tumour in the subject.

105. A method of enriching the number of T_{RM} cells in the liver of a subject, the method comprising administering to the subject a composition that comprises a first agent that comprises an iNKT cell agonist that activates or induces the production of tissue-resident memory T cells (T_{RM}), together with a second agent comprising an immune stimulator that stimulates or otherwise enhances an immune response to a target antigen in a subject or a polynucleotide sequence encoding an immune stimulator that stimulates or otherwise enhances an immune response to the target antigen in a subject, to thereby enhance or stimulate the number of T_{RM} cells in the liver of the subject.

106. The method of any one of claims 103 to 105, wherein the iNKT cell agonist does not activate or induce the production of T_{RM} cells in the spleen.

107. The method of any one of claims 103 to 106, wherein the immune stimulator is a CD8⁺ T cell epitope.

108. The method of any one of claims 103 to 107, wherein the polynucleotide is a ribonucleic acid (RNA) polynucleotide (e.g., an mRNA).

109. The method of any one of claims 103 to 109, wherein the first agent modifies a phenotype of an iNKT cell.

110. The method of claim 109, wherein the modified phenotype comprises a downregulation of TCR expression.

111. The method of claim 109 or claim 110, wherein the modified phenotype comprises an enhanced expression of PD1 on the surface of the iNKT cell.

112. The method of any one of claims 103 to 111, wherein upon administration to a subject the ratio of T_{RM} cells to central memory T (T_{CM}) cells and/or effector memory (T_{EM}) cells present in the liver is increased.

113. The method of any one of claims 103 to 112, wherein the ratio of T_{RM} cells to T_{CM} and T_{E}M cells (i.e., T_{R}M cells:T_{CM}+T_{EM} cells) is greater than about 1:1.

114. The method of claim 112 or claim 113, wherein the ratio of T_{RM} cells to T_{CM} and T_{E}M cells (i.e., T_{RM} cells:T_{CM}+T_{EM} cells) is greater than about 3:2.

115. The method of any one of claims 112 to 114, wherein the ratio of T_{RM} cells to T_{CM} and T_{E}M cells (i.e., T_{RM} cells:T_{CM}+T_{EM} cells) is greater than about 2.3:1

116. The method of claim 112, wherein at least 30%, 35%, 40%, 45%, 50%, 55%, 60%, 65%, 66%, 70%, 75% of the memory T cell population in the liver are T_{R}M cells.

117. The method of claim 116, wherein greater than about 70% of the memory T cell population in the liver are T_{RM} cells.

118. The method of any one of claims 103 to 117, wherein the iNKT cell agonist is a derivative of α-galactosylceramide, wherein the derivation is at the 6 position of the galactose ring.

119. The method of any one of claims 103 to 118, wherein the iNKT cell agonist comprises a Compound of Formula (I): 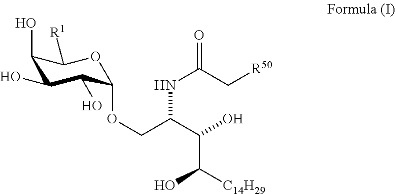
or a pharmaceutically acceptable salt thereof, wherein:
R¹ is selected from hydrogen, halo, alkyl, alkenyl, alkynyl, aryl, acyl, carbocyclyl, heterocyclyl, heteroaryl, alkyloxy, alkenyloxy, alkynyloxy, aryloxy, acyloxy, carbocyclyloxy, heterocyclyloxy, heteroaryloxy, alkylthio, alkenylthio, alkynylthio, arylthio, acylthio, carbocyclylthio, heterocyclylthio, heteroarylthio, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkyloxyacylalkyl, alkylcarbocyclyloxy, alkylheterocyclyloxy, alkylheteroaryloxy, alkylthioalkyl, alkenylthioalkyl, alkynylthioalkyl, alkenylthioalkenyl, alkenylthioalkynyl, alkynylthioalkynyl, arylthioalkyl, alkylacylthio, alkylcarbocyclylthio, alkylheterocyclylalkyl, alkenylheterocyclylalkyl, alkynylheterocyclylalkyl, alkenylheterocyclylalkenyl, alkenylheterocyclylalkynyl, alkynylheterocyclylalkynyl, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, arylacyloxy, arylcarbocyclyloxy, arylheterocyclyloxy, arylheteroaryloxy, alkylthioaryl, alkenylthioaryl, alkynylthioaryl, arylthioaryl, arylacylthio, arylcarbocyclylthio, arylheterocyclylthio, arylheteroarylthio, alkylamino, alkenylamino, alkynylamino, arylamino, acylamino, carbocyclylamino, heterocyclylamino, heteroarylamino, alkylaminoalkyl, alkenylaminoalkyl, alkynylaminoalkyl, alkenylaminoalkenyl, alkenylaminoalkynyl, alkynylaminoalkynyl, arylaminoalkyl, alkylacylamino, alkylcarbocyclylamino, alkylheterocyclylamino, alkylheteroarylamino, alkylaminoaryl, alkenylaminoaryl, alkynylaminoaryl, arylaminoaryl, arylacylamino, arylcarbocyclylamino, arylheterocyclylamino, arylheteroarylamino, alkylamido, alkenylamido, alkynylamido, arylamido, acylamido, carbocyclylamido, heterocyclylamido, heteroarylamido, alkylamidoalkyl, alkenylamidoalkyl, alkynylamidoalkyl, alkenylamidoalkenyl, alkenylamidoalkynyl, alkynylamidoalkynyl, arylamidoalkyl, alkylacylamido, alkylcarbocyclylamido, alkylheterocyclylamido, alkylheteroarylamido, alkylamidoaryl, alkenylamidoaryl, alkynylamidoaryl, arylamidoaryl, arylacylamido, arylcarbocyclylamido, arylheterocyclylamido, arylheteroarylamido, alkylcarbamyl, alkenylcarbamyl, alkynylcarbamyl, arylcarbamyl, acylcarbamyl, carbocyclylcarbamyl, heterocyclylcarbamyl, heteroarylcarbamyl, alkylcarbamylalkyl, alkenylcarbamylalkyl, alkynylcarbamylalkyl, alkenylcarbamylalkenyl, alkenylcarbamylalkynyl, alkynylcarbamylalkynyl, arylcarbamylalkyl, alkylacylcarbamyl, alkylcarbocyclylcarbamyl, alkylheterocyclylcarbamyl, alkylheteroarylcarbamyl, alkylcarbamylaryl, alkenylcarbamylaryl, alkynylcarbamylaryl, arylcarbamylaryl, arylacylcarbamyl, arylcarbocyclylcarbamyl, arylheterocyclylcarbamyl, arylheteroarylcarbamyl, alkyldisulfidyl, alkenyldisulfidyl, alkynyldisulfidyl, aryldisulfidyl, acyldisulfidyl, carbocyclyldisulfidyl, heterocyclyldisulfidyl, heteroaryldisulfidyl, alkyldisulfidylalkyl, alkenyldisulfidylalkyl, alkynyldisulfidylalkyl, alkenyldisulfidylalkenyl, alkenyldisulfidylalkynyl, alkynyldisulfidylalkynyl, aryldisulfidylalkyl, alkylacyldisulfidyl, alkylcarbocyclyldisulfidyl, alkylheterocyclyldisulfidyl, alkylheteroaryldisulfidyl, alkyldisulfidylaryl, alkenyldisulfidylaryl, alkynyldisulfidylaryl, aryldisulfidylaryl, arylacyldisulfidyl, arylcarbocyclyldisulfidyl, arylheterocyclyldisulfidyl, and arylheteroaryldisulfidyl, and wherein R¹ is optionally substituted;
and wherein R′ is not —CH₂OH;
R⁵⁰ is selected from optionally substituted alkyl, alkenyl, alkynyl.

120. The method of claim 119, wherein R′ is selected from C₁-C₁₈ alkyl, C₂-C₁₈ alkenyl, C₂-C₁₈ alkynyl, C₆-C₁₈ aryl, C₁-C₁₈ acyl, C₃-C₁₈ carbocyclyl, C₂-C₁₈ heterocyclyl, C₃-C₁₈ heteroaryl, C₁-C₁₈ alkyloxy, C₂-C₁₈ alkenyloxy, C₂-C₁₈ alkynyloxy, C₆-C₁₈ aryloxy, C₁-C₁₈ acyloxy, C₃-C₁₈ carbocyclyloxy, C₂-C₁₈ heterocyclyloxy, C₃-C₁₈ heteroaryloxy, C₁-C₁₈ alkylthio, C₂-C₁₈ alkenylthio, C₂-C₁₈ alkynylthio, C₆-C₁₈ arylthio, C₁-C₁₈ acylthio, C₃-C₁₈ carbocyclylthio, C₂-C₁₈ heterocyclylthio, C₃-C₁₈ heteroarylthio, C₃-C₁₈ alkylalkenyl, C₃-C₁₈ alkylalkynyl C₇-C₂₄ alkylaryl, C₂-C₁₈ alkylacyl, C₄-C₁₈ alkylcarbocyclyl, C₃-C₁₈ alkylheterocyclyl, C₄-C₁₈ alkylheteroaryl, C₂-C₁₈ alkyloxyalkyl, C₃-C₁₈ alkenyloxyalkyl, C₃-C₁₈ alkynyloxyalkyl, C₇-C₂₄ aryloxyalkyl, C₂-C₁₈ alkylacyloxy, C₂₋₁₈ alkyloxyacyl C₂₋₁₈ alkyl, C₄-C₁₈ alkylcarbocyclyloxy, C₃-C₁₈ alkylheterocyclyloxy, C₄-C₁₈ alkylheteroaryloxy, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄—C₁₈ alkynylthioalkynyl, C₇-C₂₄ arylthioalkyl, C₂-C₁₈ alkylacylthio, C₄-C₁₉ alkylcarbocyclylthio, C₄-C₁₈ alkylheterocyclylalkyl, C₄-C₁₈ alkenylheterocyclylalkyl, C₄-C₁₈ alkynylheterocyclylalkyl, C₅-C₁₈ alkenylheterocyclylalkenyl, C₅-C₁₈ alkenylheterocyclylalkynyl, C₅-C₁₈ alkynylheterocyclylalkynyl, C₃-C₁₈ alkylheterocyclylthio, C₄-C₁₈ alkylheteroarylthio, C₄-C₁₈ alkylalkenylalkyl, C₄-C₁₈ alkylalkynylalkyl, C₈-C₂₄ alkylarylalkyl, C₃-C₁₈ alkylacylalkyl, C₁₃-C₂₄ arylalkylaryl, C₁₄-C₂₄ arylalkenylaryl, C₁₄-C₂₄ arylalkynylaryl, C₁₃-C₂₄ arylacylaryl, C₇-C₁₈ arylacyl, C₉-C₁₈ arylcarbocyclyl, C₈-C₁₈ arylheterocyclyl, C₉-C₁₈ arylheteroaryl, C₈-C₁₈ alkenyloxyaryl, C₈-C₁₈ alkynyloxyaryl, C₁₂-C₂₄ aryloxyaryl, C₇-C₁₈ arylacyloxy, C₉-C₁₈ arylcarbocyclyloxy, C₈-C₁₈ arylheterocyclyloxy, C₉-C₁₈ arylheteroaryloxy, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylthio, C₂-C₁₈ alkenylthio, C₂-C₁₈ alkynylthio, C₅-C₁₈ arylthio, C₁-C₁₈ acylthio, C₃-C₁₈ carbocyclylthio, C₂-C₁₈ heterocyclylthio, C₃-C₁₈ heteroarylthio, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₃-C₁₈ alkylheterocyclylthio, C₄-C₁₈ alkylheteroarylthio, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylamino, C₂-C₁₈ alkenylamino, C₂-C₁₈ alkynylamino, C₆-C₁₈ arylamino, C₁-C₁₈ acylamino, C₃-C₁₈ carbocyclylamino, C₂-C₁₈ heterocyclylamino, C₃-C₁₈ heteroarylamino, C₂-C₁₈ alkylaminoalkyl, C₃-C₁₈ alkenylaminoalkyl, C₃-C₁₈ alkynylaminoalkyl, C₄-C₁₈ alkenylaminoalkenyl, C₄-C₁₈ alkenylaminoalkynyl, C₄-C₁₈ alkynylaminoalkynyl, C₇-C₂₄ arylaminoalkyl, C₂-C₁₈ alkylacylamino, C₄-C₁₈ alkylcarbocyclylamino, C₃-C₁₈ alkylheterocyclylamino, C₄-C₁₈ alkylheteroarylamino, C₈-C₁₈ alkylaminoaryl, C₈-C₁₈ alkenylaminoaryl, C₈-C₁₈ alkynylaminoaryl, C₁₂-C₂₄ arylaminoaryl, C₇-C₁₈ arylacylamino, C₉-C₁₈ arylcarbocyclylamino, C₈-C₁₈ arylheterocyclylamino, C₉-C₁₈ arylheteroarylamino, C₁-C₁₈ alkylamido, C₂-C₁₈ alkenylamido, C₂-C₁₈ alkynylamido, C₆-C₁₈ arylamido, C₁-C₁₈ acylamido, C₃-C₁₈ carbocyclylamido, C₂-C₁₈ heterocyclylamido, C₃-C₁₈ heteroarylamido, C₂-C₁₈ alkylamidoalkyl, C₃-C₁₈ alkenylamidoalkyl, C₃-C₁₈ alkynylamidoalkyl, C₄-C₁₈ alkenylamidoalkenyl, C₄-C₁₈ alkenylamidoalkynyl, C₄-C₁₈ alkynylamidoalkynyl, C₇-C₂₄ arylamidoalkyl, C₂-C₁₈ alkylacylamido, C₄-C₁₈ alkylcarbocyclylamido, C₃-C₁₈ alkylheterocyclylamido, C₄-C₁₈ alkylheteroarylamido, C₈-C₁₈ alkylamidoaryl, C₈-C₁₈ alkenylamidoaryl, C₈-C₁₈ alkynylamidoaryl, C₁₂-C₂₄ arylamidoaryl, C₇-C₁₈ arylacylamido, C₈-C₁₈ arylcarbocyclylamido, C₈-C₁₈ arylheterocyclylamido, C₉-C₁₈ arylheteroarylamido, C₁-C₁₈ alkylcarbamyl, C₂-C₁₈ alkenylcarbamyl, C₂-C₁₈ alkynylcarbamyl, C₈-C₁₈ arylcarbamyl, C₁-C₁₈ acylcarbamyl, C₃-C₁₈ carbocyclylcarbamyl, C₂-C₁₈ heterocyclylcarbamyl, C₃-C₁₈ heteroarylcarbamyl, C₂-C₁₈ alkylcarbamylalkyl, C₃-C₁₈ alkenylcarbamylalkyl, C₃-C₁₈ alkynylcarbamylalkyl, C₄-C₁₈ alkenylcarbamylalkenyl, C₄-C₁₈ alkenylcarbamylalkynyl, C₄-C₁₈ alkynylcarbamylalkynyl, C₇-C₂₄ arylcarbamylalkyl, C₂-C₁₈ alkylacylcarbamyl, C₄-C₁₈ alkylcarbocyclylcarbamyl, C₃-C₁₈ alkylheterocyclylcarbamyl, C₄-C₁₈ alkylheteroarylcarbamyl, C₈-C₁₈ alkylcarbamylaryl, C₅-C₁₈ alkenylcarbamylaryl, C₈-C₁₈ alkynylcarbamylaryl, C₁₂-C₂₄ arylcarbamylaryl, C₇-C₁₈ arylacylcarbamyl, C₉-C₁₈ arylcarbocyclylcarbamyl, C₈-C₁₈ arylheterocyclylcarbamyl, C₉-C₁₈ arylheteroarylcarbamyl, C₁-C₁₈ alkyldisulfidyl, C₂-C₁₈ alkenyldisulfidyl, C₂-C₁₈ alkynyldisulfidyl, C₆-C₁₈ aryldisulfidyl, C₁-C₁₈ acyldisulfidyl, C₃-C₁₈ carbocyclyldisulfidyl, C₂-C₁₈ heterocyclyldisulfidyl, C₃-C₁₈ heteroaryldisulfidyl, C₂—C₁₈ alkyldisulfidylalkyl, C₃-C₁₈ alkenyldisulfidylalkyl, C₃-C₁₈ alkynyldisulfidylalkyl, C₄-C₁₈ alkenyldisulfidylalkenyl, C₄-C₁₈ alkenyldisulfidylalkynyl, C₄-C₁₈ alkynyldisulfidylalkynyl, C₇-C₂₄ aryldisulfidylalkyl, C₂-C₁₈ alkylacyldisulfidyl, C₄-C₁₈ alkylcarbocyclyldisulfidyl, C₃-C₁₈ alkylheterocyclyldisulfidyl, C₄-C₁₈ alkylheteroaryldisulfidyl, C₈-C₁₈ alkyldisulfidylaryl, C₈-C₁₈ alkenyldisulfidylaryl, C₈-C₁₈ alkynyldisulfidylaryl, C₁₂-C₂₄ aryldisulfidylaryl, C₇-C₁₈ arylacyldisulfidyl, C₉-C₁₈ arylcarbocyclyldisulfidyl, C₈-C₁₈ arylheterocyclyldisulfidyl, and C₉-C₁₈ arylheteroaryldisulfidyl, and wherein R′ is optionally substituted;
and wherein R′ is not —CH₂OH.

121. The method of claim 14 or claim 15, wherein R′ is selected from alkyl (e.g., C₁-C₁₈, C₁-C₆, C₁-C₅, C₈-C₁₈, or C₉-C₁₈), aryl (e.g., C₈-C₁₈), heteroaryl (e.g., C₃-C₁₈), carbocyclyl (e.g., C₃-C₁₈), heterocyclyl (e.g., C₂-C₁₈), alkylaryl (e.g., C₇-C₂₄), alkylheteroaryl (e.g., C₄-C₁₈), alkylcarbocyclyl (e.g., C₄-C₁₈), alkylthioalkyl (e.g., wherein each alkyl is C₁-C₁₈), arylthioalkyl (e.g., wherein each alkyl is C₇-C₁₈), alkylaminoalkyl (e.g., wherein each alkyl is C₁-C₁₈), alkylamidoalkyl (e.g., wherein each alkyl is C₁-C₁₈), alkylheterocyclylalkyl (e.g., C₃-C₁₈), alkylthio (e.g., C₁-C₁₈; such as thiomethyl) and alkylheterocyclyl (e.g., C₃-C₁₈);
and wherein R¹ is not —CH₂OH.

122. The method of any one of claims 103 to 121, wherein the iNKT agonist comprises a Compound of Formula (II): 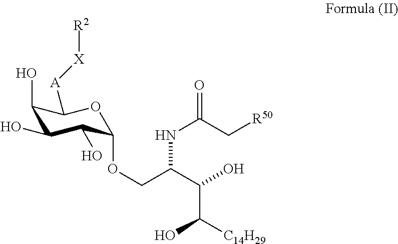
or a pharmaceutically acceptable salt thereof, wherein:
A is selected from alkyl, alkenyl, and alkynyl;
X is selected from —CR¹⁰R¹¹—, —O—, —S—, —NR^{a}—, —NR^{a}C(O)—, —NR^{a}C(O)O—, —S—S—, heterocyclyl, heteroaryl, wherein R¹⁰ and R″ where present are independently selected from H, halo, C₁₋₂alkyl, and wherein R^{a} may be selected from hydrogen, alkyl, alkenyl, alkynyl, aryl, carbocyclyl, heteroaryl, heterocyclyl, arylalkyl, and acyl;
R² is selected from hydrogen, halo, alkyl, alkenyl, alkynyl, aryl, acyl, carbocyclyl, heterocyclyl, heteroaryl, alkyloxy, alkenyloxy, alkynyloxy, aryloxy, acyloxy, carbocyclyloxy, heterocyclyloxy, heteroaryloxy, alkylthio, alkenylthio, alkynylthio, arylthio, acylthio, carbocyclylthio, heterocyclylthio, heteroarylthio, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkyloxyacylalkyl, alkylcarbocyclyloxy, alkylheterocyclyloxy, alkylheteroaryloxy, alkylthioalkyl, alkenylthioalkyl, alkynylthioalkyl, alkenylthioalkenyl, alkenylthioalkynyl, alkynylthioalkynyl, arylthioalkyl, alkylacylthio, alkylcarbocyclylthio, alkylheterocyclylalkyl, alkenylheterocyclylalkyl, alkynylheterocyclylalkyl, alkenylheterocyclylalkenyl, alkenylheterocyclylalkynyl, alkynylheterocyclylalkynyl, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, arylacyloxy, arylcarbocyclyloxy, arylheterocyclyloxy, arylheteroaryloxy, alkylthioaryl, alkenylthioaryl, alkynylthioaryl, arylthioaryl, arylacylthio, arylcarbocyclylthio, arylheterocyclylthio, arylheteroarylthio, alkylamino, alkenylamino, alkynylamino, arylamino, acylamino, carbocyclylamino, heterocyclylamino, heteroarylamino, alkylaminoalkyl, alkenylaminoalkyl, alkynylaminoalkyl, alkenylaminoalkenyl, alkenylaminoalkynyl, alkynylaminoalkynyl, arylaminoalkyl, alkylacylamino, alkylcarbocyclylamino, alkylheterocyclylamino, alkylheteroarylamino, alkylaminoaryl, alkenylaminoaryl, alkynylaminoaryl, arylaminoaryl, arylacylamino, arylcarbocyclylamino, arylheterocyclylamino, arylheteroarylamino, alkylamido, alkenylamido, alkynylamido, arylamido, acylamido, carbocyclylamido, heterocyclylamido, heteroarylamido, alkylamidoalkyl, alkenylamidoalkyl, alkynylamidoalkyl, alkenylamidoalkenyl, alkenylamidoalkynyl, alkynylamidoalkynyl, arylamidoalkyl, alkylacylamido, alkylcarbocyclylamido, alkylheterocyclylamido, alkylheteroarylamido, alkylamidoaryl, alkenylamidoaryl, alkynylamidoaryl, arylamidoaryl, arylacylamido, arylcarbocyclylamido, arylheterocyclylamido, arylheteroarylamido, alkylcarbamyl, alkenylcarbamyl, alkynylcarbamyl, arylcarbamyl, acylcarbamyl, carbocyclylcarbamyl, heterocyclylcarbamyl, heteroarylcarbamyl, alkylcarbamylalkyl, alkenylcarbamylalkyl, alkynylcarbamylalkyl, alkenylcarbamylalkenyl, alkenylcarbamylalkynyl, alkynylcarbamylalkynyl, arylcarbamylalkyl, alkylacylcarbamyl, alkylcarbocyclylcarbamyl, alkylheterocyclylcarbamyl, alkylheteroarylcarbamyl, alkylcarbamylaryl, alkenylcarbamylaryl, alkynylcarbamylaryl, arylcarbamylaryl, arylacylcarbamyl, arylcarbocyclylcarbamyl, arylheterocyclylcarbamyl, arylheteroarylcarbamyl, alkyldisulfidyl, alkenyldisulfidyl, alkynyldisulfidyl, aryldisulfidyl, acyldisulfidyl, carbocyclyldisulfidyl, heterocyclyldisulfidyl, heteroaryldisulfidyl, alkyldisulfidylalkyl, alkenyldisulfidylalkyl, alkynyldisulfidylalkyl, alkenyldisulfidylalkenyl, alkenyldisulfidylalkynyl, alkynyldisulfidylalkynyl, aryldisulfidylalkyl, alkylacyldisulfidyl, alkylcarbocyclyldisulfidyl, alkylheterocyclyldisulfidyl, alkylheteroaryldisulfidyl, alkyldisulfidylaryl, alkenyldisulfidylaryl, alkynyldisulfidylaryl, aryldisulfidylaryl, arylacyldisulfidyl, arylcarbocyclyldisulfidyl, arylheterocyclyldisulfidyl, and arylheteroaryldisulfidyl, and wherein R² is optionally substituted;
and wherein -A-X—R² is not —CH₂OH;
R⁵⁰ is selected from optionally substituted alkyl, alkenyl, alkynyl.

123. The method of claim 122, wherein R^{a} is selected from hydrogen, C₁-C₈ alkyl, C₂-C₈ alkenyl, C₂-C₈ alkynyl, C₆-C₁₈ aryl, C₃-C₁₈ carbocyclyl, C₃-C₁₈ heteroaryl, C₂-C₁₉ heterocyclyl, C₇-C₂₄ arylalkyl, and C₁-C₈ acyl.

124. The method of claim 122 or claim 123, wherein X is S or —NR^{a}—, and wherein R^{a} is selected from hydrogen, alkyl, alkenyl, alkynyl, aryl, carbocyclyl, heteroaryl, heterocyclyl, arylalkyl, and acyl.

125. The method of claim 124, wherein R^{a} is selected from hydrogen, C₁-C₈ alkyl, C₂-C₈ alkenyl, C₂-C₈ alkynyl, C₆-C₁₈ aryl, C₃-C₁₈ carbocyclyl, C₃-C₁₈ heteroaryl, C₂-C₁₈ heterocyclyl, C₇-C₂₄ arylalkyl, and C₁-C₈ acyl.

126. The method of any one of claims 119 to 125, wherein A is —CR¹²R¹³— and R¹² and R¹³ are independently selected from H, halo, C₁₋₂alkyl.

127. The method of any one of claims 119 to 126, wherein A is —CH₂—.

128. The method of any one of claims 119 to 127, wherein R² is selected from C₁-C₁₈ alkyl, C₂-C₁₈ alkenyl, C₂-C₁₈ alkynyl, C₆-C₁₅ aryl, C₁-C₁₈ acyl, C₃-C₁₈ carbocyclyl, C₂-C₁₈ heterocyclyl, C₃-C₁₈ heteroaryl, C₁-C₁₈ alkyloxy, C₂-C₁₈ alkenyloxy, C₂-C₁₈ alkynyloxy, C₆-C₁₈ aryloxy, C₁-C₁₈ acyloxy, C₃-C₁₈ carbocyclyloxy, C₂-C₁₈ heterocyclyloxy, C₃-C₁₈ heteroaryloxy, C₁-C₁₈ alkylthio, C₂-C₁₈ alkenylthio, C₂-C₁₈ alkynylthio, C₈-C₁₈ arylthio, C₁-C₁₈ acylthio, C₃-C₁₈ carbocyclylthio, C₂-C₁₈ heterocyclylthio, C₃-C₁₈ heteroarylthio, C₃-C₁₈ alkylalkenyl, C₃-C₁₈ alkylalkynyl C₇-C₂₄ alkylaryl, C₂-C₁₈ alkylacyl, C₄-C₁₈ alkylcarbocyclyl, C₃-C₁₈ alkylheterocyclyl, C₄-C₁₈ alkylheteroaryl, C₂-C₁₈ alkyloxyalkyl, C₃-C₁₈ alkenyloxyalkyl, C₃-C₁₈ alkynyloxyalkyl, C₇-C₂₄ aryloxyalkyl, C₂-C₁₈ alkylacyloxy, C₂₋₁₈ alkyloxyacyl C₂₋₁₈ alkyl, C₄-C₁₈ alkylcarbocyclyloxy, C₃-C₁₈ alkylheterocyclyloxy, C₄-C₁₈ alkylheteroaryloxy, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₄-C₁₈ alkylheterocyclylalkyl, C₄-C₁₈ alkenylheterocyclylalkyl, C₄-C₁₈ alkynylheterocyclylalkyl, C₅-C₁₈ alkenylheterocyclylalkenyl, C₅-C₁₈ alkenylheterocyclylalkynyl, C₅-C₁₈ alkynylheterocyclylalkynyl, C₃-C₁₈ alkylheterocyclylthio, C₄-C₁₈ alkylheteroarylthio, C₄-C₁₈ alkylalkenylalkyl, C₄-C₁₈ alkylalkynylalkyl, C₈-C₂₄ alkylarylalkyl, C₈-C₁₈ alkylacylalkyl, C₁₃-C₂₄ arylalkylaryl, C₁₄-C₂₄ arylalkenylaryl, C₁₄-C₂₄ arylalkynylaryl, C₁₃-C₂₄ arylacylaryl, C₇-C₁₈ arylacyl, C₉-C₁₈ arylcarbocyclyl, C₈-C₁₈ arylheterocyclyl, C₉-C₁₈ arylheteroaryl, C₈-C₁₈ alkenyloxyaryl, C₈-C₁₈ alkynyloxyaryl, C₁₂-C₂₄ aryloxyaryl, C₇-C₁₈ arylacyloxy, C₉-C₁₈ arylcarbocyclyloxy, C₈-C₁₈ arylheterocyclyloxy, C₉-C₁₈ arylheteroaryloxy, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylthio, C₂-C₁₈ alkenylthio, C₂-C₁₈ alkynylthio, C₅-C₁₈ arylthio, C₁-C₁₈ acylthio, C₃-C₁₈ carbocyclylthio, C₂-C₁₈ heterocyclylthio, C₃-C₁₈ heteroarylthio, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₃-C₁₈ alkylheterocyclylthio, C₄-C₁₈ alkylheteroarylthio, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylamino, C₂-C₁₈ alkenylamino, C₂-C₁₈ alkynylamino, C₆-C₁₈ arylamino, C₁-C₁₈ acylamino, C₃-C₁₈ carbocyclylamino, C₂-C₁₈ heterocyclylamino, C₃-C₁₈ heteroarylamino, C₂-C₁₈ alkylaminoalkyl, C₃-C₁₈ alkenylaminoalkyl, C₃-C₁₈ alkynylaminoalkyl, C₄-C₁₈ alkenylaminoalkenyl, C₄-C₁₈ alkenylaminoalkynyl, C₄-C₁₈ alkynylaminoalkynyl, C₇-C₂₄ arylaminoalkyl, C₂-C₁₈ alkylacylamino, C₄-C₁₈ alkylcarbocyclylamino, C₃-C₁₈ alkylheterocyclylamino, C₄-C₁₈ alkylheteroarylamino, C₈-C₁₈ alkylaminoaryl, C₈-C₁₈ alkenylaminoaryl, C₈-C₁₈ alkynylaminoaryl, C₁₂-C₂₄ arylaminoaryl, C₇-C₁₈ arylacylamino, C₉-C₁₈ arylcarbocyclylamino, C₈-C₁₈ arylheterocyclylamino, C₉-C₁₈ arylheteroarylamino, C₁-C₁₈ alkylamido, C₂-C₁₈ alkenylamido, C₂-C₁₈ alkynylamido, C₆—C₁₈ arylamido, C₁-C₁₈ acylamido, C₃-C₁₈ carbocyclylamido, C₂-C₁₈ heterocyclylamido, C₃-C₁₈ heteroarylamido, C₂-C₁₈ alkylamidoalkyl, C₃-C₁₈ alkenylamidoalkyl, C₃-C₁₈ alkynylamidoalkyl, C₄-C₁₈ alkenylamidoalkenyl, C₄-C₁₈ alkenylamidoalkynyl, C₄-C₁₈ alkynylamidoalkynyl, C₇-C₂₄ arylamidoalkyl, C₂-C₁₈ alkylacylamido, C₄-C₁₈ alkylcarbocyclylamido, C₃-C₁₈ alkylheterocyclylamido, C₄-C₁₈ alkylheteroarylamido, C₈-C₁₈ alkylamidoaryl, C₈-C₁₈ alkenylamidoaryl, C₈-C₁₈ alkynylamidoaryl, C₁₂-C₂₄ arylamidoaryl, C₇-C₁₈ arylacylamido, C₉-C₁₈ arylcarbocyclylamido, C₈-C₁₈ arylheterocyclylamido, C₉-C₁₈ arylheteroarylamido, C₁-C₁₈ alkylcarbamyl, C₂-C₁₈ alkenylcarbamyl, C₂-C₁₈ alkynylcarbamyl, C₅-C₁₈ arylcarbamyl, C₁-C₁₈ acylcarbamyl, C₃-C₁₈ carbocyclylcarbamyl, C₂-C₁₈ heterocyclylcarbamyl, C₃-C₁₈ heteroarylcarbamyl, C₂-C₁₈ alkylcarbamylalkyl, C₃-C₁₈ alkenylcarbamylalkyl, C₃-C₁₈ alkynylcarbamylalkyl, C₄-C₁₈ alkenylcarbamylalkenyl, C₄-C₁₈ alkenylcarbamylalkynyl, C₄-C₁₈ alkynylcarbamylalkynyl, C₇-C₂₄ arylcarbamylalkyl, C₂-C₁₈ alkylacylcarbamyl, C₄-C₁₈ alkylcarbocyclylcarbamyl, C₃-C₁₈ alkylheterocyclylcarbamyl, C₄-C₁₈ alkylheteroarylcarbamyl, C₈-C₁₈ alkylcarbamylaryl, C₈-C₁₈ alkenylcarbamylaryl, C₈-C₁₈ alkynylcarbamylaryl, C₁₂-C₂₄ arylcarbamylaryl, C₇-C₁₈ arylacylcarbamyl, C₉-C₁₈ arylcarbocyclylcarbamyl, C₈-C₁₈ arylheterocyclylcarbamyl, C₉-C₁₈ arylheteroarylcarbamyl, C₁-C₁₈ alkyldisulfidyl, C₂-C₁₈ alkenyldisulfidyl, C₂-C₁₈ alkynyldisulfidyl, C₆-C₁₈ aryldisulfidyl, C₁-C₁₈ acyldisulfidyl, C₃-C₁₈ carbocyclyldisulfidyl, C₂-C₁₈ heterocyclyldisulfidyl, C₃-C₁₈ heteroaryldisulfidyl, C₂-C₁₈ alkyldisulfidylalkyl, C₃-C₁₈ alkenyldisulfidylalkyl, C₃-C₁₈ alkynyldisulfidylalkyl, C₄-C₁₈ alkenyldisulfidylalkenyl, C₄-C₁₈ alkenyldisulfidylalkynyl, C₄-C₁₈ alkynyldisulfidylalkynyl, C₇-C₂₄ aryldisulfidylalkyl, C₂-C₁₈ alkylacyldisulfidyl, C₄-C₁₈ alkylcarbocyclyldisulfidyl, C₃-C₁₈ alkylheterocyclyldisulfidyl, C₄-C₁₈ alkylheteroaryldisulfidyl, C₈-C₁₈ alkyldisulfidylaryl, C₈-C₁₈ alkenyldisulfidylaryl, C₈-C₁₈ alkynyldisulfidylaryl, C₁₂-C₂₄ aryldisulfidylaryl, C₇-C₁₈ arylacyldisulfidyl, C₉-C₁₈ arylcarbocyclyldisulfidyl, C₈-C₁₈ arylheterocyclyldisulfidyl, and C₉-C₁₈ arylheteroaryldisulfidyl, and wherein R² is optionally substituted;
and wherein -A-X—R² is not —CH₂OH.

129. The method of any one of claims 119 to 128, wherein R² is selected from alkyl (e.g., C₁-C₁₈, C₁-C₆, C₁-C₈, C₈-C₁₈, or C₉-C₁₈), aryl (e.g., C₆-C₁₈), heteroaryl (e.g., C₃-C₁₈), carbocyclyl (e.g., C₃-C₁₈), heterocyclyl (e.g., C₂-C₁₈), alkylaryl (e.g., C₇-C₂₄), alkylheteroaryl (e.g., C₄-C₁₈), alkylcarbocyclyl (e.g., C₄-C₁₈), alkylthioalkyl (e.g., wherein each alkyl is C₁-C₁₈), arylthioalkyl (e.g., wherein each alkyl is C₇-C₁₈), alkylaminoalkyl (e.g., wherein each alkyl is C₁-C₁₈), alkylamidoalkyl (e.g., wherein each alkyl is C₁-C₁₈), alkylheterocyclylalkyl (e.g., C₃-C₁₈), alkylthio (e.g., C₁-C₁₈;
such as thiomethyl) and alkylheterocyclyl (e.g., C₃-C₁₈).

130. The method of any one of claims 103 to 128, wherein the iNKT cell agonist is a Compound of Formula (III): 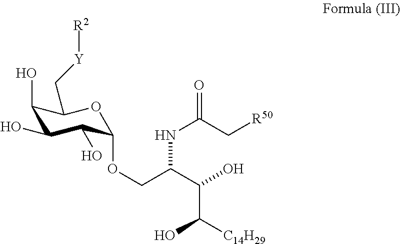
or a pharmaceutically acceptable salt thereof, wherein:
Y is selected from —S—, —NR^{a}—, —NR^{a}C(O)—, —NR^{a}C(O)O—, —S—S—, heterocyclyl, heteroaryl, wherein R^{a} may be selected from hydrogen, alkyl, alkenyl, alkynyl, aryl, carbocyclyl, heteroaryl, heterocyclyl, arylalkyl, and acyl;
R² is selected from hydrogen, halo, alkyl, alkenyl, alkynyl, aryl, acyl, carbocyclyl, heterocyclyl, heteroaryl, alkyloxy, alkenyloxy, alkynyloxy, aryloxy, acyloxy, carbocyclyloxy, heterocyclyloxy, heteroaryloxy, alkylthio, alkenylthio, alkynylthio, arylthio, acylthio, carbocyclylthio, heterocyclylthio, heteroarylthio, alkylalkenyl, alkylalkynyl, alkylaryl, alkylacyl, alkylcarbocyclyl, alkylheterocyclyl, alkylheteroaryl, alkyloxyalkyl, alkenyloxyalkyl, alkynyloxyalkyl, aryloxyalkyl, alkylacyloxy, alkyloxyacylalkyl, alkylcarbocyclyloxy, alkylheterocyclyloxy, alkylheteroaryloxy, alkylthioalkyl, alkenylthioalkyl, alkynylthioalkyl, alkenylthioalkenyl, alkenylthioalkynyl, alkynylthioalkynyl, arylthioalkyl, alkylacylthio, alkylcarbocyclylthio, alkylheterocyclylalkyl, alkenylheterocyclylalkyl, alkynylheterocyclylalkyl, alkenylheterocyclylalkenyl, alkenylheterocyclylalkynyl, alkynylheterocyclylalkynyl, alkylheterocyclylthio, alkylheteroarylthio, alkylalkenylalkyl, alkylalkynylalkyl, alkylarylalkyl, alkylacylalkyl, arylalkylaryl, arylalkenylaryl, arylalkynylaryl, arylacylaryl, arylacyl, arylcarbocyclyl, arylheterocyclyl, arylheteroaryl, alkenyloxyaryl, alkynyloxyaryl, aryloxyaryl, arylacyloxy, arylcarbocyclyloxy, arylheterocyclyloxy, arylheteroaryloxy, alkylthioaryl, alkenylthioaryl, alkynylthioaryl, arylthioaryl, arylacylthio, arylcarbocyclylthio, arylheterocyclylthio, arylheteroarylthio, alkylamino, alkenylamino, alkynylamino, arylamino, acylamino, carbocyclylamino, heterocyclylamino, heteroarylamino, alkylaminoalkyl, alkenylaminoalkyl, alkynylaminoalkyl, alkenylaminoalkenyl, alkenylaminoalkynyl, alkynylaminoalkynyl, arylaminoalkyl, alkylacylamino, alkylcarbocyclylamino, alkylheterocyclylamino, alkylheteroarylamino, alkylaminoaryl, alkenylaminoaryl, alkynylaminoaryl, arylaminoaryl, arylacylamino, arylcarbocyclylamino, arylheterocyclylamino, arylheteroarylamino, alkylamido, alkenylamido, alkynylamido, arylamido, acylamido, carbocyclylamido, heterocyclylamido, heteroarylamido, alkylamidoalkyl, alkenylamidoalkyl, alkynylamidoalkyl, alkenylamidoalkenyl, alkenylamidoalkynyl, alkynylamidoalkynyl, arylamidoalkyl, alkylacylamido, alkylcarbocyclylamido, alkylheterocyclylamido, alkylheteroarylamido, alkylamidoaryl, alkenylamidoaryl, alkynylamidoaryl, arylamidoaryl, arylacylamido, arylcarbocyclylamido, arylheterocyclylamido, arylheteroarylamido, alkylcarbamyl, alkenylcarbamyl, alkynylcarbamyl, arylcarbamyl, acylcarbamyl, carbocyclylcarbamyl, heterocyclylcarbamyl, heteroarylcarbamyl, alkylcarbamylalkyl, alkenylcarbamylalkyl, alkynylcarbamylalkyl, alkenylcarbamylalkenyl, alkenylcarbamylalkynyl, alkynylcarbamylalkynyl, arylcarbamylalkyl, alkylacylcarbamyl, alkylcarbocyclylcarbamyl, alkylheterocyclylcarbamyl, alkylheteroarylcarbamyl, alkylcarbamylaryl, alkenylcarbamylaryl, alkynylcarbamylaryl, arylcarbamylaryl, arylacylcarbamyl, arylcarbocyclylcarbamyl, arylheterocyclylcarbamyl, arylheteroarylcarbamyl, alkyldisulfidyl, alkenyldisulfidyl, alkynyldisulfidyl, aryldisulfidyl, acyldisulfidyl, carbocyclyldisulfidyl, heterocyclyldisulfidyl, heteroaryldisulfidyl, alkyldisulfidylalkyl, alkenyldisulfidylalkyl, alkynyldisulfidylalkyl, alkenyldisulfidylalkenyl, alkenyldisulfidylalkynyl, alkynyldisulfidylalkynyl, aryldisulfidylalkyl, alkylacyldisulfidyl, alkylcarbocyclyldisulfidyl, alkylheterocyclyldisulfidyl, alkylheteroaryldisulfidyl, alkyldisulfidylaryl, alkenyldisulfidylaryl, alkynyldisulfidylaryl, aryldisulfidylaryl, arylacyldisulfidyl, arylcarbocyclyldisulfidyl, arylheterocyclyldisulfidyl, and arylheteroaryldisulfidyl, and wherein R² is optionally substituted;
R⁵⁰ is selected from optionally substituted alkyl, alkenyl, alkynyl.

131. The method of claim 130, wherein R^{a} is hydrogen.

132. The method of claim 130 or claim 131, wherein Y is selected from —S—, —NH—, —NHC(O)—, —NHC(O)O—, and —S—S—, triazolyl.

133. The method of any one of claims 130 to 132, wherein A is —CR¹²R¹³—, wherein R¹² and R¹³ where present are independently selected from H, halo, C₁₋₂alkyl. More preferably A is —CH₂—.

134. The method of any one of claims 130 to 133, wherein R² is selected from C₁-C₁₈ alkyl, C₂-C₁₈ alkenyl, C₂-C₁₈ alkynyl, C₆-C₁₈ aryl, C₁-C₁₈ acyl, C₃-C₁₈ carbocyclyl, C₂-C₁₈ heterocyclyl, C₃-C₁₈ heteroaryl, C₁-C₁₈ alkyloxy, C₂-C₁₈ alkenyloxy, C₂-C₁₈ alkynyloxy, C₆-C₁₈ aryloxy, C₁-C₁₈ acyloxy, C₃-C₁₈ carbocyclyloxy, C₂-C₁₈ heterocyclyloxy, C₃-C₁₈ heteroaryloxy, C₁-C₁₈ alkylthio, C₂-C₁₈ alkenylthio, C₂-C₁₈ alkynylthio, C₆-C₁₈ arylthio, C₁-C₁₈ acylthio, C₃-C₁₈ carbocyclylthio, C₂-C₁₈ heterocyclylthio, C₃-C₁₈ heteroarylthio, C₃-C₁₈ alkylalkenyl, C₃-C₁₈ alkylalkynyl C₇-C₂₄ alkylaryl, C₂-C₁₈ alkylacyl, C₄-C₁₈ alkylcarbocyclyl, C₃-C₁₈ alkylheterocyclyl, C₄-C₁₈ alkylheteroaryl, C₂-C₁₈ alkyloxyalkyl, C₃-C₁₈ alkenyloxyalkyl, C₃-C₁₈ alkynyloxyalkyl, C₇-C₂₄ aryloxyalkyl, C₂-C₁₈ alkylacyloxy, C₂₋₁₈ alkyloxyacyl C₂₋₁₈ alkyl, C₄-C₁₈ alkylcarbocyclyloxy, C₃-C₁₈ alkylheterocyclyloxy, C₄-C₁₈ alkylheteroaryloxy, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₄-C₁₈ alkylheterocyclylalkyl, C₄-C₁₈ alkenylheterocyclylalkyl, C₄-C₁₈ alkynylheterocyclylalkyl, C₅-C₁₈ alkenylheterocyclylalkenyl, C₅-C₁₈ alkenylheterocyclylalkynyl, C₅-C₁₈ alkynylheterocyclylalkynyl, C₃-C₁₈ alkylheterocyclylthio, C₄-C₁₈ alkylheteroarylthio, C₄-C₁₈ alkylalkenylalkyl, C₄-C₁₈ alkylalkynylalkyl, C₈-C₂₄ alkylarylalkyl, C₃-C₁₈ alkylacylalkyl, C₁₃-C₂₄ arylalkylaryl, C₁₄-C₂₄ arylalkenylaryl, C₁₄-C₂₄ arylalkynylaryl, C₁₃-C₂₄ arylacylaryl, C₇-C₁₈ arylacyl, C₉-C₁₈ arylcarbocyclyl, C₈-C₁₈ arylheterocyclyl, C₉-C₁₈ arylheteroaryl, C₈-C₁₈ alkenyloxyaryl, C₈-C₁₈ alkynyloxyaryl, C₁₂-C₂₄ aryloxyaryl, C₇-C₁₈ arylacyloxy, C₉-C₁₈ arylcarbocyclyloxy, C₈-C₁₈ arylheterocyclyloxy, C₉-C₁₈ arylheteroaryloxy, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylthio, C₂-C₁₈ alkenylthio, C₂-C₁₈ alkynylthio, C₅-C₁₈ arylthio, C₁-C₁₈ acylthio, C₃-C₁₈ carbocyclylthio, C₂-C₁₈ heterocyclylthio, C₃-C₁₈ heteroarylthio, C₂-C₁₈ alkylthioalkyl, C₃-C₁₈ alkenylthioalkyl, C₃-C₁₈ alkynylthioalkyl, C₄-C₁₈ alkenylthioalkenyl, C₄-C₁₈ alkenylthioalkynyl, C₄-C₁₈ alkynylthioalkynyl, C₇-C₂₄ arylthioalkyl, C₂-C₁₈ alkylacylthio, C₄-C₁₈ alkylcarbocyclylthio, C₃-C₁₈ alkylheterocyclylthio, C₄-C₁₈ alkylheteroarylthio, C₈-C₁₈ alkylthioaryl, C₈-C₁₈ alkenylthioaryl, C₈-C₁₈ alkynylthioaryl, C₁₂-C₂₄ arylthioaryl, C₇-C₁₈ arylacylthio, C₉-C₁₈ arylcarbocyclylthio, C₈-C₁₈ arylheterocyclylthio, C₉-C₁₈ arylheteroarylthio, C₁-C₁₈ alkylamino, C₂-C₁₈ alkenylamino, C₂-C₁₈ alkynylamino, C₆-C₁₈ arylamino, C₁-C₁₈ acylamino, C₃-C₁₈ carbocyclylamino, C₂-C₁₈ heterocyclylamino, C₃-C₁₈ heteroarylamino, C₂-C₁₈ alkylaminoalkyl, C₃-C₁₈ alkenylaminoalkyl, C₃-C₁₈ alkynylaminoalkyl, C₄-C₁₈ alkenylaminoalkenyl, C₄-C₁₈ alkenylaminoalkynyl, C₄—C₁₈ alkynylaminoalkynyl, C₇-C₂₄ arylaminoalkyl, C₂-C₁₈ alkylacylamino, C₄-C₁₈ alkylcarbocyclylamino, C₃-C₁₈ alkylheterocyclylamino, C₄-C₁₈ alkylheteroarylamino, C₈-C₁₈ alkylaminoaryl, C₈-C₁₈ alkenylaminoaryl, C₈-C₁₈ alkynylaminoaryl, C₁₂-C₂₄ arylaminoaryl, C₇-C₁₈ arylacylamino, C₉-C₁₈ arylcarbocyclylamino, C₈-C₁₈ arylheterocyclylamino, C₉-C₁₈ arylheteroarylamino, C₁-C₁₈ alkylamido, C₂-C₁₈ alkenylamido, C₂-C₁₈ alkynylamido, C₆-C₁₈ arylamido, C₁-C₁₈ acylamido, C₃-C₁₈ carbocyclylamido, C₂-C₁₈ heterocyclylamido, C₃-C₁₈ heteroarylamido, C₂-C₁₈ alkylamidoalkyl, C₃-C₁₈ alkenylamidoalkyl, C₃-C₁₈ alkynylamidoalkyl, C₄-C₁₈ alkenylamidoalkenyl, C₄-C₁₈ alkenylamidoalkynyl, C₄-C₁₈ alkynylamidoalkynyl, C₇-C₂₄ arylamidoalkyl, C₂-C₁₈ alkylacylamido, C₄-C₁₈ alkylcarbocyclylamido, C₃-C₁₈ alkylheterocyclylamido, C₄-C₁₈ alkylheteroarylamido, C₈-C₁₈ alkylamidoaryl, C₈-C₁₈ alkenylamidoaryl, C₈-C₁₉ alkynylamidoaryl, C₁₂-C₂₄ arylamidoaryl, C₇-C₁₈ arylacylamido, C₈-C₁₈ arylcarbocyclylamido, C₈-C₁₈ arylheterocyclylamido, C₉-C₁₈ arylheteroarylamido, C₁-C₁₈ alkylcarbamyl, C₂-C₁₈ alkenylcarbamyl, C₂-C₁₈ alkynylcarbamyl, C₈-C₁₈ arylcarbamyl, C₁-C₁₈ acylcarbamyl, C₃-C₁₈ carbocyclylcarbamyl, C₂-C₁₈ heterocyclylcarbamyl, C₃-C₁₈ heteroarylcarbamyl, C₂-C₁₈ alkylcarbamylalkyl, C₃-C₁₈ alkenylcarbamylalkyl, C₃-C₁₈ alkynylcarbamylalkyl, C₄-C₁₈ alkenylcarbamylalkenyl, C₄-C₁₈ alkenylcarbamylalkynyl, C₄-C₁₈ alkynylcarbamylalkynyl, C₇-C₂₄ arylcarbamylalkyl, C₂-C₁₈ alkylacylcarbamyl, C₄-C₁₈ alkylcarbocyclylcarbamyl, C₃-C₁₈ alkylheterocyclylcarbamyl, C₄-C₁₈ alkylheteroarylcarbamyl, C₈-C₁₈ alkylcarbamylaryl, C₈-C₁₈ alkenylcarbamylaryl, C₈-C₁₈ alkynylcarbamylaryl, C₁₂-C₂₄ arylcarbamylaryl, C₇-C₁₈ arylacylcarbamyl, C₉-C₁₈ arylcarbocyclylcarbamyl, C₈-C₁₈ arylheterocyclylcarbamyl, C₉-C₁₈ arylheteroarylcarbamyl, C₁-C₁₈ alkyldisulfidyl, C₂-C₁₈ alkenyldisulfidyl, C₂-C₁₈ alkynyldisulfidyl, C₆-C₁₈ aryldisulfidyl, C₁-C₁₈ acyldisulfidyl, C₃-C₁₈ carbocyclyldisulfidyl, C₂-C₁₈ heterocyclyldisulfidyl, C₃-C₁₈ heteroaryldisulfidyl, C₂-C₁₈ alkyldisulfidylalkyl, C₃-C₁₈ alkenyldisulfidylalkyl, C₃-C₁₈ alkynyldisulfidylalkyl, C₄-C₁₈ alkenyldisulfidylalkenyl, C₄-C₁₈ alkenyldisulfidylalkynyl, C₄-C₁₈ alkynyldisulfidylalkynyl, C₇-C₂₄ aryldisulfidylalkyl, C₂-C₁₈ alkylacyldisulfidyl, C₄-C₁₈ alkylcarbocyclyldisulfidyl, C₃-C₁₈ alkylheterocyclyldisulfidyl, C₄-C₁₈ alkylheteroaryldisulfidyl, C₈-C₁₈ alkyldisulfidylaryl, C₈-C₁₈ alkenyldisulfidylaryl, C₈-C₁₈ alkynyldisulfidylaryl, C₁₂-C₂₄ aryldisulfidylaryl, C₇-C₁₈ arylacyldisulfidyl, C₉-C₁₈ arylcarbocyclyldisulfidyl, C₈-C₁₈ arylheterocyclyldisulfidyl, and C₉-C₁₈ arylheteroaryldisulfidyl, and wherein R² is optionally substituted.

135. The method of ay one of any one of claims 130 to 134, wherein R² in Formula (III) is selected from alkyl (e.g., C₁-C₁₈, C₁-C₆, C₁-C₅, C₈-C₁₈, or C₉-C₁₈), aryl (e.g., C₈-C₁₈), heteroaryl (e.g., C₃-C₁₈), carbocyclyl (e.g., C₃-C₁₈), heterocyclyl (e.g., C₂-C₁₈), alkylaryl (e.g., C₇-C₂₄), alkylheteroaryl (e.g., C₄-C₁₉), alkylcarbocyclyl (e.g., C₄-C₁₈), alkylthioalkyl (e.g., wherein each alkyl is C₁-C₁₈), arylthioalkyl (e.g., wherein each alkyl is C₇-C₁₈), alkylaminoalkyl (e.g., wherein each alkyl is C₁-C₁), alkylamidoalkyl (e.g., wherein each alkyl is C₁-C₁₉), alkylheterocyclylalkyl (e.g., C₃-C₁₈), alkylthio (e.g., C₁-C₁₈; such as thiomethyl) and alkylheterocyclyl (e.g., C₃-C₁₈).

136. The method of any one of claims 103 to 135, wherein the iNKT cell agonists is a compound selected from the following GROUP I compounds:

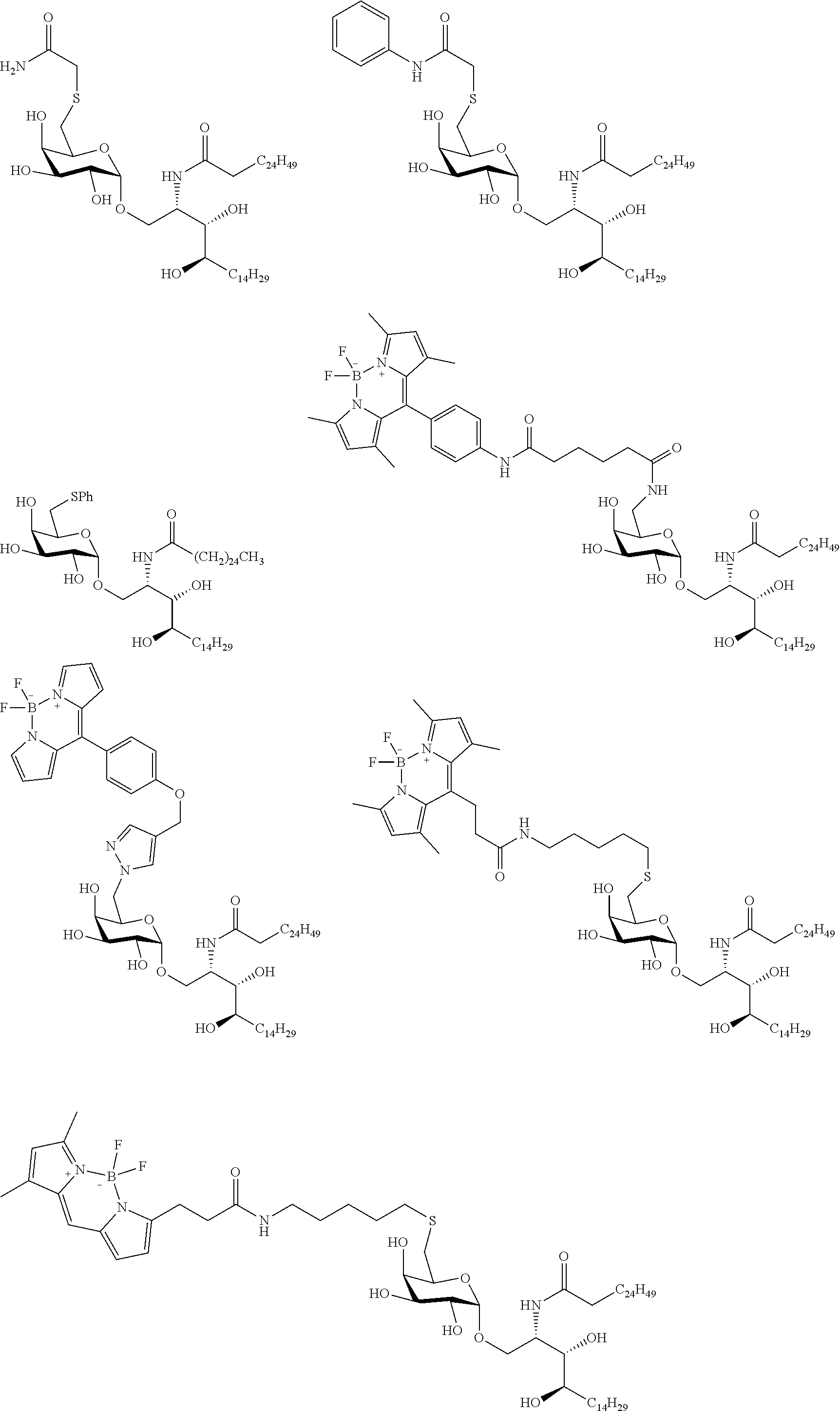

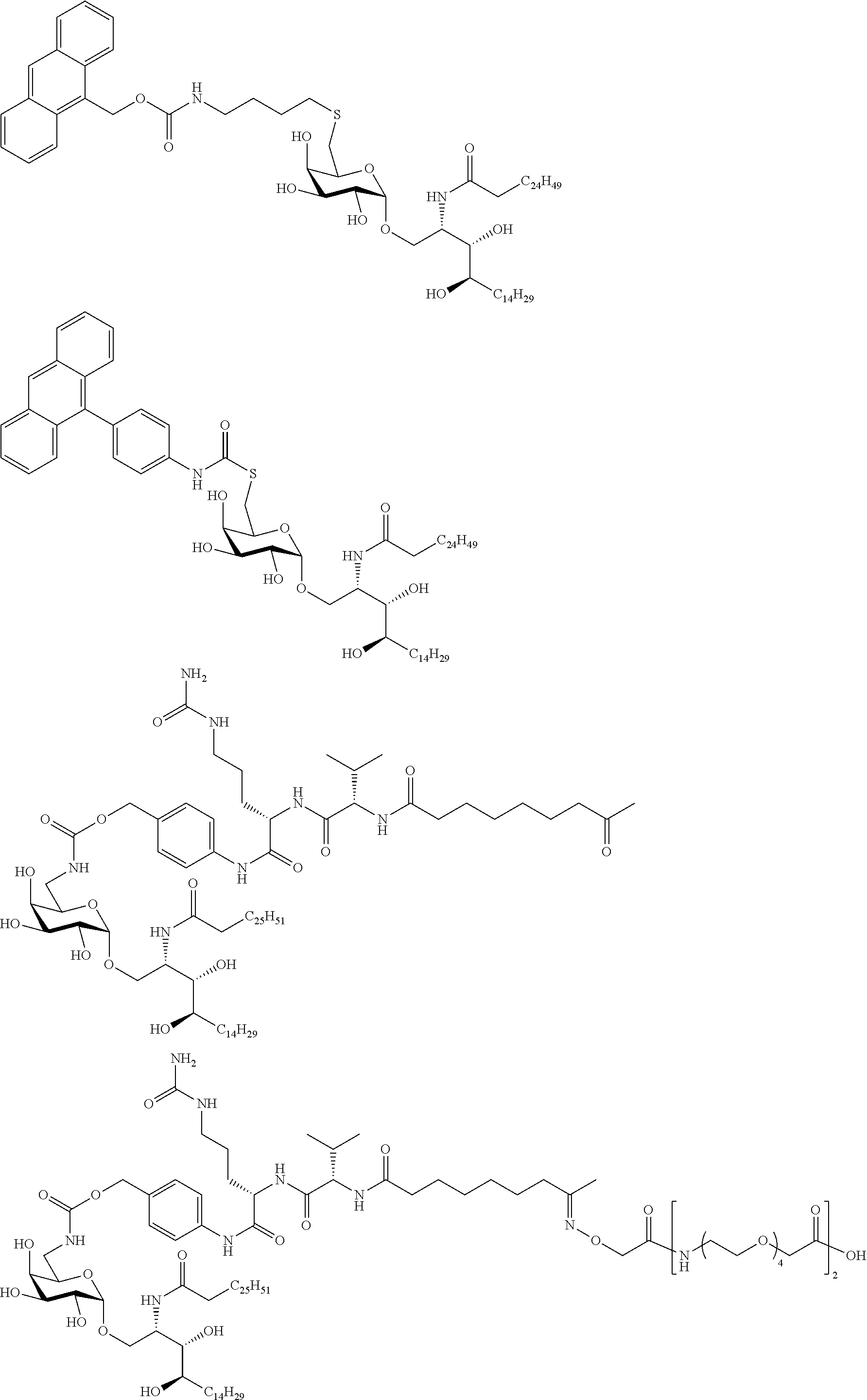

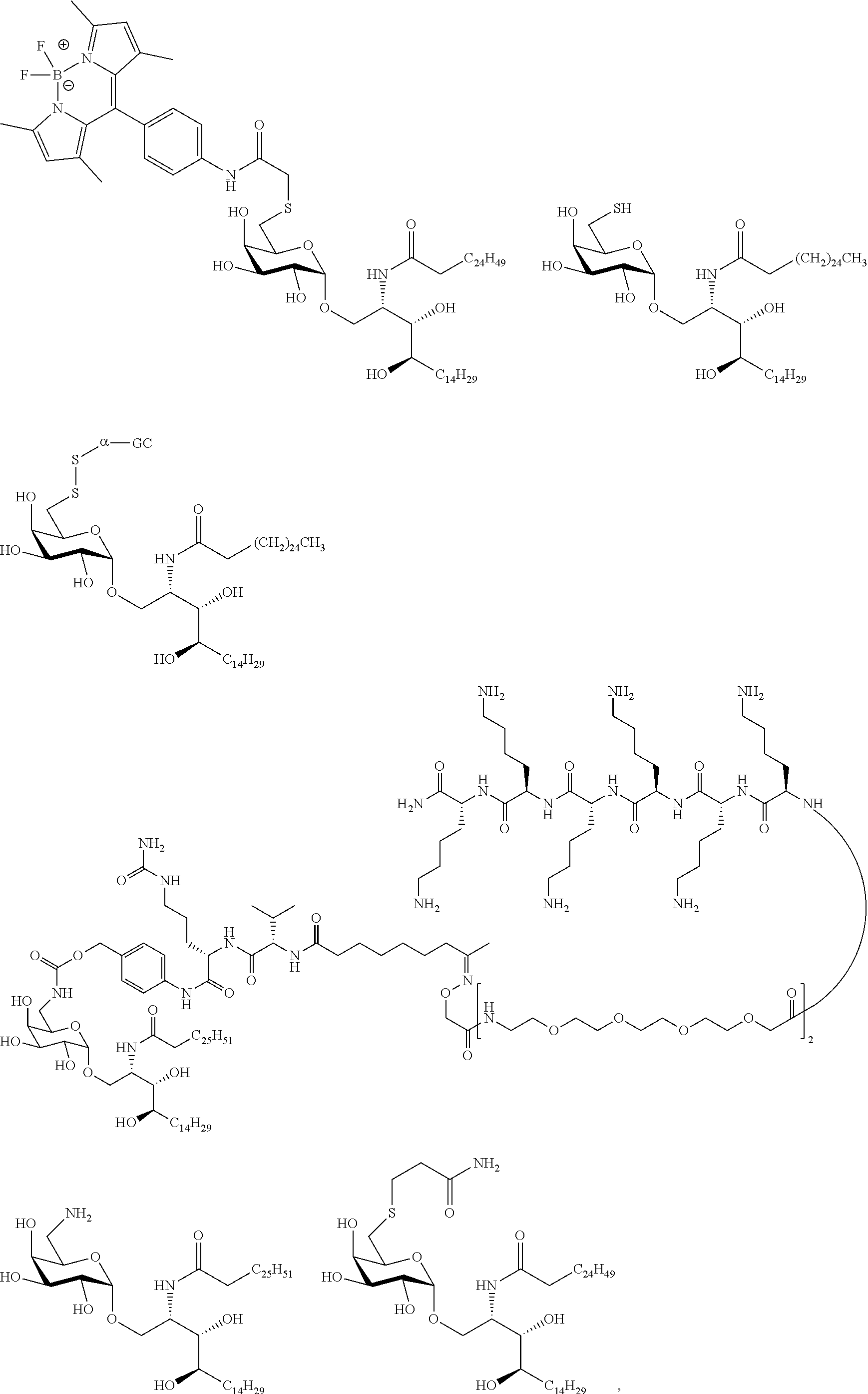

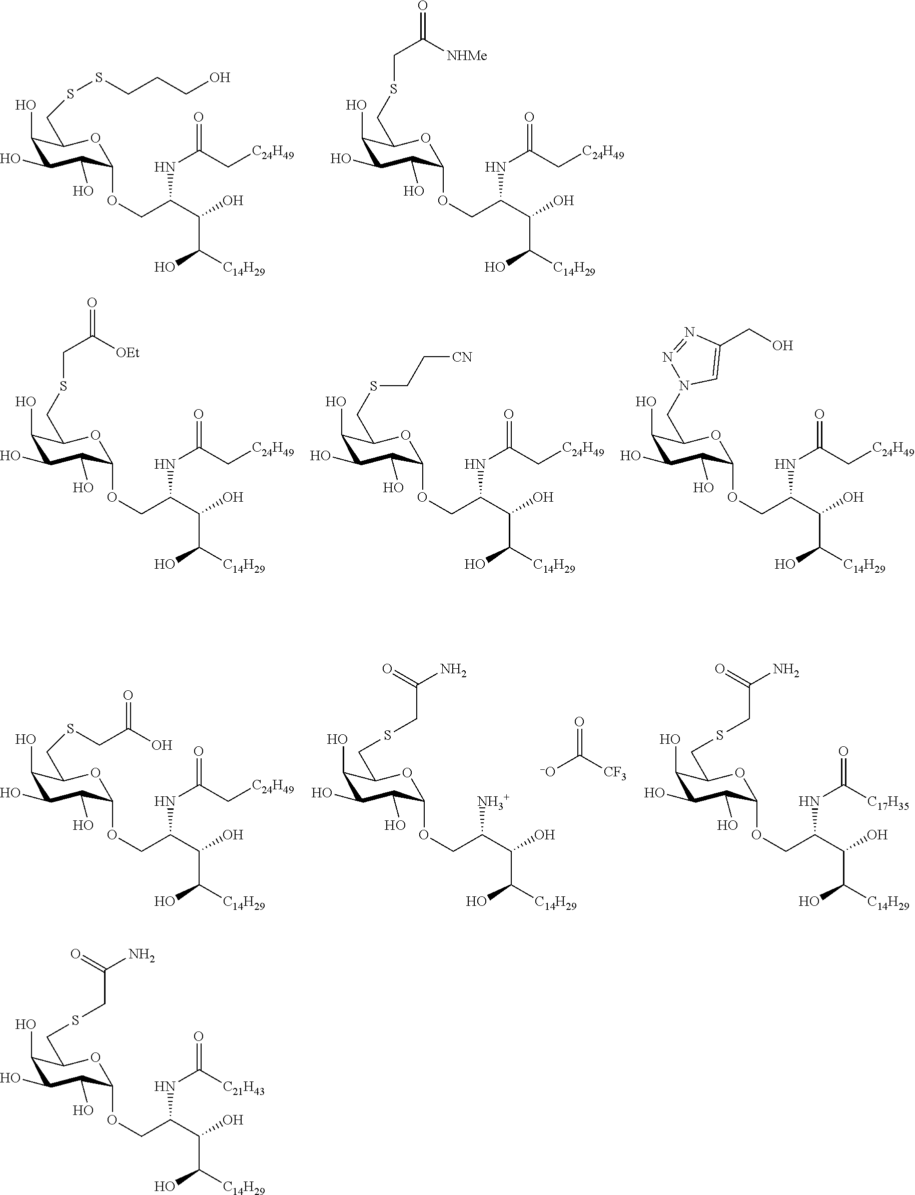

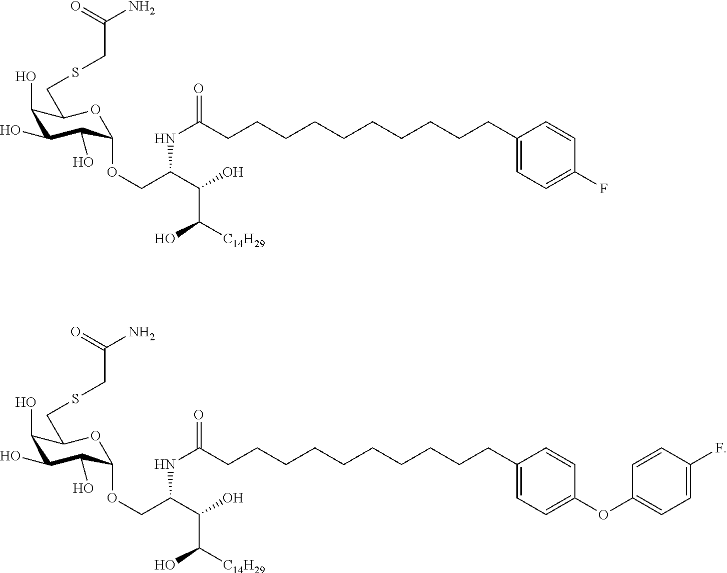

137. The method of any one of claims 103 to 136, wherein the target antigen is selected from a virus antigen, a bacterial antigen, a parasite antigen, and a cancer antigen.

138. The method of claim 103, wherein the infection is a malaria.

139. The method of claim 137 or claim 138, wherein the parasite antigen is derived from a parasite selected from Plasmodium falciparum, P. vivax, P. malariae, P. ovale, and P. knowlesi.

140. The method of claim 137, wherein the virus antigen is a hepatitis (e.g., hepatitis A, hepatitis B and hepatitis C) antigen.

141. The method of claim 137, wherein the cancer antigen is a liver cancer antigen.

142. The method of any one of claims 103 to 141, wherein the second agent is a polynucleotide sequence, and wherein the polynucleotide is a ribonucleic acid (RNA) molecule.

143. The method of any one of claims 103 to 142, wherein the polynucleotide is codon-optimised, and wherein the codon optimisation is selected to enhance translation in the subject and/or to reduce innate immunity against the polynucleotide molecule.

144. The method of any one of claims 103 to 143, wherein the polynucleotide comprises at least one chemical modification.

145. The method of claim 144, wherein the at least one chemical modification is selected from the group consisting of pseudouridine, N1-methylpseudouridine, N1-ethylpseudouridine, 2thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2thio-dihydrouridine, 2-thio-pseudouridine, 4-methoxy-2-thio-pseudouridine, 4methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

146. The method of claim 144 or claim 145, wherein the polynucleotide is fully modified.

147. The method of any one of claims 144 to 146, wherein the composition further comprises a nanoparticle and the second agent is encapsulated within the nanoparticle or complexed to the surface of the nanoparticle.

148. The method of claim 147, wherein the nanoparticle is a lipid nanoparticle (e.g., a liposome, or a lipoplex).

149. The method of claim 147 or claim 148, wherein the first agent is contained at least partially within the surface of the lipid nanoparticle.

150. The method of any one of claims 147 to 149, wherein the lipid nanoparticle has a diameter of between about 50 and 500 nm.

151. The method of any one of claims 147 to 150, wherein the nanoparticle comprises a cationic lipid, a PEG-modified lipid, a sterol, and/or a non-cationic lipid.

152. The method of claim 151, wherein the cationic lipid is an ionizable cationic lipid.

153. The method of claim 151 or claim 152, wherein the ionizable cationic lipid is selected from the group comprising or consisting of 1,2-di-O-octadecenyl-3-trimethylammonium propane (DOTMA), N-[1-(2,3-dioleyloxy)propyl]-N,N,N-trimethylammonium chloride (DOTMA), 1,2-Dioleoyl-3-Trimethylammonium-Propane (DOTAP), 5-carboxyspermylglycinedioctadecylamide (DOGS), 2,3-dioleyloxy-N-[2(spermine-carboxamido)ethyl]-N,N-dimethyl-I-propanaminium (DOSPA), 1,2-Dioleoyl-3-Dimethylammonium-Propane (DODAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), heptatriaconta-6,9,28,31-tetraen19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dilinolenyloxy-N,N-dimethyl-3-aminopropane (DLenDMA), N-dioleyl-N,N-dimethyl ammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis, cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminoethyl-[1,3]-dioxolane (DLin-K-XTC2-DMA), and C12-200.

154. The method of any one of claims 147 to 153, wherein the nanoparticle comprises a dioleoylphosphatidylethanolamine (DOPE) lipid.

155. The method of any one of claims 103 to 154, wherein the polynucleotide is codon-optimised for enhanced translation in the subject, or to modulate the immunogenicity in the subject.

156. The method of any one of claims 103 to 155, wherein the polynucleotide comprises at least one chemical modification.

157. The method of claim 156, wherein the at least one chemical modification is selected from the group consisting of pseudouridine, N1-methylpseudouridine, N1-ethylpseudouridine, 2thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2thio-dihydrouridine, 2-thio-pseudouridine, 4-methoxy-2-thio-pseudouridine, 4methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

158. The method of any one of claims 103 to 157, wherein the first agent and the second agent are formulated in a single unit dose.

159. The composition of any one of claims 1 to 102 for use in therapy.

160. The composition of any one of claims 1 to 102 for use in the treatment of malaria.

161. The composition of any one of claims 1 to 102 for use treating or preventing an infection.

162. The composition of any one of claims 1 to 102 for use in treating or preventing cancer.
